(19) United States
(12) Patent Application Publication
(10) Pub. No.: US 2023/0242939 A1
(43) Pub. Date: Aug. 3, 2023

(54) METHODS AND SYSTEMS FOR PRODUCING AAV PARTICLES

(71) Applicant: Voyager Therapeutics, Inc., Cambridge, MA (US)

(72) Inventors: Krishanu Mathur, Cambridge, MA (US); Andrade Hendricks, Cambridge, MA (US); Matthew Luther, Cambridge, MA (US); Jacob J. Cardinal, North Cambridge, MA (US); Daniel S. Hurwit, Seattle, WA (US); Lori B. Karpes, Ashland, MA (US); Christopher J. Morrison, Arlington, MA (US)

(21) Appl. No.: 17/795,250

(22) PCT Filed: Jan. 28, 2021
(86) PCT No.: PCT/US2021/015393

§ 371 Date: Jul. 26, 2022

Related U.S. Application Data
(60) Provisional application No. 62/967,538, filed on Jan. 29, 2020.
(60) Provisional application No. 62/967,540, filed on Jan. 29, 2020.
(60) Provisional application No. 63/010,420, filed on Apr. 15, 2020.
(60) Provisional application No. 63/010,434, filed on Apr. 15, 2020.

(51) Int. Cl. C12N 15/86; C12N 9/88

(52) U.S. Cl. CPC C12N 15/86; C12N 9/88; C12Y 401/01028; C12N 2710/14144; C12N 2750/14143; C12N 2750/14151

(57) ABSTRACT

The present disclosure describes methods and systems for use in the production of recombinant adeno-associated virus (rAAV) particles comprising a payload (e.g., a polynucleotide encoding aromatic L-amino acid decarboxylase (AADC) or a functional variant thereof). In certain embodiments, the production process uses Sf9 insect cells as viral production cells. In certain embodiments, the production process and system use Baculoviral Expression Vectors (BEVs) and/or Baculoviral Infected Insect Cells (BIICs) in the production of rAAV particles.

### CROSS REFERENCE TO RELATED APPLICATIONS

This application is a U.S. National Stage Application filed under 35 U.S.C. § 371, based on International Patent Application No. PCT/US2021/015393, filed on Jan. 28, 2021, which claims priority to U.S. Provisional Patent Application Nos. 62/967,538, filed on Jan. 29, 2020; 62/967,540, filed on Jan. 29, 2020; 63/010,420, filed on Apr. 15, 2020; and 63/010,434, filed on Apr. 15, 2020, the entire contents of each of the above applications are incorporated herein by reference.

### REFERENCE TO THE SEQUENCE LISTING

The instant application contains a Sequence Listing which has been submitted electronically in ASCII format and is hereby incorporated by reference in its entirety. Said ASCII copy, created on Jan. 9, 2023, is named 135333-02703_ST25 and is 6,600,291 bytes in size.

### FIELD OF THE DISCLOSURE

The present disclosure describes methods and systems for use in the production of adeno-associated virus (AAV) particles, compositions and formulations, comprising recombinant adeno-associated viruses (rAAV), wherein the AAV particles comprise a payload construct encoding aromatic L-amino acid decarboxylase (AADC). In certain embodiments, the present disclosure presents methods and systems for designing, producing, clarifying, purifying, formulating, filtering and processing rAAVs and rAAV formulations. In certain embodiments, the production process and system use Spodoptera frugiperda insect cells (such as Sf9 or Sf21) as viral production cells (VPCs). In certain embodiments, the production process and system use Baculoviral Expression Vectors (BEVs) and/or Baculoviral Infected Insect Cells (BIICs) in the production of rAAVs.

### BACKGROUND

AAVs have emerged as one of the most widely studied and utilized viral vectors for gene transfer to mammalian cells. See, e.g., Tratschin et al., Mol. Cell Biol., 5(11):3251-3260 (1985) and Grimm et al., Hum. Gene Ther., 10(15):2445-2450 (1999), the contents of which are each incorporated herein by reference in their entireties insofar as they do not conflict with the present disclosure. Adeno-associated viral (AAV) vectors are promising candidates for therapeutic gene delivery and have proven safe and efficacious in clinical trials. The design and production of improved AAV particles for this purpose is an active field of study.

There remains a need for improved systems and methods for producing AAV capsid proteins, AAV capsids, and corresponding AAV vectors (such as AAV particles), e.g., for gene therapy.

For example, there remains a need for improved systems and methods for producing AAV capsid proteins, AAV capsids, and corresponding AAV vectors (such as AAV particles) for the delivery and/or expression of aromatic L-amino acid decarboxylase (AADC) in a patient and for treatment and/or prevention of Parkinson's Disease (PD).

AADC is a homodimeric pyridoxal phosphate-dependent enzyme responsible for the synthesis of dopamine and serotonin. The enzyme catalyzes the decarboxylation of L-3,4-dihydroxyphenylalanine (L-DOPA or levodopa) to dopamine; L-5-hydroxytryptophan to serotonin; and L-tryptophan to tryptamine. Defects in this gene are the cause of aromatic L-amino-acid decarboxylase deficiency (AADCD), which is an inborn error in neurotransmitter metabolism leading to combined serotonin and catecholamine deficiency that results in severe motor and autonomic dysfunctions.

PD is a progressive neurodegenerative disease of the central nervous system (CNS) producing sensory and motor symptoms. Dopamine replacement (i.e., levodopa) has been the standard pharmacotherapy for motor impairment in PD. However, the benefit of dopamine therapy becomes less marked over time, due, in part, to the progressive death of dopamine-generating cells and corresponding loss of AADC activity. Furthermore, systemic administration of high-dose dopamine is complicated by side effects, such as fluctuations in motor performance, dyskinesias, and hallucinations, resulting from dopaminergic stimulation of the mesolimbic system. One strategy to restore dopaminergic function and minimize side effects is the use of gene therapy to deliver AADC directly to a targeted region of the CNS.

### SUMMARY

The present disclosure presents methods and systems for producing recombinant adeno-associated viruses (rAAVs).

In certain embodiments, the present disclosure encompasses a method for producing a recombinant adeno-associated virus (rAAV) comprising a polynucleotide encoding a payload (e.g., aromatic L-amino acid decarboxylase (AADC) or a functional variant thereof). In some embodiments, the method comprises the steps of culturing viral production cells (VPCs) in a bioreactor to a target cell density; introducing into the bioreactor at least one baculovirus (expressionBac) comprising a viral expression construct, and at least one baculovirus (payloadBac) comprising a payload construct, wherein the viral expression construct comprises an adeno-associated virus (AAV) viral expression construct; incubating the VPCs in the bioreactor under conditions that result in the production of one or more rAAVs within one or more VPCs, wherein one or more of the rAAVs comprise the polynucleotide encoding AADC or a functional variant thereof; harvesting a viral production pool from the bioreactor, wherein the viral production pool comprises one or more VPCs comprising one or more rAAVs; lysing the one or more VPCs in the viral production pool, thereby releasing one or more rAAVs from the one or more VPCs into a lysis medium; and processing the lysis medium. In some embodiments, the processing step comprises one or more clarifying steps; one or more immunoaffinity chromatography steps; one or more anion exchange chromatography steps; one or more tangential flow filtration (TFF) steps, wherein the one or more TFF steps comprises ultrafiltration followed by diafiltration; and one or more virus retentive filtration (VRF) steps, wherein the processing may further comprise one or more filtration steps before or after any one or more of the processing steps described above. In certain embodiments, the VPCs are insect cells, e.g., Sf9 cells. In some embodiments, the payload is AADC or a functional variant thereof. In some embodiments, the payload construct comprises the polynucleotide encoding AADC or a functional variant thereof. In some embodiments, the polynucleotide encoding AADC or a functional variant thereof encodes SEQ ID NO: 978. In some embodiments, the payload construct comprises SEQ ID NO: 979.

In certain embodiments, the payload construct comprises the polynucleotide encoding a therapeutic protein, an enzyme, an antibody or antigen-binding fragment thereof, a protein ligand, or a soluble receptor. In certain embodiments, the payload construct comprises the polynucleotide encoding a modulatory polynucleotide which interferes with a target gene expression and/or a target protein production. In certain embodiments, the modulatory polynucleotide is an antisense strand, a miRNA molecule, or a siRNA molecule.

In some embodiments, the at least one baculovirus (expressionBac) comprising a viral expression construct is comprised in at least one baculovirus infected insect cell (expressionBIIC). In some embodiments, the baculovirus infected insect cell (expressionBIIC) comprising at least one expressionBac is an Sf9 cell. In some embodiments, the at least one baculovirus (payloadBac) comprising a payload construct is comprised in at least one baculovirus infected insect cell (payloadBIIC). In some embodiments, the baculovirus infected insect cell (payloadBIIC) comprising at least one payloadBac is an Sf9 cell.

In certain embodiments, the present disclosure encompasses a method for producing a recombinant adeno-associated virus (rAAV) comprising a polynucleotide encoding a payload (e.g., aromatic L-amino acid decarboxylase (AADC) or a functional variant thereof). In some embodiments, the method comprises the steps of: (a) culturing viral production cells (VPCs) in a bioreactor to a target cell density; (b) introducing into the bioreactor at least one baculovirus (expressionBac) comprising a viral expression construct, and at least one baculovirus (payloadBac) comprising a payload construct, wherein the viral expression construct comprises an adeno-associated virus (AAV) viral expression construct; (c) incubating the VPCs in the bioreactor under conditions that result in the production of one or more rAAVs within one or more VPCs, wherein one or more of the rAAVs comprise the polynucleotide encoding AADC or a functional variant thereof; (d) harvesting a viral production pool from the bioreactor, wherein the viral production pool comprises one or more VPCs comprising one or more rAAVs; (e) lysing the one or more VPCs in the viral production pool by chemical lysis, thereby releasing one or more rAAVs from the one or more VPCs into a lysis medium; (f) clarifying the lysis medium of (e) through one or more clarifying steps, yielding a clarification pool, wherein the clarification pool is optionally filtered; (g) processing the clarification pool of (f) through one or more immunoaffinity chromatography steps, yielding an immunoaffinity chromatography pool, wherein the one or more immunoaffinity chromatography steps optionally comprises neutralizing the immunoaffinity chromatography pool, and wherein the immunoaffinity chromatography pool is optionally filtered; (h) processing the immunoaffinity chromatography pool of (g) through one or more anion exchange chromatography steps, yielding an anion exchange chromatography pool, wherein the anion exchange chromatography pool is optionally filtered; (i) processing the anion exchange chromatography pool of (h) through one or more tangential flow filtration (TFF) steps, wherein the one or more TFF steps comprises ultrafiltration followed by diafiltration, wherein the one or more TFF steps yields a concentrated, buffer-exchanged pool, wherein the concentrated, buffer-exchanged pool is optionally filtered; and (j) processing the concentrated, buffer-exchanged pool of (i) through one or more virus retentive filtration (VRF) steps, yielding a viral filtration pool comprising rAAVs comprising a payload (e.g., polynucleotide encoding AADC or a functional variant thereof), wherein the viral filtration pool is optionally filtered. In some embodiments, the viral filtration pool of step (j) is further processed through a filtration step. In certain embodiments, the VPCs are insect cells. In some embodiments, the insect cells are Sf9 cells. In some embodiments, the payload is AADC or a functional variant thereof. In some embodiments, the payload construct comprises the polynucleotide encoding AADC or a functional variant thereof. In some embodiments, the polynucleotide encoding AADC or a functional variant thereof encodes SEQ ID NO: 978.

In some embodiments, the at least one baculovirus (expressionBac) comprising a viral expression construct is comprised in at least one baculovirus infected insect cell (expressionBIIC). In some embodiments, the baculovirus infected insect cell (expressionBIIC) comprising at least one expressionBac is an Sf9 cell. In some embodiments, the at least one baculovirus (payloadBac) comprising a payload construct is comprised in at least one baculovirus infected insect cell (payloadBIIC). In some embodiments, the baculovirus infected insect cell (payloadBIIC) comprising at least one payloadBac is an Sf9 cell.

In some embodiments, the payload construct comprises a 5′ inverted terminal repeat (ITR), at least one multiple cloning site (MCS) region, a cytomegalovirus (CMV) enhancer, a CMV promoter, an intron region comprising immediate-early 1 (Ie1) exon 1, Ie1 intron 1 (partial), human beta-globin (hBglobin) intron 2, and hBglobin intron 3, a polyadenylation (poly(A)) signal, and a 3′ ITR. In some embodiments, the payload construct comprises, e.g., in order from 5′ to 3′: a 5′ ITR comprising SEQ ID NO: 980, a first MCS region comprising SEQ ID NO: 981, a CMV enhancer comprising SEQ ID NO: 982, a CMV promoter comprising SEQ ID NO: 983, an intron region comprising an Ie1 exon 1 (SEQ ID NO: 984), a partial Ie1 intron 1 (SEQ ID NO: 985), a human beta-globin (hBglobin) intron 2 (SEQ ID NO: 986), and a hBglobin intron 3 (SEQ ID NO: 987), a polynucleotide encoding an AADC amino acid sequence comprising SEQ ID NO: 978, wherein optionally the polynucleotide comprises SEQ ID NO: 988, a poly(A) signal comprising SEQ ID NO: 990, and a 3′ ITR comprising SEQ ID NO: 991. In some embodiments, the payload construct comprises a sequence at least 90%, at least 95%, at least 96%, at least 97%, at least 98%, or at least 99% identical to SEQ ID NO: 979. In some embodiments, the payload construct comprises SEQ ID NO: 979.

In some embodiments, the at least one expressionBIIC is introduced into the bioreactor at a ratio of 1:200,000 to 1:400,000 (v/v) relative to VPCs) and/or the at least one payloadBIIC is introduced into the bioreactor at a ratio of 1:25,000 to 1:200,000 payloadBIIC:VPC (v/v). In some embodiments, the at least one expressionBIIC is introduced into the bioreactor at a ratio of 1:250,000 to 1:350,000 (v/v) relative to VPCs (e.g., 1:300,000 expressionBIIC:VPC (v/v)) and/or the at least one payloadBIIC is introduced into the bioreactor at a ratio of 1:50,000 to 1:150,000 payloadBIIC:VPC (v/v) (e.g., 1:100,000 payloadBIIC:VPC (v/v)). In some embodiments, the ratio of expressionBIIC to payloadBIIC is between 1:1 to 1:5. In some embodiments, the ratio of expressionBIIC to payloadBIIC is 1:3. In some embodiments, the at least one expressionBIIC is introduced into the bioreactor at a ratio of 1:300,000 expressionBIIC:VPC (v/v)) and the at least one payloadBIIC is introduced into the bioreactor at a ratio of 1:100,000 payloadBIIC:VPC (v/v)).

In some embodiments, the VPCs are cultured in the bioreactor in insect cell culture medium. In some embodiments, the insect cell culture medium is a serum free, protein-free medium, wherein optionally the insect cell culture medium comprises L-glutamine and poloxamer 188, wherein further optionally the insect cell culture medium comprises EFS AF™ insect cell culture medium.

In some embodiments, the VPCs are cultured in the bioreactor at 26° C.-28° C. (e.g., 27° C.) and 30%-50% (e.g., 40%) dissolved oxygen.

In some embodiments, the target cell density of the VPCs (i.e., the viable cell density (VCD)) is 3.0×10⁶-3.4×10⁶ cells/mL (e.g., 3.2×10⁶-3.4×10⁶ cells/mL; e.g., 3.2×10⁶ cells/mL).

In some embodiments, the target cell density (i.e., viable cell density (VCD)) of VPCs prior to introduction of the expressionBIICs (or expressionBacs) and payloadBIICs (or payloadBacs) is about 3.0×10⁶-3.4×10⁶ cells/mL (e.g., 3.2×10⁶-3.4×10⁶ cells/mL; e.g., 3.2×10⁶ cells/mL), the at least one expressionBIIC is introduced into the bioreactor at a ratio of 1:300,000 expressionBIIC:VPC (v/v)), and the at least one payloadBIIC is introduced into the bioreactor at a ratio of 1:100,000 payloadBIIC:VPC (v/v)). In some embodiments, one or more of the VPCs, expressionBIICs, and/or payloadBIICs are Sf9 cells. In some embodiments, all of the VPCs, expressionBIICs, and payloadBIICs are Sf9 cells.

In some embodiments, the lysing step comprises a chemical lysis solution comprising a surfactant and arginine or a salt thereof, wherein optionally the surfactant is octyl phenol ethoxylate and the arginine or salt thereof is arginine hydrochloride. In some embodiments, the chemical lysis solution comprises between about 0.1-1.0% (w/v) octyl phenol ethoxylate (e.g., Triton X-100) and between about 150-250 mM arginine hydrochloride. In some embodiments, the chemical lysis solution comprises 0.5% (w/v) octyl phenol ethoxylate (e.g., Triton X-100) and 200 mM arginine hydrochloride. In some embodiments, the lysis pH is 6.8-7.5. In some embodiments, the chemical lysis solution is free of detectable nuclease. In some embodiments, the lysing is carried out for 4-6 hours (e.g., 4 hours) at 26° C.-28° C. (e.g., 27° C.). In some embodiments, the one or more clarifying steps comprises depth filtration followed by filtration through an about 0.2 μm filter.

In some embodiments, the one or more immunoaffinity chromatography steps comprises an immunoaffinity chromatography column comprising a recombinant protein ligand that binds at least one of AAV1, AAV2, AAV3, AAV5, and AAV9. In some embodiments, the one or more immunoaffinity chromatography steps comprises an immunoaffinity chromatography column comprising a recombinant protein ligand that binds at least AAV2.

In some embodiments, the immunoaffinity chromatography column is equilibrated with a solution comprising between about 25-75 mM sodium phosphate, between about 325-375 mM sodium chloride and between about 0.001-0.01% w/v poloxamer 188 (solution pH of 7.2-7.6); flushed with a solution comprising between about 25-75 mM sodium phosphate, between about 325-375 mM sodium chloride and between about 0.001-0.1% w/v poloxamer 188 (solution pH of 7.2-7.6); washed with a solution comprising between about 15-25 mM sodium citrate, between about 0.5-1.5 M sodium chloride, and between about 0.001-0.1% w/v poloxamer 188 (solution pH of 5.8-6.2); and washed a second time with a solution of between about 5-15 mM sodium citrate, between about 325-375 mM sodium chloride and between about 0.001-0.1% w/v poloxamer 188 (solution pH of 5.8-6.2); wherein the one or more immunoaffinity chromatography steps yields a immunoaffinity chromatography pool. In some embodiments, the filtered product is eluted with a solution comprising between about 15-25 mM sodium citrate, between about 325-375 mM sodium chloride and between about 0.001-0.1% w/v poloxamer 188 (solution pH of 2.8-3.2). In some embodiments, the immunoaffinity chromatography pool is neutralized with between about 1.5-2.5 M Tris Base and between about 0.001-0.01% w/v poloxamer 188 (2.0-4.0% v/v spike, pH 8.0-8.5). In some embodiments, the immunoaffinity chromatography pool is filtered through an about 0.2 μm filter.

In some embodiments, the immunoaffinity chromatography column is equilibrated with a solution comprising 50 mM sodium phosphate, 350 mM sodium chloride and 0.001% w/v poloxamer 188 (e.g., solution pH of 7.2-7.6, e.g., pH of 7.4); flushed with a solution comprising 50 mM sodium phosphate, 350 mM sodium chloride and 0.001% w/v poloxamer 188 (e.g., solution pH of 7.2-7.6, e.g., pH of 7.4); washed with a solution comprising 20 mM sodium citrate, 1 M sodium chloride and 0.001% w/v poloxamer 188 (e.g., solution pH of 5.8-6.2, e.g., pH of 6.0); and washed a second time with a solution of 10 mM sodium citrate, 350 mM sodium chloride and 0.001% w/v poloxamer 188 (e.g., solution pH of 5.8-6.2, e.g., pH of 6.0); wherein the one or more immunoaffinity chromatography steps yields a immunoaffinity chromatography pool. In some embodiments, the filtered product is eluted with a solution comprising 20 mM sodium citrate, 350 mM sodium chloride and 0.001% w/v poloxamer 188 (e.g., solution pH of 2.8-3.2, e.g., pH of 3.0). In some embodiments, the immunoaffinity chromatography pool is neutralized with 2 M Tris Base and 0.001% w/v poloxamer 188 (3.0% v/v spike, pH 8.0-8.5). In some embodiments, the immunoaffinity chromatography pool is filtered through an about 0.2 μm filter.

In some embodiments, the one or more immunoaffinity chromatography steps comprises loading the immunoaffinity chromatography column with a 1.0×10¹³-5.0×10¹³ vg/mL-r load challenge at 18-25° C.

In some embodiments, the one or more anion exchange chromatography steps comprises charging and equilibrating an anion exchange chromatography column with a solution comprising between about 15-25 mM Tris, between about 1.5-2.5 M sodium chloride and between about 0.001-0.01% w/v poloxamer 188, then a solution of between about 35-45 mM Tris, between about 150-190 mM sodium chloride and between about 0.001-0.01% w/v poloxamer 188 (solution pH of 7.8-8.2). In some embodiments, the anion exchange chromatography column is flushed and eluted with a solution comprising between about 35-45 mM Tris, between about 150-190 mM sodium chloride and between about 0.001-0.01% w/v poloxamer 188 (solution pH of 8.3-8.7), yielding an anion exchange chromatography pool. In some embodiments, the anion exchange chromatography elution pool is filtered through an about 0.2 μm filter.

In some embodiments, the one or more anion exchange chromatography steps comprises charging and equilibrating an anion exchange chromatography column with a solution comprising 20 mM Tris, 2 M sodium chloride and 0.001% w/v poloxamer 188, then a solution of 40 mM Tris, 170 mM sodium chloride and 0.001% w/v poloxamer 188 (e.g., solution pH of 7.8-8.2, e.g., pH of 8.0). In some embodiments, the anion exchange chromatography column is flushed and eluted with a solution comprising 40 mM Tris, 170 mM sodium chloride and 0.001% w/v poloxamer 188 (e.g., solution pH of 8.3-8.7, e.g., pH of 8.5), yielding an anion exchange chromatography pool. In some embodiments, the anion exchange chromatography elution pool is filtered through an about 0.2 μm filter.

In some embodiments, the one or more anion exchange chromatography steps comprises loading the anion exchange chromatography column with a 1.0×10¹³-5.0×10¹³ vg/mL-r load challenge at 18-25° C.

In some embodiments, the one or more TFF steps comprises TFF filtration with a TFF filter, yielding a TFF load pool, followed by concentration of the TFF load pool by ultrafiltration followed by diafiltration, yielding a final TFF load pool. In some embodiments, the TFF filtration comprises equilibration with a buffer (pH 8.3-8.7) comprising between about 35-45 mM Tris, between about 150-190 mM sodium chloride, and between about 0.001-0.01% (w/v) poloxamer 188. In some embodiments, after obtaining the TFF load pool, the TFF filter is subjected to a recovery flush using a buffer comprising between about 5-15 mM sodium phosphate, between about 160-200 mM sodium chloride, and between about 0.001-0.1% (w/v) poloxamer 188, yielding a TFF recovery flush pool. In some embodiments, the TFF load pool is concentrated by ultrafiltration to a viral concentration of between about 1.0×10¹²-7.0×10¹² vg/mL. In some embodiments, the diafiltration step comprises buffer exchange with a buffer comprising between about 5-15 mM sodium phosphate, between about 160-200 mM sodium chloride, and between about 0.001-0.01% (w/v) poloxamer 188 (buffer pH of 7.1-7.5). In some embodiments, the final TFF load pool is filtered through an about 0.2 μm filter, yielding a filtered final TFF load pool.

In some embodiments, the TFF filtration comprises equilibration with a buffer (e.g., pH 8.3-8.7, e.g., pH 8.5) comprising 40 mM Tris, 170 mM sodium chloride, and 0.001% (w/v) poloxamer 188. In some embodiments, after obtaining the TFF load pool, the TFF filter is subjected to a recovery flush using a buffer comprising 10 mM sodium phosphate, 180 mM sodium chloride, and 0.001% (w/v) poloxamer 188, yielding a TFF recovery flush pool. In some embodiments, the TFF load pool is concentrated by ultrafiltration to a viral concentration of about 5.0×10¹² vg/mL. In some embodiments, the diafiltration step comprises buffer exchange with a buffer comprising 10 mM sodium phosphate, 180 mM sodium chloride, and 0.001% (w/v) poloxamer 188 (buffer pH of 7.1-7.5, e.g., pH 7.3).

In some embodiments, the final TFF load pool is filtered through an about 0.2 μm filter, yielding a filtered final TFF load pool. In some embodiments, the TFF recovery flush pool is filtered through an about 0.2 μm filter, yielding a filtered TFF recovery flush pool. In some embodiments, the filtered final TFF load pool and the filtered TFF recovery flush pool are combined to form a concentrated, buffer-exchanged pool, wherein the concentrated, buffer-exchanged pool is optionally diluted using a buffer comprising 10 mM sodium phosphate, 180 mM sodium chloride, and 0.001% (w/v) poloxamer 188 (buffer pH of 7.1-7.5, e.g., pH 7.3), wherein the concentrated, buffer-exchanged pool comprises a viral concentration of 2.0×10¹²-6.0×10¹² vg/mL, e.g., 5.0×10¹² vg/mL. In some embodiments, the filtered final TFF load pool and the filtered TFF recovery flush pool are combined to form a concentrated, buffer-exchanged pool, wherein the concentrated, buffer-exchanged pool is optionally diluted using a buffer comprising between about 5-15 mM sodium phosphate, between about 160-200 mM sodium chloride, and between about 0.001-0.1% (w/v) poloxamer 188 (buffer pH of 7.1-7.5), wherein the concentrated, buffer-exchanged pool comprises a viral concentration of 1.0×10¹²-7.0×10¹² vg/mL.

In some embodiments, the one or more VRF steps comprises filtration with a VRF filter having a pore size of about 35 nm, yielding a viral filtration pool. In some embodiments, the VRF filter is flushed twice before use with a solution comprising between about 5-15 mM sodium phosphate, between about 160-200 mM sodium chloride, and between about 0.001-0.01% poloxamer 188 (solution pH of 7.1-7.5). In some embodiments, the VRF filter is flushed twice before use with a solution comprising 10 mM sodium phosphate, 180 mM sodium chloride, and 0.001% poloxamer 188 (solution pH of 7.1-7.5, e.g., pH 7.3).

In some embodiments, the viral filtration pool is filtered through a filter of about 0.2 μm. In some embodiments, the viral filtration pool comprises a viral concentration of 1.0×10¹²-7.0×10¹² vg/mL. In some embodiments, the viral filtration pool is filtered at least once (optionally at least twice) using an about 0.22 μm filter, yielding a filtered drug substance pool in a solution comprising between about 5-15 mM sodium phosphate, between about 160-200 mM sodium chloride, and between about 0.001-0.01% poloxamer 188 (solution pH of 7.1-7.5). In some embodiments, the viral filtration pool comprises a viral concentration of 3.5×10¹²-5.0×10¹² vg/mL, e.g., about 5.0×10¹² vg/mL. In some embodiments, the viral filtration pool is filtered at least once (optionally at least twice) using an about 0.22 μm filter, yielding a filtered drug substance pool in a solution comprising 10 mM sodium phosphate, 180 mM sodium chloride, and 0.001% poloxamer 188 (solution pH of 7.1-7.5, e.g., pH 7.3).

In some embodiments, the filtered drug substance pool comprises a viral concentration of 3.0×10¹²-5.0×10¹² vg/mL, e.g., about 5.0×10¹² vg/mL. In some embodiments, the VRCs, at least one expressionBac or at least one expressionBIIC, and at least one payloadBac or at least one expressionBIIC are incubated for 156-180 hours, e.g., 164-172 hours, e.g., 168 hours, prior to lysis. In some embodiments, the VRCs incubating with at least one expressionBac (e.g., expressionBIIC) and at least one payloadBac (e.g., payloadBIIC) have at least 85% viability, e.g., at least 90% viability, prior to lysis. In some embodiments, the viral production pool weighs 195-198 kg, e.g., 196 kg, prior to lysis. In some embodiments, the method produces a total process rAAV yield of 30%-50%. In some embodiments, the rAAVs comprise a capsid from AAV2. In some embodiments, the AAV2 capsid is encoded by a sequence comprising SEQ ID NO: 15. In some embodiments, the AAV2 capsid is encoded by a sequence comprising SEQ ID NO: 1778. In some embodiments, the AAV2 capsid comprises SEQ ID NO: 16.

In some embodiments, the viral expression construct comprises one or more polynucleotides encoding a VP1 capsid protein, VP2 capsid protein, VP3 capsid protein, Rep52, and Rep78. In some embodiments, the VP1 capsid protein, VP2 capsid protein, and VP3 capsid protein are encoded in one or more open reading frames and the Rep52 and Rep78 are encoded in one or more open reading frames, wherein the one or more open reading frames encoding the VP1 capsid protein, VP2 capsid protein, and VP3 capsid protein and the one or more open reading frames encoding the Rep52 and Rep78 are different open reading frames. In some embodiments, the VP1 capsid protein, VP2 capsid protein, and VP3 capsid protein are encoded in a first open reading frame and the Rep52 and Rep78 are encoded in a second open reading frame. In some embodiments, the ratio of VP1:VP2:VP3 of the rAAV produced by a method disclosed herein is about 1:1:10.

In certain embodiments, a composition comprising rAAVs comprising a polynucleotide encoding AADC or a functional variant thereof is produced by any of the methods disclosed herein. In some embodiments, the composition comprises 3.0×10¹²-5.0×10¹² vg/mL rAAVs, e.g., about 5.0×10¹² vg/mL rAAVs, in a solution comprising 10 mM sodium phosphate, 180 mM sodium chloride, and 0.001% poloxamer 188 (solution pH of 7.1-7.5, e.g., pH 7.3). In some embodiments, the composition is used in treating and/or preventing Parkinson's Disease. In some embodiments, the present disclosure comprises a method of treating Parkinson's Disease comprising administering an effective amount of the composition. In some embodiments, the composition is used for the manufacture of a medicament for treating and/or preventing Parkinson's Disease.

In certain embodiments, the present disclosure encompasses a method for producing a recombinant adeno-associated virus 2 (rAAV2) comprising a polynucleotide comprising SEQ ID NO: 979. In some embodiments, the method comprises the steps of: (a) culturing Sf9 cells (“viral production Sf9 cells”) in a bioreactor to a target cell density of 3.0×10⁶-3.4×10⁶ cells/mL; wherein the viral production Sf9 cells are cultured in serum-free, protein-free insect cell culture medium at about 26° C.-28° C. and 30%-50% dissolved oxygen, wherein the serum-free, protein-free insect cell culture medium optionally comprises L-glutamine and poloxamer 188; (b) introducing into the bioreactor baculovirus infected insect cells (expressionBIICs) comprising baculoviruses comprising a viral expression construct, and baculovirus infected insect cells (payloadBIICs) comprising baculoviruses comprising a payload construct, wherein the viral expression construct comprises one or more polynucleotides encoding capsid and replication proteins of adeno-associated virus 2 (AAV2); wherein the payload construct comprises SEQ ID NO: 979; and wherein the expressionBIICs are introduced at a ratio of about 1:300,000 expressionBIIC:Sf9 (v/v) and the payloadBIICs are introduced at a ratio of about 1:100,000 payloadBIIC:Sf9 (v/v), wherein the viral expression construct comprises one or more polynucleotides encoding capsid and replication proteins of adeno-associated virus 2 (AAV2, e.g., wild-type AAV2); wherein the expressionBIICs are optionally Sf9 cells and the payloadBIICs are optionally Sf9 cells; (c) incubating the viral production Sf9 cells in the bioreactor under conditions that result in the production of one or more rAAV2s within one or more of the viral production Sf9 cells, wherein one or more of the rAAV2s comprise a polynucleotide comprising SEQ ID NO: 979; (d) harvesting a viral production pool from the bioreactor, wherein the viral production pool comprises one or more viral production Sf9 cells comprising one or more rAAV2s, wherein the viral production pool optionally weighs 195-198 kg, e.g., 196 kg, and has a % viability of at least 85%, e.g., at least 90%; (e) lysing the viral production Sf9 cells in the viral production pool, wherein the lysing comprises a chemical lysis solution and is carried out at 26° C.-28° C. for 4-6 hours, wherein the chemical lysis solution comprises 0.5% (w/v) octyl phenol ethoxylate and 200 mM arginine hydrochloride, and lacks detectable nuclease, thereby releasing one or more rAAV2s from the viral production Sf9 cells into a lysis medium; (f) clarifying the lysis medium of (e) through a depth filter followed by an about 0.2 μm filter, yielding a clarification pool; (g) processing the clarification pool of (f) through an immunoaffinity chromatography column comprising a recombinant protein ligand that binds at least AAV2; wherein the immunoaffinity chromatography column is equilibrated with a solution comprising 50 mM sodium phosphate, 350 mM sodium chloride and 0.001% w/v poloxamer 188; loaded with a 1.0×10¹³-5.0×10¹³ vg/mL-r load challenge at 18-25° C.; flushed with a solution comprising 50 mM sodium phosphate, 350 mM sodium chloride and 0.001% w/v poloxamer 188; washed with a solution comprising 20 mM sodium citrate, 1 M sodium chloride and 0.001% w/v poloxamer 188; and washed with a solution of 10 mM sodium citrate, 350 mM sodium chloride and 0.001% w/v poloxamer 188; wherein processing the clarification pool of (f) through an immunoaffinity chromatography column yields an immunoaffinity chromatography pool, wherein the immunoaffinity chromatography pool is optionally neutralized with 2 M Tris Base and 0.001% w/v poloxamer 188 (3.0% v/v spike, pH 8.0-8.5) and optionally filtered through an about 0.2 μm filter; (h) processing the immunoaffinity chromatography pool or filtered immunoaffinity chromatography pool of (g) through an anion exchange chromatography column, e.g., a column operated in flow-through mode; wherein the one or more anion exchange chromatography columns is charged and equilibrated with a solution comprising 20 mM Tris, 2 M sodium chloride and 0.001% w/v poloxamer 188, then a solution of 40 mM Tris, 170 mM sodium chloride and 0.001% w/v poloxamer 188; loaded with a 1.0×10¹³-5.0×10¹³ vg/mL-r load challenge at 18-25° C.; and flushed and eluted with a solution comprising 40 mM Tris, 170 mM sodium chloride and 0.001% w/v poloxamer 188, yielding an anion exchange chromatography pool; wherein the anion exchange chromatography pool is filtered through an about 0.2 μm filter, yielding a filtered anion exchange chromatography pool; (i) processing the filtered anion exchange chromatography pool of (h) through a tangential flow filtration (TFF) filter yielding a TFF load pool; wherein the TFF filter is equilibrated with buffer comprising 40 mM Tris, 170 mM sodium chloride, and 0.001% (w/v) poloxamer 188; wherein the TFF load pool is concentrated by ultrafiltration to a viral concentration of about 5.0×10¹² vg/mL, followed by diafiltration comprising buffer exchange with a buffer comprising 10 mM sodium phosphate, 180 mM sodium chloride, and 0.001% (w/v) poloxamer 188 (buffer pH of 7.1-7.5, e.g., pH 7.3), followed by filtration through an about 0.2 μm filter, yielding a filtered TFF load pool; wherein a TFF recovery flush pool is prepared by subjecting the TFF filter to a recovery flush using a buffer comprising 10 mM sodium phosphate, 180 mM sodium chloride, and 0.001% (w/v) poloxamer 188 (buffer pH of 7.1-7.5, e.g., pH 7.3); wherein the TFF recovery flush pool is filtered through an about 0.2 μm filter, yielding a filtered TFF recovery flush pool; wherein the filtered TFF load pool and filtered TFF recovery flush pool are combined to form a concentrated, buffer-exchanged pool, and optionally diluted using a buffer comprising 10 mM sodium phosphate, 180 mM sodium chloride, and 0.001% (w/v) poloxamer 188 (buffer pH of 7.1-7.5, e.g., pH 7.3), wherein the concentrated, buffer-exchanged pool comprises a viral concentration of 2.0×10¹²-6.0×10¹² vg/mL; (j) processing the concentrated, buffer-exchanged pool of (i) through a viral retentive filtration (VRF) filter to yield a viral filtration pool; wherein the VRF filter comprises a pore size of about 35 nm and is flushed twice before use with a solution comprising 10 mM sodium phosphate, 180 mM sodium chloride, and 0.001% poloxamer 188 (solution pH of 7.1-7.5, e.g., pH 7.3); and wherein the viral filtration pool is filtered through a filter of about 0.2 μm, yielding a filtered viral filtration pool comprising a viral concentration of 3.5×10¹²-5.0×10¹² vg/mL; and (k) processing the viral production pool of (j) through an about 0.22 μm filter, e.g., filtering twice through an about 0.22 μm filter, to yield a purified rAAV2 composition comprising a viral concentration of 3.0×10¹²-5.0×10¹² vg/mL in a solution comprising 10 mM sodium phosphate, 180 mM sodium chloride, and 0.001% poloxamer 188 (solution pH of 7.1-7.5, e.g., pH 7.3).

In some embodiments, the viral concentration of the purified rAAV2 composition is about 5.0×10¹² vg/mL. In some embodiments, the method produces a total process rAAV yield of 30%-50%. In some embodiments, the viral expression construct comprises one or more polynucleotides encoding a VP1 capsid protein, VP2 capsid protein, VP3 capsid protein, Rep52, and Rep78. In some embodiments, the VP1 capsid protein, VP2 capsid protein, and VP3 capsid protein are encoded in one or more open reading frames and the Rep52 and Rep78 are encoded in one or more open reading frames, wherein the one or more open reading frames encoding the VP1 capsid protein, VP2 capsid protein, and VP3 capsid protein and the one or more open reading frames encoding the Rep52 and Rep78 are different open reading frames. In some embodiments, the VP1 capsid protein, VP2 capsid protein, and VP3 capsid protein are encoded in a first open reading frame and the Rep52 and Rep78 are encoded in a second open reading frame. In some embodiments, the purified rAAV2 comprise a ratio of VP1:VP2:VP3 of about 1:1:10.

In some embodiments, a composition produced by any of the methods disclosed herein comprises 3.0×10¹²-5.0×10¹² vg/mL rAAV2s, e.g., about 5.0×10¹² vg/mL rAAV2s, in a solution comprising 10 mM sodium phosphate, 180 mM sodium chloride, and 0.001% poloxamer 188 (solution pH of 7.1-7.5, e.g., pH 7.3). In some embodiments, the composition is used in treating and/or preventing Parkinson's Disease. In some embodiments, a method of treating Parkinson's Disease comprises administering an effective amount of the composition. In some embodiments, the composition is used in the manufacture of a medicament for treating and/or preventing Parkinson's Disease.

In some embodiments, the purified viral rAAV2 composition is formulated at a concentration of 3.0×1013-5.0×1013 vg/mL, e.g., about 5×1013 vg/mL. In some embodiments, the purified rAAV2 composition is formulated a concentration of 2.0×1013-3.0×1013 vg/mL, e.g., about 2.7×1013 vg/mL. In some embodiments, the purified viral rAAV2 composition comprises a dose of about 7.5×1011 vg per vial. In some embodiments, the purified viral rAAV2 composition comprises a dose of about 1.5×1012 vg per vial. In some embodiments, the purified viral rAAV2 composition comprises a dose of about 4.7×1012 vg per vial. In some embodiments, the purified viral rAAV2 composition comprises a dose of about 3.6×1012 vg per vial. In some embodiments, the purified viral rAAV2 composition comprises a dose of about 9.4×1012 vg per vial.

### BRIEF DESCRIPTION OF THE FIGURES

The foregoing and other objects, features and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure.

FIG. 1 shows a schematic for one embodiment of a system, and a flow diagram for one embodiment of a process, for producing baculovirus infected insect cells (BIICs) using Viral Production Cells (VPC) and plasmid constructs.

FIG. 2 shows a schematic for one embodiment of a system, and a flow diagram for one embodiment of a process, for producing AAV Particles using Viral Production Cells (VPC) and baculovirus infected insect cells (BIICs) or with baculovirus.

FIG. 3 shows schematic for one embodiment of a system, and a flow diagram for one embodiment of a process, for producing a Drug Substance by processing, clarifying and purifying a bulk harvest of AAV particles and Viral Production Cells.

FIG. 4A and FIG. 4B show the results of computer modeling for VPC-to-expressionBIIC ratio (v/v) vs. payloadBIIC-to-expressionBIIC ratio (v/v) in BIIC transfection of viral production cells (VPC). FIG. 4A shows AAV titer (vg/mL) using ddPCR, and FIG. 4B shows Capsid Full %.

FIG. 5A and FIG. 5B show the results of computer modeling for VPC-to-expressionBIIC ratio (v/v) vs. target VPC cell density at infection (x10⁶ cells/mL) in BIIC transfection of viral production cells (VPC). FIG. 5A shows AAV titer (vg/mL) using ddPCR, and FIG. 5B shows Capsid Full %.

FIG. 6A and FIG. 6B show the results of computer modeling for payloadBIIC-to-expressionBIIC ratio (v/v) vs. target VPC cell density at infection (x10⁶ cells/mL) in BIIC transfection of viral production cells (VPC). FIG. 6A shows AAV titer (vg/mL) using ddPCR, and FIG. 6B shows Capsid Full %.

FIG. 7 shows an embodiment of an upstream process for producing rAAVs comprising a polynucleotide encoding AADC or a functional variant thereof.

FIG. 8A and FIG. 8B shows an embodiment of a downstream process for producing rAAVs comprising a polynucleotide encoding AADC or a functional variant thereof.

### DETAILED DESCRIPTION

### I. Adeno-Associated Viruses (AAVS)

### Overview

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family comprises the Dependovirus genus which comprises AAV, capable of replication in vertebrate hosts comprising, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the content of which is incorporated herein by reference in its entirety as related to parvoviruses, insofar as it does not conflict with the present disclosure.

AAV have proven to be useful as a biological tool due to their relatively simple structure, their ability to infect a wide range of cells (comprising quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

### AAV Viral Genomes

The wild-type AAV viral genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions comprising, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the content of which is incorporated herein by reference in its entirety as related to AAV9/hu.14, insofar as it does not conflict with the present disclosure) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

For use as a biological tool, the wild-type AAV viral genome can be modified to replace the rep/cap sequences with a nucleic acid sequence comprising a payload region with at least one ITR region. Typically, in recombinant AAV viral genomes there are two ITR regions. The rep/cap sequences can be provided in trans during production to generate AAV particles.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. See Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entireties as related to AAV variants and equivalents, insofar as they do not conflict with the present disclosure.

In certain embodiments, AAV particles, viral genomes and/or payloads of the present disclosure, and the methods of their use, may be as described in WO2017189963, the content of which is incorporated herein by reference in its entirety as related to AAV particles, viral genomes and/or payloads, insofar as it does not conflict with the present disclosure.

AAV particles of the present disclosure may be formulated in any of the gene therapy formulations of the disclosure comprising any variations of such formulations apparent to those skilled in the art. The reference to “AAV particles,” “AAV particle formulations” and “formulated AAV particles” in the present application refers to the AAV particles which may be formulated and those which are formulated without limiting either.

In certain embodiments, AAV particles of the present disclosure are recombinant AAV (rAAV) viral particles which are replication defective, lacking sequences encoding functional Rep and Cap proteins within their viral genome. These defective AAV particles may lack most or all parental coding sequences and essentially carry only one or two AAV ITR sequences and the nucleic acid of interest (i.e. payload) for delivery to a cell, a tissue, an organ or an organism.

In certain embodiments, the viral genome of the AAV particles of the present disclosure comprises at least one control element which provides for the replication, transcription and translation of a coding sequence encoded therein. Not all of the control elements need always be present as long as the coding sequence is capable of being replicated, transcribed and/or translated in an appropriate host cell. Non-limiting examples of expression control elements comprise sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

In certain embodiments, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV particles of the present disclosure may be produced recombinantly and may be based on adeno-associated virus (AAV) parent or reference sequences. As used herein, a “vector” is any molecule or moiety which transports, transduces or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV vector genomes contain DNA strands which anneal together to form double stranded DNA. By skipping second strand synthesis, scAAVs allow for rapid expression in the cell.

In certain embodiments, the AAV viral genome of the present disclosure is a scAAV. In certain embodiments, the AAV viral genome of the present disclosure is a ssAAV.

AAV particles may be modified to enhance the efficiency of delivery. Such modified AAV particles can be packaged efficiently and be used to successfully infect the target cells at high frequency and with minimal toxicity. In certain embodiments the capsids of the AAV particles are engineered according to the methods described in US Publication Number US 20130195801, the content of which is incorporated herein by reference in its entirety as related to modifying AAV particles to enhance the efficiency of delivery, insofar as it does not conflict with the present disclosure.

In certain embodiments, the AAV particles comprise a payload construct and/or region encoding a polypeptide or protein of the present disclosure, and may be introduced into mammalian cells. In certain embodiments, the AAV particles comprise a payload construct and/or region encoding a polypeptide or protein of the present disclosure, and may be introduced into insect cells. For instance, the payload construct and/or region may encode an AADC protein.

### Inverted Terminal Repeats (ITRs)

The AAV particles of the present disclosure comprise a viral genome with at least one ITR region and a payload region. In certain embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes of the present disclosure may be comprised of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome comprising two ITRs. In certain embodiments, the ITRs are of the same serotype as one another. In another embodiment, the ITRs are of different serotypes. Non-limiting examples comprise zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length.

In certain embodiments, each ITR may be 141 nucleotides in length. In certain embodiments, each ITR may be 130 nucleotides in length. In certain embodiments, each ITR may be 119 nucleotides in length.

In certain embodiments, the AAV particles comprise two ITRs and one ITR is 141 nucleotides in length and the other ITR is 130 nucleotides in length. In certain embodiments, the AAV particles comprise two ITRs and both ITRs are 141 nucleotides in length. Independently, each ITR may be about 75 to about 175 nucleotides in length. As a non-limiting example, the viral genome comprises an ITR that is about 130 nucleotides in length and an ITR that is about 141 nucleotides in length. As a non-limiting example, the viral genome may comprise two ITRs, each of which are about 141 nucleotides in length. In some embodiments, the viral genome comprises two ITRs (i.e., a 5′ ITR and a 3′ITR), each of which is 141 nucleotides in length.

In some embodiments, the viral genome comprises a 5′ ITR comprising a sequence at least 95%, at least 96%, at least 97%, at least 98%, at least 99%, or 100% identical to SEQ ID NO: 980. In some embodiments, the viral genome comprises a 5′ ITR comprising SEQ ID NO: 980. In some embodiments, the viral genome comprises a 3′ ITR comprising a sequence at least 95%, at least 96%, at least 97%, at least 98%, at least 99%, or 100% identical to SEQ ID NO: 991. In some embodiments, the viral genome comprises a 3′ ITR comprising SEQ ID NO: 991.

### Promoters

In certain embodiments, the payload region of the viral genome comprises at least one element to enhance the transgene target specificity and expression (See e.g., Powell et al. Viral Expression Cassette Elements to Enhance Transgene Target Specificity and Expression in Gene Therapy, 2015; the content of which is incorporated herein by reference in its entirety as related to payload/transgene enhancer elements, insofar as it does not conflict with the present disclosure). Non-limiting examples of elements to enhance the transgene target specificity and expression comprise promoters, endogenous miRNAs, post-transcriptional regulatory elements (PREs), polyadenylation (Poly(A)) signal sequences and upstream enhancers (USEs), CMV promoters, CMV enhancers, and introns.

A person skilled in the art may recognize that expression of the polypeptides of the present disclosure in a target cell may require a specific promoter, comprising but not limited to, a promoter that is species specific, inducible, tissue-specific, or cell cycle-specific (see Parr et al., Nat. Med.3:1145-9 (1997); the content of which is incorporated herein by reference in its entirety as related to polypeptide expression promoters, insofar as it does not conflict with the present disclosure).

In certain embodiments, the promoter drives expression of the polypeptide(s) encoded in the payload region of the viral genome of the AAV particle. In certain embodiments, the promoter drives expression in the cell being targeted. In certain embodiments, the promoter has a tropism for the cell being targeted. In certain embodiments, the promoter has a tropism for a viral production cell.

In certain embodiments, the promoter drives expression of the payload (e.g., AADC) for a period of time in targeted cells or tissues. Expression driven by a promoter may be for a period of 1-31 days (or any value or range therein), 1-23 months (or any value or range therein), 2-10 years (or any value or range therein), or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years. As a non-limiting example, the promoter can be a weak promoter for sustained expression of a payload in nervous (e.g., CNS) cells or tissues.

In certain embodiments, the promoter drives expression of the polypeptides of the present disclosure for at least 1-11 months (or any individual value therein), 2-65 years (or any individual value therein), or more than 65 years.

Promoters may be naturally occurring or non-naturally occurring. Non-limiting examples of promoters comprise viral promoters, plant promoters and mammalian promoters. In certain embodiments, the promoters may be human promoters. In certain embodiments, the promoter may be truncated or mutated.

Promoters which drive or promote expression in most tissues comprise, but are not limited to, human elongation factor 1α-subunit (EF1α), cytomegalovirus (CMV) immediate-early enhancer and/or promoter, chicken β-actin (CBA) and its derivative CAG, R glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons or subtypes of neurons, astrocytes, or oligodendrocytes.

Non-limiting examples of muscle-specific promoters comprise mammalian muscle creatine kinase (MCK) promoter, mammalian desmin (DES) promoter, mammalian troponin I (TNNI2) promoter, and mammalian skeletal alpha-actin (ASKA) promoter (see, e.g., U.S. Patent Publication US 20110212529, the content of which is incorporated herein by reference in its entirety as related to muscle-specific promoters, insofar as they do not conflict with the present disclosure)

Non-limiting examples of tissue-specific expression elements for neurons comprise neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light chain (NFL) or neurofilament heavy chain (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes comprise glial fibrillary acidic protein (GFAP) and EAAT2 promoters. A non-limiting example of a tissue-specific expression element for oligodendrocytes comprises the myelin basic protein (MBP) promoter.

In certain embodiments, the promoter may be less than 1 kb. The promoter may have a length of 200-800 nucleotides (or any value or range therein), or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

The AAV particles of the present disclosure comprise a viral genome with at least one promoter region. As a non-limiting example, the viral genome comprises a promoter region that is about 204 nucleotides in length.

In certain embodiments, the promoter may be a combination of two or more components of the same or different starting or parental promoters such as, but not limited to, CMV and CBA. Each component may have a length of 200-800 nucleotides (or any value or range therein).

In certain embodiments, the viral genome comprises a ubiquitous promoter. Non-limiting examples of ubiquitous promoters comprise CMV, CBA (comprising derivatives CAG, CBh, etc.), EF-1α, PGK, UBC, GUSB (hGBp), and UCOE (promoter of HNRPA2B1-CBX3). In certain embodiments, the promoter region is derived from a CBA promoter sequence. As a non-limiting example, the promoter is 260 nucleotides in length.

Yu et al. (Molecular Pain 2011, 7:63; the content of which is incorporated herein by reference in its entirety, insofar as it does not conflict with the present disclosure) evaluated the expression of eGFP under the CAG, EFIu, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015; the contents of which are each incorporated herein by reference in its entirety) evaluated the expression of eGFP in AAV8 with CMV and UBC promoters and AAV2 with the CMV promoter after injection in the motor cortex. Intranasal administration of a plasmid containing a UBC or EFIu promoter showed a sustained airway expression greater than the expression with the CMV promoter (See e.g., Gill et al., Gene Therapy 2001, Vol. 8, 1539-1546; the content of which is incorporated herein by reference in its entirety, insofar as it does not conflict with the present disclosure). Husain et al. (Gene Therapy 2009; the content of which is incorporated herein by reference in its entirety, insofar as it does not conflict with the present disclosure) evaluated an HOH construct with a hGUSB promoter, a HSV-1LAT promoter and an NSE promoter and found that the HOH construct showed weaker expression than NSE in mouse brain. Passini and Wolfe (J. Virol. 2001, 12382-12392, the content of which is incorporated herein by reference in its entirety, insofar as it does not conflict with the present disclosure) evaluated the long term effects of the HOH vector following an intraventricular injection in neonatal mice and found that there was sustained expression for at least 1 year. Low expression in all brain regions was found by Xu et al. (Gene Therapy 2001, 8, 1323-1332; the content of which is incorporated herein by reference in its entirety, insofar as it does not conflict with the present disclosure) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL promoter is a 650-nucleotide promoter and NFH promoter is a 920-nucleotide promoter which are both absent in the liver but NFH promoter is abundant in the sensory proprioceptive neurons, brain and spinal cord and NFH promoter is present in the heart. SCN8A promoter is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. Identification of evolutionary conserved, functional noncoding elements in the promoter region of the sodium channel gene SCN8A, Mamm Genome (2007) 18:723-731; and Raymond et al. Expression of Alternatively Spliced Sodium Channel α-subunit genes, Journal of Biological Chemistry (2004) 279(44) 46234-46241; the contents of each of which are incorporated herein by reference in their entireties, insofar as they do not conflict with the present disclosure).

Any of the promoters taught by the aforementioned Yu, Soderblom, Gill, Husain, Passini, Xu, Drews or Raymond may be used in the present disclosures.

In certain embodiments, the promoter is not cell specific.

In certain embodiments, the promoter is a ubiquitin c (UBC) promoter. The UBC promoter may have a size of 300-350 nucleotides. As a non-limiting example, the UBC promoter is 332 nucleotides. In certain embodiments, the promoter is a β-glucuronidase (GUSB) promoter. The GUSB promoter may have a size of 350-400 nucleotides. As a non-limiting example, the GUSB promoter is 378 nucleotides. In certain embodiments, the promoter is a neurofilament light chain (NFL) promoter. The NFL promoter may have a size of 600-700 nucleotides. As a non-limiting example, the NFL promoter is 650 nucleotides. In certain embodiments, the promoter is a neurofilament heavy chain (NFH) promoter. The NFH promoter may have a size of 900-950 nucleotides. As a non-limiting example, the NFH promoter is 920 nucleotides. In certain embodiments, the promoter is a SCN8A promoter. The SCN8A promoter may have a size of 450-500 nucleotides. As a non-limiting example, the SCN8A promoter is 470 nucleotides.

In certain embodiments, the promoter is a frataxin (FXN) promoter. In certain embodiments, the promoter is a phosphoglycerate kinase 1 (PGK) promoter. In certain embodiments, the promoter is a chicken β-actin (CBA) promoter, or variant thereof. In certain embodiments, the promoter is a CB6 promoter. In certain embodiments, the promoter is a minimal CB promoter. In certain embodiments, the promoter is a cytomegalovirus (CMV) promoter. In certain embodiments, the promoter is a H1 promoter. In certain embodiments, the promoter is a CAG promoter. In certain embodiments, the promoter is a GFAP promoter. In certain embodiments, the promoter is a synapsin promoter. In certain embodiments, the promoter is an engineered promoter. In certain embodiments, the promoter is a liver or a skeletal muscle promoter. Non-limiting examples of liver promoters comprise human α-1-antitrypsin (hAAT) and thyroxine binding globulin (TBG). Non-limiting examples of skeletal muscle promoters comprise Desmin, MCK or synthetic C5-12. In certain embodiments, the promoter is an RNA pol III promoter. As a non-limiting example, the RNA pol III promoter is U6. As a non-limiting example, the RNA pol III promoter is H1. In certain embodiments, the promoter is a cardiomyocyte-specific promoter. Non-limiting examples of cardiomyocyte-specific promoters comprise uMHC, cTnT, and CMV-MLC2k. In certain embodiments, the viral genome comprises two promoters. As a non-limiting example, the promoters are an EFIu promoter and a CMV promoter.

In certain embodiments, the viral genome comprises an enhancer element, a promoter and/or a 5′ UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′ UTR/intron may be, but is not limited to, SV40, and CBA-MVM. As a non-limiting example, the enhancer, promoter and/or intron used in combination may be: (1) CMV enhancer, CMV promoter, SV40 5′ UTR intron; (2) CMV enhancer, CBA promoter, SV-40 5′ UTR intron; (3) CMV enhancer, CBA promoter, CBA-MVM 5′ UTR intron; (4) UBC promoter; (5) GUSB promoter; (6) NSE promoter; (7) Synapsin promoter; (8) MeCP2 promoter and (9) GFAP promoter.

In certain embodiments, the viral genome comprises an engineered promoter.

In another embodiment, the viral genome comprises a promoter from a naturally expressed protein.

In some embodiments, the promoter comprises a sequence having at least 95%, at least 96%, at least 97%, at least 98%, at least 99%, or 100% identity to SEQ ID NO: 983. In some embodiments, the promoter comprises SEQ ID NO: 983.

In certain embodiments, the AAV particles of the present disclosure comprise a viral genome with at least one enhancer region. The enhancer region(s) may, independently, have a length such as, but not limited to, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, and 400 nucleotides. The length of the enhancer region for the viral genome may be 300-310, 300-325, 305-315, 310-320, 315-325, 320-330, 325-335, 325-350, 330-340, 335-345, 340-350, 345-355, 350-360, 350-375, 355-365, 360-370, 365-375, 370-380, 375-385, 375-400, 380-390, 385-395, and 390-400 nucleotides. As a non-limiting example, the viral genome comprises an enhancer region that is about 303 nucleotides in length.

In certain embodiments, the enhancer region is derived from a CMV enhancer sequence. As a non-limiting example, the CMV enhancer is 382 nucleotides in length.

In some embodiments, the enhancer comprises a sequence having at least 95%, at least 96%, at least 97%, at least 98%, at least 99%, or 100% identity to SEQ ID NO: 982. In some embodiments, the enhancer comprises SEQ ID NO: 982.

In some embodiments, the viral genome comprises a CMV enhancer and CMV promoter. In certain embodiments, the CMV enhancer comprises SEQ ID NO: 982 and the CMV promoter comprises SEQ ID NO: 983. In certain embodiments, the CMV enhancer is 303 nucleotides in length and the CMV promoter is 204 nucleotides in length.

### Untranslated Regions (UTRs)

Wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genomes of the AAV particles of the present disclosure to enhance expression in hepatic cell lines or liver.

While not wishing to be bound by theory, wild-type 5′ untranslated regions (UTRs) may comprise features which play roles in translation initiation. Kozak sequences, which are commonly known to be involved in the process by which the ribosome initiates translation of many genes, are usually comprised in 5′ UTRs. Kozak sequences have the consensus CCR(A/G)CCAUGG, where R is a purine (adenine or guanine) three bases upstream of the start codon (ATG), which is followed by another ‘G’. In certain embodiments, the 5′ UTR in the viral genome comprises a Kozak sequence. In certain embodiments, the 5′ UTR in the viral genome does not comprise a Kozak sequence.

While not wishing to be bound by theory, wild-type 3′ UTRs often have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the content of which is incorporated herein by reference in its entirety as related to AU rich elements, insofar as it does not conflict with the present disclosure): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, (e.g., payload regions of viral genomes), one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In certain embodiments, the 3′ UTR of the viral genome may comprise an oligo(dT) sequence for templated addition of a poly-A tail.

In certain embodiments, the viral genome may comprise at least one miRNA seed, binding site or full sequence. MicroRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which sequence has perfect Watson-Crick complementarity to the miRNA target sequence of the nucleic acid.

In certain embodiments, the viral genome may be engineered to comprise, alter or remove at least one miRNA binding site, sequence or seed region.

Any UTR from any gene known in the art may be incorporated into the viral genome of the AAV particle. These UTRs, or portions thereof, may be placed in the same orientation as in the gene from which they were selected, or they may be altered in orientation or location. In certain embodiments, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In certain embodiments, the viral genome of the AAV particle comprises at least one artificial UTRs which is not a variant of a wild type UTR.

In certain embodiments, the viral genome of the AAV particle comprises UTRs which have been selected from a family of transcripts whose proteins share a common function, structure, feature or property.

### Polyadenylation Sequence

In certain embodiments, the viral genome of the AAV particles of the present disclosure comprise at least one polyadenylation sequence. The viral genome of the AAV particle may comprise a polyadenylation sequence between the 3′ end of the payload (e.g., AADC) coding region and the 5′ end of the 3′ ITR. The viral genome of the AAV particle may comprise a polyadenylation sequence between the 3′ end of a multiple cloning site region and the 5′ end of the 3′ ITR.

In certain embodiments, the polyadenylation sequence or “poly(A) sequence” (also referred to as “poly(A) signal”) may range from absent to about 500 nucleotides in length. The polyadenylation sequence may be, but is not limited to, 1-500 nucleotides in length (or any value or range therein).

In certain embodiments, the polyadenylation sequence is 127 nucleotides in length. In certain embodiments, the polyadenylation sequences is 477 nucleotides in length. In certain embodiments, the polyadenylation sequence is 552 nucleotides in length.

In certain embodiments the poly(A) signal comprises a sequence having at least 95%, at least 96%, at least 97%, at least 98%, at least 99%, or 100% identity to SEQ ID NO: 990. In some embodiments, the poly(A) signal comprises SEQ ID NO: 990.

### Linkers

Viral genomes of the present disclosure may be engineered with one or more spacer or linker regions to separate coding or non-coding regions.

In certain embodiments, the payload region of the AAV particle may optionally encode one or more linker sequences. In some cases, the linker may be a peptide linker that may be used to connect the polypeptides encoded by the payload region. Some peptide linkers may be cleaved after expression to separate polypeptide domains, allowing assembly of mature protein fragments. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains (e.g., heavy and light chain antibody domains) from a single transcript. In some cases, two or more linkers are encoded by a payload region of the viral genome.

In certain embodiments, payload regions encode linkers comprising furin cleavage sites. Furin is a calcium dependent serine endoprotease that cleaves proteins just downstream of a basic amino acid target sequence (Arg-X-(Arg/Lys)-Arg) (Thomas, G., 2002. Nature Reviews Molecular Cell Biology 3(10): 753-66; the content of which is incorporated herein by reference in its entirety as related to linker molecules or sequences, insofar as it does not conflict with the present disclosure). Furin is enriched in the trans-golgi network where it is involved in processing cellular precursor proteins. Furin also plays a role in activating a number of pathogens. This activity can be taken advantage of for expression of polypeptides of the disclosure.

In certain embodiments, payload regions encode linkers comprising 2A peptides. 2A peptides are small “self-cleaving” peptides (18-22 amino acids) derived from viruses such as foot-and-mouth disease virus (F2A), porcine teschovirus-1 (P2A), Thoseaasigna virus (T2A), or equine rhinitis A virus (E2A). The 2A designation refers specifically to a region of picornavirus polyproteins that lead to a ribosomal skip at the glycyl-prolyl bond in the C-terminus of the 2A peptide (Kim, J. H. et al., 2011. PLoS One 6(4): e18556; the content of which is incorporated herein by reference in its entirety as related to 2A peptide linkers, insofar as it does not conflict with the present disclosure). This skip results in a cleavage between the 2A peptide and its immediate downstream peptide. As opposed to IRES linkers, 2A peptides generate stoichiometric expression of proteins flanking the 2A peptide and their shorter length can be advantageous in generating viral expression vectors.

In certain embodiments, payload regions encode linkers comprising IRES. Internal ribosomal entry site (IRES) is a nucleotide sequence (>500 nucleotides) that allows for initiation of translation in the middle of an mRNA sequence (Kim, J. H. et al., 2011. PLoS One 6(4): e18556; the content of which is incorporated herein by reference in its entirety as related to IRES regions and linkers, insofar as it does not conflict with the present disclosure). Use of an IRES sequence ensures co-expression of genes before and after the IRES, though the sequence following the IRES may be transcribed and translated at lower levels than the sequence preceding the IRES sequence.

In certain embodiments, the payload region may encode one or more linkers comprising cathepsin, matrix metalloproteinases or legumain cleavage sites. Such linkers are described e.g., by Cizeau and Macdonald in International Publication No. WO2008052322, the content of which is incorporated herein by reference in its entirety as related to linker molecules and sequences, insofar as it does not conflict with the present disclosure. Cathepsins are a family of proteases with unique mechanisms to cleave specific proteins. Cathepsin B is a cysteine protease and cathepsin D is an aspartyl protease. Matrix metalloproteinases are a family of calcium-dependent and zinc-containing endopeptidases. Legumain is an enzyme catalyzing the hydrolysis of (-Asn-Xaa-) bonds of proteins and small molecule substrates.

In certain embodiments, payload regions may encode linkers that are not cleaved. Such linkers may comprise a simple amino acid sequence, such as a glycine rich sequence. In some cases, linkers may comprise flexible peptide linkers comprising glycine and serine residues. These flexible linkers are small and without side chains so they tend not to influence secondary protein structure while providing a flexible linker between antibody segments (George, R. A., et al., 2002. Protein Engineering 15(11): 871-9; Huston, J. S. et al., 1988. PNAS 85:5879-83; and Shan, D. et al., 1999. Journal of Immunology. 162(11):6589-95; the contents of which are each incorporated herein by reference in their entireties as related to linker molecules and sequences, insofar as they do not conflict with the present disclosure). Furthermore, the polarity of the serine residues improves solubility and prevents aggregation problems.

In certain embodiments, payload regions of the present disclosure may encode small and unbranched serine-rich peptide linkers, such as those described by Huston et al. in U.S. Pat. No. 5,525,491, the content of which is incorporated herein by reference in its entirety as related to linker molecules and sequences, insofar as it does not conflict with the present disclosure. Polypeptides encoded by the payload region of the present disclosure, linked by serine-rich linkers, have increased solubility.

In certain embodiments, payload regions of the present disclosure may encode artificial linkers, such as those described by Whitlow and Filpula in U.S. Pat. No. 5,856,456 and Ladner et al. in U.S. Pat. No. 4,946,778, the contents of which are each incorporated herein by reference in their entireties as related to linker molecules and sequences, insofar as they do not conflict with the present disclosure.

In certain embodiments, the linker region may be 1-50, 1-100, 50-100, 50-150, 100-150, 100-200, 150-200, 150-250, 200-250, 200-300, 250-300, 250-350, 300-350, 300-400, 350-400, 350-450, 400-450, 400-500, 450-500, 450-550, 500-550, 500-600, 550-600, 550-650, or 600-650 nucleotides in length. The linker region may have a length of 1-650 nucleotides (or any value or range therein) or greater than 650. In certain embodiments, the linker region may be 12 nucleotides in length. In certain embodiments, the linker region may be 18 nucleotides in length. In certain embodiments, the linker region may be 45 nucleotides in length. In certain embodiments, the linker region may be 54 nucleotides in length. In certain embodiments, the linker region may be 66 nucleotides in length. In certain embodiments, the linker region may be 75 nucleotides in length. In certain embodiments, the linker region may be 78 nucleotides in length. In certain embodiments, the linker region may be 87 nucleotides in length. In certain embodiments, the linker region may be 108 nucleotides in length. In certain embodiments, the linker region may be 153 nucleotides in length. In certain embodiments, the linker region may be 198 nucleotides in length. In certain embodiments, the linker region may be 623 nucleotides in length.

### Introns and Exons

In certain embodiments, the vector genome comprises at least one element to enhance the transgene target specificity and expression (See e.g., Powell et al. Viral Expression Cassette Elements to Enhance Transgene Target Specificity and Expression in Gene Therapy, 2015; the content of which is incorporated herein by reference in its entirety as related to transgene targeting enhancers, insofar as it does not conflict with the present disclosure) such as an intron. Non-limiting examples of introns comprise, MVM (67-97 bps), FIX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In certain embodiments, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500. In some embodiments, the intron or intron portion comprises a region that is about 32 nucleotides in length. In some embodiments, the intron or intron portion comprises SEQ ID NO: 985. In some embodiments, the intron or intron portion comprises a region that is about 347 nucleotides in length. In some embodiments, the intron or intron portion comprises SEQ ID NO: 986.

In certain embodiments, the AAV particles of the present disclosure can comprise a viral genome with at least one exon region. The exon region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the exon region for the viral genome may be 2-10, 5-10, 5-15, 10-20, 10-30, 10-40, 15-20, 15-25, 20-30, 20-40, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises an exon region that is about 53 nucleotides in length. As a non-limiting example, the viral genome comprises an exon region that is about 134 nucleotides in length.

In certain embodiments, the viral genome of the AAV particle comprises an intron region between the 3′ end of a promoter region and the 5′ end of a payload (e.g., AADC) coding region. In some embodiments, the intron region comprises one or more of “immediate-early” ie1 exon 1, an ie1 intron 1 (e.g., “partial ie1 intron 1”), a human beta-globin intron 2, and a human beta-globin exon 3. In some embodiments, the intron region comprises an ie1 exon 1, an ie1 intron 1 (e.g., “partial ie1 intron 1”), a human beta-globin intron 2, and a human beta-globin exon 3.

In some embodiments, the intron region comprises one or more of SEQ ID NOs: 984-987. In some embodiments, the intron region comprises, in order from 5′ to 3′, SEQ ID NOs: 984-987.

### Stuffer Sequences

In certain embodiments, the viral genome comprises at least one element to improve packaging efficiency and expression, such as a stuffer or filler sequence. Non-limiting examples of stuffer sequences comprise albumin and/or alpha-1 antitrypsin. Any known viral, mammalian, or plant sequence may be manipulated for use as a stuffer sequence.

In certain embodiments, the stuffer or filler sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000.

### Multiple Cloning Site (MCS) Region

In certain embodiments, the AAV particles of the present disclosure comprise a viral genome with at least one multiple cloning site (MCS) region. The MCS region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the MCS region for the viral genome may be 2-10, 5-10, 5-15, 10-20, 10-30, 10-40, 15-20, 15-25, 20-30, 20-40, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises an MCS region that is about 5 nucleotides in length. As a non-limiting example, the viral genome comprises an MCS region that is about 10 nucleotides in length. As a non-limiting example, the viral genome comprises an MCS region that is about 14 nucleotides in length. As a non-limiting example, the viral genome comprises an MCS region that is about 18 nucleotides in length. As a non-limiting example, the viral genome comprises an MCS region that is about 73 nucleotides in length. As a non-limiting example, the viral genome comprises an MCS region that is about 121 nucleotides in length.

In certain embodiments, the MCS region is 5 nucleotides in length.

In certain embodiments, the MCS region is 10 nucleotides in length.

In certain embodiments, the MCS region comprises an MCS sequence that is 18 nucleotides in length. In certain embodiments, a viral genome comprises a first and second MCS region, wherein each comprises an MCS sequence that is 18 nucleotides in length.

In some embodiments, the 5′ ITR is followed by a region comprising a first multiple cloning site (MCS). In some embodiments, the first MCS comprises at least 95%, at least 96%, at least 97%, at least 98%, at least 99%, or 100% identical to SEQ ID NO: 981. In certain embodiments, the first MCS comprises SEQ ID NO: 981.

In some embodiments, the 3′ ITR is preceded by a region comprising a second MCS. In some embodiments, the second MCS precedes (e.g., is 5′ to) a poly(A) signal region, which precedes (is 5′ to) the 3′ ITR. In some embodiments, the second MCS comprises at least 95%, at least 96%, at least 97%, at least 98%, at least 99%, or 100% identical to SEQ ID NO: 989. In certain embodiments, the second MCS comprises SEQ ID NO: 989.

### Genome Size

In certain embodiments, the AAV particle which comprises a payload described herein may be single stranded or double stranded vector genome. The size of the vector genome may be small, medium, large or the maximum size. Additionally, the vector genome may comprise a promoter and a poly(A) signal.

In certain embodiments, the vector genome which comprises a payload described herein may be a small single stranded vector genome. A small single stranded vector genome may be 2.1 to 3.5 kb in size such as about 2.1, 2.2, 2.3, 2.4, 2.5, 2.6, 2.7, 2.8, 2.9, 3.0, 3.1, 3.2, 3.3, 3.4, and 3.5 kb in size. As a non-limiting example, the small single stranded vector genome may be 3.2 kb in size. As another non-limiting example, the small single stranded vector genome may be 2.2 kb in size. Additionally, the vector genome may comprise a promoter and a poly(A) signal.

In certain embodiments, the vector genome which comprises a payload described herein may be a small double stranded vector genome. A small double stranded vector genome may be 1.3 to 1.7 kb in size such as about 1.3, 1.4, 1.5, 1.6, and 1.7 kb in size. As a non-limiting example, the small double stranded vector genome may be 1.6 kb in size. Additionally, the vector genome may comprise a promoter and a poly(A) signal.

In certain embodiments, the vector genome which comprises a payload described herein may be a medium single stranded vector genome. A medium single stranded vector genome may be 3.6 to 4.3 kb in size such as about 3.6, 3.7, 3.8, 3.9, 4.0, 4.1, 4.2 and 4.3 kb in size. As a non-limiting example, the medium single stranded vector genome may be 4.0 kb in size. Additionally, the vector genome may comprise a promoter and a poly(A) signal.

In certain embodiments, the vector genome which comprises a payload described herein may be a medium double stranded vector genome. A medium double stranded vector genome may be 1.8 to 2.1 kb in size such as about 1.8, 1.9, 2.0, and 2.1 kb in size. As a non-limiting example, the medium double stranded vector genome may be 2.0 kb in size. Additionally, the vector genome may comprise a promoter and a poly(A) signal.

In certain embodiments, the vector genome which comprises a payload described herein may be a large single stranded vector genome. A large single stranded vector genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded vector genome may be 4.7 kb in size. As another non-limiting example, the large single stranded vector genome may be 4.8 kb in size. As yet another non-limiting example, the large single stranded vector genome may be 6.0 kb in size. Additionally, the vector genome may comprise a promoter and a poly(A) signal.

In certain embodiments, the vector genome which comprises a payload described herein may be a large double stranded vector genome. A large double stranded vector genome may be 2.2 to 3.0 kb in size such as about 2.2, 2.3, 2.4, 2.5, 2.6, 2.7, 2.8, 2.9 and 3.0 kb in size. As a non-limiting example, the large double stranded vector genome may be 2.4 kb in size. Additionally, the vector genome may comprise a promoter and a poly(A) signal.

### Exemplary AADC Polynucleotide (Payload) Constructs

According to the present disclosure, aromatic L-amino acid decarboxylase (AADC; also known as dopa decarboxylase or DDC) polynucleotides are provided which function alone or in combination with additional nucleic acid sequence(s) to encode the AADC protein. Such polynucleotides may be included in the vectors, AAVs, and constructs discussed herein and/or produced according to the methods disclosed herein. As used herein an “AADC polynucleotide” is any nucleic acid polymer (i.e., nucleic acid sequence) which encodes an AADC protein and when present in a vector, plasmid or translatable construct, expresses such AADC protein in a cell, tissue, organ or organism. In some embodiments, the AADC polynucleotide used in the methods and systems disclosed herein encodes an AADC protein of SEQ ID NO: 978 or a functional fragment thereof. In some embodiments, the polynucleotide encoding SEQ ID NO: 978 comprises SEQ ID NO: 979.

AADC polynucleotides include precursor molecules which are processed inside the cell. AADC polynucleotides or the processed forms thereof may be encoded in a plasmid, vector, genome or other nucleic acid expression vector for delivery to a cell.

In some embodiments AADC polynucleotides are designed as components of AAV viral genomes and packaged in AAV particles which are processed within the cell to produce the wild type AADC protein.

In some embodiments, the AADC polynucleotide may be the payload of the AAV particle.

As used herein, the wild type AADC protein may be any of the naturally occurring isoforms or variants from the DDC gene. Multiple alternatively spliced transcript variants encoding different isoforms of AADC have been identified. Specifically, the DDC gene produces seven transcript variants that encode six distinct isoforms. DDC transcript variants 1 and 2 both encode AADC isoform 1. In some embodiments, the AADC polynucleotides used in the compositions, methods, and systems disclosed herein encode DDC transcript variant 2, thereby encoding a native AADC isoform 1 (NCBI Reference Sequence: NP_000781.1). This protein sequence is given here:

(SEQ ID NO: 978)
MNASEFRRRGKEMVDYVANYMEGIEGRQVYPDVEPGYLRPLIPAAAPQE
PDTFEDIINDVEKIIMPGVTHWHSPYFFAYFPTASSYPAMLADMLCGAI
GCIGFSWAASPACTELETVMMDWLGKMLELPKAFLNEKAGEGGGVIQGS
ASEATLVALLAARTKVIHRLQAASPELTQAAIMEKLVAYSSDQAHSSVE
RAGLIGGVKLKAIPSDGNFAMRASALQEALERDKAAGLIPFFMVATLGT
TTCCSFDNLLEVGPICNKEDIWLHVDAAYAGSAFICPEFRHLLNGVEFA
DSFNFNPHKWLLVNFDCSAMWVKKRTDLTGAFRLDPTYLKHSHQDSGLI
TDYRHWQIPLGRRFRSLKMWFVFRMYGVKGLQAYIRKHVQLSHEFESLV
RQDPRFEICVEVILGLVCFRLKGSNKVNEALLQRINSAKKIHLVPCHLR
DKFVLRFAICSRTVESAHVQRAWEHIKELAADVLRAERE.

Functional variants of AADC, e.g., those retaining at least 90% or at least 95% sequence identity to SEQ ID NO: 978, may also be used. Codon-optimized and other variants that encode the same or essentially the same AADC amino acid sequence (e.g., those having at least 90%, at least 95%, at least 96%, at least 97%, at least 98%, or at least 99% amino acid sequence identity) may also be used. A functional variant is a variant that retains some or all of the activity of its wild-type counterpart (e.g., SEQ ID NO: 978), so as to achieve a desired therapeutic effect. For example, in some embodiments, a functional variant is effective to be used in gene therapy to treat a disorder or condition. In a non-limiting example, a functional variant (e.g., of AADC, e.g., of SEQ ID NO: 978) may have the ability to catalyze the decarboxylation of L-3,4-dihydroxyphenylalanine (L-DOPA or levodopa) to dopamine; L-5-hydroxytryptophan to serotonin; and/or L-tryptophan to tryptamine. In some embodiments, a functional variant is effective to be used in gene therapy to treat Parkinson's Disease. In some embodiments, a functional variant is effective to be used in gene therapy to treat AADC deficiency. Unless indicated otherwise, a variant of AADC as described herein (e.g., in the context of the constructs, vectors, genomes, methods, kits, compositions, etc. of the disclosure) is a functional variant.

The AADC polynucleotides of the disclosure, may be engineered to contain modular elements and/or sequence motifs assembled to create AADC polynucleotide constructs.

According to the present disclosure, AADC polynucleotides are provided. Such polynucleotides comprise nucleic acid polymers which comprise a region of linked nucleosides encoding one or more isoforms or variants of the AADC protein.

In some embodiments, the AADC polynucleotide comprises a codon optimized transcript encoding an AADC protein.

In some embodiments, the AADC polynucleotide comprises a sequence region encoding one or more wild type isoforms or variants of an AADC protein, e.g., encoding SEQ ID NO: 978. Such polynucleotides may also comprise a sequence region encoding any one or more of the following: a 5′ ITR, a cytomegalovirus (CMV) Enhancer, a CMV Promoter, an ie1 exon 1, an ie1 intron1, an hbBglobin intron2, an hBglobin exon 3, a 5′ UTR, a 3′ UTR, an hGH poly(A) signal, and/or a 3′ ITR. Such sequence regions are taught herein or may be any of those known in the art. In some embodiments, such a polynucleotide sequence comprises SEQ ID NO: 979.

In some embodiments, a suitable AADC polynucleotide, e.g., in a payload construct and/or payload region of the present disclosure may be described by International Patent Publication WO2016073693 or US20190358306A1, the contents of which are herein incorporated by reference in their entirety.

In some embodiments, a suitable AADC polynucleotide, e.g., in a payload construct and/or payload region of the present disclosure may be described by International Patent Publication WO2018232055 or US20190343937A1, the contents of which are herein incorporated by reference in their entirety.

In some embodiments, an AADC polynucleotide, e.g., in a payload construct and/or payload region of the present disclosure, comprises the following regions or sequences with 90% identity or greater (e.g., at least 95% identity) to those listed below:

TABLE 1

| Region | Start | Stop | Length of Region | SEQ ID NO of region |
|---|---|---|---|---|
| 5′ ITR | 1 | 141 | 141 | 980 |
| MCS | 189 | 206 | 18 | 981 |
| CMV enhancer | 213 | 515 | 303 | 982 |
| CMV promoter | 516 | 719 | 204 | 983 |
| Ie1 exon 1 | 734 | 867 | 134 | 984 |
| Ie1 intron partial | 868 | 899 | 32 | 985 |
| hBglobin intron 2 | 900 | 1246 | 347 | 986 |
| hBglobin exon 3 | 1247 | 1299 | 53 | 987 |
| A ADC | 1338 | 2777 | 1440 | 988 |
| MCS | 2820 | 2837 | 18 | 989 |
| Poly(A) | 2838 | 3314 | 477 | 990 |
| 3′ ITR | 3386 | 3526 | 141 | 991 |

In some embodiments, the AADC polynucleotide comprises SEQ ID NO: 979 or a fragment or variant thereof. This AADC polynucleotide sequence is given here:

(SEQ ID NO: 979)
5’cctgcaggcagctgcgcgctcgctcgctcactgaggccgcccgggcaaagcccgggcgtcgggcgacctttggt
cgcccggcctcagtgagcgagcgagcgcgcagagagggagtggccaactccatcactaggggttccttgtagttaatgattaacccg
ccatgctacttatctacgtagccatgcgtcgacataacgcgtatatctagacgttacataacttacggtaaatggcccgcctggctgacc
gcccaacgacccccgcccattgacgtcaataatgacgtatgttcccatagtaacgccaatagggactttccattgacgtcaatgggtgg
agtatttacggtaaactgcccacttggcagtacatcaagtgtatcatatgccaagtacgccccctattgacgtcaatgacggtaaatggc
ccgcctggcattatgcccagtacatgaccttatgggactttcctacttggcagtacatctagtattagtcatcgctattaccatggtgatgcg
gttttggcagtacatcaatgggcgtggatagcggtttgactcacggggatttccaagtctccaccccattgacgtcaatgggagtttgtttt
ggcaccaaaatcaacgggactttccaaaatgtcgtaacaactccgccccattgacgcaaatgggcggtaggcgtgtacggtgggagg
tctatataagcagagctcgtttagtgaaccgtcagatcgcctggagacgccatccacgctgttttgacctccatagaagacaccgggac
cgatccagcctccgcggattcgaatcccggccgggaacggtgcattggaacgcggattccccgtgccaagagtgacgtaagtaccg
cctatagagtctataggcccacaaaaaatgctttcttcttttaatatacttttttgtttatcttatttctaatactttccctaatctctttctttca
gggcaataatgatacaatgtatcatgcctctttgcaccattctaaagaataacagtgataatttctgggttaaggcaatagcaatatttctgcatat
aaatatttctgcatataaattgtaactgatgtaagaggtttcatattgctaatagcagctacaatccagctaccattctgcttttattttatggttg
ggataaggctggattattctgagtccaagctaggcccttttgctaatcatgttcatacctcttatcttcctcccacagctcctgggcaacgtg
ctggtctgtgtgctggcccatcactttggcaaagaattgggattcgaacatcgattgaattccccggggatccaccatgaacgcaagtg
aattccgaaggagagggaaggagatggtggattacgtggccaactacatggaaggcattgagggacgccaggtctaccctgacgtg
gagcccgggtacctgcggccgctgatccctgccgctgcccctcaggagccagacacgtttgaggacatcatcaacgacgttgagaa
gataatcatgcctggggtgacgcactggcacagcccctacttcttcgcctacttccccactgccagctcgtacccggccatgcttgcgg
acatgctgtgcggggccattggctgcatcggcttctcctgggcggcaagcccagcatgcacagagctggagactgtgatgatggact
ggctcgggaagatgctggaactaccaaaggcatttttgaatgagaaagctggagaagggggaggagtgatccagggaagtgccagt
gaagccaccctggtggccctgctggccgctcggaccaaagtgatccatcggctgcaggcagcgtccccagagctcacacaggccg
ctatcatggagaagctggtggcttactcatccgatcaggcacactcctcagtggaaagagctgggttaattggtggagtgaaattaaaa
gccatcccctcagatggcaacttcgccatgcgtgcgtctgccctgcaggaagccctggagagagacaaagcggctggcctgattcct
ttctttatggttgccaccctggggaccacaacatgctgctcctttgacaatctcttagaagtcggtcctatctgcaacaaggaagacatatg
gctgcacgttgatgcagcctacgcaggcagtgcattcatctgccctgagttccggcaccttctgaatggagtggagtttgcagattcatt
caactttaatccccacaaatggctattggtgaattttgactgttctgccatgtgggtgaaaaagagaacagacttaacgggagcctttaga
ctggaccccacttacctgaagcacagccatcaggattcagggcttatcactgactaccggcattggcagataccactgggcagaagat
ttcgctctttgaaaatgtggtttgtatttaggatgtatggagtcaaaggactgcaggcttatatccgcaagcatgtccagctgtcccatgag
tttgagtcactggtgcgccaggatccccgctttgaaatctgtgtggaagtcattctggggcttgtctgctttcggctaaagggttccaaca
aagtgaatgaagctcttctgcaaagaataaacagtgccaaaaaaatccacttggttccatgtcacctcagggacaagtttgtcctgcgctt
tgccatctgttctcgcacggtggaatctgcccatgtgcagcgggcctgggaacacatcaaagagctggcggccgacgtgctgcgagc
agagagggagtaggagtgaagccaggacctgcagaagcttgcctcgagcagcgctgctcgagagatctacgggtggcatccctgt
gacccctccccagtgcctctcctggccctggaagttgccactccagtgcccaccagccttgtcctaataaaattaagttgcatcattttgtc
tgactaggtgtccttctataatattatggggtggaggggggtggtatggagcaaggggcaagttgggaagacaacctgtagggcctgc
ggggtctattgggaaccaagctggagtgcagtggcacaatcttggctcactgcaatctccgcctcctgggttcaagcgattctcctgcct
cagcctcccgagttgttgggattccaggcatgcatgaccaggctcagctaatttttgtttttttggtagagacggggtttcaccatattggc
caggctggtctccaactcctaatctcaggtgatctacccaccttggcctcccaaattgctgggattacaggcgtgaaccactgctcccttc
cctgtccttactagatttaaatatgtcgtgcatcgatgctacgtagataagtagcatggcgggttaatcattaactacagaggaaccccta
gtgatggagttggccactccctctctgcgcgctcgctcgctcactgaggccgggcgaccaaaggtcgcccgacgcccgggctttgcc
cgggcggcctcagtgagcgagcgagcgcgcagctgcctgcagg3’.

In some embodiments, an AADC polynucleotide that comprises SEQ ID NO: 979 or a fragment or variant thereof is part of an AAV particle comprising an AAV2 capsid serotype. For example, the AAV particle may comprise wild-type AAV2 capsid.

In some embodiments, the AAV2 capsid is encoded by nucleic acid sequence SEQ ID NO: 1778. This nucleic sequence encoding the AAV2 capsid is given here:

(SEQ ID NO: 1778)
5’ctggctgccgacggttatctacccgattggctcgaggacactctctctgaaggaataagacagtggtggaagctca
aacctggcccaccaccaccaaagcccgcagagcggcataaggacgacagcaggggtcttgtgcttcctgggtacaagtacctcgga
cccttcaacggactcgacaagggagagccggtcaacgaggcagacgccgcggccctcgagcacgacaaagcctacgaccggca
gctcgacagcggagacaacccgtacctcaagtacaaccacgccgacgcggagtttcaggagcgccttaaagaagatacctcttttgg
gggcaacctcggacgagcagtcttccaggcgaaaaagagggttcttgaacctctgggcctggttgaggaacctgttaagacggctcc
gggaaaaaagaggccggtagagcactctcctgtggagccagactcctcctcgggaaccggaaaggcgggccagcagcctgcaag
aaaaagattgaattttggtcagactggagacgcagactcagtacctgacccccagcctctcggacagccaccagcagccccctctggt
ctgggaactaatacgatggctacaggcagtggcgcaccaatggcagacaataacgagggcgccgacggagtgggtaattcctcgg
gaaattggcattgcgattccacatggatgggcgacagagtcatcaccaccagcacccgaacctgggccctgcccacctacaacaacc
acctctacaaacaaatttccagccaatcaggagcctcgaacgacaatcactactttggctacagcaccccttgggggtattttgacttcaa
cagattccactgccacttttcaccacgtgactggcaaagactcatcaacaacaactggggattccgacccaagagactcaacttcaagc
tctttaacattcaagtcaaagaggtcacgcagaatgacggtacgacgacgattgccaataaccttaccagcacggttcaggtgtttactg
actcggagtaccagctcccgtacgtcctcggctcggcgcatcaaggatgcctcccgccgttcccagcagacgtcttcatggtgccaca
gtatggatacctcaccctgaacaacgggagtcaggcagtaggacgctcttcattttactgcctggagtactttccttctcagatgctgcgt
accggaaacaactttaccttcagctacacttttgaggacgttcctttccacagcagctacgctcacagccagagtctggaccgtctcatg
aatcctctcatcgaccagtacctgtattacttgagcagaacaaacactccaagtggaaccaccacgcagtcaaggcttcagttttctcag
gccggagcgagtgacattcgggaccagtctaggaactggcttcctggaccctgttaccgccagcagcgagtatcaaagacatctgcg
gataacaacaacagtgaatactcgtggactggagctaccaagtaccacctcaatggcagagactctctggtgaatccgggcccggcc
atggcaagccacaaggacgatgaagaaaagttttttcctcagagcggggttctcatctttgggaagcaaggctcagagaaaacaaatg
tggacattgaaaaggtcatgattacagacgaagaggaaatcaggacaaccaatcccgtggctacggagcagtatggttctgtatctac
caacctccagagaggcaacagacaagcagctaccgcagatgtcaacacacaaggcgttcttccaggcatggtctggcaggacaga
gatgtgtaccttcaggggcccatctgggcaaagattccacacacggacggacattttcacccctctcccctcatgggtggattcggactt
aaacaccctcctccacagattctcatcaagaacaccccggtacctgcgaatccttcgaccaccttcagtgcggcaaagtttgcttccttc
atcacacagtactccacgggacaggtcagcgtggagatcgagtgggagctgcagaaggaaaacagcaaacgctggaatcccgaaa
ttcagtacacttccaactacaacaagtctgttaatgtggactttactgtggacactaatggcgtgtattcagagcctcgccccattggca
ccagatacctgactcgtaatctgtaa 3’.

In some embodiments, the AAV2 capsid comprises amino acid sequence SEQ ID NO: 16. This AAV2 capsid amino acid sequence is given here:

(SEQ ID NO: 16)
XAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNR
QAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWEL
QKENSKRWNPEIQYTSNYNKSVNVDFTVDTNGVYSEPRPIGTRYLTRN
L, where X is M, L, or absent.

In some embodiments, the first amino acid of SEQ ID NO: 16 is a methionine. In some embodiments, the first amino acid of SEQ ID NO: 16 is a leucine. In some embodiments, the first amino acid of SEQ ID NO: 16 is post-translationally cleaved. In some embodiments, AAVs of the present disclosure comprise a mixed population of AAV2 capsids of SEQ ID NO: 16, in which the first amino acid may be methionine, leucine, or absent.

In certain embodiments, an AADC polynucleotide comprises a ribonucleotide form of SEQ ID NO: 979.

In certain embodiments, the AADC polynucleotide comprises a sequence which has a percent identity to any of SEQ ID NO: 979 or a fragment or variant thereof. The AADC polynucleotide may have 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% identity to any of SEQ ID NO: 979 or a fragment or variant thereof. The AADC polynucleotide may have 1-10%, 10-20%, 30-40%, 50-60%, 50-70%, 50-80%, 50-90%, 50-99%, 50-100%, 60-70%, 60-80%, 60-90%, 60-99%, 60-100%, 70-80%, 70-90%, 70-99%, 70-100%, 80-85%, 80-90%, 80-95%, 80-99%, 80-100%, 90-95%, 90-99%, or 90-100% to any of SEQ ID NO: 979 or a fragment or variant thereof. In certain embodiments, the AADC polynucleotide comprises a sequence which has 80% identity to any of SEQ ID NO: 979 or a fragment or variant thereof. In certain embodiments, the AADC polynucleotide comprises a sequence which has 85% identity to any of SEQ ID NO: 979 or a fragment or variant thereof. In certain embodiments, the AADC polynucleotide comprises a sequence which has 90% identity to any of SEQ ID NO: 979 or a fragment or variant thereof. In certain embodiments, the AADC polynucleotide comprises a sequence which has 95% identity to any of SEQ ID NO: 979 or a fragment or variant thereof. In certain embodiments, the AADC polynucleotide comprises a sequence which has 96% identity to any of SEQ ID NO: 979 or a fragment or variant thereof. In certain embodiments, the AADC polynucleotide comprises a sequence which has 97% identity to any of SEQ ID NO: 979 or a fragment or variant thereof. In certain embodiments, the AADC polynucleotide comprises a sequence which has 98% identity to any of SEQ ID NO: 979 or a fragment or variant thereof. In certain embodiments, the AADC polynucleotide comprises a sequence which has 99% identity to any of SEQ ID NO: 979 or a fragment or variant thereof. In certain embodiments, the AADC polynucleotide comprises a sequence which has at least 95% identity to SEQ ID NO: 979 and encodes SEQ ID NO: 978.

In some embodiments, the coding region of the AADC polynucleotide is 1440 nucleotides in length. Such an AADC polynucleotide may, for example, be codon optimized over all or a portion of the polynucleotide.

In some embodiments, the AADC polynucleotide comprises any of SEQ ID NO: 979 or a fragment or variant thereof but lacking the 5′ and/or 3′ ITRs. Such a polynucleotide may be incorporated into a plasmid or vector and utilized to express the encoded AADC protein.

In certain embodiments, the AADC polynucleotides may be produced in insect cells (e.g., Sf9 cells).

In certain embodiments, the AADC polynucleotide may comprise an open reading frame of an AADC mRNA, for example, a codon optimized open reading frame of an AADC mRNA, at least one 5′ITR and at least one 3′ITR where the one or more of the 5′ITRs may be located at the 5′end of the promoter region and one or more 3′ ITRs may be located at the 3′ end of the poly(A) signal. The AADC mRNA may comprise a promoter region, a 5′ untranslated region (UTR), a 3′UTR and a poly(A) signal. The promoter region may include, but is not limited to, enhancer element, a promoter element, and an intron region. In certain embodiments, the enhancer element and the promoter element are derived from CMV. In certain embodiments, the intron region comprises ie1 exon 1 or a fragment thereof, ie1 intron 1 or a fragment thereof, hBglobin intron 2 or a fragment thereof, and hBglobin exon 3 or a fragment thereof. As yet another non-limiting example, the poly(A) signal is derived from human growth hormone.

In certain embodiments, at least one element may be used with the AADC polynucleotides described herein to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (Poly(A)) signal sequences and upstream enhancers (USEs), CMV enhancers and introns.

In certain embodiments, at least one element may be used with the AADC polynucleotides described herein to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety) such as promoters.

In certain embodiments, the AADC polynucleotide is encoded in a plasmid or vector, which may be derived from an adeno-associated virus (AAV).

In certain embodiments, the AAV particle of the disclosure comprises a recombinant AAV2 with a viral genome encoding a human AADC.

In certain embodiments, the AAV particle of the disclosure has a CAS (Chemical Abstracts Service) Registry Number of 2226647-27-2.

### AAV Serotypes

AAV particles of the present disclosure may comprise or be derived from a nucleic acid sequence encoding any natural or recombinant AAV serotype. In some embodiments, the preferred AAV serotype is AAV2. In some embodiments, an AAV particle of AAV2 serotype comprises an AAV2 capsid. According to the present disclosure, the AAV particles may utilize or be based on a serotype selected from any of the following PHP.B, PHP.A, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, ovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, PHP.B (AAV-PHP.B), PHP.A (AAV.PHP.A), G2B-26, G2B-13, TH1.1-32, TH1.1-35, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3, AAVG2B4, and/or AAVG2B5, and variants thereof.

In some embodiments, an AAV particle of the present disclosure may comprise a capsid protein having the amino acid sequence, or encoded by a nucleotide sequence, of any one of SEQ ID NOs: 1-875, 992-1374, and 1775-1777, or a functional variant thereof, e.g., a capsid protein variant comprising a sequence substantially identical to, and capable of at least one activity of, a wild-type capsid protein.

In some exemplary embodiments, a modified AAV2 serotype or nucleic acid encoding the serotype (e.g, a codon optimized nucleic acid) may be used. In some embodiments, a nucleic acid encoding an AAV2 serotype is modified to remove one or more false translation start sites. In some embodiments, a nucleic acid encoding an AAV2 serotype is modified to comprise a suboptimal start codon. In some embodiments, the nucleic acid sequence encoding the modified AAV2 serotype comprises SEQ ID NO: 1778. In certain embodiments, the AAV serotype comprises a capsid nucleic acid sequence of SEQ ID NO: 15. In certain embodiments, the AAV serotype comprises a capsid nucleic acid sequence of SEQ ID NO: 1778. In some embodiments, the AAV serotype comprises a capsid amino acid sequence of SEQ ID NO: 16. In some embodiments, the AAV serotype may be, or have, a mutation over a wild type or naturally-occurring sequence, e.g., as described in U.S. Pat. No. 9,546,112, the contents of which are herein incorporated by reference in their entirety.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; 0 (Pyl) for Pyrrolysine; U (Sec) for Selenocysteine; X (Xaa) for any amino acid; and Z (Glx) for Glutamine or Glutamic acid.

In certain embodiments, the AAV serotype may be, or may comprise a sequence as described in International Patent Publication WO2015038958, the contents of which are herein incorporated by reference in their entirety, such as, but not limited to, AAV9 (SEQ ID NO: 2 and 11 of WO2015038958 or SEQ ID NO: 132 and 131 respectively herein), PHP.B (SEQ ID NO: 8 and 9 of WO2015038958 or SEQ ID NO: 1 and 2 herein), G2B-13 (SEQ ID NO: 12 of WO2015038958 or SEQ ID NO: 3 herein), G2B-26 (SEQ ID NO: 13 of WO2015038958 or SEQ ID NO: 1 herein), TH1.1-32 (SEQ ID NO: 14 of WO2015038958 or SEQ ID NO: 4 herein), TH1.1-35 (SEQ ID NO: 15 of WO2015038958 or SEQ ID NO: 5 herein) or variants thereof. Further, any of the targeting peptides or amino acid inserts described in WO2015038958, may be inserted into any parent AAV serotype, such as, but not limited to, AAV9 (SEQ ID NO: 131 for the DNA sequence and SEQ ID NO: 132 for the amino acid sequence). In certain embodiments, the amino acid insert is inserted between amino acids 586-592 of the parent AAV (e.g., AAV9). In another embodiment, the amino acid insert is inserted between amino acids 588-589 of the parent AAV sequence.

In certain embodiments, the AAV serotype may be, or may have a sequence as described in International Patent Publication WO2017100671, the contents of which are herein incorporated by reference in their entirety, such as, but not limited to, AAV9 (SEQ ID NO: 45 of WO2017100671, herein SEQ ID NO: 875), PHP.N (SEQ ID NO: 46 of WO2017100671, herein SEQ ID NO: 873), PHP.S (SEQ ID NO: 47 of WO2017100671, herein SEQ ID NO: 874), or variants thereof. Further, any of the targeting peptides or amino acid inserts described in WO2017100671 may be inserted into any parent AAV serotype, such as, but not limited to, AAV9 (SEQ ID NO: 127 or SEQ ID NO: 875). In certain embodiments, the amino acid insert is inserted between amino acids 586-592 of the parent AAV (e.g., AAV9). In another embodiment, the amino acid insert is inserted between amino acids 588-589 of the parent AAV sequence.

In certain embodiments, the AAV serotype may be, or may have a sequence as described in U.S. Pat. No. 9,624,274, the contents of which are herein incorporated by reference in their entirety, such as, but not limited to, AAV2 (SEQ ID NO: 183 of U.S. Pat. No. 9,624,274), Further, any of the structural protein inserts described in U.S. Pat. No. 9,624,274, may be inserted into, but not limited to, I-453 and I-587 of any parent AAV serotype, such as, but not limited to, AAV2 (SEQ ID NO: 183 of U.S. Pat. No. 9,624,274).

In certain embodiments, the AAV serotype may be, or may have a sequence as described in U.S. Pat. No. 9,475,845, the contents of which are herein incorporated by reference in their entirety, such as, but not limited to, AAV capsid proteins comprising modification of one or more amino acids at amino acid positions 585 to 590 of the native AAV2 capsid protein. In certain embodiments, the amino acid modification is a substitution at amino acid positions 262 through 265 in the native AAV2 capsid protein or the corresponding position in the capsid protein of another AAV with a targeting sequence.

In certain embodiments, the AAV serotype may be, or may comprise a sequence as described in United States Publication No. US 20160369298, the contents of which are herein incorporated by reference in their entirety, such as, but not limited to, site-specific mutated capsid protein of AAV2 (SEQ ID NO: 97 of US 20160369298; herein SEQ ID NO: 1560) or variants thereof, wherein the specific site is at least one site selected from sites R447, G453, S578, N587, N587+1, S662 of VP1 or fragment thereof.

In some embodiments, the AAV serotype may be modified as described in the United States Publication US 20170145405 the contents of which are herein incorporated by reference in their entirety. AAV serotypes may include, but is not limited to, modified AAV2 (e.g., modifications at Y444F, Y500F, Y730F and/or S662V).

In some embodiments, the AAV serotype may be modified as described in the International Publication WO2017083722 the contents of which are herein incorporated by reference in their entirety. AAV serotypes may include, but are not limited to, AAV2 (Y444+500+730F+T491V).

In some embodiments, the AAV serotype is PHP.N. In certain embodiments, the AAV serotype is a serotype comprising the AAVPHP.N (PHP.N) peptide, or a variant thereof. In certain embodiments the AAV serotypes is a serotype comprising the AAVPHP.B (PHP.B) peptide, or a variant thereof. In certain embodiments, the AAV serotype is a serotype comprising the AAVPHP.A (PHP.A) peptide, or a variant thereof. In certain embodiments, the AAV serotype is a serotype comprising the PHP.S peptide, or a variant thereof. In certain embodiments, the AAV serotype is a serotype comprising the PHP.B2 peptide, or a variant thereof. In certain embodiments, the AAV serotype is a serotype comprising the PHP.B3 peptide, or a variant thereof. In certain embodiments, the AAV serotype is a serotype comprising the G2B4 peptide, or a variant thereof. In certain embodiments, the AAV serotype is a serotype comprising the G2B5 peptide, or a variant thereof. In certain embodiments the AAV capsid is one that allows for blood brain barrier penetration following intravenous administration.

In certain embodiments, the AAV serotype may comprise a capsid amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above. In certain embodiments, the AAV serotype comprises a capsid amino acid sequence at least 95% identical to SEQ ID NO: 16. In certain embodiments, the AAV serotype comprises a capsid amino acid sequence at least 99% identical to SEQ ID NO: 16. In certain embodiments, the AAV serotype comprises a capsid amino acid of SEQ ID NO: 16.

In certain embodiments, the AAV serotype may comprise a capsid nucleic acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above. In certain embodiments, the AAV serotype comprises a capsid nucleic acid sequence at least 90% identical to SEQ ID NO: 15. In certain embodiments, the AAV serotype comprises a capsid nucleic acid sequence at least 95% identical to SEQ ID NO: 15. In certain embodiments, the AAV serotype comprises a capsid nucleic acid sequence at least 99% identical to SEQ ID NO: 15. In certain embodiments, the AAV serotype comprises a capsid nucleic acid sequence of SEQ ID NO: 15. In certain embodiments, the AAV serotype comprises a capsid nucleic acid sequence of SEQ ID NO: 1778.

In some embodiments, the AAV serotype comprises AAV2. In some embodiments, the AAV serotype is AAV2. In certain embodiments, the AAV serotype comprises a capsid nucleic acid sequence of SEQ ID NO: 1778. In some embodiments, the AAV serotype comprises a capsid amino acid sequence of SEQ ID NO: 16.

In certain embodiments, the initiation codon for translation of the AAV VP1 capsid protein may be CTG, TTG, or GTG as described in U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins. In some embodiments, the AAV particles described herein comprise an AAV2 capsid wherein a first amino acid residue of VP1 has been clipped.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in its entirety.

According to the present invention, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

In certain embodiments, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1−clipping (Met−/AA1−), and combinations thereof (Met+/AA1+ and Met−/AA1−).

In certain embodiments, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Payloads

AAV particles of the present disclosure can comprise, or be produced using, at least one payload construct which comprises at least one payload region. In certain embodiments, the payload region may be located within a viral genome, such as the viral genome of a payload construct. At the 5′ and/or the 3′ end of the payload region there may be at least one inverted terminal repeat (ITR). Within the payload region, there may be additional elements as discussed above, e.g., a promoter region, an intron region, and a coding region.

In certain embodiments, a payload construct of the present disclosure can be a bacmid, also known as a baculovirus plasmid or recombinant baculovirus genome. As used herein, “payloadBac” refers to a baculovirus comprising a payload construct and/or region, e.g., a payload construct and/or region encoding AADC or a functional variant thereof. Viral production cells (e.g., Sf9 cells) may be transfected with payloadBacs and/or with BIICs comprising payloadBacs.

In certain embodiments, the payload region of the AAV particle comprises one or more nucleic acid sequences encoding a polypeptide or protein of interest.

In certain embodiments, the AAV particle comprises a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the vector genome may express each of the one or more polypeptides in the single target cell.

Where the AAV particle payload region encodes a polypeptide, the polypeptide may be a peptide, polypeptide or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which comprise the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the AAV particle comprises a viral genome with a payload region comprising a nucleic acid sequence encoding a protein of interest (i.e., a payload protein, therapeutic protein, e.g., AADC or a functional variant thereof).

In certain embodiments, the payload region comprises a nucleic acid sequence encoding AADC (e.g., SEQ ID NO: 978). In certain embodiments, the payload region comprises SEQ ID NO: 979.

In certain embodiments, the payload region comprises a nucleic acid sequence a therapeutic protein, an enzyme, an antibody or antigen-binding fragment thereof, a protein ligand, or a soluble receptor. In certain embodiments, the payload region comprises a nucleic acid sequence encoding a modulatory polynucleotide which interferes with a target gene expression and/or a target protein production. In certain embodiments, the modulatory polynucleotide is an antisense strand, a miRNA molecule, or a siRNA molecule.

In certain embodiments, the payload region comprises a nucleic acid sequence encoding a protein comprising but not limited to an antibody or antigen binding fragment thereof, an enzyme, ApoE2, Frataxin, survival motor neuron (SMN) protein, glucocerebrosidase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), MeCP2, beta-galactosidase (GLB1) and/or gigaxonin (GAN).

In certain embodiments, the payload region comprises a nucleic acid sequence encoding a modulatory polynucleotide which interferes with a target gene expression and/or a target protein production, e.g., an antisense or a siRNA molecule. In certain embodiments, the gene expression or protein production to be inhibited/modified may comprise but are not limited to superoxide dismutase 1 (SOD1), chromosome 9 open reading frame 72 (C90RF72), TAR DNA binding protein (TARDBP), ataxin-3 (ATXN3), huntingtin (HTT), amyloid precursor protein (APP), apolipoprotein E (ApoE), microtubule-associated protein tau (MAPT), alpha-synuclein (SNCA), voltage-gated sodium channel alpha subunit 9 (SCN9A), and/or voltage-gated sodium channel alpha subunit 10 (SCN10A).

In certain embodiments, a nucleic acid sequence encoding such siRNA molecules, or a single strand of the siRNA molecules, is inserted into adeno-associated viral vectors and introduced into cells, specifically cells in the central nervous system.

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, 1 or 2 nucleotide overhangs at the 3′end of each strand.

The payloads of the formulated AAV particles of the present disclosure may encode one or more agents which are subject to RNA interference (RNAi) induced inhibition of gene expression. In certain embodiments, the AAV particles of the present disclosure may encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory or motor neurons.

In certain embodiments, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In certain embodiments, the modulatory polynucleotide which comprises the payload (e.g., siRNA, miRNA or other RNAi agent described herein) comprises molecular scaffold which comprises a leading 5′ flanking sequence which may be of any length and may be derived in whole or in part from wild type microRNA sequence or be completely artificial. A 3′ flanking sequence may mirror the 5′ flanking sequence in size and origin. In certain embodiments, one or both of the 5′ and 3′ flanking sequences are absent.

In certain embodiments, the molecular scaffold may comprise one or more linkers known in the art. The linkers may separate regions or one molecular scaffold from another. As a non-limiting example, the molecular scaffold may be polycistronic.

In certain embodiments, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

In certain embodiments, the present disclosure provides methods for producing an AAV for inhibiting/silencing gene expression in a cell. In certain embodiments, the AAV comprises siRNA duplexes that may be used to reduce the expression of a gene of interest and/or transcribed mRNA in at least one region of the CNS. In certain embodiments, the present disclosure provides methods for manufacturing a pharmaceutical composition comprising at least one siRNA duplex targeting the gene of interest and a pharmaceutically acceptable carrier. The term “targeting” as used in this context refers to the process of design and selection of a nucleic acid sequence that will hybridize to a target nucleic acid and induce a desired effect. In some embodiments, the siRNA duplex is encoded by a vector genome in an AAV particle.

### II. AAV Production

### General Viral Production Process

Mammalian cells and/or insect cells are often used as viral production cells for the production of rAAV particles. In various embodiments, the methods and systems disclosed herein employ insect cells, e.g., Sf9 cells, e.g., to produce AAV2 serotype particles, e.g., those comprising an AADC payload construct.

In various embodiments, the present disclosure provides methods of producing AAV particles or viral vectors by (a) contacting a viral production cell (e.g., Sf9) with one or more viral expression constructs encoding at least one AAV capsid protein and/or at least one AAV replication protein, and one or more payload construct vectors, e.g., wherein said payload construct vector comprises a payload construct encoding AADC or a functional variant thereof; (b) culturing said viral production cell under conditions such that at least one AAV particle or viral vector is produced, and (c) isolating said at least one AAV particle or viral vector. The viral expression constructs may be comprised in one or more baculovirus (expressionBac). The expressionBacs may be comprised in one or more BIIC (e.g., expressionBIICs). The payload constructs may be comprised one or more baculovirus (payloadBac). The payloadBacs may be comprised in one or more BIICs (e.g., payloadBIICs or payload BIICs).

In these methods a viral expression construct may encode at least one structural protein and/or at least one non-structural protein. The structural protein may comprise any of the native or wild type capsid proteins VP1, VP2 and/or VP3 or a chimeric protein. The non-structural protein may comprise any of the native or wild type Rep78, Rep68, Rep52 and/or Rep40 proteins or a chimeric protein, e.g., any of the construct described above.

In certain embodiments, an rAAV production method as disclosed herein comprises transient transfection, viral transduction and/or electroporation.

In certain embodiments, the viral production cell is selected from the group consisting of a mammalian cell and an insect cell. In certain embodiments, the insect cell comprises a Spodoptera frugiperda insect cell. In certain embodiments, the insect cell comprises a Sf9 insect cell. In certain embodiments, the insect cell comprises a Sf21 insect cell.

Also provided are AAV particles and viral vectors produced according to the methods described herein.

The AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell) with at least one viral expression construct encoding at least one capsid protein and at least one AAV replication protein, and at least one payload construct vector. In some embodiments, separate constructs encoding the at least one capsid protein and at least one AAV replication protein may be used. The viral production cell may be contacted by transient transfection, viral transduction and/or electroporation. The payload construct vector may comprise a payload construct encoding a payload molecule such as AADC. The viral production cell can be cultured under conditions such that at least one AAV particle or viral vector is produced, isolated (e.g., using temperature-induced lysis, mechanical lysis and/or chemical lysis) and/or purified (e.g., using filtration, chromatography and/or immunoaffinity purification).

In certain embodiments, the AAV particles are produced in an insect cell (e.g., Spodoptera frugiperda (Sf9) cell) using the method described herein. As a non-limiting example, the insect cell is contacted using viral transduction which may comprise baculoviral transduction.

In certain embodiments, the viral expression construct may encode at least one structural protein and at least one non-structural protein. As a non-limiting example, the structural protein may comprise capsid VP1, VP2 and/or VP3. As another non-limiting example, the non-structural protein may comprise Rep78, Rep68, Rep52 and/or Rep40.

In certain embodiments, the AAV particle production method described herein produces greater than 10¹, greater than 10², greater than 10³, greater than 10⁴ or greater than 10⁵ AAV particles in a viral production cell.

In certain embodiments, a process of the present disclosure comprises production of viral particles in a viral production cell using a viral production system which comprises at least one viral expression construct and at least one payload construct. The at least one viral expression construct and at least one payload construct can be co-transfected (e.g., dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, comprising Rep proteins which replicate the payload construct and Cap proteins which assemble to form a capsid that encloses the replicated payload constructs. The resulting AAV particle is extracted from the viral production cells and processed into a pharmaceutical preparation for administration.

In certain embodiments, the process for production of viral particles utilizes seed cultures of viral production cells that comprise one or more baculoviruses (e.g., a Baculoviral Expression Vector (BEV) or baculovirus infected insect cells (BIICs) that have been transfected with a viral expression construct (e.g., comprised in an expressionBac) and a payload construct (e.g., comprised in a payloadBac)). In certain embodiments, the seed cultures are harvested, divided into aliquots and frozen, and may be used at a later time point to initiate an infection of a naïve population of production cells.

Large scale production of AAV particles may utilize a bioreactor. The use of a bioreactor allows for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In another embodiment, the bioreactor is used for continuous production in which a portion of the culture is harvested at an experimentally determined time point for purification of AAV particles, and the remaining culture in the bioreactor is refreshed with additional growth media components.

AAV viral particles may be extracted from viral production cells in a process which comprises cell lysis, clarification, sterilization and purification. Cell lysis comprises any process that disrupts the structure of the viral production cell, thereby releasing AAV particles. In certain embodiments cell lysis may comprise thermal shock, chemical, or mechanical lysis methods. In some embodiments, cell lysis is done chemically. Clarification of the lysed cells can comprise the gross purification of the mixture of lysed cells, media components, and AAV particles. In certain embodiments, clarification comprises centrifugation and/or filtration, comprising but not limited to depth end, tangential flow, and/or hollow fiber filtration.

The end result of viral production is a purified collection of AAV particles which comprise two components: (1) a payload construct (e.g., a recombinant viral genome construct comprising a sequence encoding AADC or a functional variant thereof) and (2) a viral capsid.

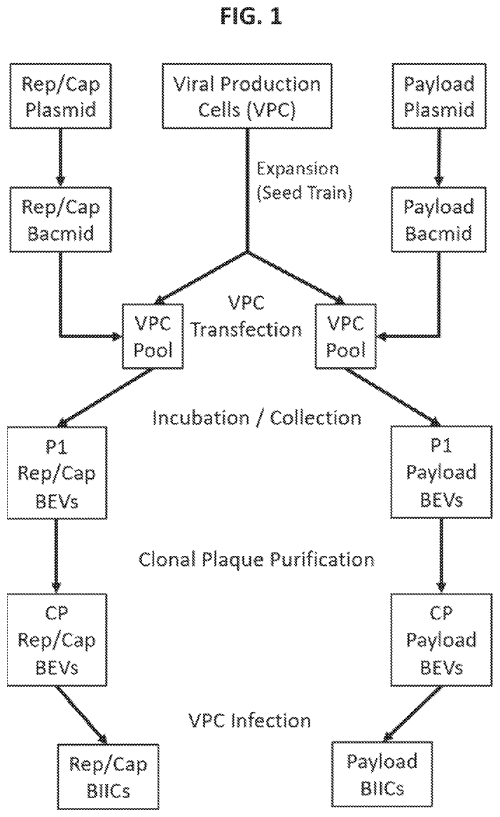

In certain embodiments, such as the embodiment presented in FIG. 1, a viral production process of the present disclosure comprises steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can comprise a single CP Purification step or can comprise multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

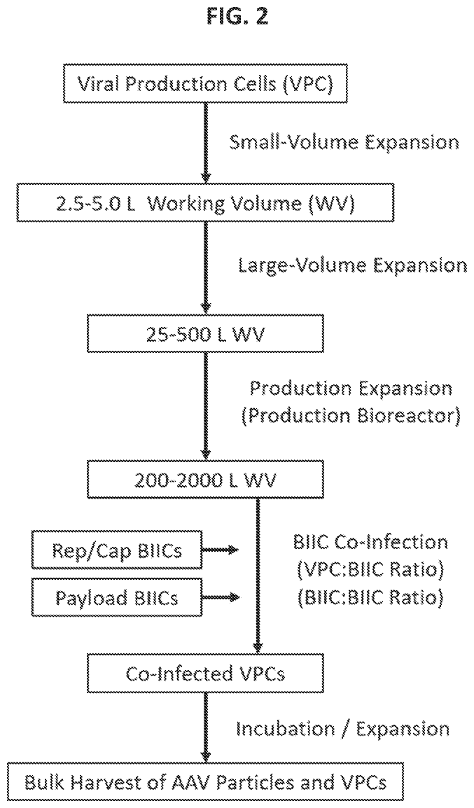

In certain embodiments, such as the embodiment presented in FIG. 2, a viral production process of the present disclosure comprises steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. The working volume of Viral Production Cells is seeded into a Production Bioreactor and can be further expanded to a working volume of 200-2000 L with a target VPC concentration for BIIC infection. The working volume of VPCs in the Production Bioreactor is then co-infected with Rep/Cap BIICs (“expressionBIICs”) and Payload BIICs (“payloadBIICs”), e.g., with a target VPC:BIIC ratio (e.g., a target VPC:expressionBIIC ratio and/or a target VPC:payloadBIIC ratio) and a target BIIC^{Rep/Cap}:BIIC^{Payload} (expressionBIIC:payloadBIIC) ratio. VCD infection can also utilize BEVs. The co-infected VPCs are incubated and expanded in the Production Bioreactor to produce a bulk harvest of AAV particles and VPCs.

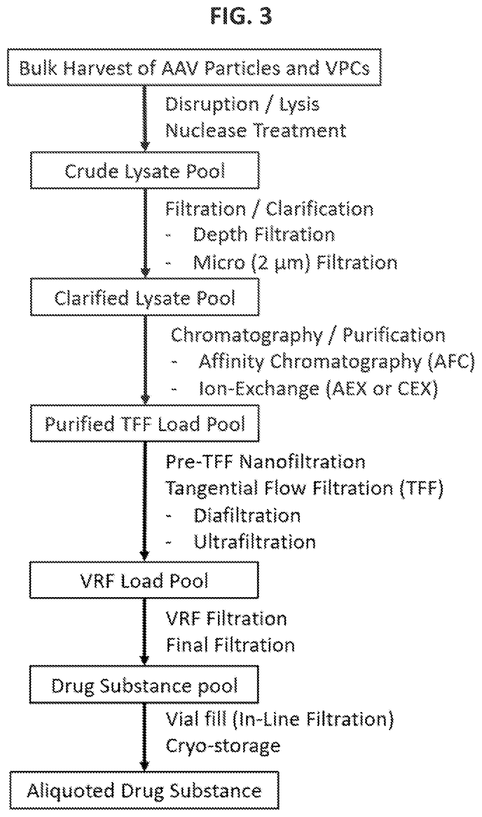

In certain embodiments, such as the embodiment presented in FIG. 3, a viral production process of the present disclosure comprises steps for producing a Drug Substance by processing, clarifying and purifying a bulk harvest of AAV particles and Viral Production Cells. A bulk harvest of AAV particles and VPCs (within a Production Bioreactor) are processed through cellular disruption and lysis (e.g., chemical lysis and/or mechanical lysis), followed by nuclease treatment of the lysis pool, thereby producing a crude lysate pool. The crude lysate pool is processed through one or more filtration and clarification steps, comprising depth filtration and microfiltration to provide a clarified lysate pool. The clarified lysate pool is processed through one or more chromatography and purification steps, comprising affinity chromatography (AFC) and ion-exchange chromatography (AEX or CEX) to provide a purified product pool. The purified product pool is then optionally processed through nanofiltration, and then through tangential flow filtration (TFF). The TFF process comprises one or more diafiltration (DF) steps and one or more ultrafiltration (UF) steps, either in series or alternating. The product pool is further processed through viral retention filtration (VRF) and another filtration step to provide a drug substance pool. The drug substance pool can be further filtered, then aliquoted into vials for storage and treatment. The drug substance may be aliquoted into vials or extractable from the vials at a volume of about 0.5-2.5 mL. For example, in some embodiments, the drug substance pool can be aliquoted at volumes of about 1.2 mL into individual vials for storage, e.g., such that about 1.0 mL of the drug substance is extractable for treatment. In some embodiments, the drug substance pool can be aliquoted at volumes of about 1.8 mL into individual vials for storage, e.g., such that about 1.6 mL of the drug substance is extractable for treatment. In some embodiments, the vials are stored at ≤65° C.

### Viral Expression Constructs

The viral production system of the present disclosure comprises one or more viral expression constructs which can be transfected/transduced into a viral production cell. In certain embodiments, a viral expression construct or a payload construct of the present disclosure can be a bacmid, also known as a baculovirus plasmid or recombinant baculovirus genome. In certain embodiments, the viral expression comprises a protein-coding nucleotide sequence and at least one expression control sequence for expression in a viral production cell. In certain embodiments, the viral expression comprises a protein-coding nucleotide sequence operably linked to least one expression control sequence for expression in a viral production cell. In certain embodiments, the viral expression construct contains parvoviral genes under control of one or more promoters. Parvoviral genes can comprise nucleotide sequences encoding non-structural AAV replication proteins, such as Rep genes which encode Rep52, Rep40, Rep68 or Rep78 proteins, e.g., a combination of Rep78 and Rep52. Parvoviral genes can comprise nucleotide sequences encoding structural AAV proteins, such as Cap genes which encode VP1, VP2 and VP3 proteins.

Viral expression constructs of the present disclosure may comprise any compound or formulation, biological or chemical, which facilitates transformation, transfection, or transduction of a cell with a nucleic acid. Exemplary biological viral expression constructs comprise plasmids, linear nucleic acid molecules, and recombinant viruses comprising baculovirus. Exemplary chemical vectors comprise lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994.); Maniatis et al., eds. Molecular Cloning. CSH Laboratory, NY, N.Y. (1982); and, Philiport and Scluber, eds. Liposomes as tools in Basic Research and Industry. CRC Press, Ann Arbor, Mich. (1995), the contents of which are each incorporated herein by reference in their entireties as related to viral expression constructs and uses thereof, insofar as they do not conflict with the present disclosure.

In certain embodiments, the viral expression construct is an AAV expression construct which comprises one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In one non-limiting example, five expression constructs may individually encode AAV VP1, AAV VP2, AAV VP3, Rep52, Rep78, and with an accompanying payload construct comprising a payload polynucleotide and at least one AAV ITR. In another embodiment, expression constructs may be employed to express, for example, Rep52 and Rep40, or Rep78 and Rep 68. Expression constructs may comprise any combination of VP1, VP2, VP3, Rep52/Rep40, and Rep78/Rep68 coding sequences.

In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may encode the components of a Parvoviral capsid with incorporated Gly-Ala repeat region, which may function as an immune invasion sequence, as described in US Patent Application 20110171262, the content of which is incorporated herein by reference in its entirety as related to Parvoviral capsid proteins, insofar as it does not conflict with the present disclosure.

In certain embodiments of the present disclosure, a viral expression construct may be used for the production of AAV particles in insect cells. In certain embodiments, modifications may be made to the wild type AAV sequences of the capsid and/or rep genes, for example to improve attributes of the viral particle, such as increased infectivity or specificity, or to enhance production yields from insect cells.

### VP-Coding Region

In certain embodiments, a viral expression construct can comprise a VP-coding region. A VP-coding region is a nucleotide sequence which comprises a VP nucleotide sequence encoding VP1, VP2, VP3, or a combination thereof. In certain embodiments, a viral expression construct can comprise a VP1-coding region; a VP1-coding region is a nucleotide sequence which comprises a VP1 nucleotide sequence encoding a VP1 protein. In certain embodiments, a viral expression construct can comprise a VP2-coding region; a VP2-coding region is a nucleotide sequence which comprises a VP2 nucleotide sequence encoding a VP2 protein. In certain embodiments, a viral expression construct can comprise a VP3-coding region; a VP3-coding region is a nucleotide sequence which comprises a VP3 nucleotide sequence encoding a VP3 protein.

In certain embodiments, a VP-coding region encodes one or more AAV capsid proteins of a specific AAV serotype, e.g., AAV2. The AAV serotypes for VP-coding regions can be the same or different. In preferred embodiments, the VP coding regions are all from AAV2. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, a nucleotide sequence encoding one or more VP capsid proteins can be codon optimized to have a nucleotide homology with the reference nucleotide sequence of less than 100%. In certain embodiments, the nucleotide homology between the codon-optimized VP nucleotide sequence and the reference VP nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

Structural VP proteins, VP1, VP2, and VP3 of a viral expression construct can be encoded in a single open reading frame regulated by utilization of both alternative splice acceptor and non-canonical translational initiation codons. VP1, VP2 and VP3 can be transcribed and translated from a single transcript in which both in-frame and/or out-of-frame start codons are engineered to control the VP1:VP2:VP3 ratio produced by the nucleotide transcript.

In certain embodiments, VP1 can be produced from a sequence which encodes for VP1 only. As use herein, the terms “only for VP1” or “VP1 only” refers to a nucleotide sequence or transcript which encodes for a VP1 capsid protein and: (i) lacks the necessary start codons within the VP1 sequence (i.e. deleted or mutated) for full transcription or translation of VP2 and VP3 from the same sequence; (ii) comprises additional codons within the VP1 sequence which prevent transcription or translation of VP2 and VP3 from the same sequence; or (iii) comprises a start codon for VP1 (e.g., ATG), such that VP1 is the primary VP protein produced by the nucleotide transcript.

In certain embodiments, VP2 can be produced from a sequence which encodes for VP2 only. As use herein, the terms “only for VP2” or “VP2 only” refers to a nucleotide sequence or transcript which encodes for a VP2 capsid protein and: (i) the nucleotide transcript is a truncated variant of a full VP capsid sequence which encodes only VP2 and VP3 capsid proteins; and (ii) which comprise a start codon for VP2 (e.g., ATG), such that VP2 is the primary VP protein produced by the nucleotide transcript.

In certain embodiments, VP1 and VP2 can be produced from a sequence which encodes for VP1 and VP2 only. As use herein, the terms “only for VP1 and VP2” or “VP1 and VP2 only” refer to a nucleotide sequence or transcript which encodes for VP1 and VP2 capsid proteins and: (i) lacks the necessary start codons within the VP sequence (i.e. deleted or mutated) for full transcription or translation of VP3 from the same sequence; (ii) comprises additional codons within the VP sequence which prevent transcription or translation of VP3 from the same sequence; (iii) comprises a start codon for VP1 (e.g., ATG) and VP2 (e.g., ATG), such that VP1 and VP2 are the primary VP protein produced by the nucleotide transcript; or (iv) comprises VP1-only nucleotide transcript and a VP2-only nucleotide transcript connected by a linker, such as an IRES region.

In certain embodiments, the viral expression construct may contain a nucleotide sequence which comprises a start codon region, such as a sequence encoding AAV capsid proteins which comprise one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG). In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the content of which is incorporated herein by reference in its entirety as related to AAV capsid proteins and the production thereof, insofar as it does not conflict with the present disclosure.

### Rep-Coding Region

In certain embodiments, a viral expression construct can comprise a Rep52-coding region. A Rep52-coding region is a nucleotide sequence which comprises a Rep52 nucleotide sequence encoding a Rep52 protein. In certain embodiments, a viral expression construct can comprise a Rep78-coding region. A Rep78-coding region is a nucleotide sequence which comprises a Rep78 nucleotide sequence encoding a Rep78 protein. In certain embodiments, a viral expression construct can comprise a Rep40-coding region. A Rep40-coding region is a nucleotide sequence which comprises a Rep40 nucleotide sequence encoding a Rep40 protein. In certain embodiments, a viral expression construct can comprise a Rep68-coding region. A Rep68-coding region is a nucleotide sequence which comprises a Rep68 nucleotide sequence encoding a Rep68 protein.

In some embodiments, non-structural proteins, Rep52 and Rep78, of a viral expression construct can be encoded in a single open reading frame regulated by utilization of both alternative splice acceptor and non-canonical translational initiation codons.

Both Rep78 and Rep52 can be translated from a single transcript: Rep78 translation initiates at a first start codon (ATG or non-ATG) and Rep52 translation initiates from a Rep52 start codon (e.g., ATG) within the Rep78 sequence. Rep78 and Rep52 can also be translated from separate transcripts with independent start codons. The Rep52 initiation codons within the Rep78 sequence can be mutated, modified or removed, such that processing of the modified Rep78 sequence will not produce Rep52 proteins.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG, and GTG, that effects partial exon skipping upon expression in insect cells, as described in U.S. Pat. No. 8,512,981, the content of which is incorporated herein by reference in its entirety as related to the promotion of less abundant expression of Rep78 as compared to Rep52 to promote high vector yields, insofar as it does not conflict with the present disclosure.

In certain embodiments, the viral expression construct may be a plasmid vector or a baculoviral construct for the expression in insect cells that contains repeating codons with differential codon biases, for example to achieve improved ratios of Rep proteins, e.g., Rep78 and Rep52 thereby improving large scale (commercial) production of viral expression construct and/or payload construct vectors in insect cells, as taught in U.S. Pat. No. 8,697,417, the content of which is incorporated herein by reference in its entirety as related to AAV replication proteins and the production thereof, insofar as it does not conflict with the present disclosure.

In certain embodiment, improved ratios of rep proteins may be achieved using the method and constructs described in U.S. Pat. No. 8,642,314, the content of which is incorporated herein by reference in its entirety as related to AAV replications proteins and the production thereof, insofar as it does not conflict with the present disclosure.

In certain embodiments, the viral expression construct may encode mutant parvoviral Rep polypeptides which have one or more improved properties as compared with their corresponding wild-type Rep polypeptide, such as the preparation of higher virus titers for large scale production. Alternatively, they may be able to allow the production of better-quality viral particles or sustain more stable production of virus. In a non-limiting example, the viral expression construct may encode mutant Rep polypeptides with a mutated nuclear localization sequence or zinc finger domain, as described in Patent Application US 20130023034, the content of which is incorporated herein by reference in its entirety as related to AAV replications proteins and the production thereof, insofar as it does not conflict with the present disclosure.

### REN Access Points and Polynucleotide Inserts

In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure (e.g., bacmid) can comprise a polynucleotide incorporated by homologous recombination (transposon donor/acceptor system) into the bacmid by standard molecular biology techniques known and performed by a person skilled in the art.

In certain embodiments, the polynucleotide incorporated into the bacmid (i.e. polynucleotide insert) can comprise an expression control sequence operably linked to a protein-coding nucleotide sequence. In certain embodiments, the polynucleotide incorporated into the bacmid can comprise an expression control sequence which comprises a promoter, such as p10 or polH, and which is operably linked to a nucleotide sequence which encodes a structural AAV capsid protein (e.g., VP1, VP2, VP3 or a combination thereof). In certain embodiments, the nucleotide sequence encoding a structural AAV capsid protein (e.g., VP1, VP2, VP3, or a combination thereof) is operably linked to a p10 promoter. In certain embodiments, the polynucleotide incorporated into the bacmid can comprise an expression control sequence which comprises a promoter, such as p10 or polH, and which is operably linked to a nucleotide sequence which encodes a non-structural AAV capsid protein (e.g., Rep78, Rep52, or a combination thereof). In certain embodiments, the nucleotide sequencing encoding a non-structural AAV protein (e.g., Rep 78, Rep 52, or a combination thereof) is operably linked to a polH promoter. In certain embodiments, the nucleotide sequence encoding a structural AAV capsid protein (e.g., VP1, VP2, VP3, or a combination thereof) is operably linked to a p10 promoter and the nucleotide sequencing encoding a non-structural AAV protein (e.g., Rep 78, Rep 52, or a combination thereof) is operably linked to a polH promoter.

In certain embodiments, the polynucleotide insert can be incorporated into the bacmid at the location of a baculoviral gene. In certain embodiments, the polynucleotide insert can be incorporated into the bacmid at the location of a non-essential baculoviral gene. In certain embodiments, the polynucleotide insert can be incorporated into the bacmid by replacing a baculoviral gene or a portion of the baculoviral gene with the polynucleotide insert. In certain embodiments, the polynucleotide insert can be incorporated into the bacmid by replacing a baculoviral gene or a portion of the baculoviral gene with a fusion-polynucleotide which comprises the polynucleotide insert and the baculoviral gene (or portion thereof) being replaced.

In certain embodiments, the polynucleotide insert can be incorporated into the bacmid by splitting a baculoviral gene with the polynucleotide insert (i.e., the polynucleotide insert is incorporated into the middle of the gene, separating a 5′-portion of the gene from a 3′-portion of the bacmid gene). In certain embodiments, the polynucleotide insert can be incorporated into the bacmid by splitting a baculoviral gene with the fusion-polynucleotide which comprises the polynucleotide insert and a portion of the baculoviral gene which was split. In certain embodiments, the 3′ end of the fusion-polynucleotide comprises the 5′-portion of the gene that was split, such that the 5′-portion of the gene in the fusion-polynucleotide and the 3′-portion of the gene remaining in the bacmid form a full or functional portion of the baculoviral gene. In certain embodiments, the 5′ end of the fusion-polynucleotide comprises the 3′-portion of the gene that was split, such that the 3′-portion of the gene in the fusion-polynucleotide and the 5′-portion of the gene remaining in the bacmid form a full or functional portion of the baculoviral gene.

In certain embodiments, the polynucleotide can be incorporated into the bacmid at the location of a restriction endonuclease (REN) cleavage site (i.e., REN access point) associated with a baculoviral gene. In certain embodiments, the REN access point in the bacmid is FseI (corresponding with the gta baculovirus gene) (ggccggcc). In certain embodiments, the REN access point in the bacmid is SdaI (corresponding with the DNA polymerase baculovirus gene) (cctgcagg). In certain embodiments, the REN access point in the bacmid is MauBI (corresponding with the lef-4 baculovirus gene) (cgcgcgcg). In certain embodiments, the REN access point in the bacmid is SbfI (corresponding with the gp64/gp67 baculovirus gene) (cctgcagg). In certain embodiments, the REN access point in the bacmid is I-CeuI (corresponding with the v-cath baculovirus gene) (SEQ ID NO: 1735). In certain embodiments, the REN access point in the bacmid is AvrII (corresponding with the egt baculovirus gene) (cctagg). In certain embodiments, the REN access point in the bacmid is NheI (gctagc). In certain embodiments, the REN access point in the bacmid is SpeI (actagt). In certain embodiments, the REN access point in the bacmid is BstZ17I (gtatac). In certain embodiments, the REN access point in the bacmid is NcoI (ccatgg). In certain embodiments, the REN access point in the bacmid is MluI (acgcgt).

In certain embodiments where the bacmid is a double-stranded construct, the REN cleavage site can comprise a cleavage sequence in one strand and the reverse complement of the cleavage sequence (which also functions as a cleavage sequence) in the other strand. A polynucleotide insert (or strand thereof) can thus comprise a REN cleavage sequence or the reverse complement REN cleavage sequence (which are generally functionally interchangeable). As a non-limiting example, a strand of a polynucleotide insert can comprise an FseI cleave sequence (ggccggcc) or its reverse complement REN cleavage sequence (ccggccgg).

Polynucleotides can be incorporated into these REN access points by: (i) providing a polynucleotide insert which has been engineered to comprise a target REN cleavage sequence (e.g., a polynucleotide insert engineered to comprise FseI REN sequences at both ends of the polynucleotide); (ii) proving a bacmid which comprises the target REN access point for polynucleotide insertion (e.g., a variant of the AcMNPV bacmid bMON14272 which comprises an FseI cleavage site (ii) digesting the REN-engineered polynucleotide with the appropriate REN enzyme (e.g., using FseI enzyme to digesting the REN-engineering polynucleotide which comprises the FseI regions at both ends, to produce a polynucleotide-FseI insert); (iii) digesting the bacmid with the same REN enzyme to produce a single-cut bacmid at the REN access point (e.g., using FseI enzyme to produce a single-cut bacmid at the FseI location); and (iv) ligating the polynucleotide insert into the single-cut bacmid using an appropriate ligation enzyme, such as T4 ligase enzyme. The result is engineered bacmid DNA which comprises the engineered polynucleotide insert at the target REN access point.

The insertion process can be repeated one or more times to incorporate other engineered polynucleotide inserts into the same bacmid at different REN access points (e.g., insertion of a first engineered polynucleotide insert at the AvrII REN access point in the egt, followed by insertion of a second engineered polynucleotide insert at the I-CeuI REN access point in the cath gene, and followed by insertion of a third engineered polynucleotide insert at the FseI REN access point in the gta gene).

In certain embodiments, restriction endonuclease (REN) cleavage can be used to remove one or more wild-type genes from a bacmid. In certain embodiments, restriction endonuclease (REN) cleavage can be used to remove one or more engineered polynucleotide insert which has been previously been inserted into the bacmid. In certain embodiments, restriction endonuclease (REN) cleavage can be used to replace one or more engineered polynucleotide inserts with a different engineered polynucleotide insert which comprises the same REN cleavage sequences (e.g., an engineered polynucleotide insert at the FseI REN access point can be replaced with a different engineered polynucleotide insert which comprises FseI REN cleavage sequences).

### Expression Control

### Expression Control Regions

The viral expression constructs of the present disclosure can comprise one or more expression control region encoded by expression control sequences. In certain embodiments, the expression control sequences are for expression in a viral production cell, such as an insect cell. In certain embodiments, the expression control sequences are operably linked to a protein-coding nucleotide sequence. In certain embodiments, the expression control sequences are operably linked to a VP coding nucleotide sequence or a Rep coding nucleotide sequence.

Herein, the terms “coding nucleotide sequence”, “protein-encoding gene” or “protein-coding nucleotide sequence” refer to a nucleotide sequence that encodes or is translated into a protein product, such as VP proteins or Rep proteins. “Operably linked” means that the expression control sequence is positioned relative to the coding sequence such that it can promote the expression of the encoded gene product.

“Expression control sequence” refers to a nucleic acid sequence that regulates the expression of a nucleotide sequence to which it is operably linked. An expression control sequence is “operably linked” to a nucleotide sequence when the expression control sequence controls and regulates the transcription and/or the translation of the nucleotide sequence. Thus, an expression control sequence can comprise promoters, enhancers, untranslated regions (UTRs), internal ribosome entry sites (IRES), transcription terminators, a start codon in front of a protein-encoding gene, splicing signal for introns, and stop codons. The term “expression control sequence” is intended to comprise, at a minimum, a sequence whose presence are designed to influence expression, and can also comprise additional advantageous components. For example, leader sequences and fusion partner sequences are expression control sequences. The term can also comprise the design of the nucleic acid sequence such that undesirable, potential initiation codons in and out of frame, are removed from the sequence. It can also comprise the design of the nucleic acid sequence such that undesirable potential splice sites are removed. It comprises sequences or polyadenylation sequences (pA) which direct the addition of a poly(A) tail, i.e., a string of adenine residues at the 3′-end of an mRNA, sequences referred to as poly(A) sequences. It also can be designed to enhance mRNA stability. Expression control sequences which affect the transcription and translation stability, e.g., promoters, as well as sequences which effect the translation, e.g., Kozak sequences, are known in insect cells. Expression control sequences can be of such nature as to modulate the nucleotide sequence to which it is operably linked such that lower expression levels or higher expression levels are achieved.

In certain embodiments, the expression control sequence can comprise one or more promoters. Promoters can comprise, but are not limited to, baculovirus major late promoters, insect virus promoters, non-insect virus promoters, vertebrate virus promoters, nuclear gene promoters, chimeric promoters from one or more species comprising virus and non-virus elements, and/or synthetic promoters. In certain embodiments, a promoter can be Ctx, Op-EI, EI, ΔEI, EI-1, pH, PIO, polH (polyhedron), ΔpolH, Dmhsp70, Hr1, Hsp70, 4×Hsp27 EcRE+minimal Hsp70, IE, IE-1, ΔIE-1, ΔIE, p10, Δp10 (modified variations or derivatives of p10), p⁵, p1⁹, p35, p40, p6.9, and variations or derivatives thereof. In certain embodiments, the nucleotide sequence encoding a structural AAV capsid protein (e.g., VP1, VP2, VP3, or a combination thereof) is operably linked to a p10 promoter, the nucleotide sequencing encoding a non-structural AAV protein (e.g., Rep 78, Rep 52, or a combination thereof) is operably linked to a polH promoter, and the nucleotide sequence encoding a payload (e.g., AADC, e.g., SEQ ID NO: 979) is operably linked to a CMV promoter. In certain embodiments, the promoter is a Ctx promoter. In certain embodiments, the promoter is a p10 promoter. In certain embodiments, the promoter is a polH promoter. In certain embodiments, a promoter can be selected from tissue-specific promoters, cell-type-specific promoters, cell-cycle-specific promoters, and variations or derivatives thereof. In certain embodiments, a promoter can be a CMV promoter, an alpha 1-antitrypsin (α1-AT) promoter, a thyroid hormone-binding globulin promoter, a thyroxine-binding globulin (LPS) promoter, an HCR-ApoCII hybrid promoter, an HCR-hAAT hybrid promoter, an albumin promoter, an apolipoprotein E promoter, an α1-AT+EaIb promoter, a tumor-selective E2F promoter, a mononuclear blood IL-2 promoter, and variations or derivatives thereof. In certain embodiments, the promoter is a low-expression promoter sequence. In certain embodiments, the promoter is an enhanced-expression promoter sequence. In certain embodiments, the promoter can comprise Rep or Cap promoters as described in US Patent Application 20110136227, the content of which is incorporated herein by reference in its entirety as related to expression promoters, insofar as it does not conflict with the present disclosure.

In certain embodiments, a viral expression construct can comprise the same promoter in all nucleotide sequences. In certain embodiments, a viral expression construct can comprise the same promoter in two or more nucleotide sequences. In certain embodiments, a viral expression construct can comprise a different promoter in two or more nucleotide sequences. In certain embodiments, a viral expression construct can comprise a different promoter in all nucleotide sequences.

In certain embodiments the viral expression construct encodes elements to improve expression in certain cell types. In a further embodiment, the expression construct may comprise polh and/or ΔIE-1 insect transcriptional promoters, CMV mammalian transcriptional promoter, and/or p10 insect specific promoters for expression of a desired gene in a mammalian or insect cell.

More than one expression control sequence can be operably linked to a given nucleotide sequence. For example, a promoter sequence, a translation initiation sequence, and a stop codon can be operably linked to a nucleotide sequence.

In certain embodiments, the viral expression construct can comprise one or more expression control sequence between protein-coding nucleotide sequences. In certain embodiments, an expression control region can comprise an IRES sequence region which comprises an IRES nucleotide sequence encoding an internal ribosome entry sight (IRES). The internal ribosome entry sight (IRES) can be selected from the group consisting or: FMDV-IRES from Foot-and-Mouth-Disease virus, EMCV-IRES from Encephalomyocarditis virus, and combinations thereof.

In certain embodiments, the viral expression construct may contain a nucleotide sequence which comprises a start codon region, such as a sequence encoding AAV capsid proteins which comprise one or more start codon regions. In certain embodiments, the start codon region can be within an expression control sequence.

In certain embodiments, the viral expression construct is as described in any of PCT/US2019/054600 and/or U.S. Provisional Patent Application No. 62/741,855, the contents of which are each incorporated by reference in their entireties.

In certain embodiments, the viral expression construct comprises a first VP-coding region which comprises a nucleotide sequence encoding one or more AAV capsid proteins selected from VP1, VP2, and VP3. In certain embodiments, the first VP-coding region comprises a nucleotide sequence encoding one or more AAV capsid proteins selected from VP2 and VP3. In certain embodiments, the first VP-coding region comprises a nucleotide sequence encoding VP1, VP2, and VP3 AAV capsid proteins. In certain embodiments, the first VP-coding region comprises a nucleotide sequence encoding VP2 and VP3 AAV capsid proteins. In certain embodiments, the first VP-coding region comprises a nucleotide sequence encoding only VP2 and VP3 AAV capsid proteins. In certain embodiments, the first VP-coding region comprises a nucleotide sequence encoding VP2 and VP3 AAV capsid proteins, but not VP1.

In certain embodiments, the nucleic acid construct comprises a second VP-coding region which comprises a nucleotide sequence encoding one or more AAV capsid proteins selected from VP1, VP2, and VP3. In certain embodiments, the second VP-coding region comprises a nucleotide sequence encoding VP1 AAV capsid proteins. In certain embodiments, the second VP-coding region comprises a nucleotide sequence encoding only VP1 AAV capsid proteins. In certain embodiments, the second VP-coding region comprises a nucleotide sequence encoding VP1 AAV capsid proteins, but not VP2 or VP3.

In certain embodiments, the viral expression construct is an engineered nucleic acid construct. In certain embodiments, the viral expression construct comprises a first nucleotide sequence which comprises the first VP-coding region and the second VP-coding region. In certain embodiments, the first nucleotide sequence comprises a first open reading frame (ORF) which comprises the first VP-coding region, and a second open reading frame (ORF) which comprises the second VP-coding region.

In certain embodiments, the viral expression construct comprises a first nucleotide sequence which comprises the first VP-coding region and a second nucleotide sequence which comprises the second VP-coding region. In certain embodiments, the first nucleotide sequence comprises a first open reading frame (ORF) which comprises the first VP-coding region, and the second nucleotide sequence comprises a second open reading frame (ORF) which comprises the second VP-coding region. In certain embodiments, the first open reading frame is different from the second open reading frame.

In certain embodiments, the viral expression construct comprises a first VP-coding region which comprises a nucleotide sequence encoding one or more AAV capsid proteins selected from VP1, VP2, and VP3; and a second VP-coding region which comprises a nucleotide sequence encoding one or more AAV capsid proteins selected from VP1, VP2, and VP3. In certain embodiments, the first VP-coding region comprises a nucleotide sequence encoding VP1, VP2 and VP3 AAV capsid proteins; and the second VP-coding region comprises a nucleotide sequence encoding only VP1 AAV capsid proteins. In certain embodiments, the first VP-coding region comprises a nucleotide sequence encoding VP1, VP2, and VP3 AAV capsid proteins; and the second VP-coding region comprises a nucleotide sequence encoding VP1 AAV capsid proteins, but not VP2 or VP3. In certain embodiments, the first VP-coding region comprises a nucleotide sequence encoding only VP2 and VP3 AAV capsid proteins; and the second VP-coding region comprises a nucleotide sequence encoding only VP1 AAV capsid proteins. In certain embodiments, the first VP-coding region comprises a nucleotide sequence encoding VP2 and VP3 AAV capsid proteins, but not VP1; and the second VP-coding region which comprises a nucleotide sequence encoding VP1 AAV capsid proteins, but not VP2 or VP3.

In certain embodiments, the viral expression construct comprises one or more start codon regions which include a start codon. In certain embodiments, the viral expression construct comprises one or more stop codon regions which include a stop codon. In certain embodiments, the viral expression construct comprises one or more start codon regions and one or more stop codon regions.

In certain embodiments, the viral expression construct comprises one or more expression control regions which comprise an expression control sequence. In certain embodiments, the expression control region comprises one or more promoter sequences. In certain embodiments, the expression control region comprises one or more promoter sequences selected from the group consisting of: baculovirus major late promoters, insect virus promoters, non-insect virus promoters, vertebrate virus promoters, nuclear gene promoters, chimeric promoters from one or more species including virus and non-virus elements, synthetic promoters, and variations or derivatives thereof. In certain embodiments, the expression control region comprises one or more promoter sequences selected from the group consisting of: Ctx promoter, polh insect transcriptional promoters, ΔIE-1 insect transcriptional promoters, p10 insect specific promoters, Δp10 insect specific promoters (variations or derivatives of p10), CMV mammalian transcriptional promoter, and variations or derivatives thereof. In certain embodiments, the expression control region comprises one or more low-expression promoter sequences. In certain embodiments, the expression control region comprises one or more enhanced-expression promoter sequences.

In certain embodiments, the first VP-coding region encodes AAV capsid proteins of an AAV serotype, e.g., AAV2. In certain embodiments, the second VP-coding region encodes AAV capsid proteins of an AAV serotype, e.g., AAV2. In certain embodiments, the AAV serotype of the first VP-coding region is the same as the AAV serotype of the second VP-coding region. In certain embodiments, the AAV serotype of the first VP-coding region is different from the AAV serotype of the second VP-coding region. In certain embodiments, a VP-coding region can be codon optimized for an insect cell. In certain embodiments, a VP-coding region can be codon optimized for a Spodoptera frugiperda cell.

In certain embodiments, a nucleotide sequence encoding a VP1 capsid protein can be codon optimized. In certain embodiments, a nucleotide sequence encoding a VP1 capsid protein can be codon optimized for an insect cell. In certain embodiments, a nucleotide sequence encoding a VP2 capsid protein can be codon optimized. In certain embodiments, a nucleotide sequence encoding a VP2 capsid protein can be codon optimized for an insect cell. In certain embodiments, a nucleotide sequence encoding a VP3 capsid protein can be codon optimized. In certain embodiments, a nucleotide sequence encoding a VP3 capsid protein can be codon optimized for an insect cell.

In certain embodiments, a nucleotide sequence encoding a VP1 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a nucleotide sequence encoding a VP2 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a nucleotide sequence encoding a VP3 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, the viral expression construct comprises: (i) a first nucleotide sequence which comprises a first expression control region comprising a first promoter sequence, and a first VP-coding region which comprises a nucleotide sequence encoding one or more AAV capsid proteins selected from VP1, VP2, and VP3; and (ii) a second nucleotide sequence which comprises a second expression control region comprising a second promoter sequence, and a second VP-coding region which comprises a nucleotide sequence encoding VP1 AAV capsid proteins, but not VP2 or VP3. In certain embodiments, the viral expression construct comprises: (i) a first nucleotide sequence which comprises a first expression control region comprising a first promoter sequence, and a first VP-coding region which comprises a nucleotide sequence encoding VP2 and VP3 AAV capsid proteins, but not VP1; and (ii) a second nucleotide sequence which comprises a second expression control region comprising a second promoter sequence, and a second VP-coding region which comprises a nucleotide sequence encoding VP1 AAV capsid proteins, but not VP2 or VP3. In certain embodiments, the nucleotide sequence of the second VP-coding region is codon optimized. In certain embodiments, the nucleotide sequence of the second VP-coding region is codon optimized for an insect cell, or more specifically for a Spodoptera frugiperda cell. In certain embodiments, the nucleotide sequence of the second VP-coding region is codon optimized codon optimized to have a nucleotide homology with the reference nucleotide sequence of less than 100%, less than 90%, or less than 80%.

In certain embodiments, the viral expression construct comprises: (i) a first nucleotide sequence which comprises a first expression control region comprising a first promoter sequence, a first start codon region which comprises a first start codon, a first VP-coding region which comprises a nucleotide sequence encoding one or more AAV capsid proteins selected from VP1, VP2 and VP3, and a first stop codon region which comprises a first stop codon; and (ii) a second nucleotide sequence which comprises a second expression control region comprising a second promoter sequence, a second start codon region which comprises a second start codon, a second VP-coding region which comprises a nucleotide sequence encoding VP1 AAV capsid proteins, but not VP2 or VP3, and a second stop codon region which comprises a second stop codon. In certain embodiments, the nucleic acid construct comprises: (i) a first nucleotide sequence which comprises a first expression control region comprising a first promoter sequence, a first start codon region which comprises a first start codon, a first VP-coding region which comprises a nucleotide sequence encoding VP2 and VP3 AAV capsid proteins, but not VP1, and a first stop codon region which comprises a first stop codon; and (ii) a second nucleotide sequence which comprises a second expression control region comprising a second promoter sequence, a second start codon region which comprises a second start codon, a second VP-coding region which comprises a nucleotide sequence encoding VP1 AAV capsid proteins, but not VP2 or VP3, and a second stop codon region which comprises a second stop codon. In certain embodiments, the first start codon is ATG, the second start codon is ATG, or both the first and second start codons are ATG.

In certain embodiments, the viral expression construct comprises a first nucleotide sequence which comprises: a Rep52-coding region which comprises a Rep52 sequence encoding a Rep52 protein, a Rep78-coding region which comprises a Rep78 sequence encoding a Rep78 protein, or a combination thereof. In certain embodiments, the first nucleotide sequence comprises both a Rep52-coding region and a Rep78-coding region. In certain embodiments, the first nucleotide sequence comprises a single open reading frame, consists essentially of a single open reading frame, or consists of a single open reading frame. In certain embodiments, the first nucleotide sequence comprises a first open reading frame which comprises a Rep52-coding region, and a second open reading frame which comprises a Rep78-coding region and which is different from the first open reading frame.

In certain embodiments, an expression control region can comprise a 2A sequence region which comprises a 2A nucleotide sequence encoding a viral 2A peptide. The sequence allows for co-translation of multiple polypeptides within a single open reading frame (ORF). As the ORF is translated, glycine and proline residues with the 2A sequence prevent the formation of a normal peptide bond, which results in ribosomal “skipping” and “self-cleavage” within the polypeptide chain. The viral 2A peptide can be selected from the group consisting of: F2A from Foot-and-Mouth-Disease virus, T2A from Thosea asigna virus, E2A from Equine rhinitis virus A, P2A from Porcine teschovirus-1, BmCPV2A from cytoplasmic polyhedrosis virus, BmIFV 2A from B. mori flacherie virus, and combinations thereof.

In certain embodiments, a first nucleotide sequence comprises a Rep52-coding region and 2A sequence region. In certain embodiments, a first nucleotide sequence comprises a Rep78-coding region and 2A sequence region. In certain embodiments, a first nucleotide sequence comprises a Rep52-coding region, a Rep78-coding region, and 2A sequence region. In certain embodiments, a first nucleotide sequence comprises a 2A sequence region located between a Rep52-coding region and a Rep78-coding region on the nucleotide sequence. In certain embodiments, a first nucleotide comprises, in order from the 5′-end to the 3′-end, a Rep52-coding region, a 2A sequence region, and a Rep78-coding region. In certain embodiments, a first nucleotide comprises, in order from the 5′-end to the 3′-end, a Rep78-coding region, a 2A sequence region, and a Rep52-coding region.

In some embodiments, the first and/or second nucleotide sequence comprises a start codon and/or stop codon and/or internal ribosome entry site (IRES). In certain embodiments, the IRES nucleotide sequence encodes an internal ribosome entry site (IRES) selected from the group consisting of: FMDV-IRES from Foot-and-Mouth-Disease virus, EMCV-IRES from Encephalomyocarditis virus, and combinations thereof.

For example, in certain embodiments, a first nucleotide sequence comprises a start codon region, a Rep52-coding region, 2A sequence region, and a stop codon region. In certain embodiments, a first nucleotide sequence comprises a start codon region, a Rep78-coding region, 2A sequence region, and a stop codon region. In certain embodiments, a first nucleotide sequence comprises a start codon region, a Rep52-coding region, a 2A sequence region, a Rep78-coding region, and a stop codon region. In certain embodiments, a first nucleotide comprises, in order from the 5′-end to the 3′-end, a start codon region, a Rep52-coding region, a 2A sequence region, a Rep78-coding region, and a stop codon region. In certain embodiments, a first nucleotide comprises, in order from the 5′-end to the 3′-end, a start codon region, a Rep78-coding region, a 2A sequence region, a Rep52-coding region, and a stop codon region.

In certain embodiments, a first nucleotide sequence comprises a Rep52-coding region, a Rep78-coding region, and an IRES sequence region. In certain embodiments, a first nucleotide sequence comprises an IRES sequence region located between a Rep52-coding region and a Rep78-coding region on the nucleotide sequence. In certain embodiments, a first nucleotide comprises, in order from the 5′-end to the 3′-end, a Rep52-coding region, an IRES sequence region, and a Rep78-coding region. In certain embodiments, a first nucleotide comprises, in order from the 5′-end to the 3′-end, a Rep78-coding region, an IRES sequence region, and a Rep52-coding region.

In certain embodiments, the first nucleotide sequence comprises a first open reading frame which comprises a Rep52-coding region, a second open reading frame which comprises a Rep78-coding region, and an IRES sequence region located between the first open reading frame and the second open reading frame. In certain embodiments, a first nucleotide sequence comprises, in order from the 5′-end to the 3′-end, a first open reading frame which comprises a Rep52-coding region, an IRES sequence region, and a second open reading frame which comprises a Rep78-coding region. In certain embodiments, a first nucleotide sequence comprises, in order from the 5′-end to the 3′-end, a first open reading frame which comprises a Rep78-coding region, an IRES sequence region, and a second open reading frame which comprises a Rep52-coding region.

In certain embodiments, a first nucleotide sequence comprises, in order from the 5′-end to the 3′-end: a first open reading frame which comprises a first start codon region, a Rep52-coding region, and a first stop codon region; an IRES sequence region; and a second open reading frame which comprises a second start codon region, a Rep78-coding region, and a second stop codon region. In certain embodiments, a first nucleotide sequence comprises, in order from the 5′-end to the 3′-end: a first open reading frame which comprises a first start codon region, a Rep78-coding region, and a first stop codon region; an IRES sequence region; and a second open reading frame which comprises a second start codon region, a Rep52-coding region, and a second stop codon region.

In certain embodiments, the nucleic acid construct comprises a first nucleotide sequence, and a second nucleotide sequence which is separate from the first nucleotide sequence within the nucleic acid construct. In certain embodiments, the nucleic acid construct comprises a first nucleotide sequence which comprises a Rep52-coding region, and a separate second nucleotide sequence which comprises a Rep78-coding region. In certain embodiments, the nucleic acid construct comprises a first nucleotide sequence and a separate second nucleotide sequence; wherein the first nucleotide sequence comprises a Rep52-coding region and a 2A sequence region; and wherein the second nucleotide sequence comprises a Rep78-coding region and a 2A sequence region.

In certain embodiments, the viral expression construct comprises one or more essential-gene regions which comprises an essential-gene nucleotide sequence encoding an essential protein for the nucleic acid construct. In certain embodiments, the essential-gene nucleotide sequence is a baculoviral sequence encoding an essential baculoviral protein. In certain embodiments, the essential baculoviral protein is a baculoviral envelope protein or a baculoviral capsid protein. For example, in certain embodiments, the nucleic acid construct comprises a first nucleotide sequence and a separate second nucleotide sequence; wherein the first nucleotide sequence comprises a Rep52-coding region and a first essential-gene region; and wherein the second nucleotide sequence comprises a Rep78-coding region and a second essential-gene region. In certain embodiments, the nucleic acid construct comprises a first nucleotide sequence and a separate second nucleotide sequence; wherein the first nucleotide sequence comprises a Rep52-coding region, a 2A sequence region, and a first essential-gene region; and wherein the second nucleotide sequence comprises a Rep78-coding region, a 2A sequence region, and a second essential-gene region. In certain embodiments, the nucleic acid construct comprises a first nucleotide sequence and a separate second nucleotide sequence; wherein the first nucleotide sequence comprises, in order, a Rep52-coding region, a 2A sequence region, and a first essential-gene region; and wherein the second nucleotide sequence comprises, in order, a Rep78-coding region, a 2A sequence region, and a second essential-gene region.

In certain embodiments, the essential baculoviral protein is a GP64 baculoviral envelope protein. In certain embodiments, the essential baculoviral protein is a VP39 baculoviral capsid protein.

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In some embodiments, a viral expression construct may further comprise a polynucleotide encoding AADC.

In certain embodiments, AAV particles, e.g., those comprising a polynucleotide encoding AADC, e.g., those comprising SEQ ID NO: 979 are produced in viral production cells (such as mammalian or insect cells) wherein all three VP proteins are expressed at a stoichiometry approaching, about or which is 1:1:10 (VP1:VP2:VP3).

In certain embodiments, AAV particles are produced in viral production cells (such as mammalian or insect cells) wherein all three VP proteins are expressed at a stoichiometry approaching, about or which is 2:2:10 (VP1:VP2:VP3).

In certain embodiments, AAV particles are produced in viral production cells (such as mammalian or insect cells) wherein all three VP proteins are expressed at a stoichiometry approaching, about or which is 2:0:10 (VP1:VP2:VP3).

In certain embodiments, AAV particles are produced in viral production cells (such as mammalian or insect cells) wherein all three VP proteins are expressed at a stoichiometry approaching, about or which is 1-2:0-2:10 (VP1:VP2:VP3).

In certain embodiments, AAV particles are produced in viral production cells (such as mammalian or insect cells) wherein all three VP proteins are expressed at a stoichiometry approaching, about or which is 1-2:1-2:10 (VP1:VP2:VP3).

In certain embodiments, AAV particles are produced in viral production cells (such as mammalian or insect cells) wherein all three VP proteins are expressed at a stoichiometry approaching, about or which is 2-3:0-3:10 (VP1:VP2:VP3).

In certain embodiments, AAV particles are produced in viral production cells (such as mammalian or insect cells) wherein all three VP proteins are expressed at a stoichiometry approaching, about or which is 2-3:2-3:10 (VP1:VP2:VP3).

In certain embodiments, AAV particles are produced in viral production cells (such as mammalian or insect cells) wherein all three VP proteins are expressed at a stoichiometry approaching, about or which is 3:3:10 (VP1:VP2:VP3).

In certain embodiments, AAV particles are produced in viral production cells (such as mammalian or insect cells) wherein all three VP proteins are expressed at a stoichiometry approaching, about or which is 3-5:0-5:10 (VP1:VP2:VP3).

In certain embodiments, AAV particles are produced in viral production cells (such as mammalian or insect cells) wherein all three VP proteins are expressed at a stoichiometry approaching, about or which is 3-5:3-5:10 (VP1:VP2:VP3).

In certain embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

In certain embodiments of the present disclosure, Rep52 or Rep78 is transcribed from the baculoviral derived polyhedron promoter (polh). Rep52 or Rep78 can also be transcribed from a weaker promoter, for example a deletion mutant of the IE-1 promoter, the ΔIE-1 promoter, has about 20% of the transcriptional activity of that IE-1 promoter. A promoter substantially homologous to the ΔIE-1 promoter may be used. In respect to promoters, a homology of at least 50%, 60%, 70%, 80%, 90% or more, is considered to be a substantially homologous promoter.

### Engineered Untranslated Regions (UTRs)

The present disclosure presents engineered untranslated regions (UTRs), comprising engineered UTR polynucleotides that function as a 5′ UTR. Engineering the features in untranslated regions (UTRs) can improve the stability and protein production capability of the viral production constructs of the present disclosure.

The present disclosure presents viral expression constructs which comprise an engineered untranslated region (UTR) of the present disclosure. In certain embodiments, the viral expression construct comprises an engineered untranslated region (UTR) of the present disclosure. In certain embodiments, the viral expression construct comprises an engineered 5′ UTR of the present disclosure.

Natural 5′ UTRs comprise features which play important roles in translation initiation. They harbor signatures such as a Kozak sequences which are known to be involved in the process by which the ribosome initiates translation of many genes. The present disclosure provides engineered polynucleotide sequences which comprise at least one 5′ UTR function. Such “engineered 5′ UTR polynucleotides” or “engineered 5′ UTRs” may also comprise the start codon of the protein whose expression is being driven, e.g., a structural AAV capsid protein (VP1, VP2 or VP3) or a non-structural AAV replication protein (Rep78 or Rep52).

According to the present disclosure, the engineered 5′ UTR polynucleotides may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides). Non-UTR sequences may be incorporated into the engineered 5′ UTRs. For example, introns or portions of introns sequences may be incorporated into the polynucleotides of the disclosure. Incorporation of intronic sequences may also increase AAV serotype protein (e.g., capsid) production.

Leader sequences may be comprised in the engineered polynucleotides. Such leader sequences may derive from or be identical to all or a portion of any AAV serotype selected from those taught herein.

According to the present disclosure, the polynucleotides may comprise a consensus sequence which is discovered through rounds of experimentation. As used herein a “consensus” sequence is a single sequence which represents a collective population of sequences allowing for variability at one or more sites.

In certain embodiments, variants of the polynucleotides of the disclosure may be generated. These variants may have the same or a similar activity as the reference polynucleotide. Alternatively, the variant may have an altered activity (e.g., increased or decreased) relative to a reference polynucleotide. Generally, variants of a particular polynucleotides of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide as determined by sequence alignment programs and parameters described herein and known to those skilled in the art. Such tools for alignment comprise those of the BLAST suite (Stephen F. Altschul, Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.) Other tools are described herein, specifically in the definition of “identity.”

The engineered polynucleotides of the present disclosure may be incorporated into a vector or plasmid alone or in combination with other polynucleotide sequences or features such as those disclosed in International Publications WO2007046703 and WO2007148971 (disclosing alternative start codons and AAV vectors produced in insect cells); WO2009104964 (disclosing optimization of expression of AAV proteins in insect cells and involving alteration of promoter strength, enhancer elements, temperature control); and WO2015137802 (disclosing alternative start codons, removal of start codons and AAV vectors produced in insect cells), the contents of which are each incorporated herein by reference in their entireties, insofar as they do not conflict with the present disclosure.

In certain embodiments, the engineered 5′ UTR comprises or consists of between 80-120 nucleotides, between 90-110 nucleotides, between 95-10⁵ nucleotides, between 98-100 nucleotides, or about 99 nucleotides. In certain embodiments, the engineered 5′ UTR comprises or consists of 24 nucleotides.

In certain embodiments, the engineered 5′ UTR is derived from AAV2. In certain embodiments, the engineered 5′ UTR is derived from AAV2. In certain embodiments, the engineered 5′ UTR is derived from AAV9. In certain embodiments, the engineered 5′ UTR is derived from AAVRh10. In certain embodiments, the engineered 5′ UTR is derived from AAVPHP.B. In certain embodiments, the engineered 5′ UTR is derived from an AAV serotype disclosed herein.

### 5′ UTR Hairpin Structure

In certain embodiments, the engineered 5′ UTR comprises a hairpin structure. In certain embodiments, the engineered 5′ UTR region comprises: a promoter 5′ (upstream) of a 5′ UTR which comprises an “A” region (a 5′ flanking region) which is 5′ (upstream) of a hairpin, a “B” region (a 3′ flanking region which can comprise a start codon and kozak nucleotides around the start codon) which is 3′ (downstream) from the stem loop, a “C” region representing the stem of a stem-loop structure, and a loop (which can range from 4-16 nucleotides). In certain embodiments, the hairpin structure can comprise all or a portion of a Kozak sequence, such as TTT. The promoter and 5′ UTR can be associated with either a CAP gene (which encodes the structural capsid proteins VP1, VP2 and/or VP3) or a REP gene (which encodes the non-structural replication proteins Rep78 and Rep52).

In certain embodiments, the engineered 5′ UTR comprises a hairpin structure encoded by a hairpin nucleotide sequence. In certain embodiments, the hairpin nucleotide sequence comprises a leader sequence. In certain embodiments, the hairpin nucleotide sequence comprises a leader sequence and a start codon (e.g., ATG). In certain embodiments, the hairpin nucleotide sequence comprises a leader sequence, and a start codon (e.g., ATG) within a Kozak sequence or modified Kozak sequence.

In certain embodiments, the engineered 5′ UTR comprises a hairpin structure having a 5′ flanking region (i.e., upstream region) encoded by a 5′ flanking sequence. In certain embodiments, the 5′ flanking sequence may be of any length and may be derived in whole or in part from wild type AAV sequence or be completely artificial.

In certain embodiments, the engineered 5′ UTR comprises a hairpin structure having a 3′ flanking region (i.e., downstream region) encoded by a 3′ flanking sequence. In certain embodiments, the 3′ flanking sequence may be of any length and may be derived in whole or in part from wild type AAV sequence or be completely artificial.

The 5′ flanking sequence and 3′ flanking sequence can have the same size and origin, a different size, a different origin, or a different size and origin. Either flanking sequence may be absent. The 5′ flanking sequence can comprise or consist of 2-50, 2-40, 2-30, 2-20, or 2-15 nucleotides. The 3′ flanking sequence can comprise or consist of 2-50, 2-40, 2-30, 2-20, or 2-15 nucleotides. The 3′ flanking sequence may optionally contain the start codon of an AAV protein or proteins as well as other sequences such as a Kozak or modified Kozak sequence.

In certain embodiments, the engineered 5′ UTR comprises a hairpin structure which comprises a step-loop structure. In certain embodiments, the hairpin structure comprises a stem region and a loop region. In certain embodiments, the hairpin structure comprises a stem region, a loop region, and a stem-complement region. The stem-loop structure can comprise a stem region encoded by a stem sequence. The stem-loop structure can comprise a loop region encoded by a loop sequence. The stem-loop structure can comprise a stem-complement region encoded by a stem-complement sequence. Forming the stem of the stem-loop structure of the hairpin are paired or substantially paired nucleobases of between 2 and 50 pairs. The stem may contain one or more mismatches, bulges or loops. In certain embodiments, the stem sequence and the stem-complement sequence are 100% complementary (i.e., zero mismatches). In certain embodiments, the stem sequence and the stem-complement sequence comprise zero, one, two, three, four or five mismatches.

In certain embodiments, the engineered 5′ UTR comprises a hairpin structure presented in Table 2, or a combination of Upstream, Stem (Upstream), Loop, Stem (Downstream) and/or Downstream components listed in Table 2. In The position of the loop portion of the hairpin is underlined in the sequence with the canonical ATG start codon in Capital Letters.

TABLE 2
5' UTR Hairpin S tructures

| | Hairpin | | | | | |
|---|---|---|---|---|---|---|
| Name | Sequence | Upstream | Stem-U | Loop | Stem-D | Downstream |
| HP1 | atacgactcgacgaa | atacgactcgacga | aaccgtcggc | tttatggct | | |
| | gacttgatcaaccgtc | agacttgatc | SEQ ID | | | |
| | ggctttATGgct | SEQ ID NO: | NO: 1756 | | | |
| | SEQ ID NO: | 1752 | | | | |
| | 1736 | | | | | |
| HP2 | atacgactcgacgaa | atacgactcgacga | aaccAtcggc | tttatggct | | |
| | gacttgatcaaccAt | agacttgatc | SEQ ID | | | |
| | cggctttATGgct | SEQ ID NO: | NO: 1757 | | | |
| | SEQ ID NO: | 1752 | | | | |
| | 1737 | | | | | |
| HP3 | atacgactcgacgaa | atacgactcgacga | aaccgtAggc | tttatggct | | |
| | gacttgatcaaccgt | agacttgatc | SEQ ID | | | |
| | AggctttATGgct | SEQ ID NO: | NO: 1758 | | | |
| | SEQ ID NO: | 1752 | | | | |
| | 1738 | | | | | |
| HP4 | atacgactcgacgaa | atacgactcgacga | cggc | tttatggct | | |
| | gacttgatcctgactc | agacttgatcctgac | | | | |
| | ggctttATGgct | t | | | | |
| | SEQ ID NO: | SEQ ID NO: | | | | |
| | 1739 | 1753 | | | | |
| HP5 | atacgactcgacgaa | atacgactcgacga | agTtaaccgt | tttatggct | | |
| | gacttagTtaaccgtc | agactt | cggc | | | |
| | ggctttATGgct | SEQ ID NO: | SEQ ID | | | |
| | SEQ ID NO: | 1754 | NO: 1759 | | | |
| | 1740 | | | | | |
| HP6 | atacgactcgacgaa | atacgactcgacga | agTtaaccgt | tttatggct | | |
| | gacttagTtaaccgtc | agactt | cCgc | | | |
| | GgctttATGgct | SEQ ID NO: | SEQ ID | | | |
| | SEQ ID NO: | 1754 | NO: 1760 | | | |
| | 1741 | | | | | |
| HP7 | atacgactcgacgaa | atacgactcgacga | agTtaacTgt | tttatggct | | |
| | gacttagTtaacTgt | agactt | cCgc | | | |
| | cCgctttATGgct | SEQ ID NO: | SEQ ID | | | |
| | SEQ ID NO: | 1754 | NO: 1761 | | | |
| | 1742 | | | | | |
| HP8 | atacgactcgacgaa | atacgactcgacga | ctgcc | atctaa | ggcag | tttatggct |
| | gacctgccatctaag | agac | | | | |
| | gcagtttATGgct | SEQ ID NO: | | | | |
| | SEQ ID NO: | 1755 | | | | |
| | 1743 | | | | | |
| HP9 | atacgactctgccagc | atacgact | ctgccagctc | atctaa | gagctggca | tttatggct |
| | tcatctaagagctggc | | SEQ ID | | g | |
| | agtttATGgct | | NO: 1762 | | SEQ ID | |
| | SEQ ID NO: | | | | NO: 1767 | |
| | 1744 | | | | | |
| HP10 | atacgactctgccTg | atacgact | ctgccTgctc | atctaa | gagctggca | tttatggct |
| | ctcatctaagagctgg | | SEQ ID | | g | |
| | cagtttATGgct | | NO: 1763 | | SEQ ID | |
| | SEQ ID NO: | | | | NO: 1767 | |
| | 1745 | | | | | |
| HP11 | atacctgccagctctt | atac | ctgccagctct | atctaa | cgaagagct | tttatggct |
| | cgatctaacgaagag | | tcg | | ggcag | |
| | ctggcagtttATGg | | SEQ ID | | SEQ ID | |
| | ct | | NO: 1764 | | NO: 1768 | |
| | SEQ ID NO: | | | | | |
| | 1746 | | | | | |
| HP12 | atacctgccTgctctt | atac | ctgccTgctct | atctaa | cgaagagct | tttatggct |
| | cgatctaacgaagag | | tcg | | ggcag | |
| | ctggcagtttATGg | | SEQ ID | | SEQ ID | |
| | ct | | NO: 1765 | | NO: 1768 | |
| | SEQ ID NO: | | | | | |
| | 1747 | | | | | |
| HP13 | atacctgccTgctcA | atac | ctgccTgctc | atctaa | cgaagagct | tttatggct |
| | tcgatctaacgaaga | | Atcg | | ggcag | |
| | gctggcagtttATG | | SEQ ID | | SEQ ID | |
| | get | | NO: 1766 | | NO: 1768 | |
| | SEQ ID NO: | | | | | |
| | 1748 | | | | | |
| HP14 | atacctgccatctaag | atac | ctgcc | atctaa | ggcag | gactcgacgaag |
| | gcaggactcgacgaa | | | | | actttatggct |
| | gactttATGgct | | | | | SEQ ID NO: |
| | SEQ ID NO: | | | | | 1769 |
| | 1749 | | | | | |
| HP15 | atacctgccagctcat | atac | ctgccagctc | atctaa | gagctggca | gacttttatggct |
| | ctaagagctggcagg | | SEQ ID | | g | SEQ ID NO: |
| | acttttATGgct | | NO: 1762 | | SEQ ID | 1770 |
| | SEQ ID NO: | | | | NO: 1767 | |
| | 1750 | | | | | |
| HP16 | atacctgccTgctcat | atac | ctgccTgctc | atctaa | gagctggca | gacttttatggct |
| | ctaagagctggcagg | | SEQ ID | | g | SEQ ID NO: |
| | acttttATGgct | | NO: 1763 | | SEQ ID | 1770 |
| | SEQ ID NO: | | | | NO: 1767 | |
| | 1751 | | | | | |

In certain embodiments, the engineered 5′ UTR comprises a hairpin structure, or a component thereof, which is encoded by a hairpin nucleotide sequence selected from SEQ ID NO:1736-1751. In certain embodiments, the engineered 5′ UTR comprises a hairpin structure, or a component thereof, encoded by a nucleotide sequence having at least 60% identity, at least 65% identity, at least 70% identity, at least 75% identity, at least 80% identity, at least 85% identity, at least 90% identity or at least 95% identity to SEQ ID NO: 1736-1751.

### G: C Content

In certain embodiments, the engineered 5′ UTRs of the present disclosure can comprise a nucleotide sequence, such as a leader sequence, which has varied G:C content or percentage. In certain embodiments, the 5′ flanking region of the 5′ UTRs have a varied G:C content. In certain embodiments, the stem of the 5′ UTRs has a varied G:C content. In certain embodiments, the 3′ flanking region of the 5′ UTRs has a varied G:C content. In certain embodiments, the G:C content of the engineered 5′ UTR is 10-80%, 20-70%, 25%-65%, or 30%-60%. In certain embodiments, the G:C content of the engineered 5′ UTR is about 25%, about 30%, 34%, about 35%, about 40%, about 45%, about 50%, about 55%, 58%, about 60%, 62% or about 65%.

In certain embodiments, the engineered 5′ UTR comprises or consists of between 98-100 nucleotides, and comprises a G:C content of about 25%, about 30%, 34%, about 35%, about 40%, about 45%, about 50%, about 55%, 58%, about 60%, 62% or about 65%.

### Modified Kozak Sequences

The translational start site of eukaryotic mRNA is controlled in part by a nucleotide sequence referred to as a Kozak sequence as described in Kozak, M Cell. 1986 Jan. 31; 44(2):283-92 and Kozak, M. J Cell Biol. 1989 February; 108(2):229-41 the content of which is incorporated herein by reference in its entirety as related to Kozak sequences and uses thereof, insofar as it does not conflict with the present disclosure. Both naturally occurring and synthetic (i.e., modified or engineered) translational start sites of the Kozak form can be used in the production of polypeptides by molecular genetic techniques, as described in Kozak, M. Mamm Genome. 1996 August; 7(8):563-74, the content of which is incorporated herein by reference in its entirety as related to Kozak sequences and uses thereof, insofar as it does not conflict with the present disclosure. The Kozak consensus sequence is generally defined as GCCRCC(NNN)GC (SEQ ID NO: 1771), wherein R is a purine (i.e., A or G) and wherein (NNN) stands for a translation initiation start codon, such as a suboptimal start codon. In certain embodiments, Kozak sequences are modified to provide leaky ribosome scanning of the VP-coding region. Any corresponding increase of VP1 production will conversely reduce VP3 translation, such that marginal changes of VP1 initiation rate can induce large changes in VP1/VP3 ratio. As used herein, the terms “modified Kozak sequence” or “engineered Kozak sequence” represent an altered Kozak sequence, such as, for example, a Kozak sequence which comprises nucleotide mutations, additions, or deletions.

In certain embodiments, the engineered 5′ UTRs of the present disclosure can comprise a modified Kozak sequence, such as a modified weak Kozak sequence. In certain embodiments, the engineered 5′ UTRs of the present disclosure can comprise a modified Kozak sequence which comprises or is associated with a VP start codon and/or VP translation initiation region. In certain embodiments, the engineered 5′ UTRs of the present disclosure can comprise a modified Kozak sequence which comprises or is associated with a VP1 start codon and/or VP1 translation initiation region. In certain embodiments, the engineered 5′ UTRs of the present disclosure can comprise a modified Kozak sequence which comprises or is associated with a VP2 start codon and/or VP2 translation initiation region. In certain embodiments, the engineered 5′ UTRs of the present disclosure can comprise a modified Kozak sequence which comprises or is associated with a VP3 start codon and/or VP3 translation initiation region.

In certain embodiments, the engineered 5′ UTRs of the present disclosure can comprise a modified Kozak sequence selected from Table 3.

TABLE 3
Modified Kozak Sequences

| Sequence | SEQ ID NO: |
|---|---|
| tttagatggct | 1772 |
| tttagatgttg | 1773 |
| tttttatgttg | 1774 |

In certain embodiments, the engineered 5′ UTRs of the present disclosure can comprise a modified Kozak sequence selected from SEQ ID NOS: 1772-1774. In certain embodiments, the engineered 5′ UTRs of the present disclosure can comprise a modified Kozak sequence which has at least 60% identity, at least 65% identity, at least 70% identity, at least 75% identity, at least 80% identity, at least 85% identity, at least 90% identity or at least 95% identity to a modified Kozak sequence selected from SEQ ID NOS: 1772-1774.

In certain embodiments, an engineered 5′ UTR of the present disclosure can comprise the modified Kozak sequence of SEQ ID NO: 1772. In certain embodiments, an engineered 5′ UTR of the present disclosure can comprise the modified Kozak sequence of SEQ ID NO: 1773. In certain embodiments, an engineered 5′ UTR of the present disclosure can comprise the modified Kozak sequence of SEQ ID NO: 1774.

In certain embodiments, the modified Kozak sequence is engineered or selected to produce a VP1:VP2:VP3 ratio selected from: about or exactly 1:1:10; about or exactly 2:2:10; about or exactly 3:3:10; about or exactly 4:4:10; about or exactly 5:5:10; about or exactly 1-2:1-2:10; about or exactly 1-3:1-3:10; about or exactly 1-4:1-4:10; about or exactly 1-5:1-5:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

In certain embodiments, the present disclosure presents a method of producing rAAV particles in a viral production cell such as an insect cell. In certain embodiments, the method comprises (i) transfecting a viral production cell (e.g., Sf9 insect cell) with a payload construct and viral expression construct which comprises a nucleotide sequence encoding a modified Kozak sequence and a sequence encoding VP1, VP2, and/or VP3 capsid proteins, and (ii) culturing the insect cell under conditions suitable to produce rAAV particles.

### Viral Production Cells and Vectors

### Mammalian Cells

Viral production of the present disclosure disclosed herein describes processes and methods for producing AAV particles or viral vector that contacts a target cell to deliver a payload construct, e.g., a recombinant AAV particle or viral construct, which comprises a nucleotide encoding a payload molecule. The viral production cell may be selected from any biological organism, comprising prokaryotic (e.g., bacterial) cells, and eukaryotic cells, comprising, insect cells, yeast cells and mammalian cells.

In certain embodiments, the AAV particles of the present disclosure may be produced in a viral production cell that comprises a mammalian cell. Viral production cells may comprise mammalian cells such as A549, WEH1, 3T3, 10T1/2, BHK, MDCK, COS 1, COS 7, BSC 1, BSC 40, BMT 10, VERO. W138, HeLa, HEK293, HEK293T (293T), Saos, C2C12, L cells, HT1080, HepG2 and primary fibroblast, hepatocyte and myoblast cells derived from mammals. Viral production cells can comprise cells derived from mammalian species comprising, but not limited to, human, monkey, mouse, rat, rabbit, and hamster or cell type, comprising but not limited to fibroblast, hepatocyte, tumor cell, cell line transformed cell, etc.

AAV viral production cells commonly used for production of recombinant AAV particles comprise, but is not limited to HEK293 cells, COS cells, C127, 3T3, CHO, HeLa cells, KB cells, BHK, and other mammalian cell lines as described in U.S. Pat. Nos. 6,156,303, 5,387,484, 5,741,683, 5,691,176, 6,428,988 and 5,688,676; U.S. patent application 2002/0081721, and International Patent Publication Nos. WO 00/47757, WO 00/24916, and WO 96/17947, the contents of which are each incorporated herein by reference in their entireties, insofar as they do not conflict with the present disclosure. In certain embodiments, the AAV viral production cells are trans-complementing packaging cell lines that provide functions deleted from a replication-defective helper virus, e.g., HEK293 cells or other Ea trans-complementing cells.

In certain embodiments, the packaging cell line 293-10-3 (ATCC Accession No. PTA-2361) may be used to produce the AAV particles, as described in U.S. Pat. No. 6,281,010, the content of which is incorporated herein by reference in its entirety as related to the 293-10-3 packaging cell line and uses thereof, insofar as it does not conflict with the present disclosure.

In certain embodiments, of the present disclosure a cell line, such as a HeLA cell line, for trans-complementing E1 deleted adenoviral vectors, which encoding adenovirus Ela and adenovirus E1b under the control of a phosphoglycerate kinase (PGK) promoter can be used for AAV particle production as described in U.S. Pat. No. 6,365,394, the content of which is incorporated herein by reference in its entirety as related to the HeLA cell line and uses thereof, insofar as it does not conflict with the present disclosure.

In certain embodiments, AAV particles are produced in mammalian cells using a triple transfection method wherein a payload construct, parvoviral Rep and parvoviral Cap and a helper construct are comprised within three different constructs. The triple transfection method of the three components of AAV particle production may be utilized to produce small lots of virus for assays comprising transduction efficiency, target tissue (tropism) evaluation, and stability.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other Ela trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the content of which is incorporated herein by reference in its entirety as related to the production and processing of recombinant AAV virus particles, insofar as it does not conflict with the present disclosure. Production may also involve methods known in the art comprising those using 293T cells, triple transfection or any suitable production method.

In certain embodiments, mammalian viral production cells (e.g., 293T cells) can be in an adhesion/adherent state (e.g., with calcium phosphate) or a suspension state (e.g., with polyethyleneimine (PEI)). The mammalian viral production cell is transfected with plasmids required for production of AAV, (i.e., AAV rep/cap construct, an adenoviral helper construct, and/or ITR flanked payload construct). In certain embodiments, the transfection process can comprise optional medium changes (e.g., medium changes for cells in adhesion form, no medium changes for cells in suspension form, medium changes for cells in suspension form if desired). In certain embodiments, the transfection process can comprise transfection mediums such as DMEM or F17. In certain embodiments, the transfection medium can comprise serum or can be serum-free (e.g., cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80° C. to 37° C.), chemical lysis (such as adding detergent triton), mechanical lysis, or by allowing the cell culture to degrade after reaching ˜0% viability. Cellular debris is removed by centrifugation and/or depth filtration. The samples are quantified for AAV particles by DNase resistant genome titration by DNA qPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278, the contents of which are each incorporated herein by reference in their entireties as related to the measurement of particle concentrations, insofar as they do not conflict with the present disclosure).

### Insect Cells

Viral production of the present disclosure comprises processes and methods for producing AAV particles or viral vectors that contact a target cell to deliver a payload construct, e.g., a recombinant viral construct, which comprises a nucleotide encoding a payload molecule. In certain embodiments, the AAV particles or viral vectors of the present disclosure may be produced in a viral production cell that comprises an insect cell.

Growing conditions for insect cells in culture, and production of heterologous products in insect cells in culture are well-known in the art, see U.S. Pat. No. 6,204,059, the content of which is incorporated herein by reference in its entirety as related to the growth and use of insect cells in viral production, insofar as it does not conflict with the present disclosure.

Any insect cell which allows for replication of parvovirus and which can be maintained in culture can be used in accordance with the present disclosure. AAV viral production cells commonly used for production of recombinant AAV particles comprise, but is not limited to, Spodoptera frugiperda, comprising, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, N J (1995); O'Reilly et al., Baculovirus Expression Vectors, A Laboratory Manual, Oxford Univ. Press (1994); Samulski et al., J. Vir.63:3822-8 (1989); Kajigaya et al., Proc. Nat'l. Acad. Sci. USA 88: 4646-50 (1991); Ruffing et al., J. Vir. 66:6922-30 (1992); Kimbauer et al.,Vir.219:37-44 (1996); Zhao et al., Vir.272:382-93 (2000); and Samulski et al., U.S. Pat. No. 6,204,059, the contents of which are each incorporated herein by reference in their entireties as related to the use of insect cells in viral production, insofar as they do not conflict with the present disclosure.

In one embodiment, the AAV particles are made using the methods described in WO2015/191508, the content of which is incorporated herein by reference in its entirety, insofar as it does not conflict with the present disclosure.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the content of which is incorporated herein by reference in its entirety, insofar as it does not conflict with the present disclosure.

Expansion, culturing, transfection, infection and storage of insect cells can be carried out in any cell culture media, cell transfection media or storage media known in the art, comprising Hyclone SFX Insect Cell Culture Media, Expression System ESF AF Insect Cell Culture Medium, Basal IPL-41 Insect Cell Culture Media, ThermoFisher Sf900II media, ThermoFisher Sf900III media, or ThermoFisher Grace's Insect Media. Insect cell mixtures of the present disclosure can also comprise any of the formulation additives or elements described in the present disclosure, comprising (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

In some embodiments, the insect cell culture medium is serum-free and protein-free. In some embodiments, the medium comprises L-glutamate and poloxamer 188. In some embodiments, the medium is ESF AF Insect Cell Culture Medium. In some embodiments, the selected medium (e.g., ESF AF Insect Cell Culture Medium) increases titer at least 1.5 fold or at least 2 fold.

### Baculovirus-Production Systems

In certain embodiments, processes of the present disclosure can comprise production of AAV particles or viral vectors in a baculoviral system using a viral expression construct and a payload construct vector. In certain embodiments, the baculoviral system comprises baculovirus expression vectors (BEVs) and/or baculovirus infected insect cells (BIICs). In certain embodiments, BIICs can be generated by infecting viral production cells (e.g., Sf9 insect cells) with one or more BEVs. In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be polynucleotide incorporated by homologous recombination (transposon donor/acceptor system) into a bacmid by standard molecular biology techniques known and performed by a person skilled in the art.

In certain embodiments, such engineering of the bacmid genome (e.g., to encode a viral expression construct and/or a viral payload construct) produces a BEV. In certain embodiments, a BEV comprising a viral expression construct is also called an “expressionBac”, and a BEV comprising a viral payload construct is also called a “payloadBac”.

In certain embodiments, the process comprises transfection of a single viral replication cell population to produce a single baculovirus (BEV) group which comprises both the viral expression construct and the payload construct. These baculoviruses may be used to infect a viral production cell for production of AAV particles or viral vector.

In certain embodiments, BEVs can be engineered using a system comprising a donor plasmid and a baculoviral construct, wherein a target sequence is transferred from the donor plasmid to the baculovirus construct (e.g., a baculovirus vector). A “target sequence” as used herein refers to a sequence comprising a nucleic acid or gene of interest that is inserted into an expressionBac or payload Bac in a BEV. In certain embodiments, the target sequence is transferred from the donor plasmid to the baculovirus construct via transposition. In certain embodiments, the target sequence comprises a sequence that encodes an AAV structural capsid protein (e.g., VP1, VP2, VP3, or a combination thereof). In certain embodiments, the target sequence comprises a sequence that encodes an AAV non-structural protein (e.g., Rep 52, Rep 78, or a combination thereof). In certain embodiments, the target sequence comprises a sequence that encodes an AAV structural capsid protein (e.g., VP1, VP2, VP3, or a combination thereof) and/or an AAV non-structural protein (e.g., Rep 52, Rep 78, or a combination thereof). In certain embodiments, the target sequence comprises a sequence that encodes a payload (e.g., SEQ ID NO: 979). In certain embodiments, BEVs can be engineered using a Bac-to-Bac™ baculovirus expression system (e.g., according to manufacturer's protocol; ThermoFisher/Invitrogen). In certain embodiments, the Bac-to-Bac™ baculovirus expression system comprises a donor vector (e.g., pFastBac™ vector or pUC57 vector). In certain embodiments, the Bac-to-Bac™ baculovirus expression system comprises a baculovirus shuttle vector (e.g., a bacmid sequence, e.g., a bMON14272 baculovirus shuttle vector). In certain embodiments, the Bac-to-Bac™ baculovirus expression system comprises chemically competent cells hosting a baculovirus shuttle vector. In certain embodiments, the chemically competent cells may be DH10Bac™ E. coli cells comprising a bMON14272 baculovirus shuttle vector.

In some embodiments, the pFastBac™ donor vector comprises a sequence that encodes an AAV structural capsid protein (e.g., VP1, VP2, VP3, or a combination thereof) and/or an AAV non-structural protein (e.g., Rep 52, Rep 78, or a combination thereof). In some embodiments, the pFastBac™ donor vector comprises SEQ ID NO: 1781. The pFastBac™ donor vector sequence is given here:

(SEQ ID NO: 1781)
5’gacgaaagggcctcgtgatacgcctatttttataggttaatgtcatgataataatggtttcttagacgtcaggtggcact
tttcggggaaatgtgcgcggaacccctatttgtttatttttctaaatacattcaaatatgtatccgctcatgagacaataaccctgataaatgc
ttcaataatattgaaaaaggaagagtatgagtattcaacatttccgtgtcgcccttattcccttttttgcggcattttgccttcctgtttttgct
cacccagaaacgctggtgaaagtaaaagatgctgaagatcagttgggtgcacgagtgggttacatcgaactggatctcaacagcggtaa
gatccttgagagttttcgccccgaagaacgttttccaatgatgagcacttttaaagttctgctatgtggcgcggtattatcccgtattgacgc
cgggcaagagcaactcggtcgccgcatacactattctcagaatgacttggttgagtactcaccagtcacagaaaagcatcttacggatg
gcatgacagtaagagaattatgcagtgctgccataaccatgagtgataacactgcggccaacttacttctgacaacgatcggaggacc
gaaggagctaaccgcttttttgcacaacatgggggatcatgtaactcgccttgatcgttgggaaccggagctgaatgaagccataccaa
acgacgagcgtgacaccacgatgcctgtagcaatggcaacaacgttgcgcaaactattaactggcgaactacttactctagcttcccg
gcaacaattaatagactggatggaggcggataaagttgcaggaccacttctgcgctcggcccttccggctggctggtttattgctgataa
atctggagccggtgagcgtgggtctcgcggtatcattgcagcactggggccagatggtaagccctcccgtatcgtagttatctacacga
cggggagtcaggcaactatggatgaacgaaatagacagatcgctgagataggtgcctcactgattaagcattggtaactgtcagacca
agtttactcatatatactttagattgatttaaaacttcatttttaatttaaaaggatctaggtgaagatcctttttgataatctcatgaccaaaa
tcccttaacgtgagttttcgttccactgagcgtcagaccccgtagaaaagatcaaaggatcttcttgagatcctttttttctgcgcgtaatctgc
tgcttgcaaacaaaaaaaccaccgctaccagcggtggtttgtttgccggatcaagagctaccaactctttttccgaaggtaactggcttc
agcagagcgcagataccaaatactgttcttctagtgtagccgtagttaggccaccacttcaagaactctgtagcaccgcctacatacctc
gctctgctaatcctgttaccagtggctgctgccagtggcgataagtcgtgtcttaccgggttggactcaagacgatagttaccggataag
gcgcagcggtcgggctgaacggggggttcgtgcacacagcccagcttggagcgaacgacctacaccgaactgagatacctacagc
gtgagctatgagaaagcgccacgcttcccgaagggagaaaggcggacaggtatccggtaagcggcagggtcggaacaggagag
cgcacgagggagcttccagggggaaacgcctggtatctttatagtcctgtcgggtttcgccacctctgacttgagcgtcgatttttgtgat
gctcgtcaggggggcggagcctatggaaaaacgccagcaacgcggcctttttacggttcctggccttttgctggccttttgctcacatgt
tctttcctgcgttatcccctgattctgtggataaccgtattaccgcctttgagtgagctgataccgctcgccgcagccgaacgaccgagc
gcagcgagtcagtgagcgaggaagcggaagagcgcccaatacgcaaaccgcctctccccgcgcgttggccgattcattaatgcag
ctggcacgacaggtttcccgactggaaagcgggcagtgagcgcaacgcaattaatgtgagttagctcactcattaggcaccccaggc
tttacactttatgcttccggctcgtatgttgtgtggaattgtgagcggataacaatttcacacaggaaacagctatgaccatgattacgcca
agcttgcatgcaggcctctgcagtcgacaccggttaagcgggtgtgggcggacaataaagtcttaaactgaacaaaatagatctaaac
tatgacaataaagtcttaaactagacagaatagttgtaaactgaaatcagtccagttatgctgtgaaaaagcatactggacttttgttatgg
ctaaagcaaactcttcattttctgaagtgcaaattgcccgtcgtattaaagaggggcgtggccaagggcatggtaaagactatattcgcg
gcgttgtgacaatttaccgaacaactccgcggccgggaagccgatctcggcttgaacgaattgttaggtggcggtacttgggtcgatat
caaagtgcatcacttcttcccgtatgcccaactttgtatagagagccactgcgggatcgtcaccgtaatctgcttgcacgtagatcacata
agcaccaagcgcgttggcctcatgcttgaggagattgatgagcgcggtggcaatgccctgcctccggtgctcgccggagactgcga
gatcatagatatagatctcactacgcggctgctcaaacttgggcagaacgtaagccgcgagagcgccaacaaccgcttcttggtcgaa
ggcagcaagcgcgatgaatgtcttactacggagcaagttcccgaggtaatcggagtccggctgatgttgggagtaggtggctacgtct
ccgaactcacgaccgaaaagatcaagagcagcccgcatggatttgacttggtcagggccgagcctacatgtgcgaatgatgcccata
cttgagccacctaactttgttttagggcgactgccctgctgcgtaacatcgttgctgctgcgtaacatcgttgctgctccataacatcaaac
atcgacccacggcgtaacgcgcttgctgcttggatgcccgaggcatagactgtacaaaaaaacagtcataacaagccatgaaaaccg
ccactgcgccgttaccaccgctgcgttcggtcaaggttctggaccagttgcgtgagcgcatacgctacttgcattacagtttacgaaccg
aacaggcttatgtcaactgggttcgtgccttcatccgtttccacggtgtgcgtcacccggcaaccttgggcagcagcgaagtcgaggc
atttctgtcctggctggcgaacgagcgcaaggtttcggtctccacgcatcgtcaggcattggcggccttgctgttcttctacggcaaggt
gctgtgcacggatctgcccttgcttcaggagatcggtagacctcggccgtcgcggcgcttgccggtggtgctgaccccggatgaagt
ggttcgcatcctcggttttctggaaggcgagcatcgtttgttcgcccaggactctagctatagttctagtggttggctacgtacccgtagtg
gctatggcagggcttgccgccccgacgttggctgcgagccctgggccttcacccgaacttgggggttggggtggggaaaaggaag
aaacgcgggcgtattggtcccaatggggtctcggtggggtatcgacagagtgccagccctgggaccgaaccccgcgtttatgaacaa
acgacccaacacccgtgcgttttattctgtctttttattgccgtcatagcgcgggttccttccggtattgtctccttccgtgtttcagttagcct
cccccatctcccggtaccgcatgctatgcatcagctgctagcttacagattacgagtcaggtatctggtgccaatggggcgaggctctg
aatacacgccattagtgtccacagtaaagtccacattaacagacttgttgtagttggaagtgtactgaatttcgggattccagcgtttgctg
ttttccttctgcagctcccactcgatctccacgctgacctgtcccgtggagtactgtgtgatgaaggaagcaaactttgccgcactgaag
gtggtcgaaggattcgcaggtaccggggtgttcttgatgagaatctgtggaggagggtgtttaagtccgaatccacccatgaggggag
aggggtgaaaatgtccgtccgtgtgtggaatctttgcccagatgggcccctgaaggtacacatctctgtcctgccagaccatgcctgga
agaacgccttgtgtgttgacatctgcggtagctgcttgtctgttgcctctctggaggttggtagatacagaaccatactgctccgtagcca
cgggattggttgtcctgatttcctcttcgtctgtaatcatgaccttttcaatgtccacatttgttttctctgagccttgcttcccaaagatgagaa
ccccgctctgaggaaaaaacttttcttcatcgtccttgtggcttgccatggccgggcccggattcaccagagagtctctgccattgaggt
ggtacttggtagctccagtccacgagtattcactgttgttgttatccgcagatgtctttgatactcgctgctggcggtaacagggtccagg
aagccagttcctagactggtcccgaatgtcactcgctccggcctgagaaaactgaagccttgactgcgtggtggttccacttggagtgtt
tgttctgctcaagtaatacaggtactggtcgatgagaggattcatgagacggtccagactctggctgtgagcgtagctgctgtggaaag
gaacgtcctcaaaagtgtagctgaaggtaaagttgtttccggtacgcagcatctgagaaggaaagtactccaggcagtaaaatgaaga
gcgtcctactgcctgactcccgttgttcagggtgaggtatccatactgtggcaccatgaagacgtctgctgggaacggcgggaggcat
ccttgatgcgccgagccgaggacgtacgggagctggtactccgagtcagtaaacacctgaaccgtgctggtaaggttattggcaatc
gtcgtcgtaccgtcattctgcgtgacctctttgacttgaatgttaaagagcttgaagttgagtctcttgggtcggaatccccagttgttgttg
atgagtctttgccagtcacgtggtgaaaagtggcagtggaatctgttgaagtcaaaatacccccaaggggtgctgtagccaaagtagtg
attgtcgttcgaggctcctgattggctggaaatttgtttgtagaggtggttgttgtaggtgggcagggcccaggttcgggtgctggtggtg
atgactctgtcgcccatccatgtggaatcgcaatgccaatttcccgaggaattacccactccgtcggcgccctcgttattgtctgccattg
gtgcgccactgcctgtagccatcgtattagttcccagaccagagggggctgctggtggctgtccgagaggctgggggtcaggtactg
agtctgcgtctccagtctgaccaaaattcaatctttttcttgcaggctgctggcccgcctttccggttcccgaggaggagtctggctccac
aggagagtgctctaccggcctcttttttcccggagccgtcttaacaggttcctcaaccaggcccagaggttcaagaaccctctttttcgcc
tggaagactgctcgtccgaggttgcccccaaaagaggtatcttctttaaggcgctcctgaaactccgcgtcggcgtggttgtacttgag
gtacgggttgtctccgctgtcgagctgccggtcgtaggctttgtcgtgctcgagggccgcggcgtctgcctcgttgaccggctctccctt
gtcgagtccgttgaagggtccgaggtacttgtacccaggaagcacaagacccctgctgtcgtccttatgccgctctgcgggctttggtg
gtggtgggccaggtttgagcttccaccactgtcttattccttcagagagagtgtcctcgagccaatcgggtagataaccgtcggcagcc
agtttaacaggatcccccgggtgatcaagtcttcgtcgagtgattgtaaataaaatgtaatttacagtatagtattttaattaatatacaaatg
atttgataataattcttatttaactataatatattgtgttgggttgaattaaaggtccgtatactccggaatattaatagatcatggagataatta
aaatgataaccatctcgcaaataaataagtattttactgttttcgtaacagttttgtaataaaaaaacctataaatattccggattattcatacc
gtcccaccatcgggcgcggatctgccgccctggcggggttttacgagattgtgattaaggtccccagcgaccttgacgagcatctgcc
cggcatttctgacagctttgtgaactgggtggccgagaaggagtgggagttgccgccagattctgacttggatctgaatctgattgagc
aggcacccctgaccgtggccgagaagctgcagcgcgactttctgacggagtggcgccgtgtgagtaaggccccggaggcccttttc
tttgtgcaatttgagaagggagagagctacttccacttacacgtgctcgtggaaaccaccggggtgaaatccttagttttgggacgtttcc
tgagtcagattcgcgaaaaactgattcagagaatttaccgcgggatcgagccgactttgccaaactggttcgcggtcacaaagaccag
aaacggcgccggaggcgggaacaaggtggtggacgagtgctacatccccaattacttgctccccaaaacccagcctgagctccagt
gggcgtggactaatttagaacagtatttaagcgcctgtttgaatctcacggagcgtaaacggttggtggcgcagcatctgacgcacgtg
tcgcagacgcaggagcagaacaaagagaatcagaatcccaattctgacgcgccggtgatcagatcaaaaacttcagccaggtacat
ggagctggtcgggtggctcgtggacaaggggattacctcggagaagcagtggatccaggaggaccaggcctcatacatctccttca
atgcggcctccaactcgcggtcccaaatcaaggctgccttggacaatgcgggaaagattatgagcctgactaaaaccgcccccgact
acctggtgggccagcagcccgtggaggacatttccagcaatcggatttataaaattttggaactaaacgggtacgatccccaatatgcg
gcttccgtctttctgggatgggccacgaaaaagttcggcaagaggaacaccatctggctgtttgggcctgcaactaccgggaagacca
acatcgcggaggccatagcccacactgtgcccttctacgggtgcgtaaactggaccaatgagaactttcccttcaacgactgtgtcgac
aagatggtgatctggtgggaggaggggaagatgaccgccaaggtcgtggagtcggccaaagccattctcggaggaagcaaggtgc
gcgtggaccagaaatgcaagtcctcggcccagatagacccgactcccgtgatcgtcacctccaacaccaacatgtgcgccgtgattg
acgggaactcaacgaccttcgaacaccagcagccgttgcaagaccggatgttcaaatttgaactcacccgccgtctggatcatgacttt
gggaaggtcaccaagcaggaagtcaaagactttttccggtgggcaaaggatcacgtggttgaggtggagcatgaattctacgtcaaa
aagggtggagccaagaaaagacccgcccccagtgacgcagatataagtgagcccaaacgggtgcgcgagtcagttgcgcagccat
cgacgtcagacgcggaagcttcgatcaactacgcagacaggtaccaaaacaaatgttctcgtcacgtgggcatgaatctgatgctgttt
ccctgcagacaatgcgagagaatgaatcagaattcaaatatctgcttcactcacggacagaaagactgtttagagtgctttcccgtgtca
gaatctcaacccgtttctgtcgtcaaaaaggcgtatcagaaactgtgctacattcatcatatcatgggaaaggtgccagacgcttgcact
gcctgcgatctggtcaatgtggatttggatgactgcatctttgaacaataaactcgaggaatcaagcttgcatgcctgcaggtcgactcta
gagcctgcagtctcgacaagcttgtcgagaagtactagaggatcataatcagccataccacatttgtagaggttttacttgctttaaaaaa
cctcccacacctccccctgaacctgaaacataaaatgaatgcaattgttgttgttaacttgtttattgcagcttataatggttacaaataaag
caatagcatcacaaatttcacaaataaagcatttttttcactgcattctagttgtggtttgtccaaactcatcaatgtatcttatcatgtctggat
ctgatcactgcttgagcctaggagatccgaaccagataagtgaaatctagttccaaactattttgtcatttttaattttcgtattagcttacgac
gctacacccagttcccatctattttgtcactcttccctaaataatccttaaaaactccatttccacccctcccagttcccaactattttgtccgc
ccacagcggggcattgcggccgcatctagatgcattcgcgaggtaccgagctcgaattcactggccgtcgttttacaacgtcgtgact
gggaaaaccctggcgttacccaacttaatcgccttgcagcacatccccctttcgccagctggcgtaatagcgaagaggcccgcaccg
atcgcccttcccaacagttgcgcagcctgaatggcgaatggcgcctgatgcggtattttctccttacgcatctgtgcggtatttcacaccg
catatggtgcactctcagtacaatctgctctgatgccgcatagttaagccagccccgacacccgccaacacccgctgacgcgccctga
cgggcttgtctgctcccggcatccgcttacagacaagctgtgaccgtctccgggagctgcatgtgtcagaggttttcaccgtcatcacc
gaaacgcgcga 3’.

In some embodiments, the pUC57 donor vector comprises a sequence that encodes a payload (e.g., SEQ ID NO: 979). In some embodiments, the pUC57 donor vector comprises SEQ ID NO: 1782. The pUC57 donor vector sequence is given here:

(SEQ ID NO: 1782)
5’tcgcgcgtttcggtgatgacggtgaaaacctctgacacatgcagctcccggagacggtcacagcttgtctgtaagc
ggatgccgggagcagacaagcccgtcagggcgcgtcagcgggtgttggcgggtgtcggggctggcttaactatgcggcatcagag
cagattgtactgagagtgcaccatatgcggtgtgaaataccgcacagatgcgtaaggagaaaataccgcatcaggcgccattcgccat
tcaggctgcgcaactgttgggaagggcgatcggtgcgggcctcttcgctattacgccagctggcgaaagggggatgtgctgcaagg
cgattaagttgggtaacgccagggttttcccagtcacgacgttgtaaaacgacggccagtgaagccccgctgtgggcggacaaaata
gttgggaactgggaggggtggaaatggagtttttaaggattatttagggaagagtgacaaaatagatgggaactgggtgtagcgtcgt
aagctaatacgaaaattaaaaatgacaaaatagtttggaactagatttcacttatctggttcggatctcgaccaattgacattattgaagcat
ttatcagggttattgtctcagacctgcaggcagctgcgcgctcgctcgctcactgaggccgcccgggcaaagcccgggcgtcgggc
gacctttggtcgcccggcctcagtgagcgagcgagcgcgcagagagggagtggccaactccatcactaggggttccttgtagttaat
gattaacccgccatgctacttatctacgtagccatgcgtcgacataacgcgtatatctagacgttacataacttacggtaaatggcccgcc
tggctgaccgcccaacgacccccgcccattgacgtcaataatgacgtatgttcccatagtaacgccaatagggactttccattgacgtc
aatgggtggagtatttacggtaaactgcccacttggcagtacatcaagtgtatcatatgccaagtacgccccctattgacgtcaatgacg
gtaaatggcccgcctggcattatgcccagtacatgaccttatgggactttcctacttggcagtacatctagtattagtcatcgctattaccat
ggtgatgcggttttggcagtacatcaatgggcgtggatagcggtttgactcacggggatttccaagtctccaccccattgacgtcaatgg
gagtttgttttggcaccaaaatcaacgggactttccaaaatgtcgtaacaactccgccccattgacgcaaatgggcggtaggcgtgtac
ggtgggaggtctatataagcagagctcgtttagtgaaccgtcagatcgcctggagacgccatccacgctgttttgacctccatagaaga
caccgggaccgatccagcctccgcggattcgaatcccggccgggaacggtgcattggaacgcggattccccgtgccaagagtgac
gtaagtaccgcctatagagtctataggcccacaaaaaatgctttcttcttttaatatacttttttgtttatcttatttctaatactttccctaatct
ctttctttcagggcaataatgatacaatgtatcatgcctctttgcaccattctaaagaataacagtgataatttctgggttaaggcaatagcaata
tttctgcatataaatatttctgcatataaattgtaactgatgtaagaggtttcatattgctaatagcagctacaatccagctaccattctgctttt
attttatggttgggataaggctggattattctgagtccaagctaggcccttttgctaatcatgttcatacctcttatcttcctcccacagctcct
gggcaacgtgctggtctgtgtgctggcccatcactttggcaaagaattgggattcgaacatcgattgaattccccggggatccaccatg
aacgcaagtgaattccgaaggagagggaaggagatggtggattacgtggccaactacatggaaggcattgagggacgccaggtcta
ccctgacgtggagcccgggtacctgcggccgctgatccctgccgctgcccctcaggagccagacacgtttgaggacatcatcaacg
acgttgagaagataatcatgcctggggtgacgcactggcacagcccctacttcttcgcctacttccccactgccagctcgtacccggcc
atgcttgcggacatgctgtgcggggccattggctgcatcggcttctcctgggcggcaagcccagcatgcacagagctggagactgtg
atgatggactggctcgggaagatgctggaactaccaaaggcatttttgaatgagaaagctggagaagggggaggagtgatccaggg
aagtgccagtgaagccaccctggtggccctgctggccgctcggaccaaagtgatccatcggctgcaggcagcgtccccagagctca
cacaggccgctatcatggagaagctggtggcttactcatccgatcaggcacactcctcagtggaaagagctgggttaattggtggagt
gaaattaaaagccatcccctcagatggcaacttcgccatgcgtgcgtctgccctgcaggaagccctggagagagacaaagcggctg
gcctgattcctttctttatggttgccaccctggggaccacaacatgctgctcctttgacaatctcttagaagtcggtcctatctgcaacaag
gaagacatatggctgcacgttgatgcagcctacgcaggcagtgcattcatctgccctgagttccggcaccttctgaatggagtggagttt
gcagattcattcaactttaatccccacaaatggctattggtgaattttgactgttctgccatgtgggtgaaaaagagaacagacttaacgg
gagcctttagactggaccccacttacctgaagcacagccatcaggattcagggcttatcactgactaccggcattggcagataccactg
ggcagaagatttcgctctttgaaaatgtggtttgtatttaggatgtatggagtcaaaggactgcaggcttatatccgcaagcatgtccagct
gtcccatgagtttgagtcactggtgcgccaggatccccgctttgaaatctgtgtggaagtcattctggggcttgtctgctttcggctaaag
ggttccaacaaagtgaatgaagctcttctgcaaagaataaacagtgccaaaaaaatccacttggttccatgtcacctcagggacaagttt
gtcctgcgctttgccatctgttctcgcacggtggaatctgcccatgtgcagcgggcctgggaacacatcaaagagctggcggccgac
gtgctgcgagcagagagggagtaggagtgaagccaggacctgcagaagcttgcctcgagcagcgctgctcgagagatctacgggt
ggcatccctgtgacccctccccagtgcctctcctggccctggaagttgccactccagtgcccaccagccttgtcctaataaaattaagtt
gcatcattttgtctgactaggtgtccttctataatattatggggtggaggggggtggtatggagcaaggggcaagttgggaagacaacct
gtagggcctgcggggtctattgggaaccaagctggagtgcagtggcacaatcttggctcactgcaatctccgcctcctgggttcaagc
gattctcctgcctcagcctcccgagttgttgggattccaggcatgcatgaccaggctcagctaatttttgtttttttggtagagacggggttt
caccatattggccaggctggtctccaactcctaatctcaggtgatctacccaccttggcctcccaaattgctgggattacaggcgtgaac
cactgctcccttccctgtccttactagatttaaatatgtcgtgcatcgatgctacgtagataagtagcatggcgggttaatcattaactacag
aggaacccctagtgatggagttggccactccctctctgcgcgctcgctcgctcactgaggccgggcgaccaaaggtcgcccgacgc
ccgggctttgcccgggcggcctcagtgagcgagcgagcgcgcagctgcctgcaggctgcctgcaggcatttaattaagcaagctgt
agccaaccactagaactatagctagagtcctgggcgaacaaacgatgctcgccttccagaaaaccgaggatgcgaaccacttcatcc
ggggtcagcaccaccggcaagcgccgcgacggccgaggtcttccgatctcctgaagccagggcagatccgtgcacagcaccttgc
cgtagaagaacagcaaggccgccaatgcctgacgatgcgtggagaccgaaaccttgcgctcgttcgccagccaggacagaaatgc
ctcgacttcgctgctgcccaaggttgccgggtgacgcacaccgtggaaacggatgaaggcacgaacccagttgacataagcctgttc
ggttcgtaaactgtaatgcaagtagcgtatgcgctcacgcaactggtccagaaccttgaccgaacgcagcggtggtaacggcgcagt
ggcggttttcatggcttgttatgactgtttttttgtacagtctatgcctcgggcatccaagcagcaagcgcgttacgccgtgggtcgatgttt
gatgttatggagcagcaacgatgttacgcagcagcaacgatgttacgcagcagggcagtcgccctaaaacaaagttaggtggctcaa
gtatgggcatcattcgcacatgtaggctcggccctgaccaagtcaaatccatgcgggctgctcttgatcttttcggtcgtgagttcggag
acgtagccacctactcccaacatcagccggactccgattacctcgggaacttgctccgtagtaagacattcatcgcgcttgctgccttcg
accaagaagcggttgttggcgctctcgcggcttacgttctgcccaagtttgagcagccgcgtagtgagatctatatctatgatctcgcag
tctccggcgagcaccggaggcagggcattgccaccgcgctcatcaatctcctcaagcatgaggccaacgcgcttggtgcttatgtgat
ctacgtgcaagcagattacggtgacgatcccgcagtggctctctatacaaagttgggcatacgggaagaagtgatgcactttgatatcg
acccaagtaccgccacctaacaattcgttcaagccgagatcggcttcccggccgcggagttgttcggtaaattgtcacaacgccgcga
atatagtctttaccatgcccttggccacgcccctctttaatacgacgggcaatttgcacttcagaaaatgaagagtttgctttagccataac
aaaagtccagtatgctttttcacagcataactggactgatttcagtttacaactattctgtctagtttaagactttattgtcatagtttagatctat
tttgttcagtttaagactttattgtccgcccacacccgcttggcgtaatcatggtcatagctgtttcctgtgtgaaattgttatccgctcacaat
tccacacaacatacgagccggaagcataaagtgtaaagcctggggtgcctaatgagtgagctaactcacattaattgcgttgcgctcac
tgcccgctttccagtcgggaaacctgtcgtgccagctgcattaatgaatcggccaacgcgcggggagaggcggtttgcgtattgggc
gctcttccgcttcctcgctcactgactcgctgcgctcggtcgttcggctgcggcgagcggtatcagctcactcaaaggcggtaatacgg
ttatccacagaatcaggggataacgcaggaaagaacatgtgagcaaaaggccagcaaaaggccaggaaccgtaaaaaggccgcgt
tgctggcgtttttccataggctccgcccccctgacgagcatcacaaaaatcgacgctcaagtcagaggtggcgaaacccgacaggac
tataaagataccaggcgtttccccctggaagctccctcgtgcgctctcctgttccgaccctgccgcttaccggatacctgtccgcctttct
cccttcgggaagcgtggcgctttctcatagctcacgctgtaggtatctcagttcggtgtaggtcgttcgctccaagctgggctgtgtgca
cgaaccccccgttcagcccgaccgctgcgccttatccggtaactatcgtcttgagtccaacccggtaagacacgacttatcgccactgg
cagcagccactggtaacaggattagcagagcgaggtatgtaggcggtgctacagagttcttgaagtggtggcctaactacggctaca
ctagaagaacagtatttggtatctgcgctctgctgaagccagttaccttcggaaaaagagttggtagctcttgatccggcaaacaaacca
ccgctggtagcggtggtttttttgtttgcaagcagcagattacgcgcagaaaaaaaggatctcaagaagatcctttgatcttttctacggg
gtctgacgctcagtggaacgaaaactcacgttaagggattttggtcatgagattatcaaaaaggatcttcacctagatccttttaaattaaa
aatgaagttttaaatcaatctaaagtatatatgagtaaacttggtctgacagttaccaatgcttaatcagtgaggcacctatctcagcgatct
gtctatttcgttcatccatagttgcctgactccccgtcgtgtagataactacgatacgggagggcttaccatctggccccagtgctgcaat
gataccgcgagacccacgctcaccggctccagatttatcagcaataaaccagccagccggaagggccgagcgcagaagtggtcct
gcaactttatccgcctccatccagtctattaattgttgccgggaagctagagtaagtagttcgccagttaatagtttgcgcaacgttgttgc
cattgctacaggcatcgtggtgtcacgctcgtcgtttggtatggcttcattcagctccggttcccaacgatcaaggcgagttacatgatcc
cccatgttgtgcaaaaaagcggttagctccttcggtcctccgatcgttgtcagaagtaagttggccgcagtgttatcactcatggttatgg
cagcactgcataattctcttactgtcatgccatccgtaagatgcttttctgtgactggtgagtactcaaccaagtcattctgagaatagtgtat
gcggcgaccgagttgctcttgcccggcgtcaatacgggataataccgcgccacatagcagaactttaaaagtgctcatcattggaaaa
cgttcttcggggcgaaaactctcaaggatcttaccgctgttgagatccagttcgatgtaacccactcgtgcacccaactgatcttcagcat
cttttactttcaccagcgtttctgggtgagcaaaaacaggaaggcaaaatgccgcaaaaaagggaataagggcgacacggaaatgttg
aatactcatactcttcctttttcaatattattgaagcatttatcagggttattgtctcatgagcggatacatatttgaatgtatttagaaaaataaa
caaataggggttccgcgcacatttccccgaaaagtgccacctgacgtctaagaaaccattattatcatgacattaacctataaaaatagg
cgtatcacgaggccctttcgtc 3’.

In some embodiments, the bMON14272 baculovirus shuttle vector comprises SEQ ID NO: 1783.

In certain embodiments, transfection of separate viral production cell (VPC) populations with one or more expressionBac and/or payloadBac produces at least one group (e.g., two groups) of baculovirus infected viral production cells, each comprising at least one viral expression construct or viral payload construct. In some embodiments, the VPC may be an insect cell (e.g., Sf9 cell) and the baculovirus infected viral production cell may be a baculovirus infected insect cell (BIICs). In some embodiments, the BIICs comprise at least one group of bacmids (e.g., expressionBacs and/or payloadBacs) which may be used to infect one or more additional viral production cells (VPC) (e.g., Sf9 cells) for expanded production of AAV particles or viral vector. In certain embodiments, one or more BIICs may comprise at least one viral payload construct (such cells may be referred to as payloadBIIC) which encodes at least one payload protein (e.g., AADC). In certain embodiments, one or more BIICs may comprise at least one viral expression construct (expressionBIIC) which encodes at least one viral capsid or replication protein (e.g., AAV2 Rep/Cap). In certain embodiments, at least one viral production cell (e.g., at least one Sf9 cell) may be co-incubated (e.g., co-infected) with at least one expressionBIIC and at least one payloadBIIC to produce at least one viral production cell comprising a viral expression construct and a viral payload construct, e.g., an AAV2 capsid containing a construct encoding an AADC protein. In certain embodiments, VPCs comprising expressionBacs and/or payloadBacs are used to infect additional VPCs to produce more expressionBacs and/or payloadBacs. In some embodiments, VPCs infected with expressionBacs and/or payloadBacs provide increased stability and prolonged storage of the expressionBacs and/or payloadBacs as compared to expressionBacs and/or payloadBacs outside of VPCs. In some embodiments, the expressionBacs and/or payloadBacs produced by infected VPCs are used to infect insect cells (e.g., Sf9 cells).

In certain embodiments, the at least one viral production cell (e.g., Sf9 cell) comprises a viral payload construct, e.g., a payloadBAC, comprising the nucleic acid sequence of SEQ ID NO: 979, which encodes the AADC amino acid sequence of SEQ ID NO: 978. In some embodiments, the at least one viral production cell (e.g., Sf9 cell) comprises a viral expression construct, e.g., an expressionBAC, encoding AAV2 capsid proteins. In some embodiments, the viral expression construct comprises the nucleic acid sequence of SEQ ID NO: 1778. In some embodiments, the viral expression construct encodes the amino acid sequence of SEQ ID NO: 16. In some embodiments, the viral expression construct encodes VP1, VP2, and VP3.

In certain embodiments, the process of preparing VPCs comprises transfection (e.g., co-transfection) of a single viral replication cell population to produce a single baculovirus (BIIC) group which comprises both the viral expression construct and the payload construct. These baculoviruses may be used to infect a viral production cell for production of AAV particles or viral vector.

In certain embodiments, BEVs are produced using a Bacmid Transfection agent, such as Promega FuGENE HD, WFI water, or ThermoFisher Cellfectin II Reagent. In certain embodiments, BEVs are produced and expanded in viral production cells, such as an insect cell.

In certain embodiments, the method utilizes seed cultures of viral production cells that comprise one or more BEVs, comprising baculovirus infected insect cells (BIICs). The seed BIICs have been transfected/transduced/infected with an Expression BEV which comprises a viral expression construct, and also a Payload BEV which comprises a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN₂ vapor.

Baculoviruses comprise several essential proteins which play a role in the function and replication of the baculovirus, including replication proteins, envelope proteins, and capsid proteins. The baculovirus genome thus comprises nucleotide sequences encoding such proteins. As a non-limiting example, the baculovirus genome can comprise nucleotide sequences which encode the protein for the baculovirus construct. In some embodiments, the baculovirus genome can encode proteins such as the GP64 baculovirus envelope protein and the VP39 baculovirus capsid protein for the baculovirus construct.

Baculovirus expression vectors (BEV) for producing AAV particles in insect cells, comprising but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the content of which is incorporated herein by reference in its entirety as related to the production and use of BEVs and viral particles, insofar as it does not conflict with the present disclosure.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture Wasilko D J et al. Protein Expr Purif. 2009 June; 65(2):122-32, the content of which is incorporated herein by reference in its entirety as related to the production and use of BEVs and viral particles, insofar as it does not conflict with the present disclosure.

A genetically stable baculovirus may be used to produce a source of the one or more of the components for producing AAV particles in invertebrate cells. In certain embodiments, defective baculovirus expression vectors may be maintained episomally in insect cells. In such an embodiment the corresponding bacmid vector is engineered with replication control elements, comprising but not limited to promoters, enhancers, and/or cell-cycle regulated replication elements.

In certain embodiments, baculoviruses may be engineered with a marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In certain embodiments, stable viral producing cells permissive for baculovirus infection are engineered with at least one stable integrated copy of any of the elements necessary for AAV replication and vector production comprising, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In certain embodiments, the Baculovirus expression vectors (BEV) are based on the AcMNPV baculovirus or BmNPV baculovirus BmNPV. In certain embodiments, a bacmid of the present disclosure is based on (i.e., engineered variant of) an AcMNPV bacmid such as bmon14272, vAce25ko or vAclef11KO.

In certain embodiments, the Baculovirus expression vectors (BEV) is a BEV in which the baculoviral v-cath gene has been partially or fully deleted (“v-cath deleted BEV”) or mutated. In certain embodiments, the BEVs lack the v-cath gene or comprise a mutationally inactivated version of the v-cath gene. In certain embodiments, the BEVs lack the v-cath gene. In certain embodiments, the BEVs comprise a mutationally inactivated version of the v-cath gene.

Viral production bacmids of the present disclosure can comprise deletion of certain baculoviral genes or loci.

In certain embodiments, baculoviral inoculum banks can be produced using small-scale shake flasks, such as 3L or 5L shake flasks. However, this process is generally limited in the maximum cell density of the BIIC cells which can be produced, and thus requires centrifugation to concentrate resulting cells into a workable concentration. This correspondingly limits the volume (i.e., quantity) of the baculoviral inoculum bank (˜600 mL) which can be produced and stored using this method. This process also presents sterility concerns due to open operation.

In certain embodiments, baculoviral inoculum banks can be produced using bioreactors, such as 20-50L bioreactors. However, this process is also generally limited in the maximum cell density of the BIIC cells which can be produced, and thus requires significant processing through Tangential Flow Filtration (TFF) and/or centrifugation to concentrate resulting cells into a workable concentration (with 3 L of culture material being required to produce about 600 mL of concentrated BIIC formulation, corresponding with a 15-25% yield). This correspondingly limits the volume (i.e. quantity) of the baculoviral inoculum bank (˜3000 mL) which can be produced and stored using this method. This process also presents sterility concerns due to open operation.

In certain embodiments, perfusion technology can be used in the production of baculoviral inoculum banks. Perfusion systems are fluid circulation systems which use combinations of pumps, filters and screens to retain cells inside a bioreactor while continually removing cell waste products and replacing media depleted of nutrients by cell metabolism. In certain embodiments, the perfusion system is an alternating tangential flow (ATF) perfusion system. In certain embodiments, a perfusion system can be used in coordination with bioreactors to manage and cycle cell culture media within a bioreactor during the production of Baculovirus Infected Insect Cells (BIICs). In certain embodiments, a perfusion system can be used to support the production of high quality BIIC banks having an unexpectedly high cell density at large-scale. In certain embodiments, a perfusion system can be used to provide an infection-cell-to-product-cell yield of greater than 70% (e.g., 75-80%, 80-85%, 85-90%, 90-95% or 95-100%). In certain embodiments, a perfusion system can be used to perform a media switch within the bioreactor, such as the replacements of a cell culture media with a cryopreservation media which allows for BIIC cells to be frozen and preserved.

The present disclosure presents methods for producing a baculovirus infected insect cell (BIIC), e.g., expressionBIICs and/or payloadBIICs. In certain embodiments, the present disclosure presents methods for producing a baculovirus infected insect cell (BIIC) which comprises the following steps: (a) introducing a volume of cell culture medium into a bioreactor; (b) introducing at least one viral production cell (VPC) into the bioreactor and expanding the number of VPCs in the bioreactor to a target VPC cell density; (c) introduction at least one Baculoviral Expression Vector (BEV) into the bioreactor, wherein the BEV comprises an AAV viral expression construct or an AAV payload construct; (d) incubating the mixture of VPCs and BEVs in the bioreactor under conditions which allow at least one BEV to infect at least one VPC to produce a baculovirus infected insect cell (BIIC); (e) incubating the bioreactor under conditions which allow the number of BIICs in the bioreactor to reach a target BIIC cell density; and (f) harvesting the BIICs from the bioreactor. In certain embodiments, the bioreactor has a volume of at least 5 L, 10 L, 20 L, 50 L, 100 L, or 200 L. In certain embodiments, the volume of cell culture medium (i.e. working volume) in the bioreactor is at least 5 L, 10 L, 20 L, 50 L, 100 L, or 200 L.

In certain embodiments, the VPC density at BEV introduction is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at BEV introduction is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the target VPC cell density at BEV introduction is 1.5-4.0×10⁶ cells/mL. In certain embodiments, the target VPC cell density at BEV introduction is 2.0-3.5×10⁶ cells/mL.

In certain embodiments, the BEVs are introduced into the bioreactor at a target Multiplicity of Infection (MOI) of BEVs to VPCs. In certain embodiments, the BEV MOI is 0.0005-0.003, or more specifically 0.001-0.002.

In certain embodiments, the bioreactor can comprise a perfusion system for managing the cell culture medium within the bioreactor. In certain embodiments, the perfusion system is an alternating tangential flow (ATF) perfusion system. In certain embodiments, the perfusion system replaces at least a portion of the culture medium in the bioreactor while retaining at least 90% of the VPCs and BIICs within the bioreactor. In certain embodiments, the perfusion system removes cell waste products from the cell culture medium within the bioreactor. In certain embodiments, the perfusion system replaces cell culture media which has been depleted of nutrients by cellular metabolism. In certain embodiments, the perfusion system replaces the cell culture media with a cryopreservation media which allows for BIIC cells to be frozen and preserved. In certain embodiments, the perfusion system replaces the cell culture media with a cell culture media supplemented with growth or production boosting factors to increase the quality and quantity of the AAV product.

In certain embodiments, the BIICs are harvested from the bioreactor at a specific BIIC cell density. In certain embodiments, the BIICs harvested from the bioreactor have a specific BIIC cell density. In certain embodiments, the BIIC cell density at harvesting is 6.0-18.0×10⁶ cells/mL, 8.0-16.5×10⁶ cells/mL, 10.0-16.5×10⁶ cells/mL.

In some embodiments, BIICs (expressionBIICs, payloadBIICs) are used to transfect viral production cells, e.g., Sf9 cells. In some embodiments, baculovirus (expressionBacs, payloadBacs) are used to transfect viral production cells, e.g., Sf9 cells. Other

In certain embodiments expression hosts comprise, but are not limited to, bacterial species within the genera Escherichia, Bacillus, Pseudomonas, or Salmonella.

In certain embodiments, a host cell which comprises AAV rep and cap genes stably integrated within the cell's chromosomes, may be used for AAV particle production. In a non-limiting example, a host cell which has stably integrated in its chromosome at least two copies of an AAV rep gene and AAV cap gene may be used to produce the AAV particle according to the methods and constructs described in U.S. Pat. No. 7,238,526, the content of which is incorporated herein by reference in its entirety as related to the production of viral particles, insofar as it does not conflict with the present disclosure.

In certain embodiments, the AAV particle can be produced in a host cell stably transformed with a molecule comprising the nucleic acid sequences which permit the regulated expression of a rare restriction enzyme in the host cell, as described in US20030092161 and EP1183380, the contents of which are each incorporated herein by reference in their entireties as related to the production of viral particles, insofar as they do not conflict with the present disclosure.

In certain embodiments, production methods and cell lines to produce the AAV particle may comprise, but are not limited to those taught in PCT/US1996/010245, PCT/US1997/015716, PCT/US1997/015691, PCT/US1998/019479, PCT/US1998/019463, PCT/US2000/000415, PCT/US2000/040872, PCT/US2004/016614, PCT/US2007/010055, PCT/US1999/005870, PCT/US2000/004755, US Patent Application Nos. U.S. Ser. No. 08/549,489, U.S. Ser. No. 08/462,014, U.S. Ser. No. 09/659,203, U.S. Ser. No. 10/246,447, U.S. Ser. No. 10/465,302, US Patent Nos. U.S. Pat. Nos. 6,281,010, 6,270,996, 6,261,551, 5,756,283 (Assigned to NIH), U.S. Pat. Nos. 6,428,988, 6,274,354, 6,943,019, 6,482,634, (Assigned to NIH: U.S. Pat. Nos. 7,238,526, 6,475,769), U.S. Pat. No. 6,365,394 (Assigned to NIH), U.S. Pat. Nos. 7,491,508, 7,291,498, 7,022,519, 6,485,966, 6,953,690, 6,258,595, EP2018421, EP1064393, EP1163354, EP835321, EP931158, EP950111, EP1015619, EP1183380, EP2018421, EP1226264, EP1636370, EP1163354, EP1064393, US20030032613, US20020102714, US20030073232, US20030040101 (Assigned to NIH), US20060003451, US20020090717, US20030092161, US20070231303, US20060211115, US20090275107, US2007004042, US20030119191, US20020019050, the contents of which are each incorporated herein by reference in their entireties, insofar as they do not conflict with the present disclosure.

### Viral Production Systems

### Large-Scale Production

In certain embodiments, AAV particle production may be modified to increase the scale of production. Large scale viral production methods according to the present disclosure may comprise any of the processes or processing steps taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety.

Methods of increasing AAV particle production scale typically comprise increasing the number of viral production cells. In certain embodiments, viral production cells comprise adherent cells. To increase the scale of AAV particle production by adherent viral production cells, larger cell culture surfaces are required. In certain embodiments, large-scale production methods comprise the use of roller bottles to increase cell culture surfaces. Other cell culture substrates with increased surface areas are known in the art. Examples of additional adherent cell culture products with increased surface areas comprise, but are not limited to iCELLis (Pall Corp, Port Wash., N.Y.), CELLSTACK®, CELLCUBE® (Coming Corp., Corning, N.Y.) and NUNC™ CELL FACTORY™ (Thermo Scientific, Waltham, Mass.) In certain embodiments, large-scale adherent cell surfaces may comprise from about 1,000 cm² to about 100,000 cm².

In certain embodiments, large-scale viral production methods of the present disclosure may comprise the use of suspension cell cultures. Suspension cell culture can allow for significantly increased numbers of cells. Typically, the number of adherent cells that can be grown on about 10-50 cm² of surface area can be grown in about 1 cm³ volume in suspension.

In certain embodiments, large-scale cell cultures may comprise from about 107 to about 10⁹ cells, from about 108 to about 10¹⁰ cells, from about 109 to about 10¹² cells or at least 10¹² cells. In certain embodiments, large-scale cultures may produce from about 109 to about 10¹², from about 10¹⁰ to about 1013, from about 10¹¹ to about 10¹⁴, from about 10¹² to about 10¹⁵ or at least 10¹⁵ AAV particles.

Transfection of replication cells in large-scale culture formats may be carried out according to any methods known in the art. For large-scale adherent cell cultures, transfection methods may comprise, but are not limited to the use of inorganic compounds (e.g., calcium phosphate,) organic compounds (e.g., polyethyleneimine (PEI)) or the use of non-chemical methods (e.g., electroporation). With cells grown in suspension, transfection methods may comprise, but are not limited to the use of inorganic compounds (e.g., calcium phosphate,) organic compounds (e.g., polyethyleneimine (PEI)) or the use of non-chemical methods (e.g., electroporation). In certain embodiments, transfection of large-scale suspension cultures may be carried out according to the section entitled “Transfection Procedure” described in Feng, L. et al., 2008. Biotechnol Appl Biochem. 50:121-32, the contents of which are herein incorporated by reference in their entirety. According to such embodiments, PEI-DNA complexes may be formed for introduction of plasmids to be transfected. In certain embodiments, cells being transfected with PEI-DNA complexes may be ‘shocked’ prior to transfection. This comprises lowering cell culture temperatures to 4° C. for a period of about 1 hour. In certain embodiments, cell cultures may be shocked for a period of from about 10 minutes to about 5 hours. In certain embodiments, cell cultures may be shocked at a temperature of from about 0° C. to about 20° C.

In certain embodiments, transfections may comprise one or more vectors for expression of an RNA effector molecule to reduce expression of nucleic acids from one or more payload construct. Such methods may enhance the production of AAV particles by reducing cellular resources wasted on expressing payload constructs. In certain embodiments, such methods may be carried according to those taught in US Publication No. US2014/0099666, the contents of which are herein incorporated by reference in their entirety.

### Bioreactors

In certain embodiments, cell culture bioreactors may be used for large scale production of AAV particles. In certain embodiments, bioreactors comprise stirred tank reactors. Such reactors generally comprise a vessel, typically cylindrical in shape, with a stirrer (e.g., impeller.) In certain embodiments, such bioreactor vessels may be placed within a water jacket to control vessel temperature and/or to minimize effects from ambient temperature changes.

Bioreactor vessel volume may range in size from about 500 ml to about 2 L, from about 1 L to about 5 L, from about 2.5 L to about 20 L, from about 10 L to about 50 L, from about 25 L to about 100 L, from about 75 L to about 500 L, from about 250 L to about 2,000 L, from about 1,000 L to about 10,000 L, from about 5,000 L to about 50,000 L or at least 50,000 L. Vessel bottoms may be rounded or flat. In certain embodiments, animal cell cultures may be maintained in bioreactors with rounded vessel bottoms.

In certain embodiments, bioreactor vessels may be warmed through the use of a thermocirculator. Thermocirculators pump heated water around water jackets. In certain embodiments, heated water may be pumped through pipes (e.g., coiled pipes) that are present within bioreactor vessels. In certain embodiments, warm air may be circulated around bioreactors, comprising, but not limited to air space directly above culture medium. Additionally, pH and CO₂ levels may be maintained to optimize cell viability.

In certain embodiments, bioreactors may comprise hollow-fiber reactors. Hollow-fiber bioreactors may support the culture of both anchorage dependent and anchorage independent cells. Further bioreactors may comprise, but are not limited to, packed-bed or fixed-bed bioreactors. Such bioreactors may comprise vessels with glass beads for adherent cell attachment. Further packed-bed reactors may comprise ceramic beads.

In certain embodiments, viral particles are produced through the use of a disposable bioreactor. In certain embodiments, bioreactors may comprise GE WAVE bioreactor, a GE Xcellerex Bioreactor, a Sartorius Biostat Bioreactor, a ThermoFisher Hyclone Bioreactor, or a Pall Allegro Bioreactor.

In certain embodiments, AAV particle production in cell bioreactor cultures may be carried out according to the methods or systems taught in U.S. Pat. Nos. 5,064,764, 6,194,191, 6,566,118, 8,137,948 or US Patent Application No. US2011/0229971, the contents of each of which are herein incorporated by reference in their entirety.

In certain embodiments, perfusion technology can be used in the production of viral particles. Perfusion systems are fluid circulation systems which use filters and screens to retain cells inside a bioreactor while continually removing cell waste products and media depleted of nutrients by cell metabolism. In certain embodiments, the perfusion system is an alternating tangential flow (ATF) perfusion system. In certain embodiments, a perfusion system can be used in coordination with bioreactors to manage and cycle cell culture media within a bioreactor during the production of viral particles, such as AAV viral particles. In certain embodiments, a perfusion system can be used to support the production of high quality AAV viral particles having an unexpectedly high cell density at large-scale. In certain embodiments, a perfusion system can be used to perform a media switch within the bioreactor, such as the replacement of a cell culture media with media supplemented with growth or production boosting factors to increase the quality and quantity of the AAV product.

It is advantageous to produce large batches of AAV particles in single production campaigns for gene therapy clinical development activities, as the large batches of therapeutic materials ensure clinical study consistency and minimize the therapeutic and statistical variability resulting from multiple smaller manufacturing campaigns. It is advantageous to produce large batches of AAV particles in single production campaigns for commercial product development activities, as the large batches of therapeutic materials minimize the variability resulting from multiple smaller manufacturing campaigns and corresponding complications in quality control and product analysis associated with small-batch production.

### Expansion of Viral Production Cell (VPC) Mixtures

In certain embodiments, an AAV particle or viral vector of the present disclosure may be produced in a viral production cell (VPC), such as an insect cell. Production cells can be sourced from a Cell Bank (CB) and are often stored in frozen cell banks.

In certain embodiments, a viral production cell from a Cell Bank is provided in frozen form. The vial of frozen cells is thawed, typically until ice crystal dissipate. In certain embodiments, the frozen cells are thawed at a temperature between 10-50° C., 15-40° C., 20-30° C., 25-50° C., 30-45° C., 35-40° C., or 37-39° C. In certain embodiments, the frozen viral production cells are thawed using a heated water bath.

In certain embodiments, a thawed CB cell mixture will have a cell density of 1.0×10⁴-1.0×10⁹ cells/mL. In certain embodiments, the thawed CB cell mixture has a cell density of 1.0×10⁴-2.5×10⁴ cells/mL, 2.5×10⁴-5.0×10⁴ cells/mL, 5.0×10⁴-7.5×10⁴ cells/mL, 7.5×10⁴-1.0×10⁵ cells/mL, 1.0×10⁵-2.5×10⁵ cells/mL, 2.5×10⁵-5.0×10⁵ cells/mL, 5.0×10⁵-7.5×10⁵ cells/mL, 7.5×10⁵-1.0×10⁶ cells/mL, 1.0×10⁶-2.5×10⁶ cells/mL, 2.5×10⁶-5.0×10⁶ cells/mL, 5.0×10⁶-7.5×10⁶ cells/mL, 7.5×10⁶-1.0×10⁷ cells/mL, 1.0×10⁷-2.5×10⁷ cells/mL, 2.5×10⁷-5.0×10⁷ cells/mL, 5.0×10⁷-7.5×10⁷ cells/mL, 7.5×10⁷-1.0×10⁸ cells/mL, 1.0×10⁸-2.5×10⁸ cells/mL, 2.5×10⁸-5.0×10⁸ cells/mL, 5.0×10⁸-7.5×10⁸ cells/mL, or 7.5×10⁸-1.0×10⁹ cells/mL.

In certain embodiments, the volume of the CB cell mixture is expanded. This process is commonly referred to as a Seed Train, Seed Expansion, or CB Cellular Expansion. Cellular/Seed expansion can comprise successive steps of seeding and expanding a cell mixture through multiple expansion steps using successively larger working volumes. In certain embodiments, cellular expansion can comprise one, two, three, four, five, six, seven, or more than seven expansion steps. In certain embodiments, the working volume in the cellular expansion can comprise one or more of the following working volumes or working volume ranges: 5 mL, 10 mL, 20 mL, 5-20 mL, 25 mL, 30 mL, 40 mL, 50 mL, 20-50 mL, 75 mL, 100 mL, 125 mL, 150 mL, 175 mL, 200 mL, 50-200 mL, 250 mL, 300 mL, 400 mL, 500 mL, 750 mL, 1000 mL, 250-1000 mL, 1250 mL, 1500 mL, 1750 mL, 2000 mL, 1000-2000 mL, 2250 mL, 2500 mL, 2750 mL, 3000 mL, 2000-3000 mL, 3500 mL, 4000 mL, 4500 mL, 5000 mL, 3000-5000 mL, 5.5 L, 6.0 L, 7.0 L, 8.0 L, 9.0 L, 10.0 L, and 5.0-10.0 L.

In certain embodiments, a volume of cells from a first expanded cell mixture can be used to seed a second, separate Seed Train/Seed Expansion (instead of using thawed CB cell mixture). This process is commonly referred to as rolling inoculum. In certain embodiments, rolling inoculum is used in a series of two or more (e.g., two, three, four or five) separate Seed Trains/Seed Expansions.

In certain embodiments, large-volume cellular expansion can comprise the use of a bioreactor, such as a GE WAVE bioreactor, a GE Xcellerex Bioreactor, a Sartorius Biostat Bioreactor, a ThermoFisher Hyclone Bioreactor, or a Pall Allegro Bioreactor.

In certain embodiments, the cell density within a working volume is expanded to a target output cell density. In certain embodiments, the output cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the output cell density of a working volume provides a seeding cell density for a larger, successive working volume. In certain embodiments, the seeding cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, cellular expansion can last for 1-50 days. Each cellular expansion step or the total cellular expansion can last for 1-10 days, 1-5 days, 1-3 days, 2-3 days, 2-4 days, 2-5 days, 2-6 days, 3-4 days, 3-5 days, 3-6 days, 3-8 days, 4-5 days, 4-6 days, 4-8 days, 5-6 days, or 5-8 days. In certain embodiments, each cellular expansion step or the total cellular expansion can last for 1-100 generations, 1-1000 generations, 100-1000 generation, 100 generations or more, or 1000 generation or more.

In certain embodiments, infected or transfected production cells can be expanded in the same manner as CB cell mixtures, as set forth in the present disclosure.

### Infection of Viral Production Cells

In certain embodiments, AAV particles of the present disclosure are produced in a viral production cell (VPC), such as an insect cell, by infecting the VPC with a viral vector which comprises an AAV expression construct and/or a viral vector which comprises an AAV payload construct. In certain embodiments, the VPC is infected with an Expression BEV, which comprises an AAV expression construct and a Payload BEV which comprises an AAV payload construct.

In certain embodiments, AAV particles are produced by infecting a VPC with a viral vector which comprises both an AAV expression construct and an AAV payload construct. In certain embodiments, the VPC is infected with a single BEV which comprises both an AAV expression construct and an AAV payload construct.

In certain embodiments, VPCs (such as insect cells) are infected using Infection BIICs in an infection process which comprises the following steps: (i) A collection of VPCs are seeded into a Production Bioreactor; (ii) The seeded VPCs can optionally be expanded to a target working volume and cell density; (iii) Infection BIICs which comprise Expression BEVs and Infection BIICs which comprise Payload BEVs are injected into the Production Bioreactor, resulting in infected viral production cells; and (iv) incubation of the infected viral production cells to produce AAV particles within the viral production cells.

In certain embodiments, the VPC density at infection is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-3.4×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at infection is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.1×10⁶, 3.2×10⁶, 3.3×10⁶, 3.4×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL. In certain embodiments, the VPC density at infection is 2.0-3.5×10⁶ cells/mL. In certain embodiments, the VPC density at infection is 3.5-5.0×10⁶ cells/mL. In certain embodiments, the VPC density at infection is 5.0-7.5×10⁶ cells/mL. In certain embodiments, the VPC density at infection is 5.0-10.0×10⁶ cells/mL.

In certain embodiments, Infection BIICs are combined with the VPCs in target ratios of VPC-to-BIIC. In certain embodiments, the VPC-to-BIIC infection ratio (volume to volume) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ (VPC volume to BIIC volume). In certain embodiments, the VPC-to-BIIC infection ratio (cell to cell) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ (VPC cells to BIIC cells).

In certain embodiments, Infection BIICs which comprise Expression BEVs are combined with the VPCs in target ratios of VPC-to-expressionBIIC. In certain embodiments, the VPC-to-expressionBIIC infection ratio (volume to volume) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ (VPC volume to BIIC volume). In certain embodiments, the VPC-to-expressionBIIC infection ratio (cell to cell) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ (VPC cells to BIIC cells).

In certain embodiments, Infection BIICs which comprise Payload BEVs are combined with the VPCs in target ratios of VPC-to-payloadBIIC. In certain embodiments, the VPC-to-payloadBIIC infection ratio (volume to volume) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ (VPC volume to BIIC volume). In certain embodiments, the VPC-to-payloadBIIC infection ratio (cell to cell) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ (VPC cells to BIIC cells).

In certain embodiments, Infection BIICs which comprise Expression BEVs and Infection BIICs which comprise Payload BEVs are combined with the VPCs in target expressionBIIC-to-payloadBIIC ratios. In certain embodiments, the ratio of expressionBIICs to payloadBIICs is 10:1, 9:1, 8:1, 7:1, 6:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:9, or 1:10. In certain embodiments, the ratio of expressionBIICs to payloadBIICs is between 6.5-7.5:1, 6-7:1, 5.5-6.5:1, 5-6:1, 4.5-5.5:1, 4-5:1, 3.5-4.5:1, 3-4:1, 2.5-3.5:1, 2-3:1, 1.5-2.5:1, 1-2:1, 1-1.5:1, 1:1-1.5, 1:1-2, 1:1.5-2.5, 1:2-3, 1:2.5-3.5, 1:3-4, 1:3.5-4.5, 1:4-5, 1:4.5-5.5, 1:5-6, 1:5.5-6.5, 1:6-7, or 1:6.5-7.5.

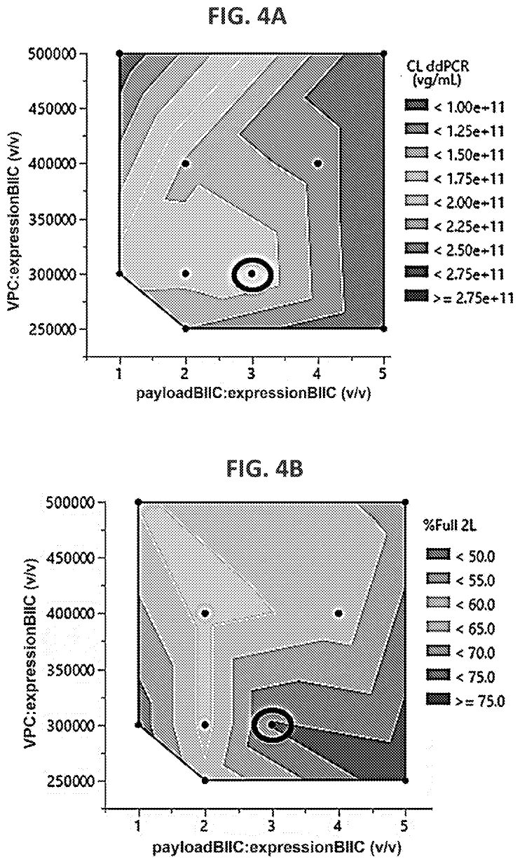

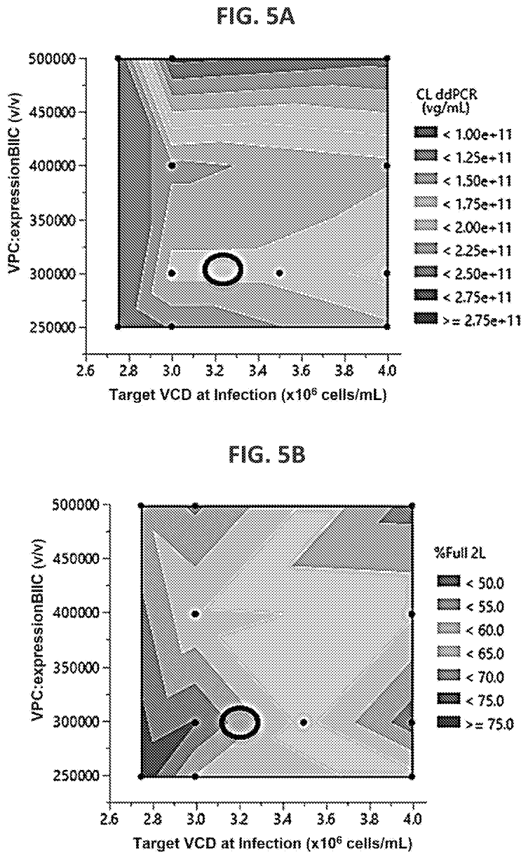

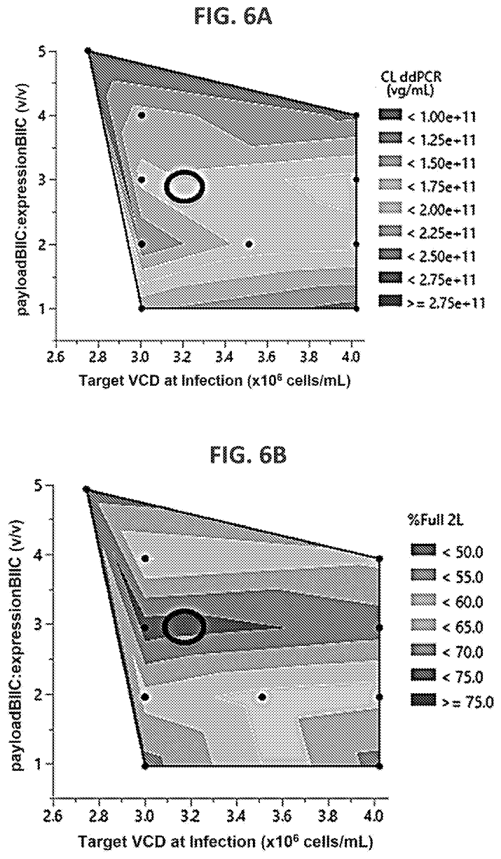

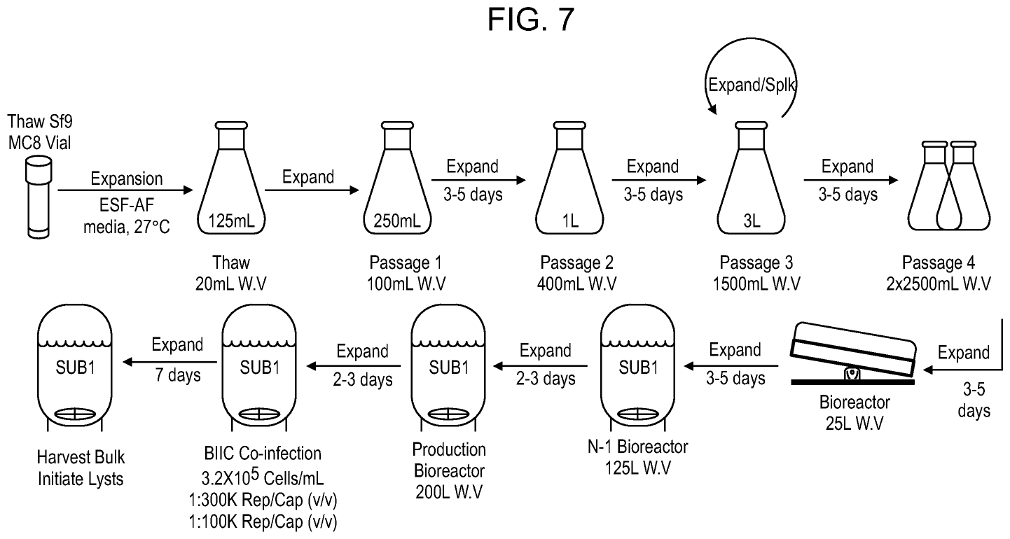

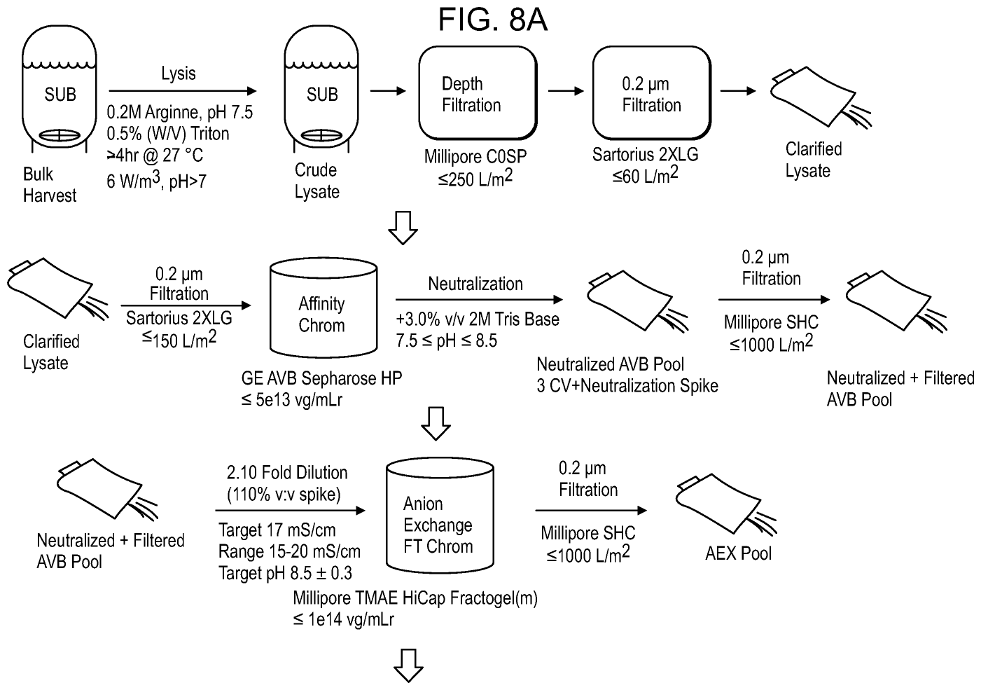

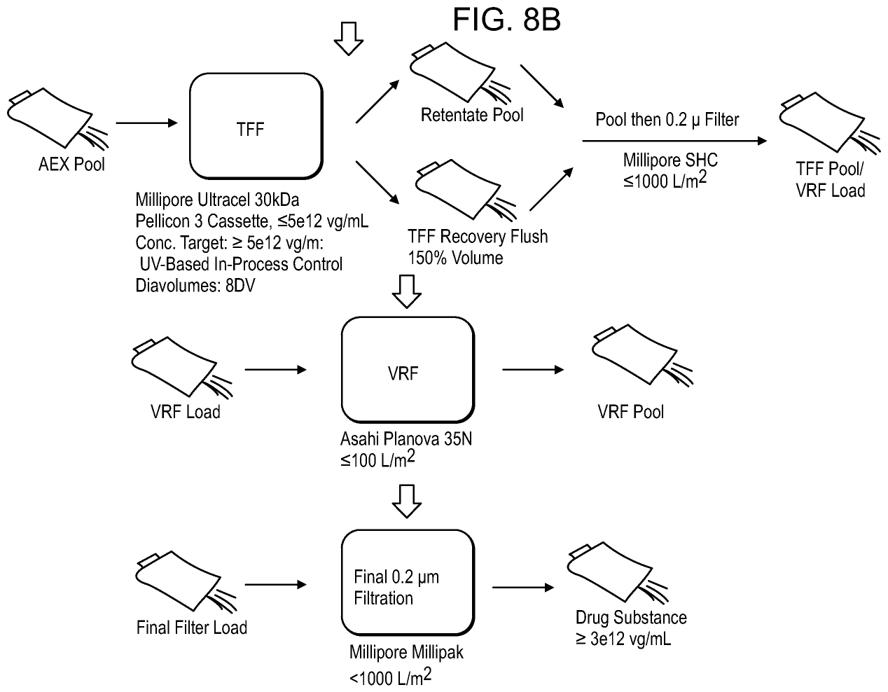

FIG. 4A, FIG. 4B, FIG. 5A, FIG. 5B, FIG. 6A, and FIG. 6B show that a VPC density at transfection/infection of between 3.0-3.4×10⁶ cells/mL (particularly 3.2×10⁶ cells/mL) combined with a VPC-to-expressionBIIC ratio of 250K-350K:1 (v/v), e.g., about 300K:1 (v/v), and a payloadBIIC-to-expressionBIIC ratio of 2.5-3.5:1 (v/v), e.g., about 3:1 (v/v) (e.g., a VPC-to-payloadBIIC ratio of about 100K:1 (v/v)) provides favorable AAV titer (vg/mL) and Capsid Full %.

In certain embodiments, infected Viral Production Cells are incubated under a certain Dissolved Oxygen (DO) Content (DO %). In certain embodiments, infected Viral Production Cells are incubated under a DO % between 10%-50%, 20%-40%, 10%-20%, 15%-25%, 20%-30%, 25%-35%, 30%-40%, 35%-45%, 40%-50%, 10%-15%, 15%-20%, 20%-25%, 25%-30%, 30%-35%, 35%-40%, 40%-45%, or 45%-50%. In certain embodiments, infected Viral Production Cells are incubated under a DO % of about 10%, about 15%, about 20%, about 25%, about 30%, about 35%, about 40%, about 45%, about 50%. In certain embodiments, infected Viral Production Cells are incubated under a DO % between 20%-30% or about 25%. In certain embodiments, infected Viral Production Cells are incubated under a DO % between 25%-35% or about 30%. In certain embodiments, infected Viral Production Cells are incubated under a DO % between 30%-40% or about 35%. In certain embodiments, infected Viral Production Cells are incubated under a DO % between 35%-45% or about 40%.

### Cell Lysis

Cells of the present disclosure, comprising, but not limited to viral production cells, may be subjected to cell lysis according to any methods known in the art. Cell lysis may be carried out to obtain one or more agents (e.g., viral particles) present within any cells of the disclosure. In certain embodiments, a bulk harvest of AAV particles and viral production cells is subjected to cell lysis according to the present disclosure.

In certain embodiments, cell lysis may be carried out according to any of the methods or systems presented in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Cell lysis methods and systems may be chemical or mechanical. Chemical cell lysis typically comprises contacting one or more cells with one or more chemical lysis agent under chemical lysis conditions. Mechanical lysis typically comprises subjecting one or more cells to cell lysis carried out by mechanical force. Lysis can also be completed by allowing the cells to degrade after reaching ˜0% viability.

In certain embodiments, chemical lysis may be used to lyse cells. As used herein, the term “chemical lysis agent” refers to any agent that may aid in the disruption of a cell. In certain embodiments, lysis agents are introduced in solutions, termed lysis solutions or lysis buffers. As used herein, the term “chemical lysis solution” refers to a solution (typically aqueous) comprising one or more lysis agent. In addition to lysis agents, lysis solutions may comprise one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may comprise one or more solubilizing agent. As used herein, the term “solubilizing agent” refers to a compound that enhances the solubility of one or more components of a solution and/or the solubility of one or more entities to which solutions are applied. In certain embodiments, solubilizing agents enhance protein solubility. In certain embodiments, solubilizing agents are selected based on their ability to enhance protein solubility while maintaining protein conformation and/or activity.

Exemplary lysis agents may comprise any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In certain embodiments, lysis agents may be selected from lysis salts, amphoteric agents, cationic agents, ionic detergents and non-ionic detergents. Lysis salts may comprise, but are not limited to, sodium chloride (NaCl) and potassium chloride (KCl.) Further lysis salts may comprise any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety.

In certain embodiments, the cell lysate solution comprises a stabilizing additive. In certain embodiments, the stabilizing additive can comprise trehalose, glycine betaine, mannitol, potassium citrate, CuCl₂, proline, xylitol, NDSB 201, CTAB and K₂PO₄. In certain embodiments, the stabilizing additive can comprise amino acids such as arginine, or acidified amino acid mixtures such as arginine HCl. In certain embodiments, the stabilizing additive can comprise 0.1 M arginine or arginine HCl. In certain embodiments, the stabilizing additive can comprise 0.2 M arginine or arginine HCl. In certain embodiments, the stabilizing additive can comprise 0.25 M arginine or arginine HCl. In certain embodiments, the stabilizing additive can comprise 0.3 M arginine or arginine HCl. In certain embodiments, the stabilizing additive can comprise 0.4 M arginine or arginine HCl. In certain embodiments, the stabilizing additive can comprise 0.5 M arginine or arginine HCl. In certain embodiments, the stabilizing additive can comprise 0.6 M arginine or arginine HCl. In certain embodiments, the stabilizing additive can comprise 0.7 M arginine or arginine HCl. In certain embodiments, the stabilizing additive can comprise 0.8 M arginine or arginine HCl. In certain embodiments, the stabilizing additive can comprise 0.9 M arginine or arginine HCl. In certain embodiments, the stabilizing additive can comprise 1.0 M arginine or arginine HCl.

Concentrations of salts may be increased or decreased to obtain an effective concentration for the rupture of cell membranes. Amphoteric agents, as referred to herein, are compounds capable of reacting as an acid or a base. Amphoteric agents may comprise, but are not limited to lysophosphatidylcholine, 3-((3-Cholamidopropyl) dimethylammonium)-1-propanesulfonate (CHAPS), ZWITTERGENT® and the like. Cationic agents may comprise, but are not limited to, cetyltrimethylammonium bromide (C (16) TAB) and Benzalkonium chloride. Lysis agents comprising detergents may comprise ionic detergents or non-ionic detergents.

Detergents may function to break apart or dissolve cell structures comprising, but not limited to cell membranes, cell walls, lipids, carbohydrates, lipoproteins and glycoproteins. Exemplary ionic detergents comprise any of those taught in U.S. Pat. Nos. 7,625,570 and 6,593,123 or US Publication No. US2014/0087361, the contents of each of which are herein incorporated by reference in their entirety. In certain embodiments, the lysis solution comprises one or more ionic detergents. Example of ionic detergents for use in a lysis solution comprise, but are not limited to, sodium dodecyl sulfate (SDS), cholate and deoxycholate. In certain embodiments, ionic detergents may be comprised in lysis solutions as a solubilizing agent. In certain embodiments, the lysis solution comprises one or more nonionic detergents. Non-ionic detergents for use in a lysis solution may comprise, but are not limited to, octylglucoside, digitonin, lubrol, C12E8, TWEEN®-20, TWEEN®-80, Triton X-100, Triton X-114, Brij-35, Brij-58, and Noniodet P-40. Non-ionic detergents are typically weaker lysis agents but may be comprised as solubilizing agents for solubilizing cellular and/or viral proteins. In certain embodiments, the lysis solution comprises one or more zwitterionic detergents. Zwitterionic detergents for use in a lysis solution may comprise, but are not limited to: Lauryl dimethylamine N-oxide (LDAO); N,N-Dimethyl-N-dodecylglycine betaine (Empigen® BB); 3-(N,N-Dimethylmyristylammonio) propanesulfonate (Zwittergent® 3-10); n-Dodecyl-N,N-dimethyl-3-ammonio-1-propanesulfonate (Zwittergent® 3-12); n-Tetradecyl-N,N-dimethyl-3-ammonio-1-propanesulfonate (Zwittergent® 3-14); 3-(N,N-Dimethyl palmitylammonio) propanesulfonate (Zwittergent® 3-16); 3-((3-cholamidopropyl) dimethylammonio)-1-propanesulfonate (CHAPS); and 3-([3-Cholamidopropyl] dimethylammonio)-2-hydroxy-1-propanesulfonate (CHAPSO).

In certain embodiments, the lysis solution comprises Triton X-100 (octyl phenol ethoxylate), such as 0.5% w/v of Triton X-100. In certain embodiments, the lysis solution comprises Lauryldimethylamine N-oxide (LDAO), such as 0.184% w/v (4×CMC) of LDAO. In certain embodiments, the lysis solution comprises a seed oil surfactant such as Ecosurf™ SA-9. In certain embodiments, the lysis solution comprises N,N-Dimethyl-N-dodecylglycine betaine (Empigen® BB). In certain embodiments, the lysis solution comprises a Zwittergent® detergent, such as Zwittergent® 3-12 (n-Dodecyl-N,N-dimethyl-3-ammonio-1-propanesulfonate), Zwittergent® 3-14 (n-Tetradecyl-N,N-dimethyl-3-ammonio-1-propanesulfonate), or Zwittergent® 3-16 (3-(N,N-Dimethyl palmitylammonio)propanesulfonate).

Further lysis agents may comprise enzymes and urea. In certain embodiments, one or more lysis agents may be combined in a lysis solution in order to enhance one or more of cell lysis and protein solubility. In certain embodiments, enzyme inhibitors may be comprised in lysis solutions in order to prevent proteolysis that may be triggered by cell membrane disruption.

In certain embodiments, the lysis solution comprises between 0.1-1.0% w/v, between 0.2-0.8% w/v, between 0.3-0.7% w/v, between 0.4-0.6% w/v, or about 0.5% w/v of a cell lysis agent (e.g., detergent). In certain embodiments, the lysis solution comprises between 0.3-0.35% w/v, between 0.35-0.4% w/v, between 0.4-0.45% w/v, between 0.45-0.5% w/v, between 0.5-0.55% w/v, between 0.55-0.6% w/v, between 0.6-0.65% w/v, or between 0.65-0.7% w/v of a cell lysis agent (e.g., detergent).

In certain embodiments, cell lysates generated from adherent cell cultures may be treated with one more nuclease, such as Benzonase nuclease (Grade I, 99% pure) or c-LEcta Denarase nuclease (formerly Sartorius Denarase). In certain embodiments, nuclease is added to lower the viscosity of the lysates caused by liberated DNA.

In certain embodiments, chemical lysis uses a single chemical lysis mixture. In certain embodiments, chemical lysis uses several lysis agents added in series to provide a final chemical lysis mixture.

In certain embodiments, a chemical lysis mixture comprises an acidified amino acid mixture (such as arginine HCl), a non-ionic detergent (such as Triton X-100), and a nuclease (such as Benzonase nuclease). In certain embodiments, the chemical lysis mixture can comprise an acid or base to provide a target lysis pH.

In certain embodiments, the lysis solution comprises 0.5% w/v Triton X-100 (octyl phenol ethoxylate) and 200 mM arginine hydrochloride. In certain embodiments, the lysis solution comprises 0.5% w/v Triton X-100 (octyl phenol ethoxylate) and 200 mM arginine hydrochloride, and lacks detectable nuclease. In certain embodiments, the lysis solution consists of 0.5% w/v Triton X-100 (octyl phenol ethoxylate) and 200 mM arginine hydrochloride.

In certain embodiments, chemical lysis is conducted under chemical lysis conditions. As used herein, the term “chemical lysis conditions” refers to any combination of environmental conditions (e.g., temperature, pressure, pH, etc) in which targets cells can be lysed by a chemical lysis agent.

In certain embodiments, the lysis pH is between 3.0-3.5, 3.5-4.0, 4.0-4.5, 4.5-5.0, 5.0-5.5, 5.5-6.0, 6.0-6.5, 6.5-7.0, 7.0-7.5, or 7.5-8.0. In certain embodiments, the lysis pH is between 6.0-7.0, 6.5-7.0, 6.5-7.5, or 7.0-7.5.

In certain embodiments, the lysis temperature is between 15-35° C., between 20-30° C., between 25-39° C., between 20-21° C., between 20-22° C., between 21-22° C., between 21-23° C., between 22-23° C., between 22-24° C., between 23-24° C., between 23-25° C., between 24-25° C., between 24-26° C., between 25-26° C., between 25-27° C., between 26-27° C., between 26-28° C., between 27-28° C., between 27-29° C., between 28-29° C., between 28-30° C., between 29-30° C., between 29-31° C., between 30-31° C., between 30-32° C., between 31-32° C., or between 31-33° C.

In certain embodiments, the lysis solution comprises 0.5% w/v Triton X-100 (octyl phenol ethoxylate) and 200 mM arginine hydrochloride, and lysis conditions comprise a duration of at least 4 hours (e.g., 4-6 hours, e.g., 4 hours) at 26° C.-28° C. (e.g., 27° C.). In certain embodiments, the lysis solution comprises 0.5% w/v Triton X-100 (octyl phenol ethoxylate) and 200 mM arginine hydrochloride, and lacks detectable nuclease, and lysis conditions comprise a duration of at least 4 hours (e.g., 4-6 hours, e.g., 4 hours) at 26° C.-28° C. (e.g., 27° C.). In certain embodiments, the lysis solution consists of 0.5% w/v Triton X-100 (octyl phenol ethoxylate) and 200 mM arginine hydrochloride, and lysis conditions comprise a duration of at least 4 hours (e.g., 4-6 hours, e.g., 4 hours) at 26° C.-28° C. (e.g., 27° C.).

In certain embodiments, mechanical cell lysis is carried out. Mechanical cell lysis methods may comprise the use of one or more lysis condition and/or one or more lysis force. As used herein, the term “lysis condition” refers to a state or circumstance that promotes cellular disruption. Lysis conditions may comprise certain temperatures, pressures, osmotic purity, salinity and the like. In certain embodiments, lysis conditions comprise increased or decreased temperatures. According to certain embodiments, lysis conditions comprise changes in temperature to promote cellular disruption. Cell lysis carried out according to such embodiments may comprise freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further comprise one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In certain embodiments, one or more cryoprotectants are comprised in cell solutions undergoing freeze-thaw lysis. As used herein, the term “cryoprotectant” refers to an agent used to protect one or more substance from damage due to freezing. Cryoprotectants may comprise any of those taught in US Publication No. US2013/0323302 or U.S. Pat. Nos. 6,503,888, 6,180,613, 7,888,096, 7,091,030, the contents of each of which are herein incorporated by reference in their entirety. In certain embodiments, cryoprotectants may comprise, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In certain embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may comprise, but are not limited to mechanical forces, sonic forces, gravitational forces, optical forces, electrical forces and the like. Cell lysis carried out by mechanical force is referred to herein as “mechanical lysis.” Mechanical forces that may be used according to mechanical lysis may comprise high shear fluid forces. According to such methods of mechanical lysis, a microfluidizer may be used. Microfluidizers typically comprise an inlet reservoir where cell solutions may be applied. Cell solutions may then be pumped into an interaction chamber via a pump (e.g., high-pressure pump) at high speed and/or pressure to produce shear fluid forces. Resulting lysates may then be collected in one or more output reservoir. Pump speed and/or pressure may be adjusted to modulate cell lysis and enhance recovery of products (e.g., viral particles.) Other mechanical lysis methods may comprise physical disruption of cells by scraping.

Cell lysis methods may be selected based on the cell culture format of cells to be lysed. For example, with adherent cell cultures, some chemical and mechanical lysis methods may be used. Such mechanical lysis methods may comprise freeze-thaw lysis or scraping. In another example, chemical lysis of adherent cell cultures may be carried out through incubation with lysis solutions comprising surfactant, such as Triton-X-100.

In certain embodiments, a method for harvesting AAV particles without lysis may be used for efficient and scalable AAV particle production. In a non-limiting example, AAV particles may be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107, the contents of which are incorporated herein by reference in their entirety.

### Clarification and Purification: General

Cell lysates comprising viral particles may be subjected to clarification and purification. Clarification generally refers to the initial steps taken in the purification of viral particles from cell lysates and serves to prepare lysates for further purification by removing larger, insoluble debris from a bulk lysis harvest. Viral production can comprise clarification steps at any point in the viral production process. Clarification steps may comprise, but are not limited to, centrifugation and filtration. During clarification, centrifugation may be carried out at low speeds to remove larger debris only. Similarly, filtration may be carried out using filters with larger pore sizes so that only larger debris is removed.

Purification generally refers to the final steps taken in the purification and concentration of viral particles from cell lysates by removing smaller debris from a clarified lysis harvest in preparing a final Pooled Drug Substance. Viral production can comprise purification steps at any point in the viral production process. Purification steps may comprise, but are not limited to, filtration and chromatography. Filtration may be carried out using filters with smaller pore sizes to remove smaller debris from the product or with larger pore sizes to retain larger debris from the product. Filtration may be used to alter the concentration and/or contents of a viral production pool or stream. Chromatography may be carried out to selectively separate target particles from a pool of impurities.

Large-scale production of high-concentration AAV formulations is complicated by the tendency for high concentrations of AAV particles to aggregate or agglomerate. Small scale clarification and concentration systems, such as dialysis cassettes or spin centrifugation, are generally not sufficiently scalable for large-scale production. The present disclosure provides embodiments of a clarification, purification and concentration system for processing large volumes of high-concentration AAV production formulations. In certain embodiments, the large-volume clarification system comprises one or more of the following processing steps: Depth Filtration, Microfiltration (e.g., 0.2 μm Filtration), Affinity Chromatography, Ion Exchange Chromatography such as anion exchange chromatography (AEX) or cation exchange chromatography (CEX), a tangential flow filtration system (TFF), Nanofiltration (e.g., Virus Retentive Filtration (VRF)), Final Filtration (FF), and Fill Filtration.

Objectives of viral clarification and purification comprise high throughput processing of cell lysates and to optimize ultimate viral recovery. Advantages of comprising clarification and purification steps of the present disclosure comprise scalability for processing of larger volumes of lysate. In certain embodiments, clarification and purification may be carried out according to any of the methods or systems presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

In certain embodiments, the compositions comprising at least one AAV particle may be isolated or purified using the methods or systems described in U.S. Pat. Nos. 6,146,874, 6,660,514, 8,283,151 or U.S. Pat. No. 8,524,446, the contents of which are herein incorporated by reference in their entirety.

### Clarification and Purification: Centrifugation

According to certain embodiments, cell lysates may be clarified by one or more centrifugation steps. Centrifugation may be used to pellet insoluble particles in the lysate. During clarification, centrifugation strength (which can be expressed in terms of gravitational units (g), which represents multiples of standard gravitational force) may be lower than in subsequent purification steps. In certain embodiments, centrifugation may be carried out on cell lysates at a gravitation force from about 200 g to about 800 g, from about 500 g to about 1500 g, from about 1000 g to about 5000 g, from about 1200 g to about 10000 g or from about 8000 g to about 15000 g. In certain embodiments, cell lysate centrifugation is carried out at 8000 g for 15 minutes. In certain embodiments, density gradient centrifugation may be carried out in order to partition particulates in the cell lysate by sedimentation rate. Gradients used according to methods or systems of the present disclosure may comprise, but are not limited to, cesium chloride gradients and iodixanol step gradients. In certain embodiments, centrifugation uses a decanter centrifuge system. In certain embodiments, centrifugation uses a disc-stack centrifuge system. In certain embodiments, centrifugation comprises ultracentrifugation, such two-cycle CsCl gradient ultracentrifugation or iodixanol discontinuous density gradient ultracentrifugation.

### Clarification and Purification: Filtration

In certain embodiments, one or more microfiltration, nanofiltration and/or ultrafiltration steps may be used during clarification, purification and/or sterilization. The one or more microfiltration, nanofiltration or ultrafiltration steps can comprise the use of a filtration system such as EMD Millipore Express SHC XL10 0.5/0.2 μm filter, EMD Millipore Express SHCXL6000 0.5/0.2 μm filter, EMD Millipore Express SHCXL150 filter, EMD Millipore Millipak Gamma Gold 0.22 μm filter (dual-in-line sterilizing grade filters), a Pall Supor EKV, 0.2 μm sterilizing-grade filter, Asahi Planova 35N, Asahi Planova 20N, Asahi Planova 75N, Asahi Planova BioEx, Millipore Viresolve NFR or a Sartorius Sartopore 2XLG, 0.8/0.2 μm.

In certain embodiments, one or more microfiltration steps may be used during clarification, purification and/or sterilization. Microfiltration utilizes microfiltration membranes with pore sizes typically between 0.1 μm and 10 μm. Microfiltration is generally used for general clarification, sterilization, and removal of microparticulates. In certain embodiments, microfiltration is used to remove aggregated clumps of viral particles. In certain embodiments, a production process or system of the present disclosure comprises at least one microfiltration step. The one or more microfiltration steps can comprise a Depth Filtration step with a Depth Filtration system, such as EMD Millipore Millistak⁺ POD filter (DOHC media series), Millipore MCOSP23CL3 filter (COSP media series), or Sartorius Sartopore filter series. Microfiltration systems of the present disclosure can be pre-rinsed, packed, equilibrated, flushed, processed, eluted, washed or cleaned with formulations known to those in the art, comprising AAV pharmaceutical, processing and storage formulations of the present disclosure. In certain embodiments, clarification comprises use of a C0SP media series filter. In some embodiments, the C0SP media series filter is effective to reduce or prevent 0.2 micron filter clogging.

In certain embodiments, one or more ultrafiltration steps may be used during clarification and purification. The ultrafiltration steps can be used for concentrating, formulating, desalting or dehydrating either processing and/or formulation solutions of the present disclosure. Ultrafiltration utilizes ultrafiltration membranes, with pore sizes typically between 0.001 and 0.1 μm. Ultrafiltration membranes can also be defined by their molecular weight cutoff (MWCO) and can have a range from 1 kD to 500 kD. Ultrafiltration is generally used for concentrating and formulating dissolved biomolecules such as proteins, peptides, plasmids, viral particles, nucleic acids, and carbohydrates. Ultrafiltration systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, one or more nanofiltration steps may be used during clarification and purification. Nanofiltration utilizes nanofiltration membranes, with pore sizes typically less than 100 nm. Nanofiltration is generally used for removal of unwanted endogenous viral impurities (e.g., baculovirus). In certain embodiments, nanofiltration can comprise viral removal filtration (VRF). VRF filters can have a filtration size typically between 15 nm and 100 nm. Examples of VRF filters comprise (but are not limited to): Planova 15N, Planova 20N, and Planova 35N (Asahi-Kasei Corp, Tokyo, Japan); and Viresolve NFP and Viresolve NFR (Millipore Corp, Billerica, Mass., USA). Nanofiltration systems of the present disclosure can be pre-rinsed, packed, equilibrated, flushed, processed, eluted, washed or cleaned with formulations known to those in the art, comprising AAV pharmaceutical, processing and storage formulations of the present disclosure. In certain embodiments, nanofiltration is used to remove aggregated clumps of viral particles.

In certain embodiments, one or more tangential flow filtration (TFF) (also known as cross-flow filtration) steps may be used during clarification and purification. Tangential flow filtration is a form of membrane filtration in which a feed stream (which comprises the target agent/particle to be clarified and concentrated) flows from a feed tank into a filtration module or cartridge. Within the TFF filtration module, the feed stream passes parallel to a membrane surface, such that one portion of the stream passes through the membrane (permeate/filtrate) while the remainder of the stream (retentate) is recirculated back through the filtration system and into the feed tank.

In certain embodiments, the TFF filtration module can be a flat plate module (stacked planar cassette), a spiral wound module (spiral-wound membrane layers), or a hollow fiber module (bundle of membrane tubes). Examples of TFF systems for use in the present disclosure comprise, but are not limited to: Spectrum mPES Hollow Fiber TFF system (0.5 mm fiber ID, 100 kDA MWCO) or Millipore Ultracel PLCTK system with Pellicon-3 cassette (0.57 m², 30 kDA MWCO).

New buffer materials can be added to the TFF feed tank as the feed stream is circulated through the TFF filtration system. In certain embodiments, buffer materials can be fully replenished as the flow stream circulates through the TFF filtration system. In this embodiment, buffer material is added to the stream in equal amounts to the buffer material lost in the permeate, resulting in a constant concentration. In certain embodiments, buffer materials can be reduced as the flow stream circulates through the filtration system. In this embodiment, a reduced amount of buffer material is added to the stream relative to the buffer material lost in the permeate, resulting in an increased concentration. In certain embodiments, buffer materials can be replaced as the flow stream circulates through the filtration system. In this embodiment, the buffer added to stream is different from buffer materials lost in the permeate, resulting in an eventual replacement of buffer material in the stream. TFF systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, a TFF load pool can be spiked with an excipient or diluent prior to filtration. In certain embodiments, a TFF load pool is spiked with a high-salt mixture (such as sodium chloride or potassium chloride) prior to filtration. In certain embodiments, a TFF load pool is spiked with a high-sugar mixture (such as 50% w/v sucrose) prior to filtration.

The effectiveness of TFF processing can depend on several factors, comprising (but not limited to): shear stress from flow design, cross-flow rate, filtrate flow control, transmembrane pressure (TMP), membrane conditioning, membrane composition (e.g., hollow fiber construction) and design (e.g., surface area), system flow design, reservoir design, and mixing strategy. In certain embodiment, the filtration membrane can be exposed to pre-TFF membrane conditioning.

In certain embodiments, TFF processing can comprise one or more microfiltration stages. In certain embodiments, TFF processing can comprise one or more ultrafiltration stages. In certain embodiments, TFF processing can comprise one or more nanofiltration stages.

In certain embodiments, TFF processing can comprise one or more concentration stages, such as an ultrafiltration (UF) or microfiltration (MF) concentration stage. In the concentration stage, a reduced amount of buffer material is replaced as the stream circulates through the filtration system (relative to the amount of buffer material lost as permeate). The failure to completely replace all of the buffer material lost in the permeate results in an increased concentration of viral particles within the filtration stream. In certain embodiments, an increased amount of buffer material is replaced as the stream circulates through the filtration system. The incorporation of excess buffer material relative to the amount of buffer material lost in the permeate results in a decreased concentration of viral particles within the filtration stream.

In certain embodiments, TFF processing can comprise one or more diafiltration (DF) stages. The diafiltration stage comprises replacement of a first buffer material (such as a high salt material) within a second buffer material (such a low-salt or zero-salt material). In this embodiment, a second buffer is added to flow stream which is different from a first buffer material lost in the permeate, resulting in an eventual replacement of buffer material in the stream.

In certain embodiments, TFF processing can comprise multiple stages in series. In certain embodiments, a TFF processing process can comprise an ultrafiltration (UF) concentration stage followed by a diafiltration stage (DF). In some embodiments, TFF comprising UF followed by DF results in increased rAAV recovery relative to TFF comprising DF followed by UF. In some embodiments, TFF comprising UF followed by DF results in about 70-80% recovery of rAAV.

In certain embodiments, a TFF processing can comprise a diafiltration stage followed by an ultrafiltration concentration stage. In certain embodiments, a TFF processing can comprise a first diafiltration stage, followed by an ultrafiltration concentration stage, followed by a second diafiltration stage. In certain embodiments, a TFF processing can comprise a first diafiltration stage which incorporates a high-salt-low-sugar buffer material into the flow stream, followed by an ultrafiltration/concentration stage which results in a high concentration of the viral material in the flow stream, followed by a second diafiltration stage which incorporates a low-salt-high-sugar or zero-salt-high-sugar buffer material into the flow stream. In certain embodiments, the salt can be sodium chloride, sodium phosphate, potassium chloride, potassium phosphate, or a combination thereof. In certain embodiments, the sugar can be sucrose, such as a 5% w/v sucrose mixture or a 7% w/v sucrose mixture.

In certain embodiments, the one or more TFF steps can comprise a formulation diafiltration step in which at least a portion of the liquid media of the viral production pool is replaced with a high-sucrose formulation buffer. In certain embodiments, the high-sucrose formulation buffer comprises between 6-8% w/v of a sugar or sugar substitute and between 90-100 mM of an alkali chloride salt. In certain embodiments, the high-sucrose formulation buffer comprises 7% w/v of sucrose and between 90-100 mM sodium chloride. In certain embodiments, the high-sucrose formulation buffer comprises 7% w/v of sucrose, 10 mM Sodium Phosphate, between 95-100 mM sodium chloride, and 0.001% (w/v) Poloxamer 188. In certain embodiments, the formulation diafiltration step is the final diafiltration step in the one or more TFF steps. In certain embodiments, the formulation diafiltration step is the only diafiltration step in the one or more TFF steps.

In certain embodiments, TFF processing can comprise multiple stages which occur contemporaneously. As a non-limiting example, a TFF clarification process can comprise an ultrafiltration stage which occurs contemporaneously with a concentration stage.

Methods of cell lysate clarification and purification by filtration are well understood in the art and may be carried out according to a variety of available methods comprising, but not limited to passive filtration and flow filtration. Filters used may comprise a variety of materials and pore sizes. For example, cell lysate filters may comprise pore sizes of from about 1 μM to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may comprise, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In certain embodiments, clarification may comprise filtration through a filter with 2.0 μM pore size to remove large debris, followed by passage through a filter with 0.45 μM pore size to remove intact cells.

Filter materials may be composed of a variety of materials. Such materials may comprise, but are not limited to, polymeric materials and metal materials (e.g., sintered metal and pored aluminum.) Exemplary materials may comprise, but are not limited to nylon, cellulose materials (e.g., cellulose acetate), polyvinylidene fluoride (PVDF), polyethersulfone, polyamide, polysulfone, polypropylene, and polyethylene terephthalate. In certain embodiments, filters useful for clarification of cell lysates may comprise, but are not limited to ULTIPLEAT PROFILE™ filters (Pall Corporation, Port Washington, N.Y.), SUPOR™ membrane filters (Pall Corporation, Port Washington, N.Y.).

In certain embodiments, flow filtration may be carried out to increase filtration speed and/or effectiveness. In certain embodiments, flow filtration may comprise vacuum filtration. According to such methods, a vacuum is created on the side of the filter opposite that of cell lysate to be filtered. In certain embodiments, cell lysates may be passed through filters by centrifugal forces. In certain embodiments, a pump is used to force cell lysate through clarification filters. Flow rate of cell lysate through one or more filters may be modulated by adjusting one of channel size and/or fluid pressure.

### Clarification and Purification: Chromatography

In certain embodiments, AAV particles in a formulation may be clarified and purified from cell lysates through one or more chromatography steps using one or more different methods of chromatography. Chromatography refers to any number of methods known in the art for selectively separating out one or more elements from a mixture. Such methods may comprise, but are not limited to, ion exchange chromatography (e.g., cation exchange chromatography and anion exchange chromatography), affinity chromatography (e.g., immunoaffinity chromatography, metal affinity chromatography, pseudo affinity chromatography such as Blue Sepharose resins), hydrophobic interaction chromatography (HIC), size-exclusion chromatography, and multimodal chromatography (MMC) (chromatographic methods that utilize more than one form of interaction between the stationary phase and analytes). In certain embodiments, methods or systems of viral chromatography may comprise any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Chromatography systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, one or more ion exchange (IEX) chromatography steps may be used to isolate viral particles. The ion exchange step can comprise anion exchange (AEX) chromatography, cation exchange (CEX) chromatography, or a combination thereof. In certain embodiments, ion exchange chromatography is used in a bind/elute mode. Bind/elute IEX can be used by binding viral particles to a stationary phase based on charge-charge interactions between capsid proteins (or other charged components) of the viral particles and charged sites present on the stationary phase. This process can comprise the use of a column through which viral preparations (e.g., clarified lysates) are passed. After application of viral preparations to the charged stationary phase (e.g., column), bound viral particles may then be eluted from the stationary phase by applying an elution solution to disrupt the charge-charge interactions. Elution solutions may be optimized by adjusting salt concentration and/or pH to enhance recovery of bound viral particles. In certain embodiments, the elution solution can comprise a nuclease such as Benzonase nuclease. Depending on the charge of viral capsids being isolated, cation or anion exchange chromatography methods may be selected. In certain embodiments, ion exchange chromatography is used in a flow-through mode. Flow-through IEX can be used by binding non-viral impurities or unwanted viral particles to a stationary phase (based on charge-charge interactions) and allowing the target viral particles in the viral preparation to “flow through” the IEX system into a collection pool.

Methods or systems of ion exchange chromatography may comprise, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In certain embodiments, the IEX process uses an AEX chromatography system such as a Sartorius Sartobind Q membrane, a Sartorius Sartobind STIC membrane, a Millipore Fractogel TMAE HiCap(m) Flow-Through membrane, a GE Q Sepharose HP membrane, Poros XQ or Poros HQ. In certain embodiments, the IEX process uses a CEX system such as a Poros XS membrane. In certain embodiments, the AEX system comprises a stationary phase which comprises a trimethylammoniumethyl (TMAE) functional group. In certain embodiments, the IEX process uses a Multimodal Chromatography (MMC) system such as a Nuvia aPrime 4A membrane.

In certain embodiments, one or more affinity chromatography steps, such as immunoaffinity chromatography, may be used to isolate viral particles. Immunoaffinity chromatography is a form of chromatography that utilizes one or more immune compounds (e.g., antibodies or antibody-related structures) to retain viral particles. Immune compounds may bind specifically to one or more structures on viral particle surfaces, comprising, but not limited to one or more viral coat protein. In certain embodiments, immune compounds may be specific for a particular viral variant. In certain embodiments, immune compounds may bind to multiple viral variants. In certain embodiments, immune compounds may comprise recombinant single-chain antibodies. Such recombinant single chain antibodies may comprise those described in Smith, R. H. et al., 2009. Mol. Ther. 17(11):1888-96, the contents of which are herein incorporated by reference in their entirety. Such immune compounds (e.g., recombinant protein ligands) are capable of binding to several AAV capsid variants, comprising, but not limited to AAV1, AAV2, AAV3, AAV5, AAV6 and/or AAV8 or any of those taught herein. In some embodiments, such immune compounds (e.g., recombinant protein ligands) are capable of binding to at least AAV2. In certain embodiments, the AFC process uses a GE AVB Sepharose HP column resin, Poros CaptureSelect AAV8 resins (ThermoFisher), Poros CaptureSelect AAV9 resins (ThermoFisher) and Poros CaptureSelect AAVX resins (ThermoFisher).

In some embodiments, one or more affinity chromatography steps precedes one or more anion exchange chromatography steps. In some embodiments, one or more anion exchange chromatography steps precedes one or more affinity chromatography steps.

In certain embodiments, one or more size-exclusion chromatography (SEC) steps may be used to isolate viral particles. SEC may comprise the use of a gel to separate particles according to size. In viral particle purification, SEC filtration is sometimes referred to as “polishing.” In certain embodiments, SEC may be carried out to generate a final product that is near-homogenous. Such final products may in certain embodiments be used in pre-clinical studies and/or clinical studies (Kotin, R. M. 2011. Human Molecular Genetics. 20(1):R2-R6, the contents of which are herein incorporated by reference in their entirety.) In certain embodiments, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In some embodiments, purification of recombinant AAV produces a total rAAV process yield of 30-50%.

### Exemplary Embodiments of rAAV Production Steps

The method steps described in the following paragraphs may be used in a method for producing rAAV comprising a polynucleotide encoding AADC or a functional variant thereof in any combination or permutation, unless otherwise specified. Non-limiting steps, combinations, and permutations are provided below.

In some embodiments, the present disclosure encompasses a method for producing rAAV comprising a polynucleotide encoding AADC or a functional variant thereof, wherein the method comprises culturing viral production cells (VPCs), such as but not limited to Sf9 cells (“viral production Sf9 cells”) to a target cell density (viable cell density (“VCD”)) of 3.0×10⁶-3.4×10⁶ cells/mL. In some embodiments, the VCD is 3.2×10⁶-3.4×10⁶ cells/mL. In some embodiments, the VCD is about 3.2×10⁶ cells/mL. In some embodiments, once the VPCs reach the target cell density (e.g., about 3.2×10⁶ cells/mL), the VPCs are incubated with baculoviruses comprising a viral expression construct (e.g., for expressing Rep and/or Cap proteins) (“expressionBacs”) and baculoviruses comprising a payload construct (e.g., for expressing AADC) (“payloadBacs”). In some embodiments, once the VPCs reach the target cell density (e.g., about 3.2×10⁶ cells/mL), the VPCs are incubated with baculovirus infected insect cells comprising expressionBacs (“expressionBIICs”) and baculovirus infected insect cells comprising payloadBacs (“payloadBIICs”). In some embodiments, the ratio of expressionBIICs to payloadBIICs is about 1:3 (v/v). In some embodiments, the ratio of expressionBIICs to VPCs is about 1:300,000 (v/v). In some embodiments, the ratio of payloadBIICs to VPCs is about 1:100,000 (v/v). In some embodiments, the VPCs are Sf9 cells (i.e., “viral production Sf9 cells”). In some embodiments the expressionBacs are Sf9 cells. In some embodiments, the payloadBacs are Sf9 cells. In some embodiments, the VPCs (e.g., Sf9 cells) are incubated in serum-free, protein-free insect cell culture medium. In some embodiments, the VPCs (e.g., Sf9 cells) are incubated in serum-free, protein-free insect cell culture medium comprising L-glutamine and poloxamer-188. In some embodiments, the VPCs (e.g., Sf9 cells) are incubated in ESF AF Insect Cell Culture Medium. In some embodiments, incubation in ESF AF Insect Cell Culture Medium increases titer at least 2-fold compared to SFX Insect Cell Culture Media.

In some embodiments, expressionBIICs are introduced to a culture of VPCs at an expressionBIIC:VPC ratio of about 1:300,000 (v/v) and payloadBIICs are introduced to the culture at a payloadBIIC:VPC ratio of about 1:100,000 (v/v) (wherein the expressionBIIC:payloadBIIC ratio is about 1:3), and these BIICs are introduced to the culture once the VPCs have reached a target cell density of about 3.2×10⁶ cells/mL. In some embodiments, introducing the expressionBIICs and payloadBIICs to the VPCs at these ratios (1:300,000 (v/v) and 1:100,000 (v/v), respectively) and when VPCs have reached a target cell density of about 3.2×10⁶ cells/mL results in an increase in rAAV productivity relative to a method comprising a lower expressionBIIC:VPC ratio, a lower payload:VPC ratio, and/or a lower VPC target cell density. In some embodiments, introducing the expressionBIICs and payloadBIICs to the VPCs at these ratios (1:300,000 (v/v) and 1:100,000 (v/v), respectively) and when VPCs have reached a target cell density of about 3.2×10⁶ cells/mL results in an increase in rAAV productivity relative to a method comprising an expressionBIIC:VPC ratio of 1:250,000 (v/v), a payload:VPC ratio of 1:50,000, and a VPC target cell density of about 2.7×10⁶-2.8×10⁶ cells/mL (e.g., 2.75×10⁶ cells/mL). In some embodiments, the relative increase in rAAV productivity is at least 10-fold. In some embodiments, the relative increase in rAAV productivity of about 10-fold.

In some embodiments, expressionBIICs are introduced to a culture of VPCs at an expressionBIIC:VPC ratio of about 1:300,000 (v/v) and payloadBIICs are introduced to the culture at a payloadBIIC:VPC ratio of about 1:100,000 (v/v) (wherein the expressionBIIC:payloadBIIC ratio is about 1:3), and these BIICs are introduced to the culture once the VPCs have reached a target cell density of about 3.2×10⁶ cells/mL, and the resulting rAAV productivity is at least 4×10¹¹ vg/mL.

In some embodiments, a method for producing rAAV comprising a polynucleotide encoding AADC or a functional variant thereof comprises introducing expressionBIICs to a culture of VPCs at an expressionBIIC:VPC ratio of about 1:300,000 (v/v) and introducing payloadBIICs to the culture at a payloadBIIC:VPC ratio of about 1:100,000 (v/v) (wherein the expressionBIIC:payloadBIIC ratio is about 1:3), such that the BIICs are introduced to the culture once the VPCs have reached a target cell density of about 3.2×10⁶ cells/mL, and further comprising incubating the VPCs under conditions that result in the production of one or more rAAVs within one or more VPCs, and lysing the VPCs. In some embodiments, lysis comprises a chemical lysis solution lacking detectable nuclease. In some embodiments, the method further comprises one or more clarification steps. In some embodiments, the method further comprises one or more chromatography steps. In some embodiments, the one or more chromatography steps comprises immunoaffinity chromatography. In some embodiments, the one or more chromatography steps comprises anion exchange chromatography. In some embodiments, the one or more chromatography steps comprises immunoaffinity chromatography and anion exchange chromatography. In some embodiments, the method further comprises tangential flow filtration (TFF). In some embodiments, the method further comprises ultrafiltration. In some embodiments, the method further comprises diafiltration. In some embodiments, the method further comprises one or more tangential flow filtration (TFF) steps, comprising ultrafiltration followed by diafiltration. In some embodiments, the method further comprises one or more viral retentive filtration (VRF) steps. In some embodiments, the method further comprises further filtration steps, which may occur before or after any of the steps or processes described above.

In some embodiments, the present disclosure encompasses a method for producing rAAV comprising a polynucleotide encoding AADC or a functional variant thereof, wherein the method comprises one or more anion exchange chromatography steps. In some embodiments, the method produces higher rAAV yield (e.g., concentration) relative to a method comprising one or more cation exchange chromatography steps. In some embodiments, the method produces higher rAAV purity (e.g., fewer contaminants, such as baculoviral contaminants or VPC protein contaminants) relative to a method comprising one or more cation exchange chromatography steps. In some embodiments, the one or more anion exchange chromatography steps follows one or more immunoaffinity chromatography steps. In some embodiments, the one or more anion exchange chromatography steps precedes one or more immunoaffinity chromatography steps. In some embodiments, prior to the one or more anion exchange chromatography steps, the method comprises introducing expressionBIICs and payloadBIICs to a culture of VPCs (e.g., incubating in serum-free, protein-free insect cell medium) once the VPCs have reached a target cell density, incubating the VPCs under conditions that result in the production of one or more rAAVs within one or more VPCs, and lysing the VPCs. In some embodiments, prior to the one or more anion exchange chromatography steps, the method comprises introducing expressionBIICs to a culture of VPCs at an expressionBIIC:VPC ratio of about 1:300,000 (v/v) and introducing payloadBIICs to the culture at a payloadBIIC:VPC ratio of about 1:100,000 (v/v) (wherein the expressionBIIC:payloadBIIC ratio is about 1:3), such that the BIICs are introduced to the culture once the VPCs have reached a target cell density of about 3.2×10⁶ cells/mL, and further comprising incubating the VPCs under conditions that result in the production of one or more rAAVs within one or more VPCs, and lysing the VPCs. In some embodiments, lysing the VPCs comprises a chemical lysis solution lacking detectable nuclease.

Further, in some embodiments, the method further comprises one or more clarification steps. In some embodiments, the one or more clarification steps comprises a C0SP filter. In some embodiments, the method further comprises tangential flow filtration (TFF). In some embodiments, the method further comprises ultrafiltration. In some embodiments, the method further comprises diafiltration. In some embodiments, the method further comprises one or more tangential flow filtration (TFF) steps, comprising ultrafiltration followed by diafiltration. In some embodiments, the method further comprises one or more viral retentive filtration (VRF) steps. In some embodiments, the method further comprises further filtration steps, which may occur before or after any of the steps or processes described above.

In some embodiments, the present disclosure encompasses a method for producing rAAV comprising a polynucleotide encoding AADC or a functional variant thereof, wherein the method comprises ultrafiltration followed by diafiltration. In some embodiments, the diafiltration comprises buffer exchange into a pharmaceutical formulation buffer. In some embodiments, the pharmaceutical formulation buffer comprises 10 mM sodium phosphate, 180 mM sodium chloride, and 0.001% poloxamer 188 (solution pH of 7.1-7.5, e.g., pH 7.3). In some embodiments, ultrafiltration comprises concentration of rAAV to a concentration of at least 5.0×10¹² vg/mL. In some embodiments, ultrafiltration comprises concentration of rAAV to a concentration of about 5.0×10¹² vg/mL. In some embodiments, the ultrafiltration followed by diafiltration results in improved recovery of rAAV compared to a method comprising diafiltration followed by ultrafiltration. In some embodiments, ultrafiltration followed by diafiltration results in about 70-80% recovery of rAAV. In some embodiments, ultrafiltration followed by diafiltration results in about 70-80% recovery of rAAV, whereas diafiltration followed by ultrafiltration results in about 40-60% recovery of rAAV. In some embodiments, prior to the one or more anion exchange chromatography steps, the method comprises introducing expressionBIICs and payloadBIICs to a culture of VPCs (e.g., incubating in serum-free, protein-free insect cell medium) once the VPCs have reached a target cell density, incubating the VPCs under conditions that result in the production of one or more rAAVs within one or more VPCs, and lysing the VPCs. In some embodiments, prior to the one or more anion exchange chromatography steps, the method comprises introducing expressionBIICs to a culture of VPCs at an expressionBIIC:VPC ratio of about 1:300,000 (v/v) and introducing payloadBIICs to the culture at a payloadBIIC:VPC ratio of about 1:100,000 (v/v) (wherein the expressionBIIC:payloadBIIC ratio is about 1:3), such that the BIICs are introduced to the culture once the VPCs have reached a target cell density of about 3.2×10⁶ cells/mL, and further comprising incubating the VPCs under conditions that result in the production of one or more rAAVs within one or more VPCs, and lysing the VPCs. In some embodiments, lysing the VPCs comprises a chemical lysis solution lacking detectable nuclease.

Further, in some embodiments, the method further comprises one or more clarification steps. In some embodiments, the one or more clarification steps comprises a C0SP filter. In some embodiments, the method further comprises one or more chromatography steps. In some embodiments, the one or more chromatography steps comprises immunoaffinity chromatography. In some embodiments, the one or more chromatography steps comprises anion exchange chromatography. In some embodiments, the one or more chromatography steps comprises immunoaffinity chromatography and anion exchange chromatography. In some embodiments, the method further comprises one or more viral retentive filtration (VRF) steps. In some embodiments, the method further comprises further filtration steps, which may occur before or after any of the steps or processes described above.

In any of the preceding embodiments, the method may produce a total rAAV process yield of 30-50%. In any of the preceding embodiments, the method may produce a total rAAV concentration of 3.0×10¹²-5.0×10¹² vg/mL. In any of the preceding embodiments, the method may produce a total rAAV concentration of about 5.0×10¹² vg/mL. In any of the preceding embodiments, the method may produce a total rAAV concentration of greater than 5.0×10¹² vg/mL. In any of the preceding embodiments, the method may produce higher rAAV yield (e.g., higher concentration) compared to a method comprising one or more of: a lower expressionBIIC:VPC ratio, a lower payload:VPC ratio, a lower VPC target cell density, one or more cation exchange chromatography steps, and/or diafiltration followed by ultrafiltration. In any of the preceding embodiments, the method may produce higher rAAV purity (e.g., fewer baculoviral contaminants, fewer VPC protein contaminants) compared to a method comprising one or more of: a lower expressionBIIC:VPC ratio, a lower payload:VPC ratio, a lower VPC target cell density, one or more cation exchange chromatography steps, and/or diafiltration followed by ultrafiltration.

For example, in any of the preceding embodiments, the method may produce higher rAAV yield (e.g., higher concentration) compared to a method comprising: introducing expressionBIICs to a culture comprising VPCs at an expressionBIIC:VPC ratio of 1:250,000 (v/v), introducing payloadBIICs to the culture at a payload:VPC ratio of 1:50,000, wherein the VPC target cell density at the time of introducing these BIICs is about 2.75×10⁶ cells/mL; incubating VPCs in SFX Insect Cell Culture Medium; lysing the VPCs in a solution comprising a nuclease; clarifying the lysed VPCs comprising depth filtration (e.g., using a DOHC filter) to yield a clarified lysate pool; processing the clarified lysate pool comprising immunoaffinity chromatography followed by cation exchange chromatography, yielding a chromatography pool; processing the chromatography pool comprising TFF comprising diafiltration followed by ultrafiltration, yielding a TFF pool; and processing the TFF pool comprising viral retentive filtration. Further, the method of any of the preceding embodiments may produce higher rAAV purity (e.g., fewer baculoviral contaminants, fewer VPC protein contaminants) compared to a method comprising: introducing expressionBIICs to a culture comprising VPCs at an expressionBIIC:VPC ratio of 1:250,000 (v/v), introducing payloadBIICs to the culture at a payload:VPC ratio of 1:50,000, wherein the VPC target cell density at the time of introducing these BIICs is about 2.75×10⁶cells/mL; incubating VPCs in SFX Insect Cell Culture Medium; lysing the VPCs in a solution comprising a nuclease; clarifying the lysed VPCs comprising depth filtration (e.g., using a DOHC filter) to yield a clarified lysate pool; processing the clarified lysate pool comprising immunoaffinity chromatography followed by cation exchange chromatography, yielding a chromatography pool; processing the chromatography pool comprising TFF comprising diafiltration followed by ultrafiltration, yielding a TFF pool; and processing the TFF pool comprising viral retentive filtration.

In any of the preceding embodiments, the rAAV produced by the method comprise a viral capsid ratio VP1:VP2:VP3 of about 1:1:10.

In any of the preceding embodiments, the method may produce a purified drug substance comprising a purified rAAV formulation, e.g., one which is aseptically filled into a glass vial, e.g., wherein the vial is a 2 mL Ompi Glass Vial. In some embodiments, the vial comprises a 1.0-2.0 mL fill volume and 0.75-1.75 mL extractable volume of the purified drug substance. In some embodiments, the vial comprises an about 1.2 mL fill volume and an about 1.0 mL extractable volume of the purified drug substance. In some embodiments, the vial is stoppered using a 13 mm West S2-F451 4432/50 stopper and sealed with a 13 mm West Pharma Flip Off Long Matte Seal.

In any of the preceding embodiments, the method may produce a total rAAV concentration of ≥3.0×10¹² vg/mL. In any of the preceding embodiments, the method may produce a total rAAV concentration of about 3.0×10¹² vg/mL to about 5.0×10¹² vg/mL. In any of the preceding embodiments, the method may produce a total rAAV concentration of about 5.0×10¹² vg/mL.

In any of the preceding embodiments, the method may produce a purified drug substance that has an infectious AAV viral titer of greater than 3×10¹⁰ TU/mL.

In any of the preceding embodiments, the method may produce a purified drug substance that has a particle-to-infectious unit ratio of less than about 100 vg/TU. An infectious titer ratio or particle-to-infectious unit ratio as used herein refers to the proportion of total viral particles that are infectious. A higher particle-to-infectious unit ratio typically indicates lower infectivity. In certain embodiments, the particle-to-infectious unit ratio may be determined by a median tissue culture infectious dose 50 (TCID50) assay.

In any of the preceding embodiment, the method may produce a purified composition of rAAV comprising a polynucleotide encoding AADC (e.g., SEQ ID NO: 979). In some embodiments, the composition has an AADC relative potency of at least 50% as compared to a viral vector reference standard (e.g., a viral vector with known AADC activity). In certain embodiments, the rAAV has an AADC relative potency of about 50% to about 250% as compared to a viral vector standard. In certain embodiments, the rAAV has an AADC relative potency of at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, or greater, as compared to a viral reference standard. In certain embodiments, the AADC relative potency may be determined by infection of permissive cells using the purified rAAV formulation and measuring AADC activity using high performance liquid chromatography (HPLC). In certain embodiments, the AADC relative potency may be determined by comparing the amount of dopamine produced by cells infected with the rAAV to the amount of dopamine produced by a viral reference standard.

In any of the preceding embodiments, the method may produce a purified rAAV composition comprising ≤40% empty viral particles. In any of the preceding embodiments, the method may produce a purified rAAV composition comprising less than about 40% empty viral capsids, less than about 30% empty viral capsids, less than about 20% empty viral capsids, less than about 10% empty viral capsids, less than about 5% empty viral capsids, less than about 2% empty viral capsids, or less. In certain embodiments, the percentage of empty viral capsids in the purified rAAV composition may be determined by gradient centrifugation or ultracentrifugation (e.g., analytical ultracentrifugation). In certain embodiments, the percentage of empty viral capsids in the purified rAAV composition may be determined by UV absorbance.

In any of the preceding embodiments, the method may produce a purified rAAV formulation comprising greater than about 60% full viral capsids, greater than about 70% full viral capsids, greater than about 80% full viral capsids, greater than about 90% full viral capsids, greater than about 95% full viral capsids, greater than about 97% full viral capsids, greater than about 99% viral capsids, or greater. In certain embodiments, the percentage of full capsids may be determined by gradient centrifugation or ultracentrifugation (e.g., analytical ultracentrifugation). In certain embodiments, the percentage of full capsids may be determined by UV absorbance.

In any of the preceding embodiments, the method may produce a purified rAAV composition that has an osmolality of 300-400 mOsm/kg. In some embodiments, the osmolality of the composition is in accordance with United States Pharmacopoiea (USP), e.g., USP <785>, Ph. Eur. 2.2.35, the content of which is incorporated herein by its entirety.

In any of the preceding embodiments, the method may produce a purified rAAV composition comprising less than about 6000 particles with a size of ≥10 μm and less than about 600 particles with a size of ≥25 μm.

In any of the preceding embodiments, the method may produce a purified rAAV formulation that has a pH of about 7.3 (e.g., pH 7.3±0.5). In some embodiments, the pH of the composition is in accordance with United States Pharmacopoiea (USP), e.g., USP <791>, Ph. Eur. 2.2.3, the content of which is incorporated herein by its entirety.

In any of the preceding embodiments, the method may produce a purified rAAV composition that has endotoxin levels of less than about 1 EU/mL. In some embodiments, the endotoxin levels of the composition is in accordance with United States Pharmacopoiea (USP), e.g., USP <85>, Ph. Eur. 2.6.14, the content of which is incorporated herein by its entirety.

In any of the preceding embodiments, the method may produce a purified rAAV composition that exhibits a bioburden of less than 1 CFU/10 mL. In some embodiments, the bioburden of the composition disclosed herein is in accordance with United States Pharmacopoiea (USP), e.g., USP <61>, Ph. Eur. 2.6.12, the content of which is incorporated herein by reference in its entirety.

In any of the preceding embodiments, the method may produce a purified drug substance that has a protein purity of greater than about 90% (e.g., product-related protein represents ≥90% of total protein and no other proteins are present at greater than about 5% as determined by Capillary Electrophoresis Sodium Dodecyl Sulfate (CE-SDS)).

In any of the preceding embodiments, the method may produce a purified drug substance comprising a purified rAAV composition, wherein the rAAV composition comprises rAAV comprising a polynucleotide encoding AADC (e.g., SEQ ID NO: 979) or a functional variant thereof and an AAV2 viral capsid and wherein the composition has an AADC relative potency of at least 50%, wherein the composition comprises greater than or equal to 3.0×10¹² vg/mL (e.g., about 5.0×10¹² vg/mL) of rAAVs. In some embodiments, the AAV2 viral capsid is encoded by the nucleic acid sequence of SEQ ID NO: 1778. In some embodiments, the AAV2 viral capsid comprises the amino acid sequence of SEQ ID NO: 16. In some embodiments, the rAAVs are present in a solution comprising 5-15 mM sodium phosphate, 150-250 mM sodium chloride, and 0.001-0.005% poloxamer (solution pH of 7.3±0.5), wherein the rAAV formulation comprises greater than about 60% full viral capsids (e.g., less than about 40% empty viral capsids), less than about 1 EU/mL endotoxin levels, greater than about 90% protein purity, and greater than about 3×10¹⁰ TU/mL infectious titer. In some embodiments, the rAAV composition has an osmolality of 300-400 mOsm/kg. In some embodiments, the rAAV composition comprises less than about 6000 particles with a size of ≥10 μm and less than about 600 particles with a size of ≥25 μm.

### Exemplary Embodiment of an rAAV Production Method

The present disclosure presents methods and systems for producing recombinant adeno-associated viruses (rAAVs).

In certain embodiments, the present disclosure encompasses a method for producing rAAV comprising a polynucleotide encoding aromatic L-amino acid decarboxylase (AADC) or a functional variant thereof. In some embodiments, the method comprises the steps of culturing viral production cells (VPCs) in a bioreactor to a target cell density; introducing into the bioreactor at least one baculovirus (expressionBac) comprising a viral expression construct, and at least one baculovirus (payloadBac) comprising a payload construct, wherein the viral expression construct comprises an adeno-associated virus (AAV) viral expression construct, and wherein the payload construct comprises the polynucleotide encoding AADC or a functional variant thereof; incubating the VPCs in the bioreactor under conditions that result in the production of one or more rAAVs within one or more VPCs, wherein one or more of the rAAVs comprise the polynucleotide encoding AADC or a functional variant thereof; harvesting a viral production pool from the bioreactor, wherein the viral production pool comprises one or more VPCs comprising one or more rAAVs; lysing the one or more VPCs in the viral production pool, thereby releasing one or more rAAVs from the one or more VPCs into a lysis medium; and processing the lysis medium. In some embodiments, the processing step comprises one or more clarifying steps; one or more immunoaffinity chromatography steps; one or more anion exchange chromatography steps; one or more tangential flow filtration (TFF) steps, wherein the one or more TFF steps comprises ultrafiltration followed by diafiltration; and one or more virus retentive filtration (VRF) steps, wherein the processing may further comprise one or more filtration steps before or after any one or more of the processing steps described above. In certain embodiments, the VPCs are insect cells, e.g., Sf9 cells. In some embodiments, the polynucleotide encoding AADC or a functional variant thereof encodes SEQ ID NO: 978.

In some embodiments, the at least one baculovirus (expressionBac) comprising a viral expression construct is comprised in at least one baculovirus infected insect cell (expressionBIIC). In some embodiments, the baculovirus infected insect cell (expressionBIIC) comprising at least one expressionBac is an Sf9 cell. In some embodiments, the at least one baculovirus (payloadBac) comprising a payload construct is comprised in at least one baculovirus infected insect cell (payloadBIIC). In some embodiments, the baculovirus infected insect cell (payloadBIIC) comprising at least one payloadBac is an Sf9 cell.

In certain embodiments, the present disclosure encompasses a method for producing a recombinant adeno-associated virus (rAAV) comprising a polynucleotide encoding aromatic L-amino acid decarboxylase (AADC) or a functional variant thereof. In some embodiments, the method comprises the steps of: (a) culturing viral production cells (VPCs) in a bioreactor to a target cell density; (b) introducing into the bioreactor at least one baculovirus (expressionBac) comprising a viral expression construct, and at least one baculovirus (payloadBac) comprising a payload construct, wherein the viral expression construct comprises an adeno-associated virus (AAV) viral expression construct, and wherein the payload construct comprises the polynucleotide encoding AADC or a functional variant thereof; (c) incubating the VPCs in the bioreactor under conditions that result in the production of one or more rAAVs within one or more VPCs, wherein one or more of the rAAVs comprise the polynucleotide encoding AADC or a functional variant thereof; (d) harvesting a viral production pool from the bioreactor, wherein the viral production pool comprises one or more VPCs comprising one or more rAAVs; (e) lysing the one or more VPCs in the viral production pool by chemical lysis, thereby releasing one or more rAAVs from the one or more VPCs into a lysis medium; (f) clarifying the lysis medium of (e) through one or more clarifying steps, yielding a clarification pool, wherein the clarification pool is optionally filtered; (g) processing the clarification pool of (f) through one or more immunoaffinity chromatography steps, yielding an immunoaffinity chromatography pool, wherein the one or more immunoaffinity chromatography steps optionally comprises neutralizing the immunoaffinity chromatography pool, and wherein the immunoaffinity chromatography pool is optionally filtered; (h) processing the immunoaffinity chromatography pool of (g) through one or more anion exchange chromatography steps, yielding an anion exchange chromatography pool, wherein the anion exchange chromatography pool is optionally filtered; (i) processing the anion exchange chromatography pool of (h) through one or more tangential flow filtration (TFF) steps, wherein the one or more TFF steps comprises ultrafiltration followed by diafiltration, wherein the one or more TFF steps yields a concentrated, buffer-exchanged pool, wherein the concentrated, buffer-exchanged pool is optionally filtered; and (j) processing the concentrated, buffer-exchanged pool of (i) through one or more virus retentive filtration (VRF) steps, yielding a viral filtration pool comprising rAAVs comprising a polynucleotide encoding AADC or a functional variant thereof, wherein the viral filtration pool is optionally filtered. In some embodiments, the viral filtration pool of step (j) is further processed through a filtration step. In certain embodiments, the VPCs are insect cells. In some embodiments, the insect cells are Sf9 cells. In some embodiments, the polynucleotide encoding AADC or a functional variant thereof encodes SEQ ID NO: 978.

In some embodiments, the at least one baculovirus (expressionBac) comprising a viral expression construct is comprised in at least one baculovirus infected insect cell (expressionBIIC). In some embodiments, the baculovirus infected insect cell (expressionBIIC) comprising at least one expressionBac is an Sf9 cell. In some embodiments, the at least one baculovirus (payloadBac) comprising a payload construct is comprised in at least one baculovirus infected insect cell (payloadBIIC). In some embodiments, the baculovirus infected insect cell (payloadBIIC) comprising at least one payloadBac is an Sf9 cell.

In some embodiments, the payload construct comprises a 5′ inverted terminal repeat (ITR), at least one multiple cloning site (MCS) region, a cytomegalovirus (CMV) enhancer, a CMV promoter, an intron region comprising immediate-early 1 (Ie1) exon 1, Ie1 intron 1 (partial), human beta-globin (hBglobin) intron 2, and hBglobin intron 3, a polyadenylation (poly(A)) signal, and a 3′ ITR. In some embodiments, the payload construct comprises, e.g., in order from 5′ to 3′: a 5′ ITR comprising SEQ ID NO: 980, a first MCS region comprising SEQ ID NO: 981, a CMV enhancer comprising SEQ ID NO: 982, a CMV promoter comprising SEQ ID NO: 983, an intron region comprising an Ie1 exon 1 (SEQ ID NO: 984), a partial Ie1 intron 1 (SEQ ID NO: 985), a human beta-globin (hBglobin) intron 2 (SEQ ID NO: 986), and a hBglobin intron 3 (SEQ ID NO: 987), a polynucleotide encoding an AADC amino acid sequence comprising SEQ ID NO: 978, wherein optionally the polynucleotide comprises SEQ ID NO: 988, a poly(A) signal comprising SEQ ID NO: 990, and a 3′ ITR comprising SEQ ID NO: 991. In some embodiments, the payload construct comprises a sequence at least 90%, at least 95%, at least 96%, at least 97%, at least 98%, or at least 99% identical to SEQ ID NO: 979. In some embodiments, the payload construct comprises SEQ ID NO: 979.

In some embodiments, the at least one expressionBIIC is introduced into the bioreactor at a ratio of 1:250,000 to 1:350,000 (v/v) relative to VPCs (e.g., 1:300,000 expressionBIIC:VPC (v/v)) and/or the at least one payloadBIIC is introduced into the bioreactor at a ratio of 1:50,000 to 1:150,000 (v/v) relative to VPCs (e.g., 1:100,000 payloadBIIC:VPC (v/v)).

In some embodiments, the VPCs are cultured in the bioreactor in insect cell culture medium. In some embodiments, the insect cell culture medium is a serum free, protein-free medium, wherein optionally the insect cell culture medium comprises L-glutamine and poloxamer 188, wherein further optionally the insect cell culture medium comprises EFS AF™ insect cell culture medium.

In some embodiments, the VPCs are cultured in the bioreactor at 26° C.-28° C. (e.g., 27° C.) and 30%-50% (e.g., 40%) dissolved oxygen.

In some embodiments, the target cell density (i.e., viable cell density (VCD)) of VPCs prior to introduction of the expressionBIICs (or expressionBacs) and payloadBIICs (or payloadBacs) is about 3.0×10⁶-3.4×10⁶ cells/mL (e.g., 3.2×10⁶-3.4×10⁶ cells/mL; e.g., 3.2×10⁶ cells/mL).

In some embodiments, the target cell density (i.e., viable cell density (VCD)) of VPCs prior to introduction of the expressionBIICs and payloadBIICs is about 3.0×10⁶-3.4×10⁶ cells/mL (e.g., 3.2×10⁶-3.4×10⁶ cells/mL; e.g., 3.2×10⁶ cells/mL), the at least one expressionBIIC is introduced into the bioreactor at a ratio of 1:300,000 expressionBIIC:VPC (v/v)), and the at least one payloadBIIC is introduced into the bioreactor at a ratio of 1:100,000 payloadBIIC:VPC (v/v)). In some embodiments, one or more of the VPCs, expressionBIICs, and/or payloadBIICs are Sf9 cells. In some embodiments, all of the VPCs, expressionBIICs, and payloadBIICs are Sf9 cells.

In some embodiments, the lysing step comprises a chemical lysis solution comprising a surfactant and arginine or a salt thereof, wherein optionally the surfactant is octyl phenol ethoxylate and the arginine or salt thereof is arginine hydrochloride. In some embodiments, the chemical lysis solution comprises 0.5% (w/v) octyl phenol ethoxylate (e.g., Triton X-100) and 200 mM arginine hydrochloride. In some embodiments, the lysis pH is 6.8-7.5. In some embodiments, the chemical lysis solution is free of detectable nuclease. In some embodiments, the lysing is carried out for 4-6 hours (e.g., 4 hours) at 26° C.-28° C. (e.g., 27° C.). In some embodiments, the one or more clarifying steps comprises depth filtration followed by filtration through an about 0.2 μm filter.

In some embodiments, the one or more immunoaffinity chromatography steps comprises an immunoaffinity chromatography column comprising a recombinant protein ligand that binds at least AAV2, and optionally binds at least AAV1, AAV2, AAV3, and AAV5. In some embodiments, the immunoaffinity chromatography column is equilibrated with a solution comprising 50 mM sodium phosphate, 350 mM sodium chloride and 0.001% w/v poloxamer 188 (e.g., solution pH of 7.2-7.6, e.g., pH of 7.4); flushed with a solution comprising 50 mM sodium phosphate, 350 mM sodium chloride and 0.001% w/v poloxamer 188 (e.g., solution pH of 7.2-7.6, e.g., pH of 7.4); washed with a solution comprising 20 mM sodium citrate, 1 M sodium chloride and 0.001% w/v poloxamer 188 (e.g., solution pH of 5.8-6.2, e.g., pH of 6.0); and washed a second time with a solution of 10 mM sodium citrate, 350 mM sodium chloride and 0.001% w/v poloxamer 188 (e.g., solution pH of 5.8-6.2, e.g., pH of 6.0); wherein the one or more immunoaffinity chromatography steps yields a immunoaffinity chromatography pool. In some embodiments, the filtered product is eluted with a solution comprising 20 mM sodium citrate, 350 mM sodium chloride and 0.001% w/v poloxamer 188 (e.g., solution pH of 2.8-3.2, e.g., pH of 3.0). In some embodiments, the immunoaffinity chromatography pool is neutralized with 2 M Tris Base and 0.001% w/v poloxamer 188 (3.0% v/v spike, pH 8.0-8.5). In some embodiments, the immunoaffinity chromatography pool is filtered through an about 0.2 μm filter.

In some embodiments, the one or more immunoaffinity chromatography steps comprises loading the immunoaffinity chromatography column with a 1.0×10¹³-5.0×10¹³ vg/mL-r load challenge at 18-25° C.

In some embodiments, the one or more anion exchange chromatography steps comprises charging and equilibrating an anion exchange chromatography column with a solution comprising 20 mM Tris, 2 M sodium chloride and 0.001% w/v poloxamer 188, then a solution of 40 mM Tris, 170 mM sodium chloride and 0.001% w/v poloxamer 188 (e.g., solution pH of 7.8-8.2, e.g., pH of 8.0). In some embodiments, the anion exchange chromatography column is flushed and eluted with a solution comprising 40 mM Tris, 170 mM sodium chloride and 0.001% w/v poloxamer 188 (e.g., solution pH of 8.3-8.7, e.g., pH of 8.5), yielding an anion exchange chromatography pool. In some embodiments, the anion exchange chromatography elution pool is filtered through an about 0.2 μm filter.

In some embodiments, the one or more anion exchange chromatography steps comprises loading the anion exchange chromatography column with a 1.0×10¹³-5.0×10¹³ vg/mL-r load challenge at 18-25° C.

In some embodiments, the one or more TFF steps comprises TFF filtration with a TFF filter, yielding a TFF load pool, followed by concentration of the TFF load pool by ultrafiltration followed by diafiltration, yielding a final TFF load pool. In some embodiments, the TFF filtration comprises equilibration with a buffer (e.g., pH 8.3-8.7, e.g., pH 8.5) comprising 40 mM Tris, 170 mM sodium chloride, and 0.001% (w/v) poloxamer 188. In some embodiments, after obtaining the TFF load pool, the TFF filter is subjected to a recovery flush using a buffer comprising 10 mM sodium phosphate, 180 mM sodium chloride, and 0.001% (w/v) poloxamer 188, yielding a TFF recovery flush pool. In some embodiments, the TFF load pool is concentrated by ultrafiltration to a viral concentration of about 5.0×10¹² vg/mL. In some embodiments, the diafiltration step comprises buffer exchange with a buffer comprising 10 mM sodium phosphate, 180 mM sodium chloride, and 0.001% (w/v) poloxamer 188 (buffer pH of 7.1-7.5, e.g., pH 7.3). In some embodiments, the final TFF load pool is filtered through an about 0.2 μm filter, yielding a filtered final TFF load pool. In some embodiments, the TFF recovery flush pool is filtered through an about 0.2 μm filter, yielding a filtered TFF recovery flush pool. In some embodiments, the filtered final TFF load pool and the filtered TFF recovery flush pool are combined to form a concentrated, buffer-exchanged pool, wherein the concentrated, buffer-exchanged pool is optionally diluted using a buffer comprising 10 mM sodium phosphate, 180 mM sodium chloride, and 0.001% (w/v) poloxamer 188 (buffer pH of 7.1-7.5, e.g., pH 7.3), wherein the concentrated, buffer-exchanged pool comprises a viral concentration of 2.0×10¹²-6.0×10¹² vg/mL, e.g., 5.0×10¹² vg/mL.

In some embodiments, the one or more VRF steps comprises filtration with a VRF filter having a pore size of about 35 nm, yielding a viral filtration pool. In some embodiments, the VRF filter is flushed twice before use with a solution comprising 10 mM sodium phosphate, 180 mM sodium chloride, and 0.001% poloxamer 188 (solution pH of 7.1-7.5, e.g., pH 7.3). In some embodiments, the viral filtration pool is filtered through a filter of about 0.2 μm. In some embodiments, the viral filtration pool comprises a viral concentration of 3.5×10¹²-5.0×10¹² vg/mL, e.g., about 5.0×10¹² vg/mL. In some embodiments, the viral filtration pool is filtered at least once (optionally at least twice) using an about 0.22 μm filter, yielding a filtered drug substance pool in a solution comprising 10 mM sodium phosphate, 180 mM sodium chloride, and 0.001% poloxamer 188 (solution pH of 7.1-7.5, e.g., pH 7.3).

In some embodiments, the filtered drug substance pool comprises a viral concentration of 3.0×10¹²-5.0×10¹² vg/mL, e.g., about 5.0×10¹² vg/mL. In some embodiments, the VRCs, at least one expressionBac or expressionBIIC, and at least one payloadBac or payloadBIIC are incubated for 156-180 hours, e.g., 164-172 hours, e.g., 168 hours, prior to lysis. In some embodiments, the VRCs incubating with at least one expressionBac (e.g., expressionBIIC) and at least one payloadBac (e.g., payloadBIIC) have at least 85% viability, e.g., at least 90% viability, prior to lysis. In some embodiments, the viral production pool weighs 195-198 kg, e.g., 196 kg, prior to lysis. In some embodiments, the method produces a total process rAAV yield of 30%-50%. In some embodiments, the rAAVs comprise a capsid from AAV2. In some embodiments, the AAV2 capsid is encoded by nucleic acid sequence comprising SEQ ID NO: 1778. In some embodiments, the AAV2 capsid comprises the amino acid sequence SEQ ID NO: 16.

In some embodiments, the viral expression construct comprises one or more polynucleotides encoding a VP1 capsid protein, VP2 capsid protein, VP3 capsid protein, Rep52, and Rep78. In some embodiments, the VP1 capsid protein, VP2 capsid protein, and VP3 capsid protein are encoded in one or more open reading frames and the Rep52 and Rep78 are encoded in one or more open reading frames, wherein the one or more open reading frames encoding the VP1 capsid protein, VP2 capsid protein, and VP3 capsid protein and the one or more open reading frames encoding the Rep52 and Rep78 are different open reading frames. In some embodiments, the VP1 capsid protein, VP2 capsid protein, and VP3 capsid protein are encoded in a first open reading frame and the Rep52 and Rep78 are encoded in a second open reading frame. In some embodiments, the VP1 protein is encoded by a sequence that is at least 99% identical to SEQ ID NO: 1778. In some embodiments, the VP1 protein is encoded by a sequence comprising SEQ ID NO: 1778. In some embodiments, the VP1 protein comprises an amino acid sequence of SEQ ID NO: 16. In some embodiments, the Rep78 protein is encoded by a sequence that is at least 99% identical to SEQ ID NO: 1779. In some embodiments, the Rep78 protein is encoded by a sequence comprising SEQ ID NO: 1779. This nucleic acid sequence encoding the Rep78 protein is given here:

(SEQ ID NO: 1779)
5’ctggcggggttttacgagattgtgattaaggtccccagcgaccttgacgagcatctgcccggcatttctgacagcttt
gtgaactgggtggccgagaaggagtgggagttgccgccagattctgacttggatctgaatctgattgagcaggcacccctgaccgtg
gccgagaagctgcagcgcgactttctgacggagtggcgccgtgtgagtaaggccccggaggcccttttctttgtgcaatttgagaagg
gagagagctacttccacttacacgtgctcgtggaaaccaccggggtgaaatccttagttttgggacgtttcctgagtcagattcgcgaaa
aactgattcagagaatttaccgcgggatcgagccgactttgccaaactggttcgcggtcacaaagaccagaaacggcgccggaggc
gggaacaaggtggtggacgagtgctacatccccaattacttgctccccaaaacccagcctgagctccagtgggcgtggactaatttag
aacagtatttaagcgcctgtttgaatctcacggagcgtaaacggttggtggcgcagcatctgacgcacgtgtcgcagacgcaggagca
gaacaaagagaatcagaatcccaattctgacgcgccggtgatcagatcaaaaacttcagccaggtacatggagctggtcgggtggct
cgtggacaaggggattacctcggagaagcagtggatccaggaggaccaggcctcatacatctccttcaatgcggcctccaactcgcg
gtcccaaatcaaggctgccttggacaatgcgggaaagattatgagcctgactaaaaccgcccccgactacctggtgggccagcagcc
cgtggaggacatttccagcaatcggatttataaaattttggaactaaacgggtacgatccccaatatgcggcttccgtctttctgggatgg
gccacgaaaaagttcggcaagaggaacaccatctggctgtttgggcctgcaactaccgggaagaccaacatcgcggaggccatagc
ccacactgtgcccttctacgggtgcgtaaactggaccaatgagaactttcccttcaacgactgtgtcgacaagatggtgatctggtggg
aggaggggaagatgaccgccaaggtcgtggagtcggccaaagccattctcggaggaagcaaggtgcgcgtggaccagaaatgca
agtcctcggcccagatagacccgactcccgtgatcgtcacctccaacaccaacatgtgcgccgtgattgacgggaactcaacgacctt
cgaacaccagcagccgttgcaagaccggatgttcaaatttgaactcacccgccgtctggatcatgactttgggaaggtcaccaagcag
gaagtcaaagactttttccggtgggcaaaggatcacgtggttgaggtggagcatgaattctacgtcaaaaagggtggagccaagaaa
agacccgcccccagtgacgcagatataagtgagcccaaacgggtgcgcgagtcagttgcgcagccatcgacgtcagacgcggaag
cttcgatcaactacgcagacaggtaccaaaacaaatgttctcgtcacgtgggcatgaatctgatgctgtttccctgcagacaatgcgaga
gaatgaatcagaattcaaatatctgcttcactcacggacagaaagactgtttagagtgctttcccgtgtcagaatctcaacccgtttctgtc
gtcaaaaaggcgtatcagaaactgtgctacattcatcatatcatgggaaaggtgccagacgcttgcactgcctgcgatctggtcaatgtg
gatttggatgactgcatctttgaacaataa 3’.

In some embodiments, the Rep78 protein comprises an amino acid sequence of SEQ ID NO: 1780. This amino acid sequence is given here:

(SEQ ID NO: 1780)
LAGFYEIVIKVPSDLDEHLPGISDSFVNWVAEKEWELPPDSDLDLNLIE
QAPLTVAEKLQRDFLTEWRRVSKAPEALFFVQFEKGESYFHLHVLVETT
GVKSLVLGRFLSQIREKLIQRIYRGIEPTLPNWFAVTKTRNGAGGGNKV
VDECYIPNYLLPKTQPELQWAWTNLEQYLSACLNLTERKRLVAQHLTHV
SQTQEQNKENQNPNSDAPVIRSKTSARYMELVGWLVDKGITSEKQWIQE
DQASYISFNAASNSRSQIKAALDNAGKIMSLTKTAPDYLVGQQPVEDIS
SNRIYKILELNGYDPQYAASVFLGWATKKFGKRNTIWLFGPATTGKTNI
AEAIAHTVPFYGCVNWTNENFPFNDCVDKMVIWWEEGKMTAKVVESAKA
ILGGSKVRVDQKCKSSAQIDPTPVIVTSNTNMCAVIDGNSTTFEHQQPL
QDRMFKFELTRRLDHDFGKVTKQEVKDFFRWAKDHVVEVEHEFYVKKGG
AKKRPAPSDADISEPKRVRESVAQPSTSDAEASINYADRYQNKCSRHVG
MNLMLFPCRQCERMNQNSNICFTHGQKDCLECFPVSESQPVSVVKKAYQ
KLCYIHHIMGKVPDACTACDLVNVDLDDCIFEQ.

In some embodiments, the first amino acid residue of the Rep78 protein is methionine. In some embodiments, the first amino acid residue of the Rep78 protein is leucine. In some embodiments, AAVs of the present disclosure comprise a mixed population of AAV2 Rep78 of SEQ ID NO: 1780, in which the first amino acid residue may be methionine or leucine.

In some embodiments, the ratio of VP1:VP2:VP3 of the rAAV produced by a method disclosed herein is about 1:1:10.

In certain embodiments, a composition comprising rAAVs comprising a polynucleotide encoding AADC or a functional variant thereof is produced by any of the methods disclosed herein. In some embodiments, the composition comprises 3.0×10¹²-5.0×10¹² vg/mL rAAVs, e.g., about 5.0×10¹² vg/mL rAAVs, in a solution comprising 10 mM sodium phosphate, 180 mM sodium chloride, and 0.001% poloxamer 188 (solution pH of 7.1-7.5, e.g., pH 7.3). In some embodiments, the composition is used in treating and/or preventing Parkinson's Disease. In some embodiments, the present disclosure comprises a method of treating Parkinson's Disease comprising administering an effective amount of the composition. In some embodiments, the composition is used for the manufacture of a medicament for treating and/or preventing Parkinson's Disease.

In certain embodiments, the present disclosure encompasses a method for producing a recombinant adeno-associated virus 2 (rAAV2) comprising a polynucleotide comprising SEQ ID NO: 979. In some embodiments, the method comprises the steps of: (a) culturing Sf9 cells (viral production Sf9 cells) in a bioreactor to a target cell density of 3.0×10⁶-3.4×10⁶ cells/mL; wherein the viral production Sf9 cells are cultured in serum-free, protein-free insect cell culture medium at about 26° C.-28° C. and 30%-50% dissolved oxygen, wherein the serum-free, protein-free insect cell culture medium optionally comprises L-glutamine and poloxamer 188; (b) introducing into the bioreactor baculovirus infected insect cells (expressionBIICs) comprising baculoviruses comprising a viral expression construct, and baculovirus infected insect cells (payloadBIICs) comprising baculoviruses comprising a payload construct, wherein the viral expression construct comprises one or more polynucleotides encoding capsid and replication proteins of adeno-associated virus 2 (AAV2); wherein the payload construct comprises SEQ ID NO: 979; and wherein the expressionBIICs are introduced at a ratio of about 1:300,000 expressionBIIC:viral production Sf9 (v/v) and the payloadBIICs are introduced at a ratio of about 1:100,000 payloadBIIC:viral production Sf9 (v/v), wherein the expressionBIICs are optionally Sf9 cells and the payloadBIICs are optionally Sf9 cells, wherein the viral expression construct comprises one or more polynucleotides encoding capsid and replication proteins of adeno-associated virus 2 (AAV2, e.g., wild-type AAV2); wherein the payload construct comprises SEQ ID NO: 979; (c) incubating the viral production Sf9 cells in the bioreactor under conditions that result in the production of one or more rAAV2s within one or more of the viral production Sf9 cells, wherein one or more of the rAAV2s comprise a polynucleotide comprising SEQ ID NO: 979; (d) harvesting a viral production pool from the bioreactor, wherein the viral production pool comprises one or more viral production Sf9 cells comprising one or more rAAV2s, wherein the viral production pool optionally weighs 195-198 kg, e.g., 196 kg, and has a % viability of at least 85%, e.g., at least 90%; (e) lysing the viral production Sf9 cells in the viral production pool, wherein the lysing comprises a chemical lysis solution and is carried out at 26° C.-28° C. for 4-6 hours, wherein the chemical lysis solution comprises 0.5% (w/v) octyl phenol ethoxylate and 200 mM arginine hydrochloride, and lacks detectable nuclease, thereby releasing one or more rAAV2s from the viral production Sf9 cells into a lysis medium; (f) clarifying the lysis medium of (e) through a depth filter followed by an about 0.2 μm filter, yielding a clarification pool; (g) processing the clarification pool of (f) through an immunoaffinity chromatography column comprising a recombinant protein ligand that binds at least AAV2; wherein the immunoaffinity chromatography column is equilibrated with a solution comprising 50 mM sodium phosphate, 350 mM sodium chloride and 0.001% w/v poloxamer 188; loaded with a 1.0×10¹³-5.0×10¹³ vg/mL-r load challenge at 18-25° C.; flushed with a solution comprising 50 mM sodium phosphate, 350 mM sodium chloride and 0.001% w/v poloxamer 188; washed with a solution comprising 20 mM sodium citrate, 1 M sodium chloride and 0.001% w/v poloxamer 188; and washed with a solution of 10 mM sodium citrate, 350 mM sodium chloride and 0.001% w/v poloxamer 188; wherein processing the clarification pool of (f) through an immunoaffinity chromatography column yields an immunoaffinity chromatography pool, wherein the immunoaffinity chromatography pool is optionally neutralized with 2 M Tris Base and 0.001% w/v poloxamer 188 (3.0% v/v spike, pH 8.0-8.5) and optionally filtered through an about 0.2 μm filter; (h) processing the immunoaffinity chromatography pool or filtered immunoaffinity chromatography pool of (g) through an anion exchange chromatography column, e.g., a column operated in flow-through mode; wherein the one or more anion exchange chromatography columns is charged and equilibrated with a solution comprising 20 mM Tris, 2 M sodium chloride and 0.001% w/v poloxamer 188, then a solution of 40 mM Tris, 170 mM sodium chloride and 0.001% w/v poloxamer 188; loaded with a 1.0×10¹³-5.0×10¹³ vg/mL-r load challenge at 18-25° C.; and flushed and eluted with a solution comprising 40 mM Tris, 170 mM sodium chloride and 0.001% w/v poloxamer 188, yielding an anion exchange chromatography pool; wherein the anion exchange chromatography pool is filtered through an about 0.2 μm filter, yielding a filtered anion exchange chromatography pool; (i) processing the filtered anion exchange chromatography pool of (h) through a tangential flow filtration (TFF) filter yielding a TFF load pool; wherein the TFF filter is equilibrated with buffer comprising 40 mM Tris, 170 mM sodium chloride, and 0.001% (w/v) poloxamer 188; wherein the TFF load pool is concentrated by ultrafiltration to a viral concentration of about 5.0×10¹² vg/mL, followed by diafiltration comprising buffer exchange with a buffer comprising 10 mM sodium phosphate, 180 mM sodium chloride, and 0.001% (w/v) poloxamer 188 (buffer pH of 7.1-7.5, e.g., pH 7.3), followed by filtration through an about 0.2 μm filter, yielding a filtered TFF load pool; wherein a TFF recovery flush pool is prepared by subjecting the TFF filter to a recovery flush using a buffer comprising 10 mM sodium phosphate, 180 mM sodium chloride, and 0.001% (w/v) poloxamer 188 (buffer pH of 7.1-7.5, e.g., pH 7.3); wherein the TFF recovery flush pool is filtered through an about 0.2 μm filter, yielding a filtered TFF recovery flush pool; wherein the filtered TFF load pool and filtered TFF recovery flush pool are combined to form a concentrated, buffer-exchanged pool, and optionally diluted using a buffer comprising 10 mM sodium phosphate, 180 mM sodium chloride, and 0.001% (w/v) poloxamer 188 (buffer pH of 7.1-7.5, e.g., pH 7.3), wherein the concentrated, buffer-exchanged pool comprises a viral concentration of 2.0×10¹²-6.0×10¹² vg/mL; (j) processing the concentrated, buffer-exchanged pool of (i) through a viral retentive filtration (VRF) filter to yield a viral filtration pool; wherein the VRF filter comprises a pore size of about 35 nm and is flushed twice before use with a solution comprising 10 mM sodium phosphate, 180 mM sodium chloride, and 0.001% poloxamer 188 (solution pH of 7.1-7.5, e.g., pH 7.3); and wherein the viral filtration pool is filtered through a filter of about 0.2 μm, yielding a filtered viral filtration pool comprising a viral concentration of 3.5×10¹²-5.0×10¹² vg/mL; and (k) processing the viral production pool of (j) through an about 0.22 μm filter, e.g., filtering twice through an about 0.22 μm filter, to yield a purified rAAV2 composition comprising a viral concentration of 3.0×10¹²-5.0×10¹² vg/mL in a solution comprising 10 mM sodium phosphate, 180 mM sodium chloride, and 0.001% poloxamer 188 (solution pH of 7.1-7.5, e.g., pH 7.3).

In some embodiments, the viral concentration of the purified rAAV2 composition is about 5.0×10¹² vg/mL. In some embodiments, the method produces a total process rAAV yield of 30%-50%. In some embodiments, the viral expression construct comprises one or more polynucleotides encoding a VP1 capsid protein, VP2 capsid protein, VP3 capsid protein, Rep52, and Rep78. In some embodiments, the VP1 capsid protein, VP2 capsid protein, and VP3 capsid protein are encoded in one or more open reading frames and the Rep52 and Rep78 are encoded in one or more open reading frames, wherein the one or more open reading frames encoding the VP1 capsid protein, VP2 capsid protein, and VP3 capsid protein and the one or more open reading frames encoding the Rep52 and Rep78 are different open reading frames. In some embodiments, the VP1 capsid protein, VP2 capsid protein, and VP3 capsid protein are encoded in a first open reading frame and the Rep52 and Rep78 are encoded in a second open reading frame. In some embodiments, the purified rAAV2 comprise a ratio of VP1:VP2:VP3 of about 1:1:10.

In some embodiments, a composition produced by any of the methods disclosed herein comprises 3.0×10¹²-5.0×10¹² vg/mL rAAV2s, e.g., about 5.0×10¹² vg/mL rAAV2s, in a solution comprising 10 mM sodium phosphate, 180 mM sodium chloride, and 0.001% poloxamer 188 (solution pH of 7.1-7.5, e.g., pH 7.3). In some embodiments, the composition is used in treating and/or preventing Parkinson's Disease. In some embodiments, a method of treating Parkinson's Disease comprises administering an effective amount of the composition. In some embodiments, the composition is used in the manufacture of a medicament for treating and/or preventing Parkinson's Disease.

In some embodiments, the purified viral rAAV2 composition is formulated at a concentration of about 3.0×10¹² to about 5.0×10¹² vg/mL, e.g., about 5.0×10¹² vg/mL. In some embodiments, the purified rAAV2 composition is formulated a concentration of about 2.0×10¹² to about 3.0×10¹² vg/mL, e.g., about 2.7×10¹² vg/mL.

In some embodiments, the purified viral rAAV2 composition is formulated at a concentration of about 5×10¹² vg/mL in a solution comprising about 10 mM sodium phosphate, about 180 mM sodium chloride, about 0.001% poloxamer 188 (solution pH of about 7.3).

In some embodiments, the purified viral rAAV2 composition comprises a polynucleotide encoding AADC (e.g., SEQ ID NO: 979) or a functional variant thereof in an AAV2 viral capsid (e.g., SEQ ID NO: 15) and has an AADC relative potency of at least 50%, wherein the rAAV composition comprises greater than or equal to 3.0×10¹² vg/mL (e.g., about 5.0×10¹² vg/mL) rAAVs in a solution comprising 10 mM sodium phosphate, 180 mM sodium chloride, and 0.001% poloxamer (solution pH of 7.3±0.5). In some embodiments, the rAAV composition comprises greater than about 60% full viral capsids (e.g., less than about 40% empty viral capsids), less than about 1 EU/mL endotoxin levels, greater than about 90% protein purity, and greater than about 3×10¹⁰ TU/mL infectious titer. In some embodiments, the rAAV composition has an osmolality of 300-400 mOsm/kg, and comprises less than about 6000 particles with a size of ≥10 μm and less than about 600 particles with a size of ≥25 μm.

### III. Compositions and Formulations

### General

Gene therapy drug products (such as rAAV particles) are challenging to incorporate into composition and formulations due to their limited stability in the liquid state and a high propensity for large-scale aggregation at low concentrations. Gene therapy drug products are often delivered directly to treatment areas (comprising CNS tissue); which requires that excipients and formulation parameters be compatible with tissue function, microenvironment, and volume restrictions.

According to the present disclosure, AAV particles may be prepared as, or comprised in, pharmaceutical compositions. It will be understood that such compositions necessarily comprise one or more active ingredients and, most often, one or more pharmaceutically acceptable excipients.

Relative amounts of the active ingredient (e.g., AAV particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, or at least 80% (w/w) active ingredient.

In certain embodiments, the AAV particle pharmaceutical compositions described herein may comprise at least one payload of the present disclosure. As a non-limiting example, the pharmaceutical compositions may contain an AAV particle with 1, 2, 3, 4 or 5 payloads.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated comprise, but are not limited to, humans and/or other primates; mammals, comprising commercially relevant mammals such as cattle, pigs, horses, sheep, cats, dogs, mice, rats, birds, comprising commercially relevant birds such as poultry, chickens, ducks, geese, and/or turkeys.

In certain embodiments, compositions are administered to humans, human patients or subjects.

Formulations of the present disclosure can comprise, without limitation, saline, liposomes, lipid nanoparticles, polymers, peptides, proteins, cells transfected with AAV particles (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods comprise the step of associating the active ingredient with an excipient and/or one or more other accessory ingredients. As used herein, the phrase “active ingredient” generally refers either to an AAV particle carrying a payload region encoding the polynucleotide or polypeptides of the present disclosure or to the end product encoded by a viral genome of an AAV particle as described herein.

In certain embodiments, the formulations may comprise at least one inactive ingredient. As used herein, the term “inactive ingredient” refers to one or more inactive agents comprised in formulations. In certain embodiments, all, none or some of the inactive ingredients which may be used in the formulations of the present disclosure may be approved by the US Food and Drug Administration (FDA).

Formulations of the AAV particles and pharmaceutical compositions described herein may be prepared by any method known or hereafter developed in the art of pharmacology. In general, such preparatory methods comprise the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In certain embodiments, formulations of the present disclosure are aqueous formulations (i.e. formulations which comprise water). In certain embodiments, formulations of the present disclosure comprise water, sanitized water, or Water-for-injection (WFI).

In certain embodiments, the AAV particles of the present disclosure may be formulated in PBS with 0.001%-0.1% (w/v) of Poloxamer 188 (e.g., Pluronic F-68) at a pH of about 7.0.

In certain embodiments, the AAV formulations described herein may contain sufficient AAV particles for expression of at least one expressed functional payload. As a non-limiting example, the AAV particles may contain viral genomes encoding 1, 2, 3, 4 or 5 functional payloads.

According to the present disclosure AAV particles may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

In certain embodiments, the AAV formulations described herein may comprise a buffering system which comprises phosphate, Tris, and/or Histidine. The buffering agents of phosphate, Tris, and/or Histidine may be independently used in the formulation in a range of 2-12 mM.

Formulations of the present disclosure can be used in any step of producing, processing, preparing, storing, expanding, or administering AAV particles and viral vectors of the present disclosure. In certain embodiments, pharmaceutical formulations and components can be use in AAV production, AAV processing, AAV clarification, AAV purification, and AAV finishing systems of the present disclosure, all of which can be pre-rinsed, packed, equilibrated, flushed, processed, eluted, washed or cleaned with formulations known to those in the art, comprising AAV pharmaceutical, processing and storage formulations of the present disclosure.

### Excipients and Diluents

The AAV particles of the present disclosure can be formulated into a pharmaceutical composition which comprises one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the present disclosure.

Relative amounts of the active ingredient (e.g., AAV particle), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. In certain embodiments, the composition may comprise between 0.001% and 99% (w/w) of the active ingredient. By way of example, the composition may comprise between 0.001% and 100%, e.g., between 0.5 and 50%, between 1-30%, between 5-80%, or at least 80% (w/w) active ingredient. In certain embodiments, the composition may comprise between 0.001% and 99% (w/w) of the excipients and diluents. By way of example, the composition may comprise between 0.001% and 100%, e.g., between 0.5 and 50%, between 1-30%, between 5-80%, or at least 80% (w/w) excipients and diluents.

In certain embodiments, a pharmaceutically acceptable excipient may be at least 95%, at least 96%, at least 97%, at least 98%, at least 99%, or 100% pure. In certain embodiments, an excipient is approved for use for humans and for veterinary use. In certain embodiments, an excipient may be approved by United States Food and Drug Administration. In certain embodiments, an excipient may be of pharmaceutical grade. In certain embodiments, an excipient may meet the standards of the United States Pharmacopoeia (USP), the European Pharmacopoeia (EP), the British Pharmacopoeia, and/or the International Pharmacopoeia.

Excipients, as used herein, comprise, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary excipients and diluents which can be comprised in formulations of the present disclosure comprise, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Pharmaceutical formulations of AAV particles disclosed herein may comprise cations or anions. In certain embodiments, the formulations comprise metal cations such as, but not limited to, Zn²⁺, Ca²⁺, Cu²⁺, Mn²⁺, Mg⁺ and combinations thereof. As a non-limiting example, formulations may comprise polymers and complexes with a metal cation (See e.g., U.S. Pat. Nos. 6,265,389 and 6,555,525, each of which is herein incorporated by reference in its entirety).

Formulations of the present disclosure may also comprise one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid).

In certain embodiments, additional excipients that may be used in formulating the pharmaceutical composition may comprise magnesium chloride (MgCl₂), arginine, sorbitol, and/or trehalose.

Formulations of the present disclosure may comprise at least one excipient and/or diluent in addition to the AAV particle. The formulation may comprise 1, 2, 3, 4, 5, 6, 7, 8, 9, 10, or more than 10 excipients and/or diluents in addition to the AAV particle.

In certain embodiments, the formulation may comprise, but is not limited to, phosphate-buffered saline (PBS). As a non-limiting example, the PBS may comprise sodium chloride, potassium chloride, disodium phosphate, monopotassium phosphate, and distilled water. In some instances, the PBS does not contain potassium or magnesium. In other instances, the PBS contains calcium and magnesium.

### Sodium Phosphate

In certain embodiments, at least one of the components in the formulation is sodium phosphate. The formulation may comprise monobasic, dibasic or a combination of both monobasic and dibasic sodium phosphate.

In certain embodiments, the concentration of sodium phosphate in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may comprise sodium phosphate in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may comprise 2-12 mM of sodium phosphate.

In certain embodiments, the formulation may comprise 10 mM of sodium phosphate.

### Potassium Phosphate

In certain embodiments, at least one of the components in the formulation is potassium phosphate. The formulation may comprise monobasic, dibasic or a combination of both monobasic and dibasic potassium phosphate.

In certain embodiments, the concentration of potassium phosphate in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may comprise potassium phosphate in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

### Sodium Chloride

In certain embodiments, at least one of the components in the formulation is sodium chloride.

In certain embodiments, the concentration of sodium chloride in a formulation may be, but is not limited to, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 101 mM, 102 mM, 103 mM, 104 mM, 105 mM, 106 mM, 107 mM, 108 mM, 109 mM, 110 mM, 111 mM, 112 mM, 113 mM, 114 mM, 115 mM, 116 mM, 117 mM, 118 mM, 119 mM, 120 mM, 121 mM, 122 mM, 123 mM, 124 mM, 125 mM, 126 mM, 127 mM, 128 mM, 129 mM, 130 mM, 131 mM, 132 mM, 133 mM, 134 mM, 135 mM, 136 mM, 137 mM, 138 mM, 139 mM, 140 mM, 141 mM, 142 mM, 143 mM, 144 mM, 145 mM, 146 mM, 147 mM, 148 mM, 149 mM, 150 mM, 151 mM, 152 mM, 153 mM, 154 mM, 155 mM, 156 mM, 157 mM, 158 mM, 159 mM, 160 mM, 161 mM, 162 mM, 163 mM, 164 mM, 165 mM, 166 mM, 167 mM, 168 mM, 169 mM, 170 mM, 171 mM, 172 mM, 173 mM, 174 mM, 175 mM, 176 mM, 177 mM, 178 mM, 179 mM, 180 mM, 181 mM, 182 mM, 183 mM, 184 mM, 185 mM, 186 mM, 187 mM, 188 mM, 189 mM, 190 mM, 191 mM, 192 mM, 193 mM, 194 mM, 195 mM, 196 mM, 197 mM, 198 mM, 199 mM, 200 mM, 201 mM, 202 mM, 203 mM, 204 mM, 205 mM, 206 mM, 207 mM, 208 mM, 209 mM, 210 mM, 211 mM, 212 mM, 213 mM, 214 mM, 215 mM, 216 mM, 217 mM, 218 mM, 219 mM, or 220 mM.

The formulation may comprise sodium chloride in a range of 75-85 mM, 80-90 mM, 85-95 mM, 90-100 mM, 95-105 mM, 100-110 mM, 105-115 mM, 110-120 mM, 115-125 mM, 120-130 mM, 125-135 mM, 130-140 mM, 135-145 mM, 140-150 mM, 145-155 mM, 150-160 mM, 155-165 mM, 160-170 mM, 165-175 mM, 170-180 mM, 175-185 mM, 180-190 mM, 185-195 mM, 190-200 mM, 75-95 mM, 80-100 mM, 85-105 mM, 90-110 mM, 95-115 mM, 100-120 mM, 105-125 mM, 110-130 mM, 115-135 mM, 120-140 mM, 125-145 mM, 130-150 mM, 135-155 mM, 140-160 mM, 145-165 mM, 150-170 mM, 155-175 mM, 160-180 mM, 165-185 mM, 170-190 mM, 175-195 mM, 180-200 mM, 75-100 mM, 80-105 mM, 85-110 mM, 90-115 mM, 95-120 mM, 100-125 mM, 105-130 mM, 110-135 mM, 115-140 mM, 120-145 mM, 125-150 mM, 130-155 mM, 135-160 mM, 140-165 mM, 145-170 mM, 150-175 mM, 155-180 mM, 160-185 mM, 165-190 mM, 170-195 mM, 175-200 mM, 75-105 mM, 80-110 mM, 85-115 mM, 90-120 mM, 95-125 mM, 100-130 mM, 105-135 mM, 110-140 mM, 115-145 mM, 120-150 mM, 125-155 mM, 130-160 mM, 135-165 mM, 140-170 mM, 145-175 mM, 150-180 mM, 155-185 mM, 160-190 mM, 165-195 mM, 170-200 mM, 75-115 mM, 80-120 mM, 85-125 mM, 90-130 mM, 95-135 mM, 100-140 mM, 105-145 mM, 110-150 mM, 115-155 mM, 120-160 mM, 125-165 mM, 130-170 mM, 135-175 mM, 140-180 mM, 145-185 mM, 150-190 mM, 155-195 mM, 160-200 mM, 75-120 mM, 80-125 mM, 85-130 mM, 90-135 mM, 95-140 mM, 100-145 mM, 105-150 mM, 110-155 mM, 115-160 mM, 120-165 mM, 125-170 mM, 130-175 mM, 135-180 mM, 140-185 mM, 145-190 mM, 150-195 mM, 155-200 mM, 75-125 mM, 80-130 mM, 85-135 mM, 90-140 mM, 95-145 mM, 100-150 mM, 105-155 mM, 110-160 mM, 115-165 mM, 120-170 mM, 125-175 mM, 130-180 mM, 135-185 mM, 140-190 mM, 145-195 mM, 150-200 mM, 75-125 mM, 80-130 mM, 85-135 mM, 90-140 mM, 95-145 mM, 100-150 mM, 105-155 mM, 110-160 mM, 115-165 mM, 120-170 mM, 125-175 mM, 130-180 mM, 135-185 mM, 140-190 mM, 145-195 mM, 150-200 mM, 75-135 mM, 80-140 mM, 85-145 mM, 90-150 mM, 95-155 mM, 100-160 mM, 105-165 mM, 110-170 mM, 115-175 mM, 120-180 mM, 125-185 mM, 130-190 mM, 135-195 mM, 140-200 mM, 75-145 mM, 80-150 mM, 85-155 mM, 90-160 mM, 95-165 mM, 100-170 mM, 105-175 mM, 110-180 mM, 115-185 mM, 120-190 mM, 125-195 mM, 130-200 mM, 75-155 mM, 80-160 mM, 85-165 mM, 90-170 mM, 95-175 mM, 100-180 mM, 105-185 mM, 110-190 mM, 115-195 mM, 120-200 mM, 75-165 mM, 80-170 mM, 85-175 mM, 90-180 mM, 95-185 mM, 100-190 mM, 105-195 mM, 110-200 mM, 75-175 mM, 80-180 mM, 85-185 mM, 90-190 mM, 95-195 mM, 100-200 mM, 80-220 mM, 90-220 mM, 100-220 mM, 110-220 mM, 120-220 mM, 130-220 mM, 140-220 mM, 150-220 mM, 160-220 mM, 170-220 mM, 180-220 mM, 190-220 mM, 200-220 mM, or 210-220 mM.

In certain embodiments, the formulation may comprise 180 mM of sodium chloride.

### Potassium Chloride

In certain embodiments, at least one of the components in the formulation is potassium chloride.

In certain embodiments, the concentration of potassium chloride in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may comprise potassium chloride in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

### Magnesium Chloride

In certain embodiments, at least one of the components in the formulation is magnesium chloride.

In certain embodiments, the concentration of magnesium chloride may be, but is not limited to, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or 100 mM.

The formulation may comprise magnesium chloride in a range of 0-5 mM, 1-5 mM, 2-5 mM, 3-5 mM, 4-5 mM, 0-10 mM, 1-10 mM, 2-10 mM, 3-10 mM, 4-10 mM, 5-10 mM, 6-10 mM, 7-10 mM, 8-10 mM, 9-10 mM, 0-25 mM, 1-25 mM, 2-25 mM, 3-25 mM, 4-25 mM, 5-25 mM, 6-25 mM, 7-25 mM, 8-25 mM, 9-25 mM, 10-25 mM, 11-25 mM, 12-25 mM, 13-25 mM, 14-25 mM, 15-25 mM, 16-25 mM, 17-25 mM, 18-25 mM, 19-25 mM, 20-25 mM, 21-25 mM, 22-25 mM, 23-25 mM, 24-25 mM, 0-50 mM, 1-50 mM, 2-50 mM, 3-50 mM, 4-50 mM, 5-50 mM, 6-50 mM, 7-50 mM, 8-50 mM, 9-50 mM, 10-50 mM, 11-50 mM, 12-50 mM, 13-50 mM, 14-50 mM, 15-50 mM, 16-50 mM, 17-50 mM, 18-50 mM, 19-50 mM, 20-50 mM, 21-50 mM, 22-50 mM, 23-50 mM, 24-50 mM, 25-50 mM, 26-50 mM, 27-50 mM, 28-50 mM, 29-50 mM, 30-50 mM, 31-50 mM, 32-50 mM, 33-50 mM, 34-50 mM, 35-50 mM, 36-50 mM, 37-50 mM, 38-50 mM, 39-50 mM, 40-50 mM, 41-50 mM, 42-50 mM, 43-50 mM, 44-50 mM, 45-50 mM, 46-50 mM, 47-50 mM, 48-50 mM, 49-50 mM, 0-75 mM, 1-75 mM, 2-75 mM, 3-75 mM, 4-75 mM, 5-75 mM, 6-75 mM, 7-75 mM, 8-75 mM, 9-75 mM, 10-75 mM, 11-75 mM, 12-75 mM, 13-75 mM, 14-75 mM, 15-75 mM, 16-75 mM, 17-75 mM, 18-75 mM, 19-75 mM, 20-75 mM, 21-75 mM, 22-75 mM, 23-75 mM, 24-75 mM, 25-75 mM, 26-75 mM, 27-75 mM, 28-75 mM, 29-75 mM, 30-75 mM, 31-75 mM, 32-75 mM, 33-75 mM, 34-75 mM, 35-75 mM, 36-75 mM, 37-75 mM, 38-75 mM, 39-75 mM, 40-75 mM, 41-75 mM, 42-75 mM, 43-75 mM, 44-75 mM, 45-75 mM, 46-75 mM, 47-75 mM, 48-75 mM, 49-75 mM, 50-75 mM, 51-75 mM, 52-75 mM, 53-75 mM, 54-75 mM, 55-75 mM, 56-75 mM, 57-75 mM, 58-75 mM, 59-75 mM, 60-75 mM, 61-75 mM, 62-75 mM, 63-75 mM, 64-75 mM, 65-75 mM, 66-75 mM, 67-75 mM, 68-75 mM, 69-75 mM, 70-75 mM, 71-75 mM, 72-75 mM, 73-75 mM, 74-75 mM, 50-100 mM, 60-100 mM, 75-100 mM, 80-100 mM, or 90-100 mM.

### Tris

In certain embodiments, at least one of the components in the formulation is Tris (also called tris(hydroxymethyl)aminomethane, tromethamine or THAM).

In certain embodiments, the concentration of Tris in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may comprise Tris in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

### Histidine

In certain embodiments, at least one of the components in the formulation is Histidine.

In certain embodiments, the concentration of Histidine in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may comprise Histidine in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

### Arginine

In certain embodiments, at least one of the components in the formulation is arginine.

In certain embodiments, the concentration of arginine may be, but is not limited to, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or 100 mM.

The formulation may comprise arginine in a range of 0-5 mM, 1-5 mM, 2-5 mM, 3-5 mM, 4-5 mM, 0-10 mM, 1-10 mM, 2-10 mM, 3-10 mM, 4-10 mM, 5-10 mM, 6-10 mM, 7-10 mM, 8-10 mM, 9-10 mM, 0-25 mM, 1-25 mM, 2-25 mM, 3-25 mM, 4-25 mM, 5-25 mM, 6-25 mM, 7-25 mM, 8-25 mM, 9-25 mM, 10-25 mM, 11-25 mM, 12-25 mM, 13-25 mM, 14-25 mM, 15-25 mM, 16-25 mM, 17-25 mM, 18-25 mM, 19-25 mM, 20-25 mM, 21-25 mM, 22-25 mM, 23-25 mM, 24-25 mM, 0-50 mM, 1-50 mM, 2-50 mM, 3-50 mM, 4-50 mM, 5-50 mM, 6-50 mM, 7-50 mM, 8-50 mM, 9-50 mM, 10-50 mM, 11-50 mM, 12-50 mM, 13-50 mM, 14-50 mM, 15-50 mM, 16-50 mM, 17-50 mM, 18-50 mM, 19-50 mM, 20-50 mM, 21-50 mM, 22-50 mM, 23-50 mM, 24-50 mM, 25-50 mM, 26-50 mM, 27-50 mM, 28-50 mM, 29-50 mM, 30-50 mM, 31-50 mM, 32-50 mM, 33-50 mM, 34-50 mM, 35-50 mM, 36-50 mM, 37-50 mM, 38-50 mM, 39-50 mM, 40-50 mM, 41-50 mM, 42-50 mM, 43-50 mM, 44-50 mM, 45-50 mM, 46-50 mM, 47-50 mM, 48-50 mM, 49-50 mM, 0-75 mM, 1-75 mM, 2-75 mM, 3-75 mM, 4-75 mM, 5-75 mM, 6-75 mM, 7-75 mM, 8-75 mM, 9-75 mM, 10-75 mM, 11-75 mM, 12-75 mM, 13-75 mM, 14-75 mM, 15-75 mM, 16-75 mM, 17-75 mM, 18-75 mM, 19-75 mM, 20-75 mM, 21-75 mM, 22-75 mM, 23-75 mM, 24-75 mM, 25-75 mM, 26-75 mM, 27-75 mM, 28-75 mM, 29-75 mM, 30-75 mM, 31-75 mM, 32-75 mM, 33-75 mM, 34-75 mM, 35-75 mM, 36-75 mM, 37-75 mM, 38-75 mM, 39-75 mM, 40-75 mM, 41-75 mM, 42-75 mM, 43-75 mM, 44-75 mM, 45-75 mM, 46-75 mM, 47-75 mM, 48-75 mM, 49-75 mM, 50-75 mM, 51-75 mM, 52-75 mM, 53-75 mM, 54-75 mM, 55-75 mM, 56-75 mM, 57-75 mM, 58-75 mM, 59-75 mM, 60-75 mM, 61-75 mM, 62-75 mM, 63-75 mM, 64-75 mM, 65-75 mM, 66-75 mM, 67-75 mM, 68-75 mM, 69-75 mM, 70-75 mM, 71-75 mM, 72-75 mM, 73-75 mM, 74-75 mM, 50-100 mM, 60-100 mM, 75-100 mM, 80-100 mM, or 90-100 mM.

### Hydrochloric Acid

In certain embodiments, at least one of the components in the formulation is hydrochloric acid.

In certain embodiments, the concentration of hydrochloric acid in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may comprise hydrochloric acid in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

### Sugar

In certain embodiments, the formulation may comprise at least one sugar and/or sugar substitute.

In certain embodiments, the formulation may comprise at least one sugar and/or sugar substitute to increase the stability of the formulation. This increase in stability may provide longer hold times for in-process pools, provide a longer “shelf-life”, increase the concentration of AAV particles in solution (e.g., the formulation is able to have higher concentrations of AAV particles without rAAV dropping out of the solution) and/or reduce the generation or formation of aggregation in the formulations.

In certain embodiments, the formulation may comprise 0-10% w/v of a sugar and/or sugar substitute.

In certain embodiments, the formulation may comprise 0-9% w/v of a sugar and/or sugar substitute.

In certain embodiments, the formulation may comprise 1% w/v of a sugar and/or sugar substitute.

In certain embodiments, the formulation may comprise 2% w/v of a sugar and/or sugar substitute.

In certain embodiments, the formulation may comprise 3% w/v of a sugar and/or sugar substitute.

In certain embodiments, the formulation may comprise 4% w/v of a sugar and/or sugar substitute.

In certain embodiments, the formulation may comprise 5% w/v of a sugar and/or sugar substitute.

In certain embodiments, the formulation may comprise 6% w/v of a sugar and/or sugar substitute.

In certain embodiments, the formulation may comprise 7% w/v of a sugar and/or sugar substitute.

In certain embodiments, the formulation may comprise 8% w/v of a sugar and/or sugar substitute.

In certain embodiments, the formulation may comprise 9% w/v of a sugar and/or sugar substitute.

In certain embodiments, the formulation may comprise 10% w/v of a sugar and/or sugar substitute.

In certain embodiments, formulations of pharmaceutical compositions described herein may comprise a disaccharide. Suitable disaccharides that may be used in the formulation described herein may comprise sucrose, lactulose, lactose, maltose, trehalose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, and xylobiose. The concentration of disaccharide (w/v) used in the formulation may be between 1%-15%, for example, between 1%-5%, between 3%-6%, between 5%-8%, between 7%-10%, or between 10%-15%.

In certain embodiments, formulations of pharmaceutical compositions described herein may comprise a sugar alcohol. As a non-limiting example, the sugar alcohol that may be used in the formulation described herein may comprise sorbitol. The concentration of sugar alcohol (w/v) used in the formulation may be between 1%-15%, for example, between 1%-5%, between 3%-6%, between 5%-8%, between 7%-10%, or between 10%-15%.

### Surfactant

In certain embodiments, formulations of pharmaceutical compositions described herein may comprise a surfactant. Surfactants may help control shear forces in suspension cultures. Surfactants used herein may be anionic, zwitterionic, or non-ionic surfactants and may comprise those known in the art that are suitable for use in pharmaceutical formulations.

Examples of anionic surfactants comprise, but are not limited to, sulfate, sulfonate, phosphate esters, and carboxylates.

Examples of nonionic surfactants comprise, but are not limited to, ethoxylates, fatty alcohol ethoxylates, alkylphenol ethoxylates (e.g., nonoxynols, Triton X-100), fatty acid ethoxylates, ethoxylated amines and/or fatty acid amides (e.g., polyethoxylated tallow amine, cocamide monoethanolamine, cocamide diethanolamine), ethylene oxide/propylene oxide copolymer (e.g., Poloxamers such as Pluronic® F-68 or F-127), esters of fatty acids and polyhydric alcohols, fatty acid alkanolamides, ethoxylated aliphatic acids, ethoxylated aliphatic alcohols, ethoxylated sorbitol fatty acid esters, ethoxylated glycerides, ethoxylated block copolymers with EDTA (ethylene diaminetetraacetic acid), ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, and polymerizable surfactants.

Examples of zwitterionic surfactants comprise, but are not limited to, alkylamido betaines and amine oxides thereof, alkyl betaines and amine oxides thereof, sulfo betaines, hydroxy sulfo betaines, amphoglycinates, amphopropionates, balanced amphopoly-carboxyglycinates, and alkyl polyaminoglycinates. Proteins have the ability of being charged or uncharged depending on the pH; thus, at the right pH, a protein, preferably with a pI of about 8 to 9, such as modified Bovine Serum Albumin or chymotrypsinogen, could function as a zwitterionic surfactant. Various mixtures of surfactants can be used if desired.

### Formulation Properties

In certain embodiments, the formulation has been optimized to have a specific pH, osmolality, concentration, concentration of AAV particle, and/or total dose of AAV particle.

pH

In certain embodiments, the formulation may be optimized for a specific pH. In certain embodiments, the formulation may comprise a pH buffering agent (also referred to herein as “buffering agent”) which is a weak acid or base that, when used in the formulation, maintains the pH of the formulation near a chosen value even after another acid or base is added to the formulation. The pH of the formulation may be, but is not limited, to 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, and 14.

In certain embodiments, the formulation may be optimized for a specific pH range. The pH range may be, but is not limited to, 0-4, 1-5, 2-6, 3-7, 4-8, 5-9, 6-10, 7-11, 8-12, 9-13, 10-14, 0-1.5, 1-2.5, 2-3.5, 3-4.5, 4-5.5, 5-6.5, 6-7.5, 7-8.5, 8-9.5, 9-10.5, 10-11.5, 11-12.5, 12-13.5, 0-1, 1-2, 2-3, 3-4, 4-5, 5-6, 6-7, 7-8, 8-9, 9-10, 10-11, 11-12, 12-13, 13-14, 0-0.5, 0.5-1, 1-1.5, 1.5-2, 2-2.5, 2.5-3, 3-3.5, 3.5-4, 4-4.5, 4.5-5, 5-5.5, 5.5-6, 6-6.5, 6.5-7, 7-7.5, 7.2-8.2, 7.2-7.6, 7.3-7.7, 7.5-8, 7.8-8.2, 8-8.5, 8.5-9, 9-9.5, 9.5-10, 10-10.5, 10.5-11, 11-11.5, 11.5-12, 12-12.5, 12.5-13, 13-13.5, or 13.5-14.

In certain embodiments, the pH of the formulation is between 6 and 8.5.

In certain embodiments, the pH of the formulation is between 7 and 7.6.

In certain embodiments, the pH of the formulation is about 7.3.

In certain embodiments, the pH is determined when the formulation is at 5° C.

In certain embodiments, the pH is determined when the formulation is at 25° C.

Suitable buffering agents may comprise, but not limited to, Tris HCl, Tris base, sodium phosphate (monosodium phosphate and/or disodium phosphate), potassium phosphate (monopotassium phosphate and/or dipotassium phosphate), histidine, boric acid, citric acid, glycine, HEPES (4-(2-hydroxyethyl)-1-piperazineethanesulfonic acid), and MOPS (3-(N-morpholino) propanesulfonic acid).

Concentration of buffering agents in the formulation may be between 1-50 mM, between 1-25 mM, between 5-30 mM, between 5-20 mM, between 5-15 mM, between 10-40 mM, or between 15-30 mM. Concentration of buffering agents in the formulation may be about 1 mM, 5 mM, 7.5 mM, 10 mM, 12.5 mM, 15 mM, 20 mM, 25 mM, 30 mM, 35 mM, 40 mM, or 50 mM.

In certain embodiments, the formulation may comprise, but is not limited to, phosphate-buffered saline (PBS). As a non-limiting example, the PBS may comprise sodium chloride, potassium chloride, disodium phosphate, monopotassium phosphate, and distilled water. In some instances, the PBS does not contain potassium or magnesium. In other instances, the PBS contains calcium and magnesium.

In certain embodiments, buffering agents used in the formulations of pharmaceutical compositions described herein may comprise sodium phosphate (monosodium phosphate and/or disodium phosphate). As a non-limiting example, sodium phosphate may be adjusted to a pH (at 5° C.) within the range of 7.4±0.2. In certain embodiments, buffering agents used in the formulations of pharmaceutical compositions described herein may comprise Tris base. Tris base may be adjusted with hydrochloric acid to any pH within the range of 7.1 and 9.1. As a non-limiting example, Tris base used in the formulations described herein may be adjusted to 8.0±0.2. As a non-limiting example, Tris base used in the formulations described herein may be adjusted to 7.5±0.2.

### Osmolality

In certain embodiments, the formulation may be optimized for a specific osmolality. The osmolality of the formulation may be, but is not limited to, 300, 310, 320, 330, 340, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 mOsm/kg (milliosmoles/kg).

In certain embodiments, the formulation may be optimized for a specific range of osmolality. The range may be, but is not limited to, 300-400, 350-360, 360-370, 370-380, 380-390, 390-400, 400-410, 410-420, 420-430, 430-440, 440-450, 450-460, 460-470, 470-480, 480-490, 490-500, 350-370, 360-380, 370-390, 380-400, 390-410, 400-420, 410-430, 420-440, 430-450, 440-460, 450-470, 460-480, 470-490, 480-500, 350-375, 375-400, 400-425, 425-450, 450-475, 475-500, 350-380, 360-390, 370-400, 380-410, 390-420, 400-430, 410-440, 420-450, 430-460, 440-470, 450-480, 460-490, 470-500, 350-390, 360-400, 370-410, 380-420, 390-430, 400-440, 410-450, 420-460, 430-470, 440-480, 450-490, 460-500, 350-400, 360-410, 370-420, 380-430, 390-440, 400-450, 410-460, 420-470, 430-480, 440-490, 450-500, 350-410, 360-420, 370-430, 380-440, 390-450, 400-460, 410-470, 420-480, 430-490, 440-500, 350-420, 360-430, 370-440, 380-450, 390-460, 400-470, 410-480, 420-490, 430-500, 350-430, 360-440, 370-450, 380-460, 390-470, 400-480, 410-490, 420-500, 350-440, 360-450, 370-460, 380-470, 390-480, 400-490, 410-500, 350-450, 360-460, 370-470, 380-480, 390-490, 400-500, 350-460, 360-470, 370-480, 380-490, 390-500, 350-470, 360-480, 370-490, 380-500, 350-480, 360-490, 370-500, 350-490, 360-500, or 350-500 mOsm/kg.

In certain embodiments, the osmolality of the formulation is between 300-400 mOsm/kg.

In certain embodiments, the osmolality of the formulation is between 350-500 mOsm/kg.

In certain embodiments, the osmolality of the formulation is between 400-500 mOsm/kg

In certain embodiments, the osmolality of the formulation is between 400-480 mOsm/kg.

### Concentration of AAV Particle

In certain embodiments, the concentration of AAV particle in the formulation may be between about 1×10⁶ VG/ml and about 1×10¹⁶ VG/ml. As used herein, “VG/ml” represents vector genomes (VG) per milliliter (ml). VG/ml also may describe genome copy per milliliter or DNase resistant particle per milliliter.

In certain embodiments, the formulation may comprise an AAV particle concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 4×10¹², 4.1×10¹², 4.2×10¹², 4.3×10¹², 4.4×10¹², 4.5×10¹²,4.6×10¹², 4.7×10¹², 4.8×10¹², 4.9×10¹², 5×10¹², 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/ml.

In certain embodiments, the concentration of AAV particle in the formulation is between 1×10¹¹ and 5×10¹³, between 1×10¹² and 5×10¹², between 2×10¹² and 1×10¹³, between 5×10¹² and 1×10¹³, between 1×10¹³ and 2×10¹³, between 2×10¹³ and 3×10¹³, between 2×10¹³ and 2.5×10¹³, between 2.5×10¹³ and 3×10¹³, or no more than 5×10¹³ VG/ml.

In certain embodiments, the concentration of AAV particle in the formulation is 2.7×10¹¹ VG/ml.

In certain embodiments, the concentration of AAV particle in the formulation is 9×10¹¹ VG/ml.

In certain embodiments, the concentration of AAV particle in the formulation is 1.2×10¹² VG/ml.

In certain embodiments, the concentration of AAV particle in the formulation is 2.7×10¹² VG/ml.

In certain embodiments, the concentration of AAV particle in the formulation is 4×10¹² VG/ml.

In certain embodiments, the concentration of AAV particle in the formulation is 6×10¹² VG/ml.

In certain embodiments, the concentration of AAV particle in the formulation is 7.9×10¹² VG/ml.

In certain embodiments, the concentration of AAV particle in the formulation is 8×10¹² VG/ml.

In certain embodiments, the concentration of AAV particle in the formulation is 1×10¹³ VG/ml.

In certain embodiments, the concentration of AAV particle in the formulation is 1.8×10¹³ VG/ml.

In certain embodiments, the concentration of AAV particle in the formulation is 2.2×10¹³ VG/ml.

In certain embodiments, the concentration of AAV particle in the formulation is 2.7×10¹³ VG/ml.

In certain embodiments, the concentration of AAV particle in the formulation is 3.5×10¹³ VG/ml.

In certain embodiments, the concentration of AAV particle in the formulation is 2.7-3.5×10¹³ VG/ml.

In certain embodiments, the concentration of AAV particle in the formulation is 7.0×10¹³ VG/ml.

In certain embodiments, the concentration of AAV particle in the formulation is 5.0×10¹² VG/mL

In certain embodiments, the concentration of AAV particle in the formulation may be between about 1×10⁶ total capsid/mL and about 1×10¹⁶ total capsid/ml. In certain embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹⁰, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ total capsid/ml. Total Dose of AAV particle

In certain embodiments, the total dose of the AAV particle in the formulation may be between about 1×10⁶ VG and about 1×10¹⁶ VG. In certain embodiments, the formulation may comprise a total dose of AAV particle of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 4×10¹², 4.1×10¹², 4.2×10¹², 4.3×10¹², 4.4×10¹², 4.5×10¹²,4.6×10¹², 4.7×10¹², 4.8×10¹², 4.9×10¹², 5×10¹², 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG.

In certain embodiments, the total dose of AAV particle in the formulation is between 1×10¹¹ and 5×10¹³ VG.

In certain embodiments, the total dose of AAV particle in the formulation is between 1×10¹¹ and 2×10¹⁴ VG.

In certain embodiments, the total dose of AAV particle in the formulation is 1.4×10¹¹ VG.

In certain embodiments, the total dose of AAV particle in the formulation is 4.5×10¹¹ VG.

In certain embodiments, the total dose of AAV particle in the formulation is 6.8×10¹¹ VG.

In certain embodiments, the total dose of AAV particle in the formulation is 1.4×10¹² VG.

In certain embodiments, the total dose of AAV particle in the formulation is 2.2×10¹² VG.

In certain embodiments, the total dose of AAV particle in the formulation is 4.6×10¹¹ VG.

In certain embodiments, the total dose of AAV particle in the formulation is 9.2×10¹² VG.

In certain embodiments, the total dose of AAV particle in the formulation is 1.0×10¹³ VG.

In certain embodiments, the total dose of AAV particle in the formulation is 2.3×10¹³ VG.

### Exemplary Formulation

In some embodiments, AAV particles of the present disclosure may be formulated in a solution comprising 10 mM sodium phosphate, 180 mM sodium chloride, and 0.001% poloxamer 188 (e.g., at a solution pH of 7.3). In some embodiments, the methods and systems to prepare AAV particles of the present disclosure, including methods comprising one or more of the steps or systems described herein, produce AAV particles formulated in a solution comprising 10 mM sodium phosphate, 180 mM sodium chloride, and 0.001% poloxamer 188 (e.g., at a solution pH of 7.3).

In some embodiments, AAV particles of the present disclosure may be formulated in a solution comprising between about 5-15 mM sodium phosphate, between about 160-200 mM sodium chloride, and between about 0.001-0.01% poloxamer 188 (solution pH between 7.1-7.5). In some embodiments, the methods and systems to prepare AAV particles of the present disclosure, including methods comprising one or more of the steps or systems described herein, produce AAV particles formulated in a solution comprising between about 5-15 mM sodium phosphate, between about 160-200 mM sodium chloride, and between about 0.001-0.01% poloxamer 188 (solution pH between 7.1-7.5).

In certain embodiments, the formulation comprises components with the following CAS (Chemical Abstracts Services) Registry Numbers, 7647-14-15 (sodium chloride), 7782-85-6 (sodium phosphate dibasic, heptahydrate), 10049-21-5 (sodium phosphate monobasic, monohydrate), and 9003-11-6 (poloxamer 188).

### Injectable Formulations

In some embodiments, an injectable preparation is provided herein. Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed comprising synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, in order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers comprise poly(orthoesters) and poly(anhydrides). Depot injectable formulations are prepared by entrapping the drug in liposomes or microemulsions which are compatible with body tissues.

### Purity of Drug Substance

The viral particles disclosed herein (e.g., AAV2 comprising nucleic acid encoding for an AADC protein) may be produced in high yield with sufficient purity that they can be administered to a human subject, e.g., using the production methods discussed herein. In some embodiments, the viral vector is formulated in a composition at a concentration of between about 3.0×10¹² to about 5.0×10¹² vg/mL, e.g., about 5.0×10¹² vg/mL.

In some embodiments, empty viral capsids that do not contain nucleic acid material may be generated during the process of production process of the viral particles. Pharmaceutical compositions comprising low amounts of empty viral capsids may be advantageous, because they avoid exposing patients, e.g., infants, with immature immune systems to antigenic material (empty capsids, host cell protein, host cell DNA) unnecessarily without therapeutic benefit. In some embodiments, such pharmaceutical compositions may reduce potential infusion reactions or broader immune responses and may improve therapeutic efficacy. Empty viral particles may be separated from full viral particles. In some embodiments, empty viral particles may be separated from full viral particles by gradient centrifugation or ultracentrifugation. In some embodiments, the ratio of empty viral particles to full viral particles may be measured by, e.g., measuring UV absorbance. In some embodiments, the ratio of empty viral particles to full viral particles may be determined using a Beckmann Optima XL-A analytical ultracentrifuge.

In some embodiments, the pharmaceutical composition produced by methods described herein comprises less than 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%,7%, 6%, 5%, 4%, 3%,2%, 1%, or less of empty particles. In some embodiments, the pharmaceutical composition produced by methods described herein comprises less than 20% empty particles. In some embodiments, the pharmaceutical composition produced by methods described herein comprises less than 10% empty particles. In some embodiments, the low percentage or ratio of empty to full viral particles produced by methods described herein may provide treatment benefits and/or reduced adverse effects after administration to a patient.

In some embodiments, a pharmaceutical composition produced by methods described herein comprises a protein purity of greater than about 90%. In some embodiments, the pharmaceutical composition produced by methods described herein comprises less than 5% contamination of proteins unrelated to the drug product. In some embodiments, the protein purity of the composition may be determined by CE-SDS.

In some embodiments, host cell (e.g., Sf9) protein contaminants may be present in the drug substance pool or purified drug substance pool. In some embodiments, host cell (e.g., Sf9) protein contaminants are minimized using the methods described herein. In some embodiments, amount of residual host cell can be distinguished between the viral capsid proteins and the residual host cell proteins. In some embodiments, the amount of residual host cell proteins can be measured by size exclusion or ion exchange chromatography. In some embodiments, the amount of residual host cell proteins can be measured by western blot using host cell-specific antibodies. In some embodiments, the amount of residual host cell protein can be measured by enzyme-linked immunosorbent assay (ELISA). In some embodiments, the methods described herein provide improved host cell contaminant levels as compared to levels in prior methods.

In some embodiments, residual host cell (e.g., Sf9 cell) DNA may be present in the drug substance pool or purified drug substance pool. In some embodiments, baculoviral DNA contaminants may be present in the drug substance pool or purified drug substance pool. In some embodiments, DNA contaminants (e.g., residual host cell, e.g., Sf9, or baculoviral) are minimized using the methods described herein. In some embodiments, the level of DNA contaminant may be detected using any method known in the art. In some embodiments, the level of DNA contaminant may be detected by PCR. In some embodiments, the level of DNA contaminant may be detected by quantitative PCR (qPCR) with primers specific for host cell sequences. In some embodiments, the level of DNA contaminant may be detected by digital droplet PCR (ddPCR). In some embodiments, the methods described herein provide improved DNA contaminant levels as compared to levels in prior methods.

In some embodiments, a pharmaceutical composition produced by the methods described herein comprises less than 10 ng/mL Sf9 residual host protein contaminants. In some embodiments, a pharmaceutical composition produced by the methods described herein comprises less than 7 ng/mL Sf9 residual host protein contaminants. In some embodiments, a pharmaceutical composition produced by the methods described herein comprises less than 5 ng/mL Sf9 residual host protein contaminants. In some embodiments, a pharmaceutical composition produced by the methods described herein comprises less than 2 ng/mL Sf9 residual host protein contaminants. In some embodiments, a pharmaceutical composition produced by the methods described herein comprises less than 1 ng/mL Sf9 residual host protein contaminants. In some embodiments, a pharmaceutical composition produced by the methods described herein comprises less than 5 ng/mL residual baculoviral DNA contaminants. In some embodiments, a pharmaceutical composition produced by the methods described herein comprises less than 2 ng/mL residual baculoviral DNA contaminants. In some embodiments, a pharmaceutical composition produced by the methods described herein comprises less than 1 ng/mL residual baculoviral DNA contaminants.

In some embodiments, a pharmaceutical composition produced by the methods described herein comprises no detectable levels of replication-competent baculovirus. In some embodiments, levels of replication-competent baculovirus in the pharmaceutical composition may be detected using a plaque assay.

In some embodiments, a pharmaceutical composition produced by the methods described herein comprises endotoxin levels of less than or equal to about 5 EU/mL, less than about 1 EU/mL, less than about 0.75 EU/mL, less than about 0.5 EU/mL, less than about 0.4 EU/mL, less than about 0.35 EU/mL, less than about 0.3 EU/mL, less than about 0.25 EU/mL, less than about 0.2 EU/mL, less than about 0.15 EU/mL, less than about 0.1 EU/mL, less than about 0.05 EU/mL, or, less than about 0.02 EU/mL. Exemplary methods for determining the amount of endotoxin include a limulus amoebocyte lysate (LAL) test.

In some embodiments, a pharmaceutical composition disclosed herein comprises a genomic titer of greater than 3×10¹² vg/mL. In some embodiments, a pharmaceutical composition disclosed herein comprises a genomic titer of about 2.0×10¹² vg/mL to about 8×10¹² vg/mL. In some embodiments, genomic titer may be measured using droplet digital PCR (ddPCR) and/or quantitative PCR (qPCR).

In one embodiment, a pharmaceutical composition disclosed herein exhibits a bioburden of less than 10 CFU/mL. In one embodiment, a pharmaceutical composition disclosed herein exhibits a bioburden of less than 1 CFU/mL. In some embodiments, the bioburden of a pharmaceutical composition disclosed herein is in accordance with United States Pharmacopoiea (USP), e.g., USP <61>, the contents of which are incorporated herein by reference in its entirety.

In some embodiments, the osmolality of a pharmaceutical composition disclosed herein in accordance with USP, e.g., USP <785> (incorporated by reference in its entirety) specifying 300-400 mOsm/kg.

In some embodiments, the pharmaceutical composition contains fewer than 6000 particles that are greater than 10 μm per container. In some embodiments, the pharmaceutical composition contains fewer than 600 particles that are greater than 25 μm per container.

### IV. Administration and Dosing

### Administration

In certain embodiments, the AAV particle may be administered to a subject (e.g., to the CNS of a subject) in a therapeutically effective amount to reduce the symptoms of the disease of the central nervous system (e.g., Parkinson's Disease) of a subject (e.g., determined using a known evaluation method).

In certain embodiments, the AAV particles of the present disclosure (e.g., as produced according to the present disclosure) may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), sub-pial (between pia and CNS parenchyma), intracarotid arterial (into the intracarotid artery), epicutaneous (application onto the skin), intradermal, (into the skin itself), subcutaneous (under the skin), nasal administration (through the nose), intravenous (into a vein), intravenous bolus, intravenous drip, intra-arterial (into an artery), systemic, intramuscular (into a muscle), intracardiac (into the heart), intraosseous infusion (into the bone marrow), intrathecal (into the spinal canal), intraparenchymal (the body of a tissue or organ, e.g., brain, spinal cord, etc.), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cistema magna cerebellomedularis), intracomeal (within the cornea), dental intracoronal, intracoronary (within the coronary arteries), intracorporus cavemosum (within the dilatable spaces of the corporus cavemosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

In some embodiments, compositions may be administered in a way which allows them to cross the blood-brain barrier, vascular barrier, or other epithelial barrier. The AAV particles of the present disclosure may be administered in any suitable form, either as a liquid solution or suspension, as a solid form suitable for liquid solution or suspension in a liquid solution. The AAV particles may be formulated with any appropriate and pharmaceutically acceptable excipient.

In certain embodiments, the AAV particles of the present disclosure (e.g., as produced according to the present disclosure) may be administered intracranially. In certain embodiments, the AAV particles of the present disclosure (e.g., AAV2 particles) may be administered into the putamen of patients having a disease of the central nervous system (e.g., Parkinson's Disease).

In certain embodiments, the AAV particles of the present disclosure may be delivered to a subject via a single route administration.

In certain embodiments, the AAV particles of the present disclosure may be delivered to a subject via a multi-site route of administration. A subject may be administered at 2, 3, 4, 5 or more than 5 sites.

In certain embodiments, a subject may be administered the AAV particles of the present disclosure using a bolus infusion.

In certain embodiments, the AAV particles of the present disclosure may be administered via a single dose intravenous delivery. In certain embodiments, the single dose intravenous delivery may be a one-time treatment. In the context of diseases of the central nervous system (e.g., Parkinson's Disease), the single dose intravenous delivery can produce durable relief for subjects with central nervous system (e.g., Parkinson's Disease) and/or related symptoms. The relief may last for minutes such as, but not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 minutes or more than 59 minutes; hours such as, but not limited to, 1, 2, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more than 48 hours; days such as, but not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or more than 31 days; weeks such as, but not limited to, 1, 2, 3, 4, 5, 6, 7, 8, 9, 10, 11, 12, 13, 14, 15, 16, or more than 16 weeks; months such as, but not limited to, 1, 2, 3, 4, 5, 6, 7, 8, 9, 10, 11, 12, 13, 14, 15, 16, 17, 18, 19, 20, 21, 22, 23, 24, or more than 24 months; years such as, but not limited to, 1, 2, 3, 4, 5, 6, 7, 8, 9, 10, 11, 12, 13, 14, 15, or more than 15 years.

In some embodiments, the AAV particles of the present disclosure may be administered by intrathecal injection. In certain embodiments, the AAV particles of the present disclosure may be administered by intrathecal injection.

In certain embodiments, the AAV particle may be administered to the cisterna magna in a therapeutically effective amount to transduce spinal cord motor neurons and/or astrocytes. In certain embodiments, the AAV particle may be administered intrathecally.

In certain embodiments, the AAV particle may be administered using intrathecal infusion in a therapeutically effective amount to transduce spinal cord motor neurons and/or astrocytes.

In certain embodiments, the AAV particle may be administered to the CNS in a therapeutically effective amount to improve function and/or survival for a subject with diseases of the central nervous system (e.g., Parkinson's Disease). In certain embodiments, the vector may be administered by direct infusion into the striatum.

The AAV particle may be administered in a “therapeutically effective” amount, i.e., an amount that is sufficient to alleviate and/or prevent at least one symptom associated with the disease, or provide improvement in the condition of the subject.

### Delivery to the Central Nervous System

In certain embodiments, delivery of the pharmaceutical compositions comprising AAV particles to cells of the central nervous system (e.g., parenchyma) comprises infusion of up to 1 mL. In certain embodiments, delivery of the pharmaceutical compositions comprising AAV particles to cells of the central nervous system (e.g., parenchyma) may comprise infusion of 0.1, 0.2, 0.3, 0.4, 0.5, 0.6, 0.7, 0.8, or 0.9 mL.

In certain embodiments, delivery of pharmaceutical composition comprising AAV particles to cells of the central nervous system (e.g., parenchyma) comprises infusion of between about 1 mL to about 120 mL. In certain embodiments, delivery of AAV particles to cells of the central nervous system (e.g., parenchyma) comprises infusion of at least 3 mL. In certain embodiments, delivery of AAV particles to cells of the central nervous system (e.g., parenchyma) consists of infusion of 3 mL. In certain embodiments, delivery of AAV particles to cells of the central nervous system (e.g., parenchyma) comprises infusion of at least 10 mL. In certain embodiments, delivery of AAV particles to cells of the central nervous system (e.g., parenchyma) consists of infusion of 10 mL.

In certain embodiments, a composition comprises at least one payload described herein (e.g., AADC, e.g., SEQ ID NO: 979) and the payloads are components of a viral genome packaged in an AAV particle. The percent (%) ratio of AAV particles comprising the payload (also referred to herein as full capsids) to the AAV particles without the payload (also referred to herein as empty capsids) in the composition may be 25:75, 30:70, 40:60, 50:50, 60:40, 70:30, or 75:25. In certain embodiments, the percent ratio of AAV particles comprising the payload to empty capsids is 60:40. In certain embodiments, the percent ratio of AAV particles comprising the payload to empty capsids is 70:30. In certain embodiments, the percent ratio of AAV particles comprising the payload to empty capsids is 85:15.

In certain embodiments, the composition comprises at least 50% AAV particles comprising the payload. In certain embodiments, the composition comprises at least 60% AAV particles comprising the payload. In certain embodiments, the composition comprises at least 70% AAV particles comprising the payload. In certain embodiments, the composition comprises at least 85% AAV particles comprising the payload. In certain embodiments, the composition comprises at least 99% AAV particles comprising the payload. In certain embodiments, the composition comprises 100% AAV particles comprising the payload.

In certain embodiments, the composition described herein comprises 60-70%, 60-80%, 60-90%, 60-99%, 60-100%, 70-80%, 70-90%, 70-99%, 70-100%, 80-85%, 80-90%, 80-95%, 80-99%, 80-100%, 90-95%, 90-99%, or 90-100% AAV particles comprising the payload. In certain embodiments, the composition described herein comprises 60-100% AAV particles comprising the payload.

In certain embodiments, the composition comprises less than 50% empty particles. In certain embodiments, the composition comprises less than 40% empty particles. In certain embodiments, the composition comprises less than 35% empty particles. In certain embodiments, the composition comprises less than 30% empty particles. In certain embodiments, the composition comprises less than 25% empty particles. In certain embodiments, the composition comprises less than 20% empty particles. In certain embodiments, the composition comprises less than 15% empty particles. In certain embodiments, the composition comprises less than 10% empty particles. In certain embodiments, the composition comprises less than 5% empty particles. In certain embodiments, the composition comprises less than 1% empty particles.

In certain embodiments, a subject who may be administered a dose of the AAV particles described herein may have advanced PD and still respond to levodopa therapy but the subject also experiences medically refractory motor complications (e.g., sever motor fluctuations and/or dyskinesias that occur during levodopa and other dopaminergic therapies despite adjustments in and optimization of medication). The subject may be healthy enough to undergo a neurosurgical procedure which may be determined by methods known in the art. In certain embodiments, the subject may meet the selection criteria for deep brain stimulation (DBS). The subject may have idiopathic PD, younger than 69 years of age, have pronounced responses to levodopa, have medication-refractory symptoms (e.g., motor fluctuation and/or dyskinesia) and/or have little or no cognitive dysfunction.

In certain embodiments, a subject who may be administered a dose of the AAV particles described herein may also suffer from dementia or cognitive impairment.

In certain embodiments, a subject who may be administered a dose of the AAV particles described herein may have been previously treated with the same or similar therapeutic. In another embodiment, a subject may have been treated with a therapeutic which has been shown to reduce the symptoms of Parkinson's Disease.

In certain embodiments, a subject may be administered a dose of the AAV particles described herein in combination with at least one other agent. In certain embodiments, the subject may be administered a dose of the AAV particles described herein in combination with Levodopa for treating and/or preventing Parkinson's Disease.

### V. Treatments

### General

The present disclosure provides a method for treating a disease, disorder and/or condition in a mammalian subject, comprising a human subject, comprising administering to the subject any of the viral particles or formulations described herein or administering to the subject any of the described compositions, comprising pharmaceutical compositions or formulations, described herein.

In certain embodiments, administration of the formulated AAV particles to a subject with not change the course of the underlying disease but will ameliorate symptoms in a subject.

In certain embodiments, the viral particles of the present disclosure are administered to a subject prophylactically.

In certain embodiments, the viral particles of the present disclosure are administered to a subject having at least one of the diseases described herein.

In certain embodiments, the viral particles of the present disclosure are administered to a subject to treat a disease or disorder described herein. The subject may have the disease or disorder or may be at-risk to developing the disease or disorder.

The present disclosure provides a method for administering to a subject in need thereof, comprising a human subject, a therapeutically effective amount of the AAV particles of the present disclosure to slow, stop or reverse disease progression. As a non-limiting example, disease progression may be measured by tests or diagnostic tool(s) known to those skilled in the art. As another non-limiting example, disease progression may be measured by change in the pathological features of the brain, CSF or other tissues of the subject.

In certain embodiments, various non-infectious diseases, e.g., neurological diseases, e.g., Parkinson's Disease, may be treated with pharmaceutical compositions of the present disclosure. AAV particles, especially blood brain barrier crossing AAV particles of the present disclosure, may be useful in treating various neurological diseases. In certain embodiments, neurological disorders treated according to the methods described herein comprise, but are not limited to Parkinson's Disease, Amyotrophic lateral sclerosis (ALS), Huntington's Disease (HD), and/or Friedreich's Ataxia (FA).

### Parkinson's Disease

In certain embodiments, recombinant AAVs of the present disclosure (e.g., as produced according to the present disclosure) are used for treating, preventing, and/or ameliorating one or more symptoms of Parkinson's disease. In certain embodiments the AAV particle used to treat Parkinson's disease comprises a payload such as, but not limited to, SEQ ID NO: 979 or a fragment or variant thereof. In some embodiments, the payload comprises SEQ ID NO: 979.

In certain embodiments, the subject is a human patient who has a minimum motor score of about 30 to a maximum score of about 100, about 10 to a maximum score of about 100, about 20 to a maximum score of about 100 in the Unified Parkinson's Disease Rating Scale.

In certain embodiments, the subject has been diagnosed with Parkinson's disease within the past 5 years prior to treatment with the compositions described herein. In certain embodiments, the subject may have been diagnosed with Parkinson's disease within a week, 2 weeks, 3 weeks, 4 weeks, 5 weeks, 6 weeks, 7 weeks, 8 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 1 year, 2 years, 3 years, 4 years or less than 5 years prior to treatment with the compositions described herein.

In certain embodiments, the subject has been diagnosed with Parkinson's disease between 5 and 10 years prior to treatment with the compositions described herein. In certain embodiments, the subject may have been diagnosed with Parkinson's disease 5, 5.5., 6, 6.5, 7, 7.5, 8, 8.5, 9, 9.5 or 10 years prior to treatment with the compositions described herein.

In certain embodiments, the subject has been diagnosed with Parkinson's disease more than 10 years prior to treatment with the compositions described herein. In certain embodiments, the subject may have been diagnosed with Parkinson's disease 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24 or more than 24 years prior to treatment with the compositions described herein.

In certain embodiments, a subject is 50-65 years of age. In certain embodiments, the subject is 50, 51, 52, 53, 54, 55, 56, 57, 58, 59, 60, 61, 62, 63, 64, or 65 years of age. In certain embodiments, the subject is 50 years of age. In certain embodiments, the subject is 51 years of age. In certain embodiments, the subject is 52 years of age. In certain embodiments, the subject is 53 years of age. In certain embodiments, the subject is 54 years of age. In certain embodiments, the subject is 55 years of age. In certain embodiments, the subject is 56 years of age. In certain embodiments, the subject is 57 years of age. In certain embodiments, the subject is 58 years of age. In certain embodiments, the subject is 59 years of age. In certain embodiments, the subject is 60 years of age. In certain embodiments, the subject is 61 years of age. In certain embodiments, the subject is 62 years of age. In certain embodiments, the subject is 63 years of age. In certain embodiments, the subject is 64 years of age. In certain embodiments, the subject is 65 years of age.

In certain embodiments, a subject is 30 to 50 years of age. In certain embodiments, the subject is 30 years of age. In certain embodiments, the subject is 31 years of age. In certain embodiments, the subject is 32 years of age. In certain embodiments, the subject is 33 years of age. In certain embodiments, the subject is 34 years of age. In certain embodiments, the subject is 35 years of age. In certain embodiments, the subject is 36 years of age. In certain embodiments, the subject is 37 years of age. In certain embodiments, the subject is 38 years of age. In certain embodiments, the subject is 39 years of age. In certain embodiments, the subject is 40 years of age. In certain embodiments, the subject is 41 years of age. In certain embodiments, the subject is 42 years of age. In certain embodiments, the subject is 43 years of age. In certain embodiments, the subject is 44 years of age. In certain embodiments, the subject is 45 years of age. In certain embodiments, the subject is 46 years of age. In certain embodiments, the subject is 47 years of age. In certain embodiments, the subject is 48 years of age. In certain embodiments, the subject is 49 years of age. In certain embodiments, the subject is 50 years of age.

In certain embodiments, a subject is 65 to 85 years of age. In certain embodiments, the subject is 65 years of age. In certain embodiments, the subject is 66 years of age. In certain embodiments, the subject is 67 years of age. In certain embodiments, the subject is 68 years of age. In certain embodiments, the subject is 69 years of age. In certain embodiments, the subject is 70 years of age. In certain embodiments, the subject is 71 years of age. In certain embodiments, the subject is 72 years of age. In certain embodiments, the subject is 73 years of age. In certain embodiments, the subject is 74 years of age. In certain embodiments, the subject is 75 years of age. In certain embodiments, the subject is 76 years of age. In certain embodiments, the subject is 77 years of age. In certain embodiments, the subject is 78 years of age. In certain embodiments, the subject is 79 years of age. In certain embodiments, the subject is 80 years of age. In certain embodiments, the subject is 81 years of age. In certain embodiments, the subject is 82 years of age. In certain embodiments, the subject is 83 years of age. In certain embodiments, the subject is 84 years of age. In certain embodiments, the subject is 85 years of age.

In certain embodiments, a subject has seen a change in motor symptoms such as tremors and movements prior to administration of the composition described herein. Non-limiting examples of tremors include, unilateral or bilateral mild tremors, bilateral or midline moderate tremors or intractable tremors. Non-limiting examples of movements include mild bradykinesia, moderate bradykinesia, severe bradykinesia and morning akinesia.

In certain embodiments, a subject may have changes in balance such as, but not limited to, impaired balance, impaired righting reflexes, significant balance disorder or falling.

In certain embodiments, a subject may have a reduced quality of life. In certain embodiments, the subject may have a moderate impact on their quality of life such as experiencing some limitations to activities of daily living. In certain embodiments, the subject may have a quality of life which has been diminished by illness.

In certain embodiments, a subject has seen a change in non-motor symptoms prior to administration of the composition described herein. In certain embodiments, the subject may have mild to moderate cognitive impairment prior to administration to the composition described herein. In certain embodiments, the subject may have significant cognitive impairment such as dementia which may also include behavioral disturbances such as hallucinations.

In certain embodiments, a subject may have a satisfactory response with limited fluctuations on one or more dopaminergic medications prior to administration of the compositions described herein.

In certain embodiments, a subject may have motor fluctuations causing mild to moderate disability on one or more dopaminergic medications prior to administration of the compositions described herein.

In certain embodiments, a subject may have medically refractory motor fluctuations consisting of “wearing off” and/or levodopa-induced dyskinesias causing significant disability prior to administration of the compositions described herein.

In certain embodiments, a subject may have mild symptoms associated with Parkinson's disease such as, but not limited to, no cognitive impairment, diagnosed within the past 5 years, satisfactory response with limited fluctuations on one or more dopaminergic medications, unilateral or bilateral mild tremors, little to no impact on the quality of life, and/or no balance impairment.

In certain embodiments, a subject may have moderate symptoms associated with Parkinson's disease such as, but not limited to, mild to moderate cognitive impairment, first signs of impaired balance and righting reflexes, motor fluctuations causing mild-moderate disability on one or more dopaminergic medications, diagnosed within the past 5 to 10 years, bilateral or midline moderate tremors, moderate bradykinesia and/or subject experiencing some limitations to activities of daily living.

In certain embodiments, a subject may have advanced symptoms associated with Parkinson's disease such as, but not limited to, being diagnosed with Parkinson's more than 10 years, medium refractory motor fluctuations wearing off and/or levodopa-induced dyskinesia causing significant disability, intractable tremors, significant balance disorder and/or falling, significant cognitive impairment (such as dementia with or without behavioral disturbances), sever bradykinesia, quality of life markedly diminished by illness and/or morning akinesia.

In certain embodiments, a subject has been referred to a movement disorder specialist (MDS) but has not undergone deep brain stimulation.

In certain embodiments, a subject is using DUOPA™ in combination with the compositions described herein. In certain embodiments, the subject may have success with using DUOPA™ alone. In certain embodiments, the subject may not have any success or limited success using DUOPA™ alone.

In certain embodiments, a subject is one who was a candidate for surgical intervention including, but not limited to, deep-brain stimulation. In certain embodiments, deep-brain stimulation was suggested due to disabling motor complications despite treatment with optimal anti-Parkinsonian medication.

### Kits and Devices

In some embodiments, the disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the AAV particles of the present disclosure may be comprised in a kit. In some embodiments, kits may further comprise reagents and/or instructions for creating and/or synthesizing compounds and/or compositions of the present disclosure. In some embodiments, kits may also comprise one or more buffers. In some embodiments, kits of the disclosure may comprise components for making protein or nucleic acid arrays or libraries and thus, may comprise, for example, solid supports.

In some embodiments, kit components may be packaged either in aqueous media or in lyophilized form. The container means of the kits will generally comprise at least one vial, test tube, flask, bottle, syringe or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically comprise means for containing compounds and/or compositions of the present disclosure, e.g., proteins, nucleic acids, and any other reagent containers in close confinement for commercial sale. Such containers may comprise injection or blow-molded plastic containers into which desired vials are retained.

In some embodiments, the vial is capable of holding at least 0.5 mL of drug substance. In some embodiments, the vial is capable of holding at least 0.8 mL of drug substance. In some embodiments, the vial is capable of holding at least 1.2 mL of drug substance. In some embodiments, the vial is capable of holding at least 1.8 mL of drug substance. In some embodiments, the vial is a 2 mL vial or a 3 mL vial, e.g., a 2 mL or 3 mL Ompi glass vial. In some embodiments, the vial further comprises a seal and stopper. For example, the seal may be a 13 mm West Pharma Flip Off Long Matte Seal and the stopper may be a 13 mm West S2-F451 4432/50. In some embodiments, the extractable volume of drug substance from the vial is about 1.0 mL. In some embodiments, the extractable volume of drug substance from the vial is about 1.6 mL. In some embodiments, the vial is stored (e.g., prior to treatment) at ≤65° C.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may comprise instructions for employing kit components as well the use of any other reagent not comprised in the kit. Instructions may comprise variations that may be implemented.

### VI. Definitions

At various places in the present disclosure, substituents or properties of compounds of the present disclosure are disclosed in groups or in ranges. It is specifically intended that the present disclosure comprise each and every individual or sub-combination of the members of such groups and ranges. All ranges include the endpoints unless stated otherwise.

Unless stated otherwise, the following terms and phrases have the meanings described below. The definitions are not meant to be limiting in nature and serve to provide a clearer understanding of certain aspects of the present disclosure.

AAV Particle: As used herein, an “AAV particle” is a virus which comprises a capsid and a viral genome with at least one payload region and at least one ITR region. AAV particles of the present disclosure may be produced recombinantly and may be based on adeno-associated virus (AAV) parent or reference sequences. AAV particle may be derived from any serotype, described herein or known in the art, comprising combinations of serotypes (i.e., “pseudotyped” AAV) or from various genomes (e.g., single stranded or self-complementary). In addition, the AAV particle may be replication defective and/or targeted.

Administered in combination: As used herein, the term “administered in combination” or “combined administration” means that two or more agents are administered to a subject at the same time or within an interval such that there may be an overlap of an effect of each agent on the patient. In certain embodiments, they are administered within about 60, 30, 15, 10, 5, or 1 minute of one another. In certain embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration comprises the reduction of neuron loss. Amelioration includes but does not require complete amelioration.

Animal: As used herein, the term “animal” refers to any member of the animal kingdom. In certain embodiments, “animal” refers to humans at any stage of development. In certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In certain embodiments, animals comprise, but are not limited to, mammals, birds, reptiles, amphibians, fish, and worms. In certain embodiments, the animal is a transgenic animal, genetically-engineered animal, or a clone.

Approximately or about: As used herein, the term “approximately” or “about,” as applied to one or more values of interest, refers to a value that is similar to a stated reference value. As used herein, the term “about” means+/−10% of the recited value.

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Baculoviral expression vector (BEV): As used herein a BEV is a baculoviral expression vector, i.e., a polynucleotide vector of baculoviral origin. Systems using BEVs are known as baculoviral expression vector systems (BEVSs).

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle of the present disclosure may be considered biologically active if even a portion of the encoded payload is biologically active or mimics an activity considered biologically relevant.

Capsid: As used herein, the term “capsid” refers to the exterior of a virus particle, e.g., a shell of a virus particle comprising protein (e.g., >80%, >90%, >95%, >99%, or 100% protein). In some embodiments, the capsid is an AAV capsid comprising at least one AAV capsid protein described herein, e.g., a VP1, VP2, and/or VP3 polypeptide.

Codon optimized: As used herein, the terms “codon optimized” or “codon optimization” refers to a modified nucleic acid sequence which encodes the same amino acid sequence as a parent/reference sequence, but which has been altered such that the codons of the modified nucleic acid sequence are optimized or improved for expression in a particular system (such as a particular species or group of species). As a non-limiting example, a nucleic acid sequence which comprises an AAV capsid protein can be codon optimized for expression in insect cells or in a particular insect cell such Spodopterafrugiperda cells. Codon optimization can be completed using methods and databases known to those in the art.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity.

Compound: Compounds of the present disclosure comprise all of the isotopes of the atoms occurring in the intermediate or final compounds. “Isotopes” refers to atoms having the same atomic number but different mass numbers resulting from a different number of neutrons in the nuclei. For example, isotopes of hydrogen comprise tritium and deuterium.

The compounds and salts of the present disclosure can be prepared, in various embodiments, in combination with solvent or water molecules to form solvates and hydrates by routine methods.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Delivery: As used herein, “delivery” refers to the act or manner of delivering an AAV particle, a compound, substance, entity, moiety, cargo or payload.

Delivery Agent: As used herein, “delivery agent” refers to any substance which facilitates, at least in part, the in vivo delivery of an AAV particle to targeted cells.

Destabilized: As used herein, the term “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art comprising radiography, fluorescence, chemiluminescence, enzymatic activity, absorbance and the like. Detectable labels comprise radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Engineered: As used herein, embodiments of the present disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g., between polynucleotide molecules (e.g., DNA molecules and/or RNA molecules) and/or between polypeptide molecules. In certain embodiments, polymeric molecules are considered to be “homologous” to one another if their sequences are at least 90% identical or similar, e.g., using an alignment program such as BLAST (e.g., BLASTP or BLASTN).

Identity: As used herein, the term “identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of which are each incorporated herein by reference in their entireties, insofar as they do not conflict with the present disclosure. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences comprise, but are not limited to those disclosed in Carillo, H., and Lipman, D., SIAM J Applied Math., 48:1073 (1988); the contents of which are each incorporated herein by reference in their entireties, insofar as they does not conflict with the present disclosure. Techniques for determining identity are codified in publicly available computer programs. Exemplary computer software to determine homology between two sequences (e.g., for local alignment and/or for global alignment) comprise, but are not limited to, GCG program package, Devereux, J., et al., Nucleic Acids Research, 12(1), 387 (1984)); BLASTP, BLASTN, and FASTA Altschul, S. F. et al., J. Molec. Biol., 215, 403 (1990)); EMBOSS Needle; and/or Needleman-Wunsch algorithm (e.g., NWSgapdna.CMP).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated.

Linker: As used herein “linker” refers to a molecule or group of molecules which connects two molecules. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule of the present disclosure. Molecules may be modified in many ways comprising chemically, structurally, and functionally. As used herein, embodiments of the disclosure are “modified” when they have or possess a feature or property, whether structural or chemical, that varies from a starting point, wild type or native molecule. Mutation: As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes. In certain embodiments, a variant can be a mutant sequence. In certain embodiments, a variant may comprise at least about 90% identity to a native sequence. As used herein, a native or starting sequence refers to an original molecule against which a comparison may be made, such as a wild-type or naturally-occurring sequence known to one of skill in the art. “Native” or “starting” sequences or molecules may represent the wild-type (the sequence found in nature) but do not have to be the wild-type sequence.

Naturally Occurring: As used herein, “naturally occurring” or “wild-type” means existing in nature without artificial aid, or involvement of the hand of man.

Open reading frame: As used herein, “open reading frame” or “ORF” refers to a sequence which does not contain a stop codon within the given reading frame, other than at the end of the reading frame.

Operably linked: As used herein, the phrase “operably linked” refers to a functional connection between two or more molecules, constructs, transcripts, entities, moieties or the like. As a non-limiting example, a promoter is “operably linked” to a nucleotide sequence when the promoter sequence controls and/or regulates the transcription of the nucleotide sequence.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, or a modulatory nucleic acid or regulatory nucleic acid.

Payload construct: As used herein, a “payload construct” is one or more vector construct which comprises a polynucleotide region encoding or comprising a payload (e.g., a nucleic acid encoding an AADC protein or a functional variant) that is flanked on one or both sides by an inverted terminal repeat (ITR) sequence. Additional nucleic acid components may be present between the ITRs and the payload sequence. The payload construct may present a template that is replicated in a viral production cell to produce a therapeutic viral genome.

Payload construct vector: As used herein, “payload construct vector” is a vector encoding or comprising a payload construct, and regulatory regions for replication and expression of the payload construct in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient.

Pharmaceutically acceptable salts: The present disclosure also comprises pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts comprise, but are not limited to, mineral or organic acid salts of basic residues such as amines; alkali or organic salts of acidic residues such as carboxylic acids; and the like. In certain embodiments, the pharmaceutically acceptable salts of the present disclosure comprise the conventional non-toxic salts of the parent compound formed, for example, from non-toxic inorganic or organic acids. In certain embodiments, the pharmaceutically acceptable salts of the present disclosure can be synthesized from the parent compound which contains a basic or acidic moiety by conventional chemical methods. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P.H. Stahl and C.G. Wermuth (eds.), Wiley-VCH, 2008, and Berge et al., Journal of Pharmaceutical Science, 66, 1-19 (1977), the contents of which are each incorporated herein by reference in their entireties, insofar as they do not conflict with the present disclosure.

Pharmaceutically acceptable solvate: The term “pharmaceutically acceptable solvate,” as used herein, means a compound of the present disclosure wherein molecules of a suitable solvent are incorporated in the crystal lattice. In certain embodiments, a suitable solvent is physiologically tolerable at the dosage administered.

Pharmacokinetic: As used herein, “pharmacokinetic” refers to any one or more properties of a molecule or compound as it relates to the determination of the fate of substances administered to a living organism. Pharmacokinetics is divided into several areas comprising the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Protein of interest: As used herein, the terms “proteins of interest” or “desired proteins” comprise those provided herein and fragments, mutants, variants, and alterations thereof. In some embodiments, the protein of interest is AADC or a functional variant thereof.

Proximal: As used herein, the term “proximal” means situated nearer to the center or to a point or region of interest.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

Sample: As used herein, the term “sample” or “biological sample” refers to a subset of its tissues, cells or component parts (e.g., body fluids, comprising but not limited to blood, mucus, lymphatic fluid, synovial fluid, cerebrospinal fluid, saliva, amniotic fluid, amniotic cord blood, urine, vaginal fluid and semen). A sample further may comprise a homogenate, lysate or extract prepared from a whole organism or a subset of its tissues, cells or component parts, or a fraction or portion thereof, comprising but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a polynucleotide sequence encoding a self-complementary genome enclosed within the capsid.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In certain embodiments, a single unit dose is provided as a discrete dosage form (e.g., a tablet, capsule, patch, loaded syringe, vial, etc.).

Stable: As used herein “stable” refers to a compound that is sufficiently robust to survive isolation to a useful degree of purity from a reaction mixture for a set period of time, and in certain embodiments, capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject or patient may seek or need treatment, require treatment, is receiving treatment, will receive treatment, or is under care by a trained professional for a particular disease or condition.

Sufferingfrom: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In certain embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In certain embodiments, an individual who is susceptible to a disease, disorder, and/or condition will develop the disease, disorder, and/or condition. In certain embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present disclosure may be chemical or enzymatic.

Targeted Cells: As used herein, “targeted cells” refers to any one or more cells of interest that may be treated by an AAV disclosed herein. The cells may be found in vitro, in vivo, in situ or in the tissue or organ of an organism. The organism may be an animal, such as a mammal, a human, or a human patient.

Terminal region: As used herein, the term “terminal region” refers to a region on the 5′ or 3′ end of a region of linked nucleosides or amino acids (polynucleotide or polypeptide, respectively).

Terminally optimized: The term “terminally optimized” when referring to nucleic acids means the terminal regions of the nucleic acid are improved in some way, e.g., codon optimized, over the native or wild type terminal regions.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In certain embodiments, a therapeutically effective amount is provided in a single dose. In certain embodiments, a therapeutically effective amount is administered in a dosage regimen comprising a plurality of doses. Those skilled in the art will appreciate that in certain embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Transfection: As used herein, the term “transfection” refers to methods to introduce exogenous nucleic acids into a cell. Methods of transfection comprise, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) sequence(s), e.g., those comprising or derived from wildtype, naturally-occurring, or engineered AAV sequences known in the art. In some embodiments, a vector may comprise sequences to which a payload is included (e.g., a shuttle plasmid sequence which includes a heterologous payload sequence). In some embodiments, a vector comprising a payload sequence can be considered a vector genome when encapsulated in a particle, e.g., an AAV viral genome in an AAV particle

Viral genome: As used herein, a “viral genome” or “vector genome” refers to the nucleic acid sequence(s) encapsulated in an AAV particle.

### VII. Equivalents and Scope

Those skilled in the art will recognize or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that comprise “or” between one or more members of a group are considered satisfied if one, more than one, or all of the group members are present in, employed in, or otherwise relevant to a given product or process unless indicated to the contrary or otherwise evident from the context. The present disclosure comprises embodiments in which exactly one member of the group is present in, employed in, or otherwise relevant to a given product or process. The present disclosure comprises embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are comprised. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

All terms used herein should be given their ordinary meaning by those of skill in the art unless the disclosure indicates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, comprising definitions, will control. In addition, section headings, the materials, methods, and examples are illustrative only and not intended to be limiting.

### VIII. EXAMPLES

### Example 1. Production of Source Rep/Cap BIICs (A)

### CP BEV Pool

One vial of the Sf9 CB was thawed in a 125 mL shaker flask (37° C. using waterbath, 1-5 minutes until ice crystals dissipate), and then diluted into 19-20 mL working volume of Hyclone SFX Insect Cell Culture Media. The shaker flask was incubated at 27° C. (135 rpm shaking, 0% v/v of CO₂) in a first expansion (P0, 3-4 days) until the cell density of the Sf9 cell mixture was expanded to 4.0×10⁶ cells/mL.

The cell mixture was then seeded and expanded through multiple additional expansion steps using larger shaker flasks, with a target output density of 4.0×10⁶ cells/mL for each expansion step to allow for a consistent seeding density of 0.5-1.5×10⁶ cells/mL in subsequent expansion steps. Expansions were completed at 27° C. for 3-5 days (0% v/v of CO₂) with 135 rpm shaking (≤2 L working volume) or 90 rpm shaking (>2 L working volume). The following additional expansions were completed: (i) expansion up to 200 mL working volume in a 1.0 L flask; and (ii) expansion up to 1000 mL working volume in a 3 L flask.

A Rep/Cap Transfection Mixture was prepared by combining 5 μg of Rep/Cap Bacmid material with 375 μL of WFI water. The diluted Bacmid mixture was combined with 30 μL of Promega FuGENE HD (Transfection Agent) and an additional 345 μL of WFI water, and then incubated at 27° C. for 15 minutes to provide a Transfection Cocktail.

25 mL of expanded Sf9 cell mixture was seeded into a 125 mL flask (1.0×10⁶ cells/mL seeding concentration) and expanded to a target infection density of 2.5-4.0×10⁶ cells/mL. The Transfection Cocktail was added to the 125 mL flask and incubated at 27° C. for 5-7 days (0% v/v of CO₂, 135 rpm agitation). The resulting mixture was centrifuged in 50 mL conical tubes for 5 minutes, and the supernatant containing P1 BEVs was collected and pooled with other P1 BEV supernatants. The P1 BEV pool was stored at 5° C.

Expanded Sf9 cell mixture was seeded into a Cellstar 6-well Cell Polystyrene Culture Plate (2 mL per well, 0.5-1.0×10⁶ cells/mL seeding concentration) with gentle rocking to evenly distribute cells, followed by incubation at 27° C. for 90 minutes (0% v/v of CO₂, 0 rpm agitation). P1 BEVs were serial diluted with Hyclone SFX Insect Cell Culture Media to a target dilution of 1.0×10⁷ BEVs/mL, and then 1 mL of diluted P1 BEV mixture was added to each well with gentle rocking to evenly distribute P1 BEVs. The infection mixture was incubated at 27° C. for 90 minutes (0% v/v of CO₂, 0 rpm agitation).

Agarose gel was prepared by combining 4% w/v agarose 1:3 with Life Technologies Sf-900 Medium overlay (melt agarose at 70° C., cool to 37° C. for combination). 2 mL of Agarose Overlay was then added to each well, and the plates were maintained at room temperature for 15-20 minutes for the agarose gel to harden. Overlaid plates were then incubated at 27° C. for 5-14 days (0% v/v of CO₂, 0 rpm agitation) until plaque formation was observed. Plaques in each well were processed through testing and Plaque Picking to provide a single Plaque for Clonal Plaque Purification (i.e. Single Plaque Expansion). The Single Plaque was expanded using Sf9 cell mixture and incubated at 27° C. for 3-5 days (0% v/v of CO₂, 0 rpm agitation). The resulting CP1 BEVs were harvested using centrifugation in 50 mL conical tubes for 5 minutes and collection of supernatant containing CP1 BEVs into a CP1 BEV pool.

### BEV Infection/BIIC Production

Sf9 cell mixture was seeded and expanded through multiple expansion steps using larger shaker flasks, with a target output density of 4.0×10⁶ cells/mL for each expansion step to allow for a consistent seeding density of 0.5-1.0×10⁶ cells/mL in subsequent expansion steps. Expansions were completed at 27° C. for 3-5 days (0% v/v of CO₂) with 135 rpm shaking (≤2 L working volume) or 90 rpm shaking (>2 L working volume). The following expansions were completed: (i) expansion up to 200 mL working volume in a 1.0 L flask; (ii) expansion up to 1000 mL working volume in a 3 L flask; and (iii) expansion up to 2500 mL working volume in a 5 L flask, with a final output density of 2.0×10⁶ cells/mL.

200 mL of expanded Sf9 cell mixture was seeded into a 1.0 L flask (1.0×10⁶ cells/mL seeding density) and expanded to a viable infection cell density of ≥2.0×10⁶ cells/mL, and then infected with 0.01 MOI of CP1 BEV. Infected cells were then incubated and expanded at 27° C. for 48-80 hours (0% v/v of CO₂, 135 rpm) until cells reach ≥2.0×10⁶ cells/mL (VCD), ≥16.5 μm cell diameter and ≥75% cell viability. The infected cells were harvested by spinning down (polypropylene centrifuge tubes, 5 min) and resuspending the cell pellet at 4.0×10⁷ cells/mL in Hyclone SFX Insect Cell Culture Media, followed by the addition of 300 mM Trehalose, 14% v/v of DMSO, and additional SFX Culture Media to provide target VCD of 2.0×10⁶ cells/mL. Rep/Cap Source BIICs were aliquoted into 2 mL or 5 mL cryovials and frozen down to ≤−65° C. using control rate freezer, and then stored at −80° C. or in LN₂ vapor.

### Example 2. Production of Source Transgene BIICs (A)

Transgene Source BIICs were produced according to Example 1, with Transgene Bacmid material used for P1 BEV production instead of Rep/Cap Bacmid material.

### Example 3. Production Source Rep/Cap BIICs (B)

### CP BEV Pool

One vial of the Sf9 CB was thawed in a 125 mL shaker flask (37° C. using waterbath, 1-5 minutes until ice crystals dissipate), and then diluted into 40 mL working volume of Hyclone SFX Insect Cell Culture Media. The shaker flask was incubated for a first expansion (P0, 3-4 days) until the cell density of the Sf9 cell mixture was expanded to 4.0×10⁶ cells/mL.

The culture was then seeded and expanded through multiple additional expansion steps using larger shaker flasks, with a target output density of 4.0×10⁶ cells/mL for each expansion step to allow for a consistent seeding density of 0.5-1.0×10⁶ cells/mL in subsequent expansion steps. Expansions were completed at 27° C. for 3-5 days and comprised: (i) expansion up to 200 mL working volume in a 1.0 L flask (P1); and (ii) expansion up to 1000 mL working volume in a 3 L flask (P2).

A Rep/Cap Transfection Mixture was prepared by combining 30 μg of Rep/Cap Bacmid material with 0.6 mL of ThermoFisher Grace's Insect Media (Transfection Media). The diluted Bacmid mixture was combined with 30 μL of ThermoFisher Cellfectin II Reagent (Transfection Agent) and an additional 0.6 mL of Transfection Media, followed by incubation at 18-25° C. for 25-35 minutes, and then further dilution with 4.8 mL of Transfection Media to provide a Transfection Cocktail.

60 mL of expanded Sf9 cell mixture was seeded into a 125 mL flask and expanded up to 1.0×10⁶ cells/mL seeding concentration. The Sf9 cell mixture was then seeded into a 6-well Cell Culture Plate (2 mL per well, 1.0×10⁶ cells/mL seeding concentration). 1 mL of Transfection Cocktail was added to each well, and the plate was incubated at 27° C. for 4-5 hours. 2 mL of Hyclone SFX Insect Cell Culture Media was added to each well, and the plates were then incubated at 27° C. for 3-4 days. The resulting mixtures were centrifuged in 50 mL conical tubes for 5 minutes, and supernatant containing P1 BEVs was collected and pooled with other P1 BEV supernatants. The P1 BEV pool was stored at 4-8° C.

Expanded Sf9 cell mixture was seeded into a 6-well Cell Culture Plate (2 mL per well, 0.5-1.0 cells/mL seeding concentration) with gentle rocking to evenly distribute cells, followed by incubation at 27° C. for 90 minutes. P1 BEVs were serial diluted with Hyclone SFX Insect Cell Culture Media to a target dilution of 1.0-5.0×10⁷ BEVs, and then 1 mL of diluted P1 BEV mixture was added to each well with gentle rocking to evenly distribute P1 BEVs. The infection mixture was incubated at 27° C. for 90 minutes.

Agarose gel was prepared by combining 4% w/v agarose 1:3 with Life Technologies Sf-900 Medium overlay. Agarose Overlay was added to each well, and the plates were maintained at room temperature for 15-20 minutes for the agarose gel to harden. Overlaid plates were then incubated at 27° C. for 10 days until plaque formation was observed. Plaques in each well were processed through testing and Plaque Picking to provide a single Plaque for Clonal Plaque Purification (i.e. Single Plaque Expansion). The Single Plaque was expanded using Sf9 cell mixture of 120 mL pools in 500 mL flask, with incubation at 27° C. for 4 days. The resulting CP2 BEVs were harvested using centrifugation in 50 mL conical tubes for 5 minutes and collection of supernatant containing CP2 BEVs into a CP2 BEV pool.

### BEV Infection/BIIC Production

Sf9 cell mixture was seeded and expanded through multiple expansion steps up to 3000 mL working volume in a 5 L flask, with a final infection density of 1.0×10⁶ cells/mL. The expanded Sf9 cell mixture was then infected with 0.01 MOI of CP2 BEV. Infected cells were incubated and expanded at 27° C. for 48-36 hours, then harvested by spinning down (polypropylene centrifuge tubes, 5 min) and resuspending the cell pellet at 2.0×10⁷ cells/mL in Hyclone SFX Insect Cell Culture Media, followed by the addition of 300 mM Trehalose, 14% v/v of DMSO, and additional SFX Culture Media to provide target VCD of 2.0×10⁶ cells/mL. Rep/Cap Source BIICs were aliquoted into 2 mL or 5 mL cryovials and frozen down to ≤−65° C. using control rate freezer, and then stored at −80° C. or in LN₂ vapor.

### Example 4. Production of Source Transgene BIICs (B)

Transgene Source BIICs were produced according to Example 3, with Transgene Bacmid material used for P1 BEV production instead of Rep/Cap Bacmid material.

### Example 5. Exemplary Method for Producing rAAVs

### Generation of Baculovirus Expression Vectors

Baculovirus expression vectors were generated using a Bac-to-Bac™ expression system according to manufacturer's protocol (ThermoFisher/Invitrogen). Briefly, an AAV2 rep2/cap2 construct encoding AAV2 non-structural capsid proteins (Rep 52 and Rep 78) and structural capsid proteins (VP1, VP2, and VP3) was cloned into a pFastBac™ plasmid to produce a pVOY-rep2/cap2 donor plasmid (SEQ ID NO: 1781). The pVOY-rep2/cap2 plasmid was then transformed into chemically competent DH10Bac™ E. coli cells, which contains a bMON14272 baculovirus shuttle vector (bacmid) and a helper plasmid. The AAV2 rep2/cap2 construct was inserted into the bMON14272 baculovirus vector (SEQ ID NO: 1783) via transposition between the Tn7 region of the donor plasmid and the mini-attTn7 target site of the bacmid.

A baculovirus payload vector encoding an AADC payload was generated using similar steps. Briefly, a human AADC construct encoding SEQ ID NO: 979 was cloned into a pUC57 plasmid to produce a pMP-hAADC donor plasmid (SEQ ID NO: 1782). The pMP-hAADC donor plasmid was then transformed into chemically competent DH10Bac™ E. coli cells, wherein the AADC construct was inserted into the mini-attTn7 target site of bMON14272 baculovirus shuttle vector via the transposition mechanism described above.

Production of Infection BIICs from Source BIICs

Rep/Cap Source BIICs and Rep/Cap Infection BIICs referenced in this Example are also referred to as “expressionBIICs,” which comprise one or more baculoviruses (“expressionBacs”), which in turn comprise a viral expression construct encoding capsid (VP1, VP2, VP3) and replication (Rep52, Rep78) proteins for wild-type AAV2. Transgene Source BIICs and Transgene Infection BIICs referenced in this Example are also referred to as “payloadBIICs,” which comprise one or more baculoviruses (“payloadBacs”), which in turn comprise a payload construct comprising SEQ ID NO: 979.

One vial of Sf9 9f4 CB was thawed in a 125 mL shaker flask (37° C. in a waterbath, 1-5 minutes until ice crystals dissipate), and then diluted into 20 mL working volume of ESF-AF culture medium. The shaker flask was incubated at 27° C. (100 rpm shaking, 2-inch orbital diameter) in a non-humidified, ambient air, temperature regulated incubator in a first expansion until the cell density reached between 5.0-8.0×10⁶ cells/mL.

The culture was then seeded and expanded through multiple additional expansion steps using larger shaker flasks, with a target output density of 5.0-8.0×10⁶ cells/mL for each expansion step to allow for a consistent seeding density of 1.0-1.5×10⁶ cells/mL in subsequent expansion steps. Expansions were completed at 27° C. for 3-5 days with 100 rpm shaking (≤2 L working volume) or 80 rpm shaking (>2 L working volume).

The following additional expansions were completed: (i) expansion up to a 100 mL working volume in a 500 mL flask; (ii) expansion up to a 400 mL working volume in a 1.0 L flask; (iii) expansion up to a 1500 mL working volume in a 3 L flask; and (iv) expansion up to a 2500 mL working volume in each of two 5 L Production Flasks (Rep/Cap Production Flask and Transgene Production Flask).

The Rep/Cap Production Flask was incubated until cell concentration expanded to 1.8-2.5×10⁶ cells/mL and was then infected with Rep/Cap Source BIICs (Sf9:BIIC Infection Ratio of 1.0×10⁴ cell-to-cell (c/c), equivalent to 1.0×10⁵ (v/v) infection ratio). The infected cells were incubated for 72 hours (target cell diameter of ≥19.0 μm, cell culture density target of ≥3.0×10⁶ cells/mL), and then harvested by spinning down (polypropylene centrifuge tubes, 5 min at 4.0° C.) and resuspending the cell pellet at 2.0×10⁷ cells/mL in 50% 2× Freezing media (858 mL/L of ESF-AF Media, 140 mL/L of Dimethyl Sulfoxide, 113 mL/L of Trehalose, dihydrate) and 50% ESF-AF Media. The resuspended culture of Rep/Cap Infection BIICs was aliquoted into 2 mL or 5 mL cryovials and stored in LN₂ vapor.

The Transgene Production Flask was incubated until the cell concentration was expanded to 1.8-2.5×10⁶ cells/mL and was then infected with Transgene Source BIICs (Sf9:BIIC Infection Ratio of 1.0×10⁴ cell-to-cell (c/c), equivalent to 1.0×10⁵ v/v infection ratio). The infected cells were incubated for 96-100 hours (target cell diameter of ≥19.0 μm, cell culture density target of ≥3.0×10⁶ cells/mL), and then harvested by spinning down (polypropylene centrifuge tubes, 5 min at 4.0° C.) and resuspending the cell pellet at 2.0×10⁷ cells/mL in 50% 2× Freezing media (858 mL/L of ESF-AF Media, 140 mL/L of Dimethyl Sulfoxide, 113 mL/L of Trehalose, dihydrate) and 50% ESF-AF Media. The resuspended culture of Transgene Infection BIICs was aliquoted into 2 mL or 5 mL cryovials and stored in LN₂ vapor.

### Upstream—Production of Bulk Particle Pool

One vial of Sf9 9f4 CB was thawed in a 125 mL shaker flask (37° C. in a waterbath, 1-5 minutes until ice crystals dissipate), and then diluted into 20 mL working volume of ESF-AF culture medium. The shaker flask was incubated at 27° C. (130-150 rpm shaking, 25 mm orbital diameter) for about 48 hours in a non-humidified, ambient air, temperature regulated incubator in a first expansion until the cell density was expanded to between 5.0×10⁶-8.0×10⁶ cells/mL. The culture was then seeded and expanded through multiple additional expansion steps using larger shaker flasks, with a target output density of 5.0×10⁶-1.0×10⁷ cells/mL for each expansion step to allow for a consistent target seeding density of 1.0-1.5×10⁶ cells/mL in subsequent expansion steps. Expansions were completed at 27° C. for 3-5 days with 130-150 rpm shaking (≤400 mL working volume) or 100-120 rpm shaking (>400 mL working volume).

The following additional expansions were completed: (i) expansion up to 100 mL working volume in a 250 or 500 mL flask; (ii) expansion up to 400 mL working volume in a 1.0 L flask; (iii) expansion up to 1500 mL working volume in a 3 L flask; and (iv) expansion up to 2500 mL working volume in each of two 5 L flasks (5000 mL total working volume).

The expanded culture mixture was transferred to a Pall Allegro XRS 25L Bioreactor for an additional expansion (25 cpm agitation, cascading oxygen on demand up to 40% dissolved 02, 0.3 m/min fixed air sparge, 0.5 mL/min headspace flow rate 3-5 days at 27° C.) up to a 10 L working volume with a target output density of 5.0×10⁶-1.0×10⁷ cells/mL.

The Production Bioreactor was a Pall 200L Allegro Bioreactor (60 rpm agitation, cascading oxygen on demand up to 40% dissolved O₂, 1.2 L/min air overlay, 27±1° C. vessel temp, 2.5 L/min O₂ flow rate). The culture mixture was seeded into the Production Bioreactor with a target seeding density of about 1.0×10⁶ cells/mL and a 200 L working volume. The culture medium was further expanded in the Production Bioreactor up to about 3.2×10⁶ cells/mL in a 200 L working volume. The cells in the Production Bioreactor were then co-infected with expressionBIICs (1:300k expressionBIIC:Sf9 v/v) and payloadBIICs (1:100k payloadBIIC:Sf9 v/v). Infected cells were incubated for 168 hours (7 days). Post-infection, the bioreactor conditions were adjusted as follows: 70 rpm agitation, cascading oxygen on demand up to 40% dissolved O₂, 1.2 L/min air overlay, 27° C. vessel temp, 3.0 L/min O₂ flow rate. The bulk harvest was collected for lysis and processing through Downstream processing.

Samples were taken at each of the expansion and bioreaction steps to monitor cell density and viability (e.g., at least 85%, at least 90%) throughout the upstream process. Monitoring included measuring viability %, viable cell density (VCD), and average cell diameter, e.g., using ViCell.

### Downstream—Cell Lysis

Chemical Lysis was initiated on the 200 L bulk harvest in the Production Bioreactor by adding 0.2 M Arginine HCl (pH 7.5, 11.43% v/v), followed by 10% Triton X-100 surfactant (5.3% v/v, for 0.5% w/v Triton X-100 in final lysis mixture) to provide a lysis pH of 6.8-7.5. The lysis mixture was held at 27° C. for 4.0-6.0 hours at 6 W/m3 agitation, until a crude lysate pool was generated.

### Downstream—Depth Filtration

The crude lysate pool from Chemical Lysis was processed through Depth Filtration using an EMD Millipore Millistak⁺+POD filter (C0SP media series) (≤250 L/m² load challenge, inlet pressure 18 psig, differential pressure 18 psig, flow rate 1.28-2.56 L/min (e.g., 1.9 L/min)).

A filter recovery flush (10-20 L/m² load challenge, 150-200 LMH load flux) using 50 mM sodium phosphate, 350 mM sodium chloride and 0.001% w/v Pluronic F-68 (mixture pH of 7.4) was passed through the depth filter, with the flushed recovery being added to the depth filtered pool.

### Downstream—0.2 μm Filtration

A depth filtered pool from the Depth Filtration was processed through a 0.2 μm Filtration using Sartorius Sartopore 2XLG, 0.8/0.2 μm filter with a 300 L/m² load challenge (inlet pressure 18 psig, differential pressure 18 psig, flow rate 1.28-2.56 L/min (e.g., 1.9 L/min)).

A filter recovery flush (inlet pressure 18 psig, differential pressure 18 psig, flow rate 1.28-2.56 L/min (e.g., 1.9 L/min)) using 50 mM sodium phosphate, 350 mM sodium chloride and 0.001% w/v Pluronic F-68 (mixture pH of 7.4) was passed through the 0.2 μm filter, with the flushed recovery being added to the 0.2 μm filtered pool.

### Downstream—Affinity Chromatography

A clarified lysate pool from the 0.2 μm Filtration (following depth filtration) was processed through Affinity Chromatography (AFC) using a GE AVB Sepharose HP column resin (3.0-3.5 L column volume). The column resin was equilibrated (5-7 CV, 150 cm/hr) and flushed (2 CV, 150 cm/hr) with a mixture of 50 mM sodium phosphate, 350 mM sodium chloride and 0.001% w/v Pluronic F-68 (mixture pH of 7.4). The column resin was loaded with the clarified lysate pool (≤5.0×1013 VG/mL-r load challenge) at 18-25° C., then flushed with a mixture of 50 mM sodium phosphate, 350 mM sodium chloride and 0.001% w/v Pluronic F-68 (mixture pH of 7.4).

This was followed by a first wash of the column resin (5 CV, 150 cm/hr) with a mixture of 20 mM sodium citrate, 1 M sodium chloride and 0.001% w/v Pluronic F-68 (mixture pH of 6.0); and a second wash of the column resin (5 CV, 150 cm/hr) with a mixture of 10 mM sodium citrate, 350 mM sodium chloride and 0.001% w/v Pluronic F-68 (mixture pH of 6.0). The filtered product was then eluted from the column resin (2-3 CV, 150 cm/hr) using a mixture of 20 mM sodium citrate, 350 mM sodium chloride and 0.001% w/v Pluronic F-68 (mixture pH of 3.0), with a target elution pool of 2.5-3.0 CV.

The resulting elution pool was neutralized with 2 M Tris Base and 0.001% w/v Pluronic F-68 (3.0% v/v spike, target final pH of 8.0-8.5) and filtered through a Millipore SHC 0.2 μm filter (flow rate 0.35-0.583 L/min (e.g., 0.467 L/min) or 1500-2500 LMH (e.g., 2000 LMH)) (filter area 0.014 m2, filter inlet pressure 18 psi).

### Anion-Exchange Chromatography

The neutralized, filtered affinity chromatography elution pool was processed through AEX using a Millipore Fractogel TMAE HiCap(m) Pow-Through membrane resin. The AEX membrane was charged and equilibrated (5 CV, 150 cm/hr) with a first mixture of 20 mM Tris, 2 M sodium chloride and 0.001% w/v Pluronic F-68 (mixture pH of 8.0), and then a second mixture of 40 mM Tris, 170 mM sodium chloride and 0.001% w/v Pluronic F-68 (mixture pH of 8.5). The load pool (the neutralized, filtered elution pool from affinity chromatography) was load adjusted using a 100-110% v/v spike of 10 mM Tris and 0.001% w/v Pluronic F-68 (mixture pH of 8.0-8.5), with a target pool conductivity of 17 mS/cm (adjusted with 5 M NaCl) and a target pool pH of 8.5 (adjust with 2 M Tris Base). The AEX membrane system was then loaded with the adjusted neutralized, filtered affinity chromatography elution pool (1.0-5.0×10¹³ VG/mL-r load challenge) at 18-25° C. The system was flushed and eluted (2 CV, 150 cm/hr) with a mixture of 40 mM Tris, 170 mM sodium chloride and 0.001% w/v Pluronic F-68 (mixture pH of 8.5), with the entire elution being collected. The AEX elution pool was then processed through 0.2 μm Filtration using an EMD Millipore Express SHCXL150 filter (≤1000 L/m² load challenge, ≤30 psi), resulting in an AEX pool.

### Tangential Flow Filtration (TFF)

The neutralized AEX pool was processed through TFF using a Millipore Ultracel PLCTK system with Pellicon-3 cassette (0.57 m², 30 kDA MWCO, 2-3 L/m²load challenge, 6 psi TMP, 5-10 psi inlet feed). The TFF system was first rinsed (25 L/m²) with WFI water, then sanitized (25 L/m², 45 min hold) with 0.25 M NaOH, then equilibrated (25 L/m²) with an equilibration buffer (pH 8.5) comprising 40 mM Tris, 170 mM sodium chloride, and 0.001% (w/v) Pluronic F-68, with equilibration continuing until both permeate and retentate effluents were at pH 8.5. The TFF Load pool was concentrated through ultrafiltration (2.6 L/min) to a target concentration of 5.0×10¹² VG/mL (confirmed by ddPCR), and then a diafiltration step (8 DV, 2.6 L/min) using a diafiltration buffer which comprises 10 mM Sodium Phosphate, 180 mM Sodium Chloride, and 0.001% (w/v) Pluronic F-68 (mixture pH of 7.3). The TFF system was subjected to a Recovery Flush (110% v/v of system holdup, 5-10 min recirculation) using the same diafiltration buffer. The Final TFF Recovery Flush was collected separately from the Final TFF Pool, and each pool was processed separately through 0.2 μm Filtration using an EMD Millipore Express SHCXL150 filter (≤1000 L/m² load challenge, ≤400 L/m²/hr flow rate, ≤30 psi). Filtered TFF Recovery Flush was added to Filtered TFF Pool, and then diluted as needed with diafiltration buffer, to provide a VRF Load Pool with viral concentration of 2.0-6.0×10¹² VG/mL, as measured by ddPCR.

### Virus Retentive Filtration (VRF)

A VRF Load Pool was processed through the Virus Retentive Filtration (VRF) using an Asahi Kasei Planova 35N filter (50.0-100.0 L/m² load challenge, 10 psi). The VRF filter and FF filters were both flushed before use (10 L/m², 240-300 LMH, 14 psi) with WFI water, then with 10 mM sodium phosphate, 180 mM sodium chloride, and 0.001% Pluronic F68 (mixture pH of 7.3).

The VRF filtration was followed by processing through a 0.2 μm Filtration using an EMD Millipore Express SHCXL150 filter (≤1000 L/m² load challenge, ≤30 psi), resulting in an VRF pool with a working viral concentration of 3.5-5.0×10¹² VG/mL, as measured by ddPCR.

### Downstream —Fill and Finish

A Pooled Drug Substance was transferred to a Biosafety Cabinet (BSC) and filtered through a EMD Millipore Millipak Gamma Gold 0.22 μm filter (dual-in-line sterilizing grade filters, ≤1000 L/m² load challenge, 200 LMH load flux, ≤60 psi differential pressure, ≤75 psi inlet pressure). The filtered Drug Substance pool comprised 10 mM sodium phosphate, 180 mM sodium chloride, and 0.001% Pluronic F68 (mixture pH of 7.3), with a target AAV concentration of 3.0-5.0×10¹² VG/mL, as measured by ddPCR. The filtered Drug Substance pool was then aseptically filled into 2 ml Cryovials (e.g., 2 mL Ompi Glass Vial, 1.8 ml fill volume, 1.6 ml extractable; or 1.2 ml fill volume, 1.0 ml extractable) utilizing a programmable Peristaltic dispensing pump within the BSC. Product vials were stoppered using a 13 mm West S2-F451 4432/50 stopper, seal with a 13 mm West Pharma Flip Off Long Matte Seal cap, 100% visually inspected and labeled (at 25° C.), and then stored at ≤−65° C.

Exemplary rAAV recoveries yielded by various steps of Example 5 are summarized below in Table 4:

TABLE 4
Exemplary rAAV recoveries

| | Concentration | | Recovery | | Batch Size |
|---|---|---|---|---|---|
| | (ddPCR, vg/mL) | | (ddPCR, % vg | | (L) |
| Process Pool | Expected | Range | Expected | Range | Expected |
| Crude Lysate, | 2 × 10¹¹ | 5 × l0¹⁰-5 × 10¹¹ | N/A | N/A | 235 |
| Post-Lysis | | | | | |
| Clarified Lysate | 2 × 10¹¹ | 4 × 10¹⁰-6 × 10¹¹ | 100% | 90%-110% | 250 |
| Immunoaffinity | 2 × 10¹² | 5 × 10¹¹-5 × 10¹² | 70% | 50%-90% | 10 |
| Chromatography | | | | | |
| Pool | | | | | |
| Anion Exchange | 2 × 10¹² | 5 × 10¹¹-5 × 10¹² | 95% | 80%-100% | 22 |
| Chromatography | | | | | |
| Pool | | | | | |
| Tangential Flow | 5 × 10¹² | 2.5 × 10¹²-6 × 10¹² | 70% | 50%-90% | 5 |
| Filtration Pool | | | | | |
| Viral Retentive | 5 × 10¹² | 2.5 × 10¹²-6 × 10¹² | 95% | 90%-100% | 5 |
| Filtration Pool | | | | | |
| Final Filtration | 5 × 10¹² | 2.5 × 10¹²-6 × 10¹² | 100% | 90%-100% | 5 |
| and Fill/DS | | | | | |

We claim:

1. A method for producing a recombinant adeno-associated virus (rAAV) comprising a polynucleotide encoding a payload, comprising:
(a) culturing viral production cells (VPCs) in a bioreactor to a target cell density;
(b) introducing into the bioreactor at least one baculovirus (expressionBac) comprising a viral expression construct, and at least one baculovirus (payloadBac) comprising the polynucleotide encoding the payload, and wherein the viral expression construct comprises an adeno-associated virus (AAV) viral expression construct encoding a viral capsid and at least one viral replication protein;
(c) incubating the VPCs in the bioreactor under conditions that result in the production of one or more rAAVs within one or more VPCs, wherein one or more of the rAAVs comprise the polynucleotide encoding a payload;
(d) harvesting a viral production pool from the bioreactor, wherein the viral production pool comprises one or more VPCs comprising one or more rAAVs;
(e) lysing the one or more VPCs in the viral production pool, thereby releasing one or more rAAVs from the one or more VPCs into a lysis medium;
(f) processing the lysis medium, wherein the processing comprises (optionally in the following order):
(i) one or more clarifying steps;
(ii) one or more immunoaffinity chromatography steps;
(iii) one or more anion exchange chromatography steps;
(iv) one or more tangential flow filtration (TFF) steps, wherein the one or more TFF steps comprises ultrafiltration followed by diafiltration; and
(v) one or more virus retentive filtration (VRF) steps;
wherein the processing optionally further comprises one or more filtration steps before or after any one or more of steps (i)-(v);
wherein the method yields a purified drug substance pool comprising a purified drug substance.

2. A method for producing a recombinant adeno-associated virus (rAAV) comprising a polynucleotide comprising a payload, comprising:
(a) culturing viral production cells (VPCs) in a bioreactor to a target cell density;
(b) introducing into the bioreactor at least one baculovirus (expressionBac) comprising a viral expression construct, and at least one baculovirus (payloadBac) comprising the polynucleotide encoding the payload, and wherein the viral expression construct comprises an adeno-associated virus (AAV) viral expression construct encoding a viral capsid and at least one viral replication protein;
(c) incubating the VPCs in the bioreactor under conditions that result in the production of one or more rAAVs within one or more VPCs, wherein one or more of the rAAVs comprise the polynucleotide encoding a payload;
(d) harvesting a viral production pool from the bioreactor, wherein the viral production pool comprises one or more VPCs comprising one or more rAAVs;
(e) lysing the one or more VPCs in the viral production pool by chemical lysis, thereby releasing one or more rAAVs from the one or more VPCs into a lysis medium;
(f) clarifying the lysis medium of (e) through one or more clarifying steps, yielding a clarification pool, wherein the clarification pool is optionally filtered;
(g) processing the clarification pool of(f) through one or more immunoaffinity chromatography steps, yielding an immunoaffinity chromatography pool, wherein the one or more immunoaffinity chromatography steps optionally comprises neutralizing the immunoaffinity chromatography pool, and wherein the immunoaffinity chromatography pool is optionally filtered;
(h) processing the immunoaffinity chromatography pool of (g) through one or more anion exchange chromatography steps, yielding an anion exchange chromatography pool, wherein the anion exchange chromatography pool is optionally filtered;
(i) processing the anion exchange chromatography pool of (h) through one or more tangential flow filtration (TFF) steps, wherein the one or more TFF steps comprises ultrafiltration followed by diafiltration, wherein the one or more TFF steps yields a concentrated, buffer-exchanged pool, wherein the concentrated, buffer-exchanged pool is optionally filtered;
(j) processing the concentrated, buffer-exchanged pool of (i) through one or more virus retentive filtration (VRF) steps, yielding a viral filtration pool comprising rAAVs comprising a payload, wherein the viral filtration pool is optionally filtered;
wherein the method yields a purified drug substance pool comprising a purified drug substance.

3. The method of claim 2, wherein, following (j), the viral filtration pool is processed through a further filtration step.

4. The method of any one of claims 1-3, wherein the viral capsid comprises an amino acid sequence or is encoded by a nucleotide sequence of any one of SEQ ID NOs: 1-875, 992-1374, and 1775-1777, or a functional variant thereof.

5. The method of any one of claims 1-3, wherein the viral capsid is AAV2.

6. The method of any one of claims 1-5, wherein the viral capsid is encoded by SEQ ID NO: 1778.

7. The method of any one of claims 1-6, wherein the viral capsid comprises SEQ ID NO: 16.

8. The method of any one of claims 1-7, wherein the payload comprises a polynucleotide encoding aromatic L-amino acid decarboxylase (AADC) or a functional variant thereof.

9. The method of any one of claims 1-7, wherein the payload comprises a polynucleotide encoding a therapeutic protein, an enzyme, an antibody or antigen-binding fragment thereof, a protein ligand, or a soluble receptor.

10. The method of any one of claims 1-7, wherein the payload comprises a polynucleotide encoding a modulatory polynucleotide which interferes with a target gene expression and/or a target protein production; optionally wherein the modulatory polynucleotide is an antisense strand, a miRNA molecule, or a siRNA molecule.

11. The method of any one of claims 1-10, wherein the VPCs are insect cells.

12. The method of claim 11, wherein the insect cells are Sf9 cells.

13. The method of claim 8, wherein the polynucleotide encoding AADC or a functional variant thereof encodes SEQ ID NO: 978.

14. The method of claim 13, wherein the polynucleotide further comprises a 5′ inverted terminal repeat (ITR); at least one multiple cloning site (MCS) region; a CMV enhancer; a cytomegalovirus (CMV) promoter; an intron region comprising immediate-early 1 (Ie1) exon 1, Ie1 intron 1 (partial), human beta-globin (hBglobin) intron 2, and hBglobin intron 3; a polyadenylation (poly(A)) signal, and a 3′ ITR.

15. The method of claim 14, wherein the polynucleotide comprises, e.g., from 5′ to 3′:
(a) a 5′ ITR comprising SEQ ID NO: 980;
(b) a first MCS region comprising SEQ ID NO: 981;
(c) a CMV enhancer comprising SEQ ID NO: 982;
(d) a CMV promoter comprising SEQ ID NO: 983;
(e) an intron region comprising an Ie1 exon 1 (SEQ ID NO: 984), a partial Ie1 intron 1 (SEQ ID NO: 985), a human beta-globin (hBglobin) intron 2 (SEQ ID NO: 986), and a hBglobin intron 3 (SEQ ID NO: 987);
(f) a polynucleotide encoding an AADC amino acid sequence comprising SEQ ID NO: 978, wherein optionally the polynucleotide comprises SEQ ID NO: 988;
(g) a second MCS region comprising SEQ ID NO: 989;
(h) a poly(A) signal comprising SEQ ID NO: 990; and
(i) a 3′ ITR comprising SEQ ID NO: 991.

16. The method of claim 15, wherein the polynucleotide comprises a sequence at least 90%, at least 95%, at least 97%, at least 98%, or at least 99% identical to SEQ ID NO: 979.

17. The method of claim 16, wherein the polynucleotide comprises SEQ ID NO: 979.

18. The method of any one of claims 1-17, wherein the viral expression construct comprises a nucleic acid sequence of SEQ ID NO: 1778.

19. The method of any one of claims 1-18, wherein the viral expression construct encodes SEQ ID NO: 16.

20. The method of any one of claims 1-19, wherein the at least one expressionBac is comprised in at least one baculovirus infected insect cell (expressionBIIC) and/or the at least one payloadBac is comprised in at least one baculovirus infected insect cell (payloadBIIC), wherein optionally the at least one expressionBac is comprised in at least one expressionBIIC and the at least one payloadBac is comprised in at least one payloadBIIC.

21. The method of claim 20, wherein the at least one expressionBIIC is introduced into the bioreactor at a ratio of 1:200,000 to 1:400,000 expressionBIIC:VPC (v/v) and/or the at least one payloadBIIC is introduced into the bioreactor at a ratio of 1:25,000 to 1:200,000 payloadBIIC:VPC (v/v), wherein the ratio of expressionBIIC:payloadBIIC is between about 1:1-1:5.

22. The method of claim 20, wherein the at least one expressionBIIC is introduced into the bioreactor at a ratio of 1:250,000 to 1:350,000 expressionBIIC:VPC (v/v) (e.g., 1:300,000 expressionBIIC:VPC (v/v)) and/or the at least one payloadBIIC is introduced into the bioreactor at a ratio of 1:50,000 to 1:150,000 payloadBIIC:VPC (v/v) (e.g., 1:100,000 payloadBIIC:VPC (v/v)), wherein the ratio of expressionBIIC:payloadBIIC is about 1:3.

23. The method of any one of claims 1-22, wherein the VPCs are cultured in the bioreactor in insect cell culture medium.

24. The method of claim 23, wherein the insect cell culture medium is a serum free, protein-free medium, wherein optionally the insect cell culture medium comprises L-glutamine and poloxamer 188, wherein further optionally the insect cell culture medium comprises EFS AF™ insect cell culture medium.

25. The method of any one of claims 1-24, wherein the VPCs of step (a) are cultured in the bioreactor and/or the VPCs, at least one expressionBac, and at least one payloadBac of step (c) are incubated in the bioreactor at 26° C.-28° C. (e.g., 27° C.) and 30%-50% (e.g., 40%) dissolved oxygen.

26. The method of any one of claims 1-25, wherein the target cell density of the VPCs of step (a) is 3.0×10⁶-3.4×10⁶ cells/mL (e.g., 3.2×10⁶-3.4×10⁶ cells/mL; e.g., 3.2×10⁶ cells/mL).

27. The method of any one of claims 1-26, wherein the lysing comprises a chemical lysis solution comprising a surfactant and arginine or a salt thereof, wherein optionally the surfactant is octyl phenol ethoxylate and the arginine or salt thereof is arginine hydrochloride.

28. The method of claim 27, wherein the chemical lysis solution comprises between about 0.1-1.0% (w/v) octyl phenol ethoxylate and between about 150-250 mM arginine hydrochloride.

29. The method of claim 28, wherein the chemical lysis solution comprises 0.5% (w/v) octyl phenol ethoxylate and 200 mM arginine hydrochloride.

30. The method of any one of claims 27-29, wherein the chemical lysis solution is free of detectable nuclease.

31. The method of any one of claims 27-30, wherein the lysing is carried out for 4-6 hours (e.g., 4 hours) at 26° C.-28° C. (e.g., 27° C.).

32. The method of any one of claims 1-31, wherein the one or more clarifying steps comprises depth filtration followed by filtration through an about 0.2 μm filter.

33. The method of any one of claims 1-32, wherein the one or more immunoaffinity chromatography steps comprises an immunoaffinity chromatography column comprising a recombinant protein ligand that binds at least one of AAV1, AAV2, AAV3, AAV5, and AAV9; and optionally binds at least AAV2.

34. The method of claim 33, wherein the immunoaffinity chromatography column is equilibrated with a solution comprising between about 25-75 mM sodium phosphate, between about 325-375 mM sodium chloride, and between about 0.001-0.01% w/v poloxamer 188; flushed with a solution comprising between about 25-75 mM sodium phosphate, between about 325-375 mM sodium chloride and between about 0.001-0.01% w/v poloxamer 188; washed with a solution comprising between about 15-25 mM sodium citrate, between about 0.5-1.5 M sodium chloride and between about 0.001-0.01% w/v poloxamer 188; and washed with a solution of between about 5-15 mM sodium citrate, between about 325-375 mM sodium chloride and between about 0.001-0.01% w/v poloxamer 188; wherein the one or more immunoaffinity chromatography steps yields a immunoaffinity chromatography pool.

35. The method of claim 34, wherein the immunoaffinity chromatography column is equilibrated with a solution comprising 50 mM sodium phosphate, 350 mM sodium chloride and 0.001% w/v poloxamer 188; flushed with a solution comprising 50 mM sodium phosphate, 350 mM sodium chloride and 0.001% w/v poloxamer 188; washed with a solution comprising 20 mM sodium citrate, 1 M sodium chloride and 0.001% w/v poloxamer 188; and washed with a solution of 10 mM sodium citrate, 350 mM sodium chloride and 0.001% w/v poloxamer 188; wherein the one or more immunoaffinity chromatography steps yields a immunoaffinity chromatography pool.

36. The method of claim 34 or claim 35, wherein the immunoaffinity chromatography pool is neutralized with between about 1.5-2.5 M Tris Base and between about 0.001-0.01% w/v poloxamer 188 (2.5-3.5% v/v spike, pH 8.0-8.5).

37. The method of any one of claims 34-36, wherein the immunoaffinity chromatography pool is neutralized with 2 M Tris Base and 0.001% w/v poloxamer 188 (3.0% v/v spike, pH 8.0-8.5).

38. The method of any one of claims 34-37, wherein the immunoaffinity chromatography pool is filtered through an about 0.2 μm filter.

39. The method of any one of claims 1-38, wherein the one or more anion exchange chromatography steps comprises charging and equilibrating an anion exchange chromatography column with a solution comprising between about 15-25 mM Tris, between about 1.5-2.5 M sodium chloride and between about 0.001-0.01% w/v poloxamer 188, then a solution of between about 35-45 mM Tris, between about 150-190 mM sodium chloride and between about 0.001-0.01% w/v poloxamer 188.

40. The method of claim 39, wherein the one or more anion exchange chromatography steps comprises charging and equilibrating the anion exchange chromatography column with a solution comprising 20 mM Tris, 2 M sodium chloride and 0.001% w/v poloxamer 188, then a solution of 40 mM Tris, 170 mM sodium chloride and 0.001% w/v poloxamer 188.

41. The method of claim 39 or claim 40, wherein the anion exchange chromatography column is flushed and eluted with a solution comprising between about 35-45 mM Tris, between about 150-190 mM sodium chloride and between about 0.001-0.01% w/v poloxamer 188, yielding an anion exchange chromatography pool.

42. The method of claim 41, wherein the anion exchange chromatography column is flushed and eluted with a solution comprising 40 mM Tris, 170 mM sodium chloride and 0.001% w/v poloxamer 188, yielding an anion exchange chromatography pool.

43. The method of claim 41 or claim 42, wherein the anion exchange chromatography pool is filtered through an about 0.2 μm filter.

44. The method of any one of claims 1-43, wherein:
the one or more immunoaffinity chromatography steps comprises loading the immunoaffinity chromatography column with a 1.0×10¹³-5.0×10¹³ vg/mL-r load challenge at 18-25° C.; and/or
the one or more anion exchange chromatography steps comprises loading the anion exchange chromatography column with a 1.0×10¹³-5.0×10¹³ vg/mL-r load challenge at 18-25° C.

45. The method of any one of claims 1-44, wherein the one or more TFF steps comprises TFF filtration with a TFF filter, yielding a TFF load pool, followed by concentration of the TFF load pool by ultrafiltration followed by diafiltration, yielding a final TFF load pool.

46. The method of claim 45, wherein the TFF filtration comprises equilibration with a buffer comprising about 35-45 mM Tris, between about 150-190 mM sodium chloride and between about 0.001-0.01% w/v poloxamer 188.

47. The method of claim 46, wherein the TFF filtration comprises equilibration with a buffer comprising 40 mM Tris, 170 mM sodium chloride, and 0.001% (w/v) poloxamer 188.

48. The method of any one of claims 45-47, wherein, after obtaining the TFF load pool, the TFF filter is subjected to a recovery flush using a buffer comprising between about 15-25 mM sodium phosphate, between about 160-200 mM sodium chloride, and between about 0.001-0.01% w/v poloxamer 188, yielding a TFF recovery flush pool.

49. The method of claim 48, wherein, after obtaining the TFF load pool, the TFF filter is subjected to a recovery flush using a buffer comprising 10 mM sodium phosphate, 180 mM sodium chloride, and 0.001% (w/v) poloxamer 188, yielding a TFF recovery flush pool.

50. The method of any one of claims 45-49, wherein the TFF load pool is concentrated by ultrafiltration to a viral concentration of between about 3.0×10¹²-7.0×10¹² vg/mL.

51. The method of claim 50, wherein the TFF load pool is concentrated by ultrafiltration to a viral concentration of about 5.0×10¹² vg/mL.

52. The method of any one of claims 45-51, wherein diafiltration comprises buffer exchange with a buffer comprising between about 5-15 mM sodium phosphate, between about 160-200 mM sodium chloride, and between about 0.001-0.01% w/v poloxamer 188 (buffer pH of 7.1-7.5).

53. The method of claim 52, wherein diafiltration comprises buffer exchange with a buffer comprising 10 mM sodium phosphate, 180 mM sodium chloride, and 0.001% (w/v) poloxamer 188 (buffer pH of 7.1-7.5, e.g., pH 7.3).

54. The method of any one of claims 45-53, wherein the final TFF load pool is filtered through an about 0.2 μm filter, yielding a filtered final TFF load pool.

55. The method of any one of claims 48-54, wherein the TFF recovery flush pool is filtered through an about 0.2 μm filter, yielding a filtered TFF recovery flush pool.

56. The method of claim 55, wherein the filtered final TFF load pool and the filtered TFF recovery flush pool are combined to form a concentrated, buffer-exchanged pool, wherein the concentrated, buffer-exchanged pool is optionally diluted using a buffer comprising between about 5-15 mM sodium phosphate, between about 160-200 mM sodium chloride, and between about 0.001-0.01% w/v poloxamer 188 (buffer pH of 7.1-7.5), wherein the concentrated, buffer-exchanged pool comprises a viral concentration of 1.0×10¹²-7.0×10¹² vg/mL.

57. The method of claim 56, wherein the filtered final TFF load pool and the filtered TFF recovery flush pool are combined to form a concentrated, buffer-exchanged pool, wherein the concentrated, buffer-exchanged pool is optionally diluted using a buffer comprising 10 mM sodium phosphate, 180 mM sodium chloride, and 0.001% (w/v) poloxamer 188 (buffer pH of 7.1-7.5, e.g., pH 7.3), wherein the concentrated, buffer-exchanged pool comprises a viral concentration of 2.0×10¹²-6.0×10¹² vg/mL, e.g., 5.0×10¹² vg/mL.

58. The method of any one of claims 1-57, wherein the one or more VRF steps comprises filtration with a VRF filter having a pore size of about 35 nm, yielding a viral filtration pool.

59. The method of claim 58, wherein the VRF filter is flushed twice before use with a solution comprising between about 5-15 mM sodium phosphate, between about 160-200 mM sodium chloride, and between about 0.001-0.01% w/v poloxamer 188 (buffer pH of 7.1-7.5).

60. The method of claim 59, wherein the VRF filter is flushed twice before use with a solution comprising 10 mM sodium phosphate, 180 mM sodium chloride, and 0.001% poloxamer 188 (solution pH of 7.1-7.5, e.g., pH 7.3).

61. The method of any one of claims 58-60, wherein the viral filtration pool is filtered through a filter of about 0.2 μm.

62. The method of any one of claims 58-61, wherein the viral filtration pool comprises a viral concentration of 10×10¹²-7.0×10¹² vg/mL.

63. The method of claim 62, wherein the viral filtration pool comprises a viral concentration of 3.5×10¹²-5.0×10¹² vg/mL, e.g., about 5.0×10¹² vg/mL.

64. The method of any one of claims 58-63, wherein the viral filtration pool is filtered at least once (optionally at least twice) using an about 0.22 μm filter, yielding a filtered drug substance pool in a solution comprising between about 5-15 mM sodium phosphate, between about 160-200 mM sodium chloride, and between about 0.001-0.01% w/v poloxamer 188 (buffer pH of 7.1-7.5).

65. The method of claim 64, wherein the viral filtration pool is filtered at least once (optionally at least twice) using an about 0.22 μm filter, yielding a filtered drug substance pool in a solution comprising 10 mM sodium phosphate, 180 mM sodium chloride, and 0.001% poloxamer 188 (solution pH of 7.1-7.5, e.g., pH 7.3).

66. The method of claim 64 or claim 65, wherein the filtered drug substance pool comprises a viral concentration of 1.0×10¹²-7.0×10¹² vg/mL.

67. The method of claim 66, wherein the filtered drug substance pool comprises a viral concentration of 3.0×10¹²-5.0×10¹² vg/mL, e.g., about 5.0×10¹² vg/mL.

68. The method of any one of claims 1-67, wherein the purified drug substance comprises greater than about 3.0×10¹² vg/mL (e.g., between about 3.0×10¹² vg/mL to about 7.0×10¹² vg/mL) rAAVs in a solution comprising 5-15 mM sodium phosphate, 160-200 mM sodium chloride, and 0.001-0.01% poloxamer (solution pH of 7.1-7.5).

69. The method of claim 68, wherein the purified drug substance comprises greater than about 5.0×10¹² vg/mL rAAVs in a solution comprising about 10 mM sodium phosphate, about 180 mM sodium chloride, and about 0.001% poloxamer 188 (solution pH of about 7.3).

70. The method of any one of claims 1-69, wherein the purified drug substance comprises less than about 20% empty viral capsids.

71. The method of claim 70, wherein the purified drug substance comprises less than about 10% empty viral capsids.

72. The method of any one of claims 1-71, wherein the purified drug substance has an AADC relative potency of at least 50%.

73. The method of any one of claims 1-72, wherein the VRCs, at least one expressionBac (e.g., at least one expressionBIIC), and at least one payload Bac (e.g., at least one payloadBIIC) are incubated for 156-180 hours, e.g., 164-172 hours, e.g., 168 hours, prior to lysis.

74. The method of any one of claims 1-73, wherein the VRCs incubating with at least one expressionBac (e.g., at least one expressionBIIC) and at least one payloadBac (e.g., at least one payloadBIIC) have at least 85% viability, e.g., at least 90% viability, prior to lysis.

75. The method of any one of claims 1-74, wherein the viral production pool weighs 195-198 kg, e.g., 196 kg, prior to lysis.

76. The method of any one of claims 1-75, wherein the method produces a total process rAAV yield of 30%-50%.

77. The method of any one of claims 1-76, wherein the purified drug substance comprises one or more (e.g., all) of the following: a payload, greater than about 60% full viral capsids (e.g., viral capsids containing the polynucleotide encoding the payload), less than about 5% VPC (e.g., Sf9 cell) protein contaminants, less than 2 ng/mL DNA contaminants from the VPCs, an osmolality of 300-400 mOsm/kg, less than about 6000 particles with a size of ≥10 μm, less than about 600 particles with a size of ≥25 μm, and/or a solution pH of 7.3±0.5.

78. The method of any one of claims 1-77, wherein the rAAVs comprise a capsid from AAV2, e.g., an AAV2 capsid encoded by a nucleic acid sequence comprising SEQ ID NO: 1778, e.g., an AAV2 capsid comprising an amino acid sequence SEQ ID NO: 16.

79. The method of any one of claims 1-78, wherein the viral expression construct comprises one or more polynucleotides encoding a VP1 capsid protein, VP2 capsid protein, VP3 capsid protein, Rep52, and Rep78.

80. The method of any one of claims 1-79, wherein the viral expression construct comprises a nucleic acid sequence of SEQ ID NO: 1778, e.g., encoding an amino acid sequence SEQ ID NO: 16.

81. The method of claim 79 or claim 80, wherein the viral expression construct comprises a nucleic acid sequence of SEQ ID NO: 1779, e.g., encoding an amino acid sequence of SEQ ID NO: 1780.

82. The method of any one of claims 79-81, wherein the VP1 capsid protein, VP2 capsid protein, and VP3 capsid protein are encoded in one or more open reading frames and the Rep52 and Rep78 are encoded in one or more open reading frames, wherein the one or more open reading frames encoding the VP1 capsid protein, VP2 capsid protein, and VP3 capsid protein and the one or more open reading frames encoding the Rep52 and Rep78 are different open reading frames.

83. The method of claim 82, wherein VP1 capsid protein, VP2 capsid protein, and VP3 capsid protein are encoded in a first open reading frame and the Rep52 and Rep78 are encoded in a second open reading frame.

84. The method of any one of claims 1-83, wherein the method further comprises aliquoting the purified drug substance into one or more vials at a volume of about 1.2 mL, wherein the one or more vials have an extractable volume of about 1.0 mL.

85. The method of claim 84, wherein the method further comprises storing the one or more vials at ≤65° C.

86. A composition comprising rAAVs comprising a payload, produced by the method of any one of claims 1-85, wherein the composition comprises greater than about 60% full viral capsids (e.g., less than about 40% empty viral capsids), less than about 5% protein contaminants, less than 2 ng/mL Sf9 residual host DNA contaminants, an osmolality of 300-400 mOsm/kg, less than about 6000 particles with a size of ≥10 μm, less than about 600 particles with a size of ≥25 μm, and/or a solution pH of 7.3±0.5.

87. The composition of claim 86, wherein the payload is AADC or a functional variant thereof.

88. The composition of claim 86 or claim 87, wherein the rAAV comprises an AAV2 capsid.

89. The composition of claim 88, wherein the AAV2 capsid is encoded by SEQ ID NO: 1778.

90. The composition of claim 88 or claim 89, wherein the AAV2 capsid comprises SEQ ID NO: 16.

91. The composition of any one of claims 86-90, wherein the composition comprises 1.0×10¹²-7.0×10¹² vg/mL rAAVs, in a solution comprising between about 5-15 mM sodium phosphate, between about 160-200 mM sodium chloride, and between about 0.001-0.01% poloxamer 188 (solution pH of 7.1-7.5).

92. The composition of claim 91, wherein the composition comprises 3.0×10¹²-5.0×10¹² vg/mL rAAVs, e.g., about 5.0×10¹² vg/mL rAAVs, in a solution comprising 10 mM sodium phosphate, 180 mM sodium chloride, and 0.001% poloxamer 188 (solution pH of 7.1-7.5, e.g., pH 7.3).

93. The composition of any one of claims 86-92, wherein the composition comprises less than about 20% empty viral capsids.

94. The composition of claim 93, wherein the composition comprises less than about 10% empty viral capsids.

95. The composition of any one of claims 86-94, wherein the composition has an AADC relative potency of at least 50%.

96. The composition of any one of claims 86-95, for use in treating and/or preventing Parkinson's Disease.

97. A method of treating Parkinson's Disease comprising administering an effective amount of the composition of any one of claims 86-95.

98. Use of the composition of any one of claims 86-95 in the manufacture of a medicament for treating and/or preventing Parkinson's Disease.

99. A method for producing a recombinant adeno-associated virus 2 (rAAV2) comprising a polynucleotide comprising SEQ ID NO: 979, wherein the method comprises:
(a) culturing Sf9 cells (viral production Sf9 cells) in a bioreactor to a target cell density of 3.0×10⁶-3.4×10⁶ cells/mL; wherein the viral production Sf9 cells are cultured in serum-free, protein-free insect cell culture medium at about 26° C.-28° C. and 30/6-50% dissolved oxygen, wherein the serum-free, protein-free insect cell culture medium optionally comprises L-glutamine and poloxamer 188;
(b) introducing into the bioreactor baculovirus infected insect cells (expressionBIICs) comprising baculoviruses comprising a viral expression construct, and baculovirus infected insect cells (payloadBIICs) comprising baculoviruses comprising the polynucleotide comprising SEQ ID NO: 979, wherein the viral expression construct comprises one or more polynucleotides encoding a viral capsid and at least one viral replication protein of adeno-associated virus 2 (AAV2); wherein the polynucleotide comprises SEQ ID NO: 979; and wherein the expressionBIICs are introduced at a ratio of about 1:300.000 expressionBIIC:viral production Sf9 (v/v) and the payloadBIICs are introduced at a ratio of about 1:100,000 payloadBIIC:viral production Sf9 (v/v), wherein the expressionBIICs and/or payload BIICs are optionally Sf9 cells;
(c) incubating the viral production Sf9 cells in the bioreactor under conditions that result in the production of one or more rAAV2s within one or more of the viral production Sf9 cells, wherein one or more of the rAAV2s comprise a polynucleotide comprising SEQ ID NO: 979;
(d) harvesting a viral production pool from the bioreactor, wherein the viral production pool comprises one or more viral production Sf9 cells comprising one or more rAAV2s, wherein the viral production pool optionally weighs 195-198 kg, e.g., 196 kg, and has a % viability of at least 85%, e.g., at least 90%;
(e) lysing the viral production Sf9 cells in the viral production pool, wherein the lysing comprises a chemical lysis solution and is carried out at 26° C.-28° C. for 4-6 hours, wherein the chemical lysis solution comprises 0.5% (w/v) octyl phenol ethoxylate and 200 mM arginine hydrochloride, and lacks detectable nuclease, thereby releasing one or more rAAV2s from the viral production Sf9 cells into a lysis medium;
(f) clarifying the lysis medium of (e) through a depth filter followed by an about 0.2 μm filter, yielding a clarification pool;
(g) processing the clarification pool of (f) through an immunoaffinity chromatography column comprising a recombinant protein ligand that binds at least AAV2; wherein the immunoaffinity chromatography column is equilibrated with a solution comprising 50 mM sodium phosphate, 350 mM sodium chloride and 0.001% w/v poloxamer 188; loaded with a 1.0×10¹³-5.0×10¹³ vg/mL-r load challenge at 18-25° C.; flushed with a solution comprising 50 mM sodium phosphate, 350 mM sodium chloride and 0.001% w/v poloxamer 188; washed with a solution comprising 20 mM sodium citrate, 1 M sodium chloride and 0.001% w/v poloxamer 188; and washed with a solution of 10 mM sodium citrate, 350 mM sodium chloride and 0.001% w/v poloxamer 188; wherein processing the clarification pool of(f) through an immunoaffinity chromatography column yields an immunoaffinity chromatography pool, wherein the immunoaffinity chromatography pool is optionally neutralized with 2 M Tris Base and 0.001% w/v poloxamer 188 (3.0% v/v spike, pH 8.0-8.5) and optionally filtered through an about 0.2 μm filter;
(h) processing the immunoaffinity chromatography pool or filtered immunoaffinity chromatography pool of (g) through an anion exchange chromatography column, e.g., a column operated in flow-through mode; wherein the one or more anion exchange chromatography columns is charged and equilibrated with a solution comprising 20 mM Tris, 2 M sodium chloride and 0.001% w/v poloxamer 188, then a solution of 40 mM Tris, 170 mM sodium chloride and 0.001% w/v poloxamer 188; loaded with a 1.0×10¹³-5.0×10¹³ vg/mL-r load challenge at 18-25° C.; and flushed and eluted with a solution comprising 40 mM Tris, 170 mM sodium chloride and 0.001% w/v poloxamer 188, yielding an anion exchange chromatography pool; wherein the anion exchange chromatography pool is filtered through an about 0.2 μm filter, yielding a filtered anion exchange chromatography pool;
(i) processing the filtered anion exchange chromatography pool of(h) through a tangential flow filtration (TFF) filter yielding a TFF load pool; wherein the TFF filter is equilibrated with buffer comprising 40 mM Tris, 170 mM sodium chloride, and 0.001% (w/v) poloxamer 188; wherein the TFF load pool is concentrated by ultrafiltration to a viral concentration of about 5.0×10¹² vg/mL, followed by diafiltration comprising buffer exchange with a buffer comprising 10 mM sodium phosphate, 180 mM sodium chloride, and 0.001% (w/v) poloxamer 188 (buffer pH of 7.1-7.5, e.g., pH 7.3), followed by filtration through an about 0.2 μm filter, yielding a filtered TFF load pool;
wherein a TFF recovery flush pool is prepared by subjecting the TFF filter to a recovery flush using a buffer comprising 10 mM sodium phosphate, 180 mM sodium chloride, and 0.001% (w/v) poloxamer 188 (buffer pH of 7.1-7.5, e.g., pH 7.3); wherein the TFF recovery flush pool is filtered through an about 0.2 μm filter, yielding a filtered TFF recovery flush pool;
wherein the filtered TFF load pool and filtered TFF recovery flush pool are combined to form a concentrated, buffer-exchanged pool, and optionally diluted using a buffer comprising 10 mM sodium phosphate, 180 mM sodium chloride, and 0.001% (w/v) poloxamer 188 (buffer pH of 7.1-7.5, e.g., pH 7.3), wherein the concentrated, buffer-exchanged pool comprises a viral concentration of 2.0×10¹²-6.0×10¹² vg/mL;
(j) processing the concentrated, buffer-exchanged pool of (i) through a viral retentive filtration (VRF) filter to yield a viral filtration pool; wherein the VRF filter comprises a pore size of about 35 nm and is flushed twice before use with a solution comprising 10 mM sodium phosphate, 180 mM sodium chloride, and 0.001% poloxamer 188 (solution pH of 7.1-7.5, e.g., pH 7.3); and wherein the viral filtration pool is filtered through a filter of about 0.2 μm, yielding a filtered viral filtration pool comprising a viral concentration of 3.5×10¹²-5.0×10¹² vg/mL; and
(k) processing the viral production pool of (j) through an about 0.22 μm filter, e.g., filtering twice through an about 0.22 μm filter;
wherein the method yields a purified rAAV2 composition comprising AAV2 capsid protein and a polynucleotide encoding AADC (e.g., SEQ ID NO: 979) or a functional variant thereof, wherein the composition comprises a viral concentration of 3.0×10¹²-5.0×10¹² vg/mL in a solution comprising 10 mM sodium phosphate, 180 mM sodium chloride, and 0.001% poloxamer 188 (solution pH of 7.1-7.5, e.g., pH 7.3).

100. The method of claim 99, wherein the viral expression construct comprises SEQ ID NO: 1778.

101. The method of claim 99 or 100, wherein the viral expression construct encodes for an AAV2 VP1 polypeptide comprising the amino acid sequence of SEQ ID NO: 16.

102. The method of any one of claims 99-101, wherein the viral concentration of the purified rAAV2 composition is about 5.0×10¹² vg/mL.

103. The method of any one of claims 99-102, wherein the method produces a total process rAAV yield of 30%-50%.

104. The method of any one of claims 99-103, wherein the purified rAAV2 composition comprises greater than about 60% full viral capsids (e.g., capsids comprising the polynucleotide encoding AADC), less than about 5% protein contaminants from the VPCs, less than 2 ng/mL Sf9 residual host DNA contaminants, an osmolality of 300-400 mOsm/kg, less than about 6000 particles with a size of ≥10 μm, less than about 600 particles with a size of ≥25 μm, and/or a solution pH of 7.3±0.5

105. The method of any one of claims 99-104, wherein the viral expression construct comprises one or more polynucleotides encoding an AAV2 VP1 capsid protein, an AAV2 VP2 capsid protein, an AAV2 VP3 capsid protein, an AAV2 Rep52 protein, and/or an AAV2 Rep78 protein.

106. The method of claim 105, wherein the AAV2 VP1 capsid protein, AAV2 VP2 capsid protein, and AAV2 VP3 capsid protein are encoded in one or more open reading frames and the AAV2 Rep52 and AAV2 Rep78 are encoded in one or more open reading frames, wherein the one or more open reading frames encoding the AAV2 VP1 capsid protein, AAV2 VP2 capsid protein, and AAV2 VP3 capsid protein and the one or more open reading frames encoding the AAV2 Rep52 and AAV2 Rep78 are different open reading frames.

107. The method of claim 106, wherein AAV2 VP1 capsid protein, AAV2 VP2 capsid protein, and AAV2 VP3 capsid protein are encoded in a first open reading frame and the AAV2 Rep52 and AAV2 Rep78 are encoded in a second open reading frame.

108. The method of any one of claims 99-107, wherein the method further comprises aliquoting the purified rAAV2 composition into one or more vials at a volume of about 1.2 mL, wherein the one or more vials have an extractable volume of about 1.0 mL.

109. The method of claim 108, wherein the method further comprises storing the one or more vials at ≤65° C.

110. A composition comprising a polynucleotide encoding AADC (e.g., SEQ ID NO: 979) or a functional variant thereof, wherein the composition comprises 3.0×10¹²-5.0×10¹² vg/mL rAAV2s, e.g., about 5.0×10¹² vg/mL rAAV2s, in a solution comprising 10 mM sodium phosphate, 180 mM sodium chloride, and 0.001% poloxamer 188 (solution pH of 7.1-7.5, e.g., pH 7.3).

111. The composition of claim 110, wherein the composition is produced by the method of any one of claims 99-109.

112. The composition of claim 110 or claim 111, wherein the composition comprises greater than about 60% full viral capsids (e.g., less than about 40% empty viral capsids), less than about 5% protein contaminants, less than 2 ng/mL Sf9 residual host DNA contaminants, an osmolality of 300-400 mOsm/kg, less than about 6000 particles with a size of ≥10 μm, less than about 600 particles with a size of ≥25 μm, and/or a solution pH of 7.3±0.5.

113. The composition of any one of claims 110-112, wherein the composition comprises less than 20% empty viral capsids.

114. The composition of claim 113, wherein the composition comprises less than 10% empty viral capsids.

115. The composition of any one of claims 110-114, wherein the composition has an AADC relative potency of at least 50%.

116. The composition of any one of claims 110-115, wherein the rAAV2s comprise an AAV2 capsid protein comprising SEQ ID NO: 1778, e.g., encoding SEQ ID NO: 16.

117. The composition of any one of claims 110-116, for use in treating and/or preventing Parkinson's Disease.

118. A method of treating Parkinson's Disease comprising administering an effective amount of the composition of any one of claims 110-116.

119. Use of the composition of any one of claims 110-116 in the manufacture of a medicament for treating and/or preventing Parkinson's Disease.

120. A nucleic acid sequence encoding an AAV2 capsid protein, wherein the sequence comprises SEQ ID NO: 1778.

121. The nucleic acid sequence of claim 120, wherein the AAV2 capsid protein comprises the amino acid sequence of SEQ ID NO: 16.
